(19) United States
(12) Patent Application Publication
(10) Pub. No.: US 2021/0332122 A1
(43) Pub. Date: Oct. 28, 2021

(54) DYSREGULATION OF COVID-19 RECEPTOR ASSOCIATED WITH IBD

(71) Applicant: Cedars-Sinai Medical Center, Los Angeles, CA (US)

(72) Inventors: Dermot MCGOVERN, Los Angeles, CA (US); Alka POTDAR, Cumming, GA (US); Shishir DUBE, Los Angeles, CA (US)

(21) Appl. No.: 17/232,987

(22) Filed: Apr. 16, 2021

Related U.S. Application Data
(60) Provisional application No. 63/011,963, filed on Apr. 17, 2020.

(51) Int. Cl. C07K 16/24; G01N 33/68; C12Q 1/6883

(52) U.S. Cl. CPC C07K 16/244; C07K 16/241; G01N 33/6893; C12Q 1/6883; C12Q 2600/158; G01N 2800/52; G01N 2333/705; G01N 2333/912; G01N 2333/948; G01N 2800/065

(57) ABSTRACT

Provided herein are methods, systems and kits for use in identifying a subject with an increased risk of developing severe forms of inflammatory bowel disease (IBD), based at least in part, on an expression of one or more biomarkers detected in a biological sample obtained from the subject. Also provided are methods, systems and kits for treating, or optimizing the treatment for, the IBD based, at least in part, on the expression the one or more biomarkers. In some embodiments, the one or more biomarkers is angiotensin-converting enzyme 2 (ACE2), the host receptor for severe acute respiratory syndrome (SARS) coronavirus 2 (SARS-CoV-2).

### CROSS-REFERENCE

This application claims the benefit of U.S. Provisional Application No. 63/011,963, filed Apr. 17, 2020, which is hereby incorporated by reference in its entirety.

### STATEMENT AS TO FEDERALLY SPONSORED RESEARCH

This invention was made with government support under grant number DK046763 and DK062413 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy created Apr. 13, 2021, is named 56884-772_201_SL, and is 295,071 bytes in size.

### BACKGROUND

As of April 2021, more than 120 million people worldwide have confirmed Coronavirus disease 2019 (COVID-19) infection with current (and likely conservative) estimates implicating the virus in more than 2.67 million deaths. COVID-19 most commonly presents with respiratory symptoms although recent reports have suggested that patients often present with both respiratory and gastrointestinal (GI) symptoms (predominantly diarrhea and nausea) and in a proportion of patients, GI symptoms alone may be the presenting symptoms. There has also been concern that detection of the virus in stool may implicate the fecal-oral route as an important mode of transmission.

There is very significant variation in outcomes from COVID-19 with the majority having mild symptoms, a minority having respiratory compromise, and a small percentage dying as a consequence of secondary cytokine storm or superimposed infection. Increasing age, being male, smoking, co-morbidities, and an elevated body mass index (BMI) have all been implicated in increased morbidity and mortality, but it is likely that other factors also contribute to the variability in response. For example, it is believed that immunosuppressive medications commonly used to treat immune-mediated diseases may play a role on the susceptibility and natural history of COVID-19.

### SUMMARY

Aspects disclosed herein provide methods of treating an inflammatory, fibrostenotic, or fibrotic disease or condition in a subject, the method comprising: administering a therapeutic agent to the subject based, at least in part, on an expression level of a biomarker comprising angiotensin-converting enzyme 2 (ACE2), transmembrane serine protease 2 (TMPRSS2), transmembrane serine protease 4 (TMPRSS4), solute carrier family 6 member 19 (SLC6A19), Sigma Non-Opioid Intracellular Receptor 1 (SIGMAR1), or Janus kinase 1 (JAK1), or a combination thereof, as compared to an expression level of the biomarker in a control sample obtained from a subject that does not have the inflammatory, fibrostenotic, or fibrotic disease or condition. In some embodiments, the expression level of the biomarker in the biological sample is lower than the expression level of the biomarker in the control sample. In some embodiments, the expression level of the biomarker in the biological sample is higher than the expression level of the biomarker in the control sample when the inflammatory, fibrostenotic, or fibrotic disease or condition is Crohn's disease; and wherein the expression level of the biomarker in the biological sample is higher than the expression level of the biomarker in the control sample when the inflammatory, fibrostenotic, or fibrotic disease or condition is ulcerative colitis. In some embodiments, the biomarker is ACE2. In some embodiments, the biomarker is TMPRSS2. In some embodiments, the biomarker is TMPRSS4. In some embodiments, the biomarker is SLC6A19. In some embodiments, the biomarker is JAK1. In some embodiments, the biomarker is SIGMAR1. In some embodiments, the biomarker comprises two biomarkers comprising ACE2, TMPRSS2, TMPRSS4, SLC6A19, JAK1, or SIGMAR1. In some embodiments, the biomarker comprises three biomarkers comprising ACE2, TMPRSS2, TMPRSS4, SLC6A19, JAK1, or SIGMAR1. In some embodiments, the biomarker comprises four biomarkers comprising ACE2, TMPRSS2, TMPRSS4, SLC6A19, JAK1, or SIGMAR1. In some embodiments, the biomarker is RNA or protein. In some embodiments, the biomarker is encoded by a nucleic acid sequence that is at least 90% identical to any one of SEQ ID NOS: 1-48. In some embodiments, the biomarker is encoded by a nucleic acid sequence that is at least 95% identical to any one of SEQ ID NOS: 1-48. In some embodiments, the biomarker is encoded by a nucleic acid sequence provided in any one of SEQ ID NOS: 1-48. In some embodiments, the inflammatory, fibrostenotic, or fibrotic disease or condition comprises inflammatory bowel disease (IBD), Crohn's disease (CD), or ulcerative colitis (UC), or a combination thereof. In some embodiments, the expression level of the biomarker in the biological sample that is lower than the expression level of the biomarker in the control sample is indicative of the subject having a high risk of a non-response to an inhibitor of Tumor Necrosis Factor (TNF), interleukin 12 (IL-12), or interleukin 23 (IL-23) when the inflammatory, fibrostenotic, or fibrotic disease or condition is Crohn's disease. In some embodiments, the expression level of the biomarker in the biological sample that is higher than the expression level of the biomarker in the control sample is indicative of the subject having a high risk of a non-response to an inhibitor of TNF, IL-12, or IL-23 when the inflammatory, fibrostenotic, or fibrotic disease or condition is ulcerative colitis. In some embodiments, the inhibitor of IL-12 comprises ustekinumab. In some embodiments, the inhibitor of TNF comprises infliximab. In some embodiments, methods further comprise: (a) determining that the subject has a high risk of having or developing a non-response to an inhibitor of Tumor Necrosis Factor (TNF), interleukin 12 (IL-12), or interleukin 23 (IL-23), when (i) the expression level of the biomarker in the biological sample is lower than the expression level of the biomarker in the control sample and (ii) the inflammatory, fibrostenotic, or fibrotic disease or condition is Crohn's disease; or (b) determining that the subject has a high risk of a non-response to an inhibitor of TNF, IL-12, or IL-23 when (i) the expression level of the biomarker in the biological sample is higher than the expression level of the biomarker in the control sample and (ii) the inflammatory, fibrostenotic, or fibrotic disease or condition is ulcerative colitis. In some embodiments, the biological sample is a tissue sample obtained from the small intestine or large intestine of the subject. In some embodiments, the biological sample is a tissue sample obtained from the ileum of the subject. In some embodiments, the biological sample is a tissue sample obtained from the colon. In some embodiments, the expression level of the biomarker in the biological sample that is lower or higher than the expression level of the biomarker in the control sample is indicative of disease a severe form of the inflammatory, fibrostenotic, or fibrotic disease or condition characterized by at least one of: (a) high risk for relapse of the inflammatory, fibrostenotic, or fibrotic disease or condition; and (b) a high risk for developing intestinal fibrosis. In some embodiments, the expression of the biomarker is determined using quantitative polymerase chain reaction (qPCR), nucleic acid sequencing, gene array analysis, single molecule detection, immunohistochemistry (IHC), enzyme linked-immunosorbent assay (ELISA), or flow cytometry. In some embodiments, the therapeutic agent is a modulator of Tumor Necrosis Factor (TNF), interleukin 12 (IL-12), interleukin 23 (IL-23), ACE2, ACE, angiotensin-2 receptor (AGTR1), TMPRSS2, TMPRSS4, SLC6A19, or JAK1, or a combination thereof. In some embodiments, the modulator of IL-12 comprises ustekinumab. In some embodiments, the modulator of TNF comprises infliximab. In some embodiments, the subject is a human subject.

Aspects disclosed herein provide methods of optimizing a treatment regimen, the method comprising: (a) providing a biological sample from a subject that was administered a first dosage amount of a therapeutic agent targeting Tumor Necrosis Factor (TNF), interleukin 12 (IL-12), or interleukin 23 (IL-23); (b) measuring an expression level of a biomarker comprising angiotensin-converting enzyme 2 (ACE2), transmembrane serine protease 2 (TMPRSS2), transmembrane serine protease 4 (TMPRSS4), solute carrier family 6 member 19 (SLC6A19), Sigma Non-Opioid Intracellular Receptor 1 (SIGMAR1), or Janus kinase 1 (JAK1), or a combination thereof; (c) comparing the expression level of the biomarker from (b) to an expression level of the biomarker in a control sample obtained from a subject that was not administered the therapeutic agent; and (d) administering a second dosage amount that is the same as, or higher than, the first dosage amount of the therapeutic agent based, at least in part, on the expression level of the biomarker in the biological sample measured in (b) when the expression level is higher than the expression level of the biomarker in the control sample; or (e) administering a second dosage amount that is lower than the first dosage amount of the therapeutic agent based, at least in part, on the expression level of the biomarker in the biological sample measured in (b) when the expression level is lower than the expression level of the biomarker in the control sample. In some embodiments, the biomarker is ACE2. In some embodiments, the biomarker is TMPRSS2. In some embodiments, the biomarker is TMPRSS4. In some embodiments, the biomarker is SLC6A19. In some embodiments, the biomarker is JAK1. In some embodiments, the biomarker is SIGMAR1. In some embodiments, the biomarker comprises two biomarkers comprising ACE2, TMPRSS2, TMPRSS4, SLC6A19, JAK1, or SIGMAR1. In some embodiments, the biomarker comprises three biomarkers comprising ACE2, TMPRSS2, TMPRSS4, SLC6A19, JAK1, or SIGMAR1. In some embodiments, the biomarker comprises four biomarkers comprising ACE2, TMPRSS2, TMPRSS4, SLC6A19, JAK1, or SIGMAR1. In some embodiments, the biomarker is RNA or protein. In some embodiments, the biomarker is encoded by a nucleic acid sequence that is at least 90% identical to any one of SEQ ID NOS: 1-48. In some embodiments, the biomarker is encoded by a nucleic acid sequence that is at least 95% identical to any one of SEQ ID NOS: 1-48. In some embodiments, the biomarker is encoded by a nucleic acid sequence provided in any one of SEQ ID NOS: 1-48. In some embodiments, the subject has an inflammatory, fibrostenotic, or fibrotic disease or condition. In some embodiments, the inflammatory, fibrostenotic, or fibrotic disease or condition comprises inflammatory bowel disease (IBD), Crohn's disease (CD), or ulcerative colitis (UC), or a combination thereof. In some embodiments, the expression level of the biomarker in the biological sample that is lower than the expression level of the biomarker in the control sample is indicative of disease a severe form of the inflammatory, fibrostenotic, or fibrotic disease or condition characterized by at least one of: (a) high risk for relapse of the inflammatory, fibrostenotic, or fibrotic disease or condition; and (b) a high risk for developing intestinal fibrosis. In some embodiments, the expression level of the biomarker in the biological sample that is lower than the expression level of the biomarker in the control sample is indicative of the subject having a high risk of a non-response to the therapeutic agent. In some embodiments, the therapeutic agent targeting IL-12 comprises ustekinumab. In some embodiments, the therapeutic agent targeting TNF comprises infliximab. In some embodiments, the biological sample is a tissue sample obtained from the small intestine or large intestine of the subject. In some embodiments, the biological sample is a tissue sample obtained from the ileum of the subject. In some embodiments, the biological sample is a tissue sample obtained from the colon. In some embodiments, the expression of the biomarker is measured using quantitative polymerase chain reaction (qPCR), nucleic acid sequencing, gene array analysis, single molecule detection, immunohistochemistry (IHC), enzyme linked-immunosorbent assay (ELISA), or flow cytometry. In some embodiments, the methods further comprises: (f) administering a second therapeutic agent targeting activity or expression of ACE2, ACE, angiotensin-2 receptor (AGTR1), TMPRSS2, TMPRSS4, SLC6A19, or JAK1, or a combination thereof. In some embodiments, the subject is a human subject.

Aspects disclosed herein provide methods of enriching a target nucleic acid in a sample, the method comprising: (a) providing a biological sample from a subject with an inflammatory, fibrostenotic, or fibrotic disease or condition, wherein the biological sample comprises a target nucleic acid molecule comprising a nucleic acid sequence encoding angiotensin-converting enzyme 2 (ACE2), transmembrane serine protease 2 (TMPRSS2), transmembrane serine protease 4 (TMPRSS4), solute carrier family 6 member 19 (SLC6A19), Sigma Non-Opioid Intracellular Receptor 1 (SIGMAR1), or Janus kinase 1 (JAK1), or a combination thereof; (b) bringing a fluid reaction formulation comprising a synthetic oligonucleotide molecule in contact with the biological sample; (c) hybridizing the synthetic oligonucleotide molecule and the target nucleic acid molecule; (d) amplifying the hybridized synthetic oligonucleotide molecule and the target nucleic acid molecule, thereby enriching the target nucleic acid in the fluid reaction formulation; (e) detecting the enriched target nucleic acid molecule. In some embodiments, the nucleic acid sequence encodes ACE2. In some embodiments, the nucleic acid sequence encodes TMPRSS2. In some embodiments, the nucleic acid sequence encodes TMPRSS4. In some embodiments, the nucleic acid sequence encodes SLC6A19. In some embodiments, the nucleic acid sequence encodes JAK1. In some embodiments, the nucleic acid sequence encodes SIGMAR1. In some embodiments, the target nucleic acid molecule comprises two or more target nucleic acid molecules comprising ACE2, TMPRSS2, TMPRSS4, SLC6A19, JAK1, or SIGMAR1. In some embodiments, the target nucleic acid molecule comprises three or more target nucleic acid molecules comprising ACE2, TMPRSS2, TMPRSS4, SLC6A19, JAK1, or SIGMAR1. In some embodiments, the target nucleic acid molecule comprises four or more target nucleic acid molecules comprising ACE2, TMPRSS2, TMPRSS4, SLC6A19, JAK1, or SIGMAR1. In some embodiments, the target nucleic acid molecule is RNA. In some embodiments, the nucleic acid sequence comprises a nucleic acid sequence is at least 90% identical to any one of SEQ ID NOS: 1-48. In some embodiments, the nucleic acid sequence comprises a nucleic acid sequence is at least 95% identical to any one of SEQ ID NOS: 1-48. In some embodiments, the nucleic acid sequence comprises a nucleic acid sequence provided in any one of SEQ ID NOS: 1-48. In some embodiments, the inflammatory, fibrostenotic, or fibrotic disease or condition comprises inflammatory bowel disease (IBD), Crohn's disease (CD), or ulcerative colitis (UC), or a combination thereof. In some embodiments, methods further comprise treating the inflammatory, fibrostenotic, or fibrotic disease or condition in the subject by administering to the subject a modulator of ACE2, TMPRSS2, TMPRSS4, SLC6A19, or JAK1, or a combination thereof. In some embodiments, methods further comprise treating the inflammatory, fibrostenotic, or fibrotic disease or condition in the subject by administering to the subject hydroxychloroquine. In some embodiments, detecting in (e) is indicative of the subject having a high risk of a non-response to an inhibitor of Tumor Necrosis Factor (TNF), interleukin 12 (IL-12), or interleukin 23 (IL-23). In some embodiments, the inhibitor of IL-12 comprises ustekinumab. In some embodiments, the inhibitor of TNF comprises infliximab. In some embodiments, the biological sample is a tissue sample obtained from the small intestine or large intestine of the subject. In some embodiments, the biological sample is a tissue sample obtained from the ileum of the subject. In some embodiments, the biological sample is a tissue sample obtained from the colon. In some embodiments, detecting in (e) is indicative of disease a severe form of the inflammatory, fibrostenotic, or fibrotic disease or condition characterized by at least one of: (a) high risk for relapse of the inflammatory, fibrostenotic, or fibrotic disease or condition; and (b) a high risk for developing intestinal fibrosis. In some embodiments, methods further comprise quantifying an expression level of in target nucleic acid molecule relative to an expression level of the target nucleic acid molecule in a control sample derived from one or more subjects that do not have the inflammatory, fibrostenotic, or fibrotic disease or condition. In some embodiments, the expression level of the target nucleic acid molecule detected in the biological sample is lower relative to the expression level of the target nucleic acid molecule in the control sample. In some embodiments, the expression level of the target nucleic acid molecule detected in the biological sample is higher relative to the expression level of the target nucleic acid molecule in the control sample. In some embodiments, the quantifying comprises quantitative polymerase chain reaction (qPCR), nucleic acid sequencing, or gene array analysis. In some embodiments, the subject is a human subject. In some embodiments, the inflammatory, fibrostenotic, or fibrotic disease or condition subject was treated with an inhibitor of Tumor Necrosis Factor (TNF), interleukin 12 (IL-12), or interleukin 23 (IL-23). In some embodiments, the inhibitor of IL-12 comprises ustekinumab. In some embodiments, the inhibitor of TNF comprises infliximab. In some embodiments, methods further comprise monitoring response to the inhibitor of TNF, IL-12, or IL-23 based, at least in part, on the expression level of the target nucleic acid molecule detected in the biological sample.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-1B shows details of the small bowel (SB) and colon (CO) transcriptomic cohorts with available demographics and disease status. FIG. 1A provides numbers of subjects in each cohort. FIG. 1B provides meta-data availability for some the subjects in each cohort.

FIG. 2A-2C show the association of ACE2 with age across the different cohorts. FIG. 2A shows the association of ACE2 with age at collection for the WashU cohort. FIG. 2B shows the association of ACE2 with age at collection for the RISK cohort. FIG. 2C shows the association of ACE2 with age at collection across a combination of three SB cohorts (RISK, SB139 and WashU).

FIG. 3 shows a univariate association of ACE2 with age at specimen collection, gender and smoking status in SB139 cohort.

FIG. 4 shows an association of ACE2 with BMI in WashU cohort using linear regression.

FIG. 5A-5B show ACE2 levels and demographics. FIG. 5A shows a univariate association of ACE2 in Cedars100 cohort with gender indicating lower expression in males (p=0.01, Mann-Whitney test). FIG. 5B shows an analysis with smoking status indicating higher expression if prior or current smoker (p=0.15, Mann-Whitney test).

FIG. 6A-6B show an association of ACE2 with disease status. FIG. 6A depicts the WashU cohort where ACE2 expression was downregulated in CD compared to controls (Mann-Whitney test, error bars indicate mean=+/−SD). FIG. 6B depicts the RISK cohort where differences were seen in median ACE2 expression in CD, UC and control (p<0.0001, Kruskal-Wallis, error bars in red indicate mean=/−SD).

FIG. 7A-7H shows the association of ACE2 with disease sub-types. FIG. 7A shows RISK, median ACE2 in control, UC, iCD and cCD (p<0.0001, K-W), iCD versus cCD (p_{adj}=0.01), iCD versus control (p_{adj}<0.0001). FIG. 7B shows SB139, lower ACE2 expression associated with disease recurrence after surgery (p=0.05, adjusted for age, gender and 2 principal components (PCs)). FIG. 7C shows RISK, ACE2 at diagnosis classified according to development of complicated disease (structuring, B2 or penetrating, B3) or not (inflammatory, B1) at 3 year and 5 year follow-up (B2+B3 versus B1, p=0.017; B2 versus B1, p=0.007, adjusted for age and gender). FIG. 7D shows PROTECT, ACE2 was elevated in UC compared to control (p=0.0039, M-W). FIG. 7E shows PROTECT, ACE2 was elevated in UC subjects that needed oral steroid by week (wk) 52 (p=0.0006, M-W). FIG. 7F shows PROTECT, ACE2 was elevated in UC subjects that subsequently needed anti-TNF by wk 52 (p=0.0039, M-W). FIG. 7G shows Cedars119, ACE2 was elevated in UC subjects with active disease (p=0.0002, M-W). FIG. 7H shows Cedars119, ACE2 was positively correlated with Mayo endoscopy score in UC (p<0.0001, Spearman r=0.358).

FIG. 8A-8B show clinical data for 8 subjects with one of the five high CADD ACE2 variants identified by whole-exome sequencing. Ch: chromosome; BP: base pair; CADD score: Combined Annotation Dependent Depletion Score; MAF: mean allele frequency; EIM: extra-intestinal manifestation; Ciclo: ciclosporin; IFX: infliximab; Thio: thiopurine; Dx: diagnosis; EN: erythema nodosum; AA: alopecia areata; DVT: deep vein thrombosis; GMN: glomerulonephritis; Ca: carcinoma; UC: ulcerative colitis; CD: Crohn's disease; IBD: inflammatory bowel disease. M; Male; F: Female; SNV; single nucleotide variant. FIG. 8A shows one-half of the clinical data for the 8 subjects. FIG. 8B shows the second half of the clinical data for the 8 subjects.

FIG. 9A-9H depicts a univariate analysis of ACE2 and other biomarkers and IBD medication. FIG. 9A depicts ACE2 levels in an initial cohort of subjects in a clinical trial for ustekinumab in ileal inflamed samples before (week (wk) 0) and after (wk 6) treatment were trending (p=0.06, t test). FIG. 9B depicts ACE2 levels in an initial cohort of subjects in a clinical trial for infliximab in controls are significantly higher (p=0.03, t test) than in Crohn's ileitis responders before (CDiR_before) treatment. Six weeks after (CDiR_after) infliximab treatment the levels are significantly restored in responders compared to before treatment (CDiR_before) (p=0.03, t test). No significant difference was seen in Crohn's ileitis non-responders before (CDiNR_before) and 6 weeks after (CDiNR_after) infliximab treatment. FIG. 9C shows IFX trial (ileum CD), ACE2 was elevated in non-IBD controls compared to CD responders pre-treatment (CDiR_beforeT) (p=0.03, t test). Post-treatment, ACE2 was restored in responders (CDiR_afterT) compared to pre-treatment (p=0.03, t test); FIG. 9D shows CERTIFI (ileum CD), ACE2 pre- and post-treatment levels in inflamed and uninvolved samples. FIG. 9E show UNITI-2 (ileum CD), lower ACE2 levels at baseline in CD compared to non-IBD in both UST induction group (I) (130 mg I_wk0, p=0.034, t test) and maintenance group (M) (UST 90 mg SC q8w I_wk0, p=0.0004, M-W test). Both post-induction therapy, (130 mg I_wk8, p=0.008, t test) and post-maintenance therapy (UST 90 mg SC q8w M-wk44, p=0.037, M-W), ACE2 levels are restored. FIG. 9F shows IFX trial (colon CD), lower ACE2 levels in non-IBD compared to Crohn's colitis responders (p=0.03, t test) pre-treatment (CDcR_beforeT). FIG. 9F shows IFX trial (colon UC), ACE2 was lower in non-IBD compared to UC responders pre-treatment (UC_R_before) (p=0.0017, t test). Post-treatment the levels are restored to non-IBD in responders (UC_R_after, p=0.0013, t test) as well as combined UC (p=0.03, t test). FIG. 9H shows CERTIFI (colon CD), ACE2 pre- and post-treatment levels in inflamed and uninvolved samples.

FIG. 10A-10B show directionality of fold change in CD and UC as compared with non-IBD control. FIG. 10A shows direction of fold change in CD versus non-IBD for some canonical interferon stimulated genes (ISGs) in ileal biopsies from IFX drug trial is opposite to that of ACE2. FIG. 10B shows direction of fold change in UC versus non-IBD for some canonical interferon stimulated genes (ISGs) in colonic biopsies from IFX drug trial is same as ACE2.

FIG. 11A-11D show an inverse correlation between ACE2 expression and increasing severity of inflammation as measured by macroscopic and microscopic criteria (ileal GHAS and SES-CD). FIG. 11A shows the inverse correlation between ACE2 expression and increasing severity of inflammation as measured at baseline (0 weeks) by Simple endoscopic score for crohn's disease (SES-CD). FIG. 11B shows the inverse correlation between ACE2 expression and increasing severity of inflammation as measured at 8 weeks after induction (Ustekinumab or placebo) by SES-CD. FIG. 11C shows the inverse correlation between ACE2 expression and increasing severity of inflammation as measured at 0 weeks following diagnosis by Global Histologic Disease Activity Score (GHAS). FIG. 11C shows the inverse correlation between ACE2 expression and increasing severity of inflammation as measured at 8 weeks after induction by GHAS.

FIG. 12 provides a schematic illustration, according to some embodiments described herein, of the observation that reduced small bowel but elevated colonic ACE2 levels in IBD are associated with inflammation and severe disease, but normalized after anti-cytokine therapy (e.g., infliximab, ustekinumab).

### DETAILED DESCRIPTION

Provided herein are methods, systems, and kits for characterizing a disease or a condition, as well as monitoring treatment for, or treating, the disease or the condition in a subject. In some embodiments, the subject is selected for treatment based, at least in part, on an expression level of one or more biomarkers described herein. The inventors of the present disclosure have identified one or more biomarkers that, when detected in a biological sample obtained from the subject, indicate that the subject is at high risk for having or developing a severe form of the disease, and/or that the subject is suitable for a particular treatment (e.g., targeted therapeutic agent) to treat the disease or the condition. In some embodiments, the one or more biomarkers is Angiotensin-Converting Enzyme 2 (ACE2), which is the host receptor for Severe acute respiratory syndrome (SARS) coronavirus 2 (SARS-COV-2). In some embodiments, the one or more biomarkers comprise other molecules that interact with ACE2, and which have been implicated in Coronavirus Disease 2019 (COVID-19) biology including: the transmembrane serine proteases (TMPRSS2 and TMPRSS4) that help prime SARS-COV-2 spike protein for host cell entry; the ACE2 paralog in the renin-angiotensin-aldosterone system (RAAS), angiotensin I converting enzyme (ACE); and solute carrier family 6 member 19 (SLC6A19), expression of which is dependent on ACE2.

The inventors of the present disclosure identified factors, including inflammation and drug treatment that influence expression of ACE2, as well as other biomarkers disclosed herein, in the small bowel and colon of Crohn's Disease (CD) patients and colon of ulcerative colitis (UC) patients, as well as non-inflammatory bowel disease (IBD) controls. Without being bound by any particular theory, it is believed that ACE2 and the other biomarkers disclosed herein may be used to identify a subject that is prone to developing a disease or a condition, or a severe form of the disease or the condition, characterized as involving inflammation, as well as to select the subject for treatment with a particular therapy, or optimize a treatment regimen including such therapy, to treat the disease or the condition in the subject.

Provided herein are methods of monitoring and, optionally, optimizing a treatment regimen provided to the subject for treatment of the disease or the condition, based at least in part, on the express level of the one or more biomarkers. For example, the subject may be receiving a treatment for a disease or a condition (e.g., IBD), such as an inhibitor of tumor necrosis factor (TNF) therapy (e.g., infliximab) or an interleukin 12 (IL-12) or interleukin 23 (IL-23), such as ustekinumab. The inventors of the present disclosure discovered that an expression level of the one or more biomarkers disclosed herein (e.g., ACE2), when measured during a treatment course of a subject receiving such inhibitor, may predict whether the inhibitor is therapeutically effective to treat the disease or the condition. In some embodiments, the dosage amount or frequency of the inhibitor is modified, based at least in part, on the expression level of the one or more biomarkers such that the treatment regimen is optimized for the subject.

Further provided are methods of characterizing a disease or a condition in a subject based on the presence or a level of the one or more biomarkers detected in a sample obtained from the subject. Suitable methods of detecting the one or more biomarkers are provided herein, which include quantitative polymerase chain reaction (qPCR) in the case of RNA detection, and single molecule detection (e.g., SIMOA®) in the case of protein detection. In some cases, the subject is treated with a therapeutic agent described herein, based at least in part, on the characterization of the disease or the condition. In some embodiments, the disease or the condition in an IBD, such as CD or UC. In some embodiments, the IBD is characterized as severe or refractory.

### A. Methods

I. Methods of Detection

Disclosed herein, in some embodiments, are methods of detecting a presence or absence, as well as a level of a biomarkers disclosed herein. In some embodiments, the methods of detection are useful for the diagnosis, prognosis, monitoring of a treatment regimen or disease progression, selection for treatment, and/or treatment of a disease or condition (e.g., IBD, CD, UC) described herein.

In some embodiments, an expression level of the one or more biomarkers is detected in a tissue sample obtained from a subject. In some embodiments, the expression level of the one or more biomarkers is higher or lower than the expression level of the one or more biomarkers in control sample. In some embodiments, the control sample is obtained from a subject that does not have the disease or the condition. In some embodiments, the control sample is obtained from a normal or a healthy individual. In some embodiments, methods further comprise comparing the expression level of the one or more biomarkers in the tissue sample with the expression level of the one or more biomarkers in the control sample.

In some embodiments, biomarker expression is absolute. In some embodiments, an absolute level of the biomarker is measured, which is calculated by the ratio between the expression of the biomarker (e.g., number of copies) and the expression of one or more reference genes (e.g., a house-keeping gene). In some embodiments, the absolute numbers of copies of the biomarker are between about 1,5000 and 6,500, 2,000 and 6,000, 2,500 and 5,500, 3,000 and 5,000, 3,500 and 4,500, or 3,000 and 4,000, copies. In some embodiment, the absolute numbers of copies of the biomarker are between about 150 and 450, 200 and 400, or 250 and 350, copies. In some embodiments, the absolute number of copies of the biomarker is at most or equal to about 2,000, 4,000, 5,000, 6,000, 8,000, 9,000, or 10,000 copies. In some embodiments, the absolute number of copies of the biomarker is at least or equal to about 2,000, 4,000, 5,000, 6,000, 8,000, 9,000, or 10,000 copies.

In some embodiments, biomarker expression is relative, for example, as an expression of fold change between two or more samples (e.g., two patient samples at different time points, a control sample and a patient sample collected at the same time point, two different types of samples taken from the same patient at the same timepoint, and so on). In some embodiments, the expression of the biomarker is about 1-fold, 2-fold, 3-fold, 4-fold, 5-fold, 6-fold, 7-fold, 8-fold, 9-fold, or 10-fold lower than an expression of the biomarker in a control sample. In some embodiments, the expression of the biomarker is about 1-fold, 2-fold, 3-fold, 4-fold, 5-fold, 6-fold, 7-fold, 8-fold, 9-fold, or 10-fold higher than an expression of the biomarker in a control sample. In some embodiments, the expression of the biomarker is about 1-fold, 2-fold, 3-fold, 4-fold, 5-fold, 6-fold, 7-fold, 8-fold, 9-fold, or 10-fold lower than an expression of the biomarker in a biological sample obtained from the subject or patient at a different timepoint (e.g., during treatment course). In some embodiments, the expression of the biomarker is about 1-fold, 2-fold, 3-fold, 4-fold, 5-fold, 6-fold, 7-fold, 8-fold, 9-fold, or 10-fold higher than an expression of the biomarker in a biological sample obtained from the subject or patient at a different timepoint (e.g., during treatment course). In some embodiments, the expression of the biomarker is about 1-fold, 2-fold, 3-fold, 4-fold, 5-fold, 6-fold, 7-fold, 8-fold, 9-fold, or 10-fold lower than an expression of the biomarker in a different biological sample obtained from the same subject. In some embodiments, the expression of the biomarker is about 1-fold, 2-fold, 3-fold, 4-fold, 5-fold, 6-fold, 7-fold, 8-fold, 9-fold, or 10-fold higher than an expression of the biomarker in a different biological sample obtained from the same subject. In some embodiments, the expression of the biomarker in a biological sample obtained from the small bowel is at least 10-fold higher than the expression of the biomarker in the colon.

Non-limiting examples of “biological sample” include any material from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. In various embodiments, the sample comprises tissue from the large and/or small intestine. In various embodiments, the large intestine sample comprises the cecum, colon (the ascending colon, the transverse colon, the descending colon, and the sigmoid colon), rectum and/or the anal canal. In some embodiments, the small intestine sample comprises the duodenum, jejunum, and/or the ileum. Alternatively, a sample can be obtained through primary patient derived cell lines, or archived patient samples in the form of preserved samples, or fresh frozen samples.

In some embodiments, methods involve detecting a nucleic acid sequence from, for example, a biological sample. In some cases, the nucleic acid sequence comprises deoxyribonucleic acid (DNA). In some embodiments, the nucleic acid sequence comprises a denatured DNA molecule or fragment thereof. In some embodiments, the nucleic acid sequence comprises DNA selected from: genomic DNA, viral DNA, mitochondrial DNA, plasmid DNA, amplified DNA, circular DNA, circulating DNA, cell-free DNA, or exosomal DNA. In some embodiments, the DNA is single-stranded DNA (ssDNA), double-stranded DNA, denaturing double-stranded DNA, synthetic DNA, and combinations thereof. The circular DNA may be cleaved or fragmented. In some embodiments, the nucleic acid sequence comprises ribonucleic acid (RNA). In some embodiments, the nucleic acid sequence comprises fragmented RNA. In some embodiments, the nucleic acid sequence comprises partially degraded RNA. In some embodiments, the nucleic acid sequence comprises a microRNA or portion thereof. In some embodiments, the nucleic acid sequence comprises an RNA molecule or a fragmented RNA molecule (RNA fragments) selected from: a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, an RNA transcript, a synthetic RNA, and combinations thereof.

In some embodiments, the one or more biomarkers is detected using a nucleic acid-based detection assay. In some embodiments, the nucleic acid-based detection assay comprises quantitative polymerase chain reaction (qPCR), gel electrophoresis (including for e.g., Northern or Southern blot), immunochemistry, in situ hybridization such as fluorescent in situ hybridization (FISH), cytochemistry, or sequencing. In some embodiments, the sequencing technique comprises next generation sequencing. In some embodiments, the methods involve a hybridization assay such as fluorogenic qPCR (e.g., TaqMan™, SYBR green, SYBR green I, SYBR green II, SYBR gold, ethidium bromide, methylene blue, Pyronin Y, DAPI, acridine orange, Blue View or phycoerythrin), which involves a nucleic acid amplification reaction with a specific primer pair, and hybridization of the amplified nucleic acid probes comprising a detectable moiety or molecule that is specific to a target nucleic acid sequence. In some embodiments, a number of amplification cycles for detecting a target nucleic acid in a qPCR assay is about 5 to about 30 cycles. In some embodiments, the number of amplification cycles for detecting a target nucleic acid is at least about 5 cycles. In some embodiments, the number of amplification cycles for detecting a target nucleic acid is at most about 30 cycles. In some embodiments, the number of amplification cycles for detecting a target nucleic acid is about 5 to about 10, about 5 to about 15, about 5 to about 20, about 5 to about 25, about 5 to about 30, about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 cycles. For TaqMan™ methods, the probe may be a hydrolysable probe comprising a fluorophore and quencher that is hydrolyzed by DNA polymerase when hybridized to a target nucleic acid. In some cases, the presence of a target nucleic acid is determined when the number of amplification cycles to reach a threshold value is less than 30, 29, 28, 27, 26, 25, 24, 23, 22, 21, or 20 cycles. In some embodiments, hybridization may occur at standard hybridization temperatures, e.g., between about 35° C. and about 65° C. in a standard PCR buffer.

In some embodiments, the nucleic acid-based detection assay comprises the use of nucleic acid probes conjugated or otherwise immobilized on a bead, multi-well plate, or other substrate, wherein the nucleic acid probes are configured to hybridize with a target nucleic acid sequence. In some embodiments, the nucleic acid probe is specific to one or more biomarkers disclosed herein is used. In some embodiments, the biomarker comprises a transcribed polynucleotide sequence (e.g., RNA, cDNA). In some embodiments, the nucleic acid probe can be, for example, a full-length cDNA, or a portion thereof, such as an oligonucleotide of at least about 7, 8, 9, 10, 11, 12, 13, 14, 15, 20, 25, 30, 35, 40, 45, or 50 nucleotides in length and sufficient to specifically hybridize under standard hybridization conditions to the target nucleic acid sequence. In some embodiments, the target nucleic acid sequence is immobilized on a solid surface and contacted with a probe, for example by running the isolated target nucleic acid sequence on an agarose gel and transferring the target nucleic acid sequence from the gel to a membrane, such as nitrocellulose. In some embodiments, the probe(s) are immobilized on a solid surface, for example, in an Affymetrix gene chip array, and the probe(s) are contacted with the target nucleic acid sequence.

In an aspect, provided herein, are methods of enriching a target nucleic acid in a sample, the method comprising: (a) providing a biological sample from a subject with an inflammatory, fibrostenotic, or fibrotic disease or condition, wherein the biological sample comprises a target nucleic acid molecule comprising a nucleic acid sequence encoding angiotensin-converting enzyme 2 (ACE2), transmembrane serine protease 2 (TMPRSS2), transmembrane serine protease 4 (TMPRSS4), solute carrier family 6 member 19 (SLC6A19), Sigma Non-Opioid Intracellular Receptor 1 (SIGMAR1), or Janus kinase 1 (JAK1), or a combination thereof; (b) bringing a fluid reaction formulation comprising a synthetic oligonucleotide molecule in contact with the biological sample; (c) hybridizing the synthetic oligonucleotide molecule and the target nucleic acid molecule; (d) amplifying the hybridized synthetic oligonucleotide molecule and the target nucleic acid molecule, thereby enriching the target nucleic acid in the fluid reaction formulation; (e) detecting the enriched target nucleic acid molecule. In some embodiments, the quantifying comprises performing an assay comprising quantitative polymerase chain reaction (qPCR), nucleic acid sequencing, or gene array analysis. In some embodiments, the assay is performed under standard conditions. In the case of qPCR, the standard hybridization conditions may comprise an annealing temperature between about 30° C. and about 65° C.

In an aspect, provided herein, the detection of the biomarker involves amplification of the subject's nucleic acid by the polymerase chain reaction (PCR). In some embodiments, the PCR assay involves use of a pair of primers capable of amplifying at least about 10 contiguous nucleobases within a nucleic acid sequence provided in SEQ ID NOS: 1-48. In fluorogenic quantitative PCR, quantitation is based on amount of fluorescence signals (TaqMan and SYBR green). In some embodiments, the nucleic acid probe is conjugated to a detectable molecule. The detectable molecule may be a fluorophore. The nucleic acid probe may also be conjugated to a quencher.

In some embodiments, the term “probe” with regards to nucleic acids, refers to any nucleic acid molecule that is capable of selectively binding to a specifically intended target nucleic acid sequence. In some embodiments, probes are specifically designed to be labeled, for example, with a radioactive label, a fluorescent label, an enzyme, a chemiluminescent tag, a colorimetric tag, or other labels or tags that are known in the art. In some embodiments, the fluorescent label comprises a fluorophore. In some embodiments, the fluorophore is an aromatic or heteroaromatic compound. In some embodiments, the fluorophore is a pyrene, anthracene, naphthalene, acridine, stilbene, benzoxaazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno[2,3,4-ij: 5,6,7-i′j′]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3,6,7,12,13,16,17-octahydro-inner salt (TR or Texas Red); or BODIPY™ dyes. In some cases, the probe comprises FAM as the dye label.

In some embodiments, the biomarker is detected by subjecting a sample obtained from the subject to a nucleic acid amplification assay. In some embodiments, the amplification assay comprises polymerase chain reaction (PCR), qPCR, self-sustained sequence replication, transcriptional amplification system, Q-Beta Replicase, rolling circle replication, or any suitable other nucleic acid amplification technique. A suitable nucleic acid amplification technique is configured to amplify a region of a nucleic acid sequence comprising one or more genetic risk variants disclosed herein. In some embodiments, the amplification assays requires primers. The nucleic acid sequence for the genetic risk variants and/or genes known or provided herein is sufficient to enable one of skill in the art to select primers to amplify any portion of the gene or genetic variants. A DNA sample suitable as a primer may be obtained, e.g., by polymerase chain reaction (PCR) amplification of genomic DNA, fragments of genomic DNA, fragments of genomic DNA ligated to adaptor sequences or cloned sequences. A person of skill in the art would utilize computer programs to design of primers with the desired specificity and optimal amplification properties, such as Oligo version 7.0 (National Biosciences). Controlled robotic systems are useful for isolating and amplifying nucleic acids and can be used.

The methods described herein, in some embodiments, comprise detecting a protein-coding sequence, such as mRNA or cDNA. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to any one of SEQ ID NOS: 1-6 when the biomarker comprises ACE2. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to any one of SEQ ID NOS: 12-14 when the biomarker comprises TMPRSS2. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to any one of SEQ ID NOS: 18-23 when the biomarker comprises TMPRSS4. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to SEQ ID NO: 30 when the biomarker comprises SLC6A19. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to any one of SEQ ID NOS: 32-39 when the biomarker comprises JAK1. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to SEQ ID NO: 47 when the biomarker comprises SIGMAR1. In some embodiments, more than one biomarker is detected using the methods disclosed herein, such as at least two, three, four, five, six, seven, eight, nine, 10, 11, 12, 13, 14, 15, 16, 17, 18, 19, 20, 21, 22, 23, 24, 25, 26, 27, 28, 29, 30 biomarkers.

In some embodiments, methods comprise sequencing genetic material obtained from a biological sample from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods available to one of skill in the art may also be employed.

In some embodiments, a number of nucleotides that are sequenced are at least 5, 10, 15, 20, 25, 30, 35, 40, 45, 50, 100, 150, 200, 300, 400, 500, 2000, 4000, 6000, 8000, 10000, 20000, 50000, 100000, or more than 100000 nucleotides. In some embodiments, the number of nucleotides sequenced is in a range of about 1 to about 100000 nucleotides, about 1 to about 10000 nucleotides, about 1 to about 1000 nucleotides, about 1 to about 500 nucleotides, about 1 to about 300 nucleotides, about 1 to about 200 nucleotides, about 1 to about 100 nucleotides, about 5 to about 100000 nucleotides, about 5 to about 10000 nucleotides, about 5 to about 1000 nucleotides, about 5 to about 500 nucleotides, about 5 to about 300 nucleotides, about 5 to about 200 nucleotides, about 5 to about 100 nucleotides, about 10 to about 100000 nucleotides, about 10 to about 10000 nucleotides, about 10 to about 1000 nucleotides, about 10 to about 500 nucleotides, about 10 to about 300 nucleotides, about 10 to about 200 nucleotides, about 10 to about 100 nucleotides, about 20 to about 100000 nucleotides, about 20 to about 10000 nucleotides, about 20 to about 1000 nucleotides, about 20 to about 500 nucleotides, about 20 to about 300 nucleotides, about 20 to about 200 nucleotides, about 20 to about 100 nucleotides, about 30 to about 100000 nucleotides, about 30 to about 10000 nucleotides, about 30 to about 1000 nucleotides, about 30 to about 500 nucleotides, about 30 to about 300 nucleotides, about 30 to about 200 nucleotides, about 30 to about 100 nucleotides, about 50 to about 100000 nucleotides, about 50 to about 10000 nucleotides, about 50 to about 1000 nucleotides, about 50 to about 500 nucleotides, about 50 to about 300 nucleotides, about 50 to about 200 nucleotides, or about 50 to about 100 nucleotides.

In some embodiments, a transcriptomic risk signature is developed, based at least in part, on the expression levels of the one or more biomarkers disclosed herein. In such a case, a transcriptomic risk profile of the biological sample obtained from the subject may be detected using the methods disclosed herein. In some embodiments, the presence, level, or activity of two or more biomarkers in the biological sample is determined by detecting a transcribed or reverse transcribed polynucleotide, or portion thereof (e.g., mRNA, or cDNA), of a target gene making up the transcriptomic risk signature or transcriptomic risk profile. Any suitable method of detecting a biomarker, such as those disclosed herein, may be utilized to detect a transcriptomic risk signature or transcriptomic risk profile, such as those disclosed herein. A transcriptomic risk signature or transcriptomic risk profile can also be detected at the protein level, using a detection reagent that detects the protein product encoded by the mRNA of the biomarker, directly or indirectly, such the detection reagents disclosed herein.

In some embodiments, methods comprise detecting a polypeptide or a fragment thereof using an immuno-assay. Suitable immuno-assays include immunohistochemistry, enzyme linked-immunosorbent assay (ELISA), flow cytometry, mass spectrometry, Matrix assisted laser desorption/ionization (MALDI), surface enhanced laser desorption/ionization time-of-flight mass spectrometry (SELDI-TOF), proximity assays (e.g., Fluorescence Resonance Energy Transfer (FRET)), and single molecule detection (e.g., SIMOA®). Additional suitable immuno-assays can be found in Powers et al., Protein analytical assays for diagnosing, monitoring, and choosing treatment for cancer patients. J Healthc Eng. 2012 December; 3(4): 503-534, which is hereby incorporated by reference in its entirety.

In some embodiments, such immuno-assays are used to detect a biomarker comprising a particular sequence. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to any one of SEQ ID NOS: 7-11 when the biomarker comprises ACE2. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to any one of SEQ ID NOS: 15-17 when the biomarker comprises TMPRSS2. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to any one of SEQ ID NOS: 24-29 when the biomarker comprises TMPRSS4. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to SEQ ID NO: 31 when the biomarker comprises SLC6A19. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to any one of SEQ ID NOS: 40-46 when the biomarker comprises JAK1. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to SEQ ID NO: 48 when the biomarker comprises SIGMAR1. In some embodiments, more than one biomarker is detected using the methods disclosed herein, such as at least two, three, four, five, six, seven, eight, nine, 10, 11, 12, 13, 14, 15, 16, 17, 18, 19, 20, 21, 22, 23, 24, 25, 26, 27, 28, 29, 30 biomarkers.

2. Methods of Treatment

Disclosed herein, in some embodiments, are methods of treating a disease or a condition disclosed herein in a subject. In some embodiments, methods comprise administering to the subject a therapeutic agent disclosed herein for treatment of the disease or the condition. In some embodiments, the subject is selected for treatment, based at least in part, on the expression level of one or more biomarkers detected in a biological sample obtained from the subject. In some embodiments, the one or more biomarkers comprises angiotensin-converting enzyme 2 (ACE2), transmembrane serine protease 2 (TMPRSS2), transmembrane serine protease 4 (TMPRSS4), solute carrier family 6 member 19 (SLC6A19), Sigma Non-Opioid Intracellular Receptor 1 (SIGMAR1), or Janus kinase 1 (JAK1), or a combination thereof. In some embodiments, the therapeutic agent is a targets expression or activity of the one or more biomarkers. In some embodiments, the therapeutic agent comprise an anti-inflammatory mediator, a steroid, and interleukin 12 (IL-12) or interleukin 23 (IL-23) inhibitor (e.g., ustekinumab), an α4β7 integrin inhibitor (e.g., vedolizumab), or a tumor necrosis factor (TNF) inhibitor (e.g., infliximab), or a combination thereof.

In some embodiments, the diseases or conditions disclosed herein are an inflammatory disease, a fibrostenotic disease, or a fibrotic disease. Non-limiting examples of inflammatory diseases include diseases of the gastrointestinal (GI) tract, liver, gallbladder, and joints. In some cases, the inflammatory disease inflammatory bowel disease (IBD), Crohn's disease (CD), or ulcerative colitis (UC), systemic lupus erythematosus (SLE), or rheumatoid arthritis. A subject may suffer from fibrosis, fibrostenosis, or a fibrotic disease, either isolated or in combination with an inflammatory disease. In some cases, the CD is obstructive CD. The obstructive CD may result from inflammation that has led to the formation of scar tissue in the intestinal wall (fibrostenosis) and/or swelling. In some cases, the CD is characterized by the presence of fibrotic and/or inflammatory strictures. The strictures may be determined by computed tomography enterography (CTE), and magnetic resonance imaging enterography (MRE). In some embodiments, the disease is primary sclerosing cholangitis (PSC). Exemplary methods of diagnosing PSC include magnetic resonance cholangiopancreatography (MRCP), liver function tests, and histology. Liver function tests are valuable in the laboratory workup, and may include measurement of levels of serum alkaline phosphatase, serum aminotransferase, gamma glutamyl transpeptidase, and the presence of hypergammaglobulinemia. The disease or condition may comprise thiopurine toxicity, or a disease caused by thiopurine toxicity (such as pancreatitis or leukopenia). In further embodiments provided, the subject experiences non-response to an induction of a therapy, or a loss-of-response to the therapy after a successful induction of the therapy. Non-limiting examples of standard treatment include glucocorticosteriods, anti-TNF therapy (e.g., infliximab), anti-a4-b7 therapy (vedolizumab), anti-IL12p40 therapy (ustekinumab), Thalidomide, and Cytoxin.

In some embodiments, the subject disclosed herein is a mammal, such as for example a mouse, rat, guinea pig, rabbit, non-human primate, or farm animal. In some embodiments, the subject is human. In some embodiments, the subject is a patient who is diagnosed with the disease or condition disclosed herein. In some embodiments, the subject is not diagnosed with the disease or condition. In some embodiments, the subject is suffering from a symptom related to a disease or condition disclosed herein (e.g., abdominal pain, cramping, diarrhea, rectal bleeding, fever, weight loss, fatigue, loss of appetite, dehydration, and malnutrition, anemia, or ulcers). In some embodiments, the subject has, or is suspected of having, Coronavirus Disease 2019 (COVID-19), or an infection caused by severe acute respiratory syndrome (SARS) coronavirus 2 (SARS-CoV-2).

In some embodiments, the subject is susceptible to, or is inflicted with, thiopurine toxicity, or a disease caused by thiopurine toxicity (such as pancreatitis or leukopenia). The subject may experience, or is suspected of experiencing, non-response or loss-of-response to a standard treatment (e.g., anti-TNF therapy, anti-a4-b7 therapy (vedolizumab), anti-IL12p40 therapy (ustekinumab), Thalidomide, or Cytoxin). In some embodiments, the subject is determined to be responsive to a standard treatment.

In some embodiment, one or more biomarkers are provided that are useful for identifying whether a subject is has, or is prone to developing, a severe form of a disease or a condition disclosed herein; and/or is suitable for treatment of the disease or the condition with a particular therapy, such a one or more therapeutic agents disclosed herein. In some embodiments, the one or more biomarkers is selected from Table 1. In some embodiments, the one or more biomarkers comprises angiotensin-converting enzyme 2 (ACE2), transmembrane serine protease 2 (TMPRSS2), transmembrane serine protease 4 (TMPRSS4), solute carrier family 6 member 19 (SLC6A19), Sigma Non-Opioid Intracellular Receptor 1 (SIGMAR1), or Janus kinase 1 (JAK1), or a combination thereof. In some embodiments, the biomarker comprises ACE2. In some embodiments, the biomarker comprises TMPRSS2. In some embodiments, the biomarker comprises TMPRSS4. In some embodiments, the biomarker comprises SLC6A19. In some embodiments, the biomarker comprises SIGMAR1. In some embodiments, the biomarker comprises JAK1.

In some embodiments, the biomarker comprises a polypeptide or ribonucleic acid (RNA). In some embodiments, the polypeptide is a protein, or a fragment thereof. In some embodiments comprises fragmented RNA. In some embodiments, the biomarker comprises partially degraded RNA. In some embodiments, the biomarker comprises a microRNA or portion thereof. In some embodiments, the biomarker comprises an RNA molecule or a fragmented RNA molecule (RNA fragments) selected from: a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, an RNA transcript, a synthetic RNA, and combinations thereof. In some embodiments, the biomarker is a transcribed polynucleotide comprising DNA or complementary DNA (cDNA) of the mRNA encoding the biomarker.

In some embodiments, the biomarker comprises, or is encoded by, a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to a sequence provided in any one of SEQ ID NOS: 1-48. In some embodiments, the biomarker is more than or equal to about 90% identical to a sequence provided in any one of SEQ ID NOS: 1-48. In some embodiments, the biomarker is more than or equal to about 95% identical to a sequence provided in any one of SEQ ID NOS: 1-48. In some embodiments, the biomarker is more than or equal to about 97% identical to a sequence provided in any one of SEQ ID NOS: 1-48. In some embodiments, the biomarker is more than or equal to about 98% identical to a sequence provided in any one of SEQ ID NOS: 1-48. In some embodiments, the biomarker is more than or equal to about 99% identical to a sequence provided in any one of SEQ ID NOS: 1-48.

In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to any one of SEQ ID NOS: 1-6 when the biomarker comprises ACE2. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to any one of SEQ ID NOS: 12-14 when the biomarker comprises TMPRSS2. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to any one of SEQ ID NOS: 18-23 when the biomarker comprises TMPRSS4. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to SEQ ID NO: 30 when the biomarker comprises SLC6A19. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to any one of SEQ ID NOS: 32-39 when the biomarker comprises JAK1. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to SEQ ID NO: 47 when the biomarker comprises SIGMAR1. In some embodiments, more than one biomarker is detected using the methods disclosed herein, such as at least two, three, four, five, six, seven, eight, nine, 10, 11, 12, 13, 14, 15, 16, 17, 18, 19, 20, 21, 22, 23, 24, 25, 26, 27, 28, 29, 30 biomarkers.

In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to any one of SEQ ID NOS: 7-11 when the biomarker comprises ACE2. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to any one of SEQ ID NOS: 15-17 when the biomarker comprises TMPRSS2. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to any one of SEQ ID NOS: 24-29 when the biomarker comprises TMPRSS4. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to SEQ ID NO: 31 when the biomarker comprises SLC6A19. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to any one of SEQ ID NOS: 40-46 when the biomarker comprises JAK1. In some embodiments, the biomarker comprises a sequence that is more than or equal to about 70%, 75%, 80%, 85%, 86%, 87%, 88%, 89%, 90%, 91%, 92%, 93%, 94%, 95%, 96%, 97%, 98%, 99%, or 100% identical to SEQ ID NO: 48 when the biomarker comprises SIGMAR1. In some embodiments, more than one biomarker is detected using the methods disclosed herein, such as at least two, three, four, five, six, seven, eight, nine, 10, 11, 12, 13, 14, 15, 16, 17, 18, 19, 20, 21, 22, 23, 24, 25, 26, 27, 28, 29, 30 biomarkers.

In some embodiments, the expression of the one or more biomarkers detected are higher or lower than a control or a reference sample. In some embodiments, the control is derived from a non-diseased subject. In some embodiments, the reference sample is a sample obtained from the subject prior to, during or after a treatment described herein. In some embodiments, the reference sample is a sample obtained from the subject from a different tissue, such as the small bowel or the colon.

In some embodiments, biomarker expression is absolute. In some embodiments, an absolute level of the biomarker is measured, which is calculated by the ratio between the expression of the biomarker (e.g., number of copies) and the expression of one or more reference genes (e.g., a house-keeping gene). In some embodiments, the absolute numbers of copies of the biomarker are between about 1,5000 and 6,500, 2,000 and 6,000, 2,500 and 5,500, 3,000 and 5,000, 3,500 and 4,500, or 3,000 and 4,000, copies. In some embodiment, the absolute numbers of copies of the biomarker are between about 150 and 450, 200 and 400, or 250 and 350, copies. In some embodiments, the absolute number of copies of the biomarker is at most or equal to about 2,000, 4,000, 5,000, 6,000, 8,000, 9,000, or 10,000 copies. In some embodiments, the absolute number of copies of the biomarker is at least or equal to about 2,000, 4,000, 5,000, 6,000, 8,000, 9,000, or 10,000 copies.

In some embodiments, biomarker expression is relative, for example, as an expression of fold change between two or more samples (e.g., two patient samples at different time points, a control sample and a patient sample collected at the same time point, two different types of samples taken from the same patient at the same timepoint, and so on). In some embodiments, the expression of the biomarker is about 1-fold, 2-fold, 3-fold, 4-fold, 5-fold, 6-fold, 7-fold, 8-fold, 9-fold, or 10-fold lower than an expression of the biomarker in a control sample. In some embodiments, the expression of the biomarker is about 1-fold, 2-fold, 3-fold, 4-fold, 5-fold, 6-fold, 7-fold, 8-fold, 9-fold, or 10-fold higher than an expression of the biomarker in a control sample. In some embodiments, the expression of the biomarker is about 1-fold, 2-fold, 3-fold, 4-fold, 5-fold, 6-fold, 7-fold, 8-fold, 9-fold, or 10-fold lower than an expression of the biomarker in a biological sample obtained from the subject or patient at a different timepoint (e.g., during treatment course). In some embodiments, the expression of the biomarker is about 1-fold, 2-fold, 3-fold, 4-fold, 5-fold, 6-fold, 7-fold, 8-fold, 9-fold, or 10-fold higher than an expression of the biomarker in a biological sample obtained from the subject or patient at a different timepoint (e.g., during treatment course). In some embodiments, the expression of the biomarker is about 1-fold, 2-fold, 3-fold, 4-fold, 5-fold, 6-fold, 7-fold, 8-fold, 9-fold, or 10-fold lower than an expression of the biomarker in a different biological sample obtained from the same subject. In some embodiments, the expression of the biomarker is about 1-fold, 2-fold, 3-fold, 4-fold, 5-fold, 6-fold, 7-fold, 8-fold, 9-fold, or 10-fold higher than an expression of the biomarker in a different biological sample obtained from the same subject. In some embodiments, the expression of the biomarker in a biological sample obtained from the small bowel is at least 10-fold higher than the expression of the biomarker in the colon.

In some embodiments, the therapeutic agent is useful for treating the disease or conditions disclosed herein, such as inflammatory bowel disease (IBD). Non-limiting examples of classes of therapeutic agents useful for this purpose include anti-inflammatory mediators (e.g., small molecule and large molecule), steroids, interleukin 12 (IL-12) or interleukin 23 (IL-23) inhibitors (e.g., ustekinumab), α4β7 integrin inhibitors (e.g., vedolizumab), and tumor necrosis factor (TNF) inhibitors (e.g., infliximab). Non-limiting examples of therapeutic agents used to treat IBD include azathioprine, methotrexate, 6-mercaptopurine, prednisone, mesalazine, budesonide, corticosteriods, aminosalicylates, mesalamine, balsalazide (Colazal), and olsalazine (Dipentum).

In some embodiments, the therapeutic agent comprises an immunosuppressant, or a class of drugs that suppress, or reduce, the strength of the immune system. In some embodiments, the immunosuppressant is an antibody. Non-limiting examples of immunosuppressant therapeutic agents include STELARA® (ustekinumab) azathioprine (AZA), 6-mercaptopurine (6-MP), methotrexate, cyclosporin A. (CsA).

In some embodiments, the therapeutic agent comprises a selective anti-inflammatory drug, or a class of drugs that specifically target pro-inflammatory molecules in the body. In some embodiments, the anti-inflammatory drug comprises an antibody. In some embodiments, the anti-inflammatory drug comprises a small molecule. Non-limiting examples of anti-inflammatory drugs include ENTYVIO (vedolizumab), corticosteroids, aminosalicylates, mesalamine, balsalazide (Colazal) and olsalazine (Dipentum).

In some embodiments, the therapeutic agent comprises a small molecule. The small molecule may be used to treat inflammatory diseases or conditions, or fibrostenonic or fibrotic disease. Non-limiting examples of small molecules include Otezla® (apremilast), alicaforsen, or ozanimod (RPC-1063).

In some embodiments, the therapeutic agent targets the activity or the expression of the one or more biomarkers provided in Table 1. Such targeted therapeutic agents are particularly useful for treating the disease or the condition in a subject that has been selected for treatment with that targeted therapeutic agent, based at least in part, on the expression level of the one or more biomarkers described herein. For example, in some embodiments, the subject is identified as a responder for a particular targeted therapeutic agent disclosed herein, and subsequently treated with that targeted therapeutic agent. In some embodiments, the therapeutic agent modulates the expression or activity of ACE2. In some embodiments, the therapeutic agent modulates the expression or activity of TMPRSS2. In some embodiments, the therapeutic agent modulates the expression or activity of TMPRSS4. In some embodiments, the therapeutic agent modulates the expression or activity of SLC6A19. In some embodiments, the therapeutic agent modulates the expression or activity of SIGMAR1. In some embodiments, the therapeutic agent modulates the expression or activity of JAK1. Non-limiting examples of JAK1 inhibitors include Ruxolitinib (INCB018424), S-Ruxolitinib (INCB018424), Baricitinib (LY3009104, INCB028050), Filgotinib (GLPG0634), Momelotinib (CYT387), Cerdulatinib (PRT062070, PRT2070),

LY2784544, NVP-BSK805, 2HCl, Tofacitinib (CP-690550, Tasocitinib), XL019, Pacritinib (SB1518), or ZM 39923 HCl.

In some embodiments, the therapeutic agent inhibits the expression of the activity of Angiotensin converting enzyme (ACE) (an ACE inhibitor). In some embodiments, the ACE inhibitor comprises Benazepril (Lotensin). In some embodiments, the ACE inhibitor comprises Captopril. In some embodiments, the ACE inhibitor comprises Enalapril (Vasotec). In some embodiments, the ACE inhibitor comprises Fosinopril. In some embodiments, the ACE inhibitor comprises Lisinopril (Prinivil, Zestril). In some embodiments, the ACE inhibitor comprises Moexipril. In some embodiments, the ACE inhibitor comprises Perindopril. In some embodiments, the ACE inhibitor comprises Quinapril (Accupril). In some embodiments, the ACE inhibitor comprises Ramipril (Altace). In some embodiments, the ACE inhibitor comprises Trandolapril.

In some embodiments, the therapeutic agent targets the RAS pathway. In some embodiments, the therapeutic agent inhibits the expression of the activity of angiotensinogen. In some embodiments, the therapeutic agent inhibits the expression of the activity of Angiotensin-II or its receptor, Angiotensin-II Receptor. In some embodiments, the therapeutic agent is an Angiotensin II receptor blockers (ARBs). In some embodiments, the ARB comprises Valsartan, Losartan, Azilsartan, Irbesartan, Olmesartan, Telmisartan, or Fimasartan, or a combination thereof.

In some embodiments, the therapeutic agent is formulated in a pharmaceutical composition or formulation. In some embodiments, the pharmaceutical composition comprises a mixture of the therapeutic agent and another chemical components (e.g., pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compositions include two or more therapeutic agent (e.g., one or more therapeutic agents and one or more additional agents) as discussed herein. In practicing the methods of treatment or use provided herein, therapeutically effective amounts of therapeutic agents described herein are administered in a pharmaceutical composition to a mammal having a disease, disorder, or condition to be treated, e.g., an inflammatory disease, fibrostenotic disease, and/or fibrotic disease. In some embodiments, the mammal is a human. A therapeutically effective amount can vary widely depending on the severity of the disease, the age and relative health of the subject, the potency of the therapeutic agent used and other factors. The therapeutic agents can be used singly or in combination with one or more therapeutic agents as components of mixtures.

In some embodiments, the pharmaceutical formulations described herein are administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a therapeutic agent are manufactured in a conventional manner, such as, by way of example only, by means of conventional mixing, dissolving, granulating, dragee-making, levigating, emulsifying, encapsulating, entrapping or compression processes.

The pharmaceutical compositions may include at least a therapeutic agent as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, therapeutic agents exist in unsolvated form or in solvated forms with pharmaceutically acceptable solvents such as water, ethanol, and the like. The solvated forms of the therapeutic agents are also considered to be disclosed herein.

In some embodiments, a therapeutic agent exists as a tautomer. All tautomers are included within the scope of the agents presented herein. As such, it is to be understood that a therapeutic agent or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound.

In some embodiments, a therapeutic agent exists as an enantiomer, diastereomer, or other stereoisomeric form. The agents disclosed herein include all enantiomeric, diastereomeric, and epimeric forms as well as mixtures thereof.

In some embodiments, therapeutic agents described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a therapeutic agent described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the therapeutic agent. In certain embodiments, a prodrug is enzymatically metabolized by one or more steps or processes to the biologically, pharmaceutically or therapeutically active form of the therapeutic agent.

Prodrug forms of the therapeutic agents, wherein the prodrug is metabolized in vivo to produce an agent as set forth herein are included within the scope of the claims. Prodrug forms of the herein described therapeutic agents, wherein the prodrug is metabolized in vivo to produce an agent as set forth herein are included within the scope of the claims. In some cases, some of the therapeutic agents described herein may be a prodrug for another derivative or active compound. In some embodiments described herein, hydrazones are metabolized in vivo to produce a therapeutic agent.

In certain embodiments, compositions provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

The pharmaceutical compositions described herein are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some cases it is desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections or drips or infusions, a therapeutic agent described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a therapeutic agent is formulated for use as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the therapeutic agent described herein and a suitable powder base such as lactose or starch.

Representative intranasal formulations are described in, for example, U.S. Pat. Nos. 4,476,116, 5,116,817 and 6,391,452. Formulations that include a therapeutic agent are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the therapeutic agents described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active therapeutic agent doses.

In some embodiments, pharmaceutical formulations of a therapeutic agent are in the form of a capsules, including push fit capsules made of gelatin, as well as soft, sealed capsules made of gelatin and a plasticizer, such as glycerol or sorbitol. The push fit capsules contain the active ingredients in admixture with filler such as lactose, binders such as starches, and/or lubricants such as talc or magnesium stearate and, optionally, stabilizers. In soft capsules, the active therapeutic agent is dissolved or suspended in suitable liquids, such as fatty oils, liquid paraffin, or liquid polyethylene glycols. In some embodiments, stabilizers are added. A capsule may be prepared, for example, by placing the bulk blend of the formulation of the therapeutic agent inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

All formulations for oral administration are in dosages suitable for such administration. In one aspect, solid oral dosage forms are prepared by mixing a therapeutic agent with one or more of the following: antioxidants, flavoring agents, and carrier materials such as binders, suspending agents, disintegration agents, filling agents, surfactants, solubilizers, stabilizers, lubricants, wetting agents, and diluents. In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder. Compressed tablets are solid dosage forms prepared by compacting the bulk blend of the formulations described above. In various embodiments, tablets will include one or more flavoring agents. In other embodiments, the tablets will include a film surrounding the final compressed tablet. In some embodiments, the film coating can provide a delayed release of a therapeutic agent from the formulation. In other embodiments, the film coating aids in patient compliance (e.g., Opadry® coatings or sugar coating). Film coatings including Opadry® typically range from about 1% to about 3% of the tablet weight. In some embodiments, solid dosage forms, e.g., tablets, effervescent tablets, and capsules, are prepared by mixing particles of a therapeutic agent with one or more pharmaceutical excipients to form a bulk blend composition. The bulk blend is readily subdivided into equally effective unit dosage forms, such as tablets, pills, and capsules. In some embodiments, the individual unit dosages include film coatings. These formulations are manufactured by conventional formulation techniques.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents. Exemplary useful microencapsulation materials include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to therapeutic agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Buccal formulations that include a therapeutic agent are administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, a therapeutic agent is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a pharmaceutical composition described herein is in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

Suitable disintegrants for use in the solid dosage forms described herein include, but are not limited to, natural starch such as corn starch or potato starch, a pregelatinized starch, or sodium starch glycolate, a cellulose such as methylcrystalline cellulose, methylcellulose, microcrystalline cellulose, croscarmellose, or a cross-linked cellulose, such as cross-linked sodium carboxymethylcellulose, cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a gum such as agar, guar, locust bean, Karaya, pectin, or tragacanth, sodium starch glycolate, bentonite, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose, hydroxypropylmethylcellulose, hydroxypropylmethylcellulose acetate stearate, hydroxyethylcellulose, hydroxypropylcellulose, ethylcellulose, and microcrystalline cellulose, microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose, glucose, dextrose, molasses, mannitol, sorbitol, xylitol, lactose, a natural or synthetic gum such as acacia, tragacanth, ghatti gum, mucilage of isapol husks, starch, polyvinylpyrrolidone, larch arabogalactan, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Binder levels of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms of the pharmaceutical compositions described herein. The amounts of such additives can be readily determined by one skilled in the art, according to the particular properties desired.

In various embodiments, the particles of a therapeutic agents and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In other embodiments, a powder including a therapeutic agent is formulated to include one or more pharmaceutical excipients and flavors. Such a powder is prepared, for example, by mixing the therapeutic agent and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared. Effervescent salts have been used to disperse medicines in water for oral administration.

In some embodiments, the pharmaceutical dosage forms are formulated to provide a controlled release of a therapeutic agent. Controlled release refers to the release of the therapeutic agent from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein are formulated as enteric coated delayed release oral dosage forms, i.e., as an oral dosage form of a pharmaceutical composition as described herein which utilizes an enteric coating to affect release in the small intestine or large intestine. In one aspect, the enteric coated dosage form is a compressed or molded or extruded tablet/mold (coated or uncoated) containing granules, powder, pellets, beads or particles of the active ingredient and/or other composition components, which are themselves coated or uncoated. In one aspect, the enteric coated oral dosage form is in the form of a capsule containing pellets, beads or granules, which include a therapeutic agent that are coated or uncoated.

Any coatings should be applied to a sufficient thickness such that the entire coating does not dissolve in the gastrointestinal fluids at pH below about 5, but does dissolve at pH about 5 and above. Coatings are typically selected from any of the following: Shellac—this coating dissolves in media of pH >7; Acrylic polymers—examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine; Poly Vinyl Acetate Phthalate (PVAP)—PVAP dissolves in pH >5, and it is much less permeable to water vapor and gastric fluids. Conventional coating techniques such as spray or pan coating are employed to apply coatings. The coating thickness must be sufficient to ensure that the oral dosage form remains intact until the desired site of topical delivery in the intestinal tract is reached.

In other embodiments, the formulations described herein are delivered using a pulsatile dosage form. A pulsatile dosage form is capable of providing one or more immediate release pulses at predetermined time points after a controlled lag time or at specific sites. Exemplary pulsatile dosage forms and methods of their manufacture are disclosed in U.S. Pat. Nos. 5,011,692, 5,017,381, 5,229,135, 5,840,329 and 5,837,284. In one embodiment, the pulsatile dosage form includes at least two groups of particles, (i.e. multiparticulate) each containing the formulation described herein. The first group of particles provides a substantially immediate dose of a therapeutic agent upon ingestion by a mammal. The first group of particles can be either uncoated or include a coating and/or sealant. In one aspect, the second group of particles comprises coated particles. The coating on the second group of particles provides a delay of from about 2 hours to about 7 hours following ingestion before release of the second dose. Suitable coatings for pharmaceutical compositions are described herein or known in the art.

In some embodiments, pharmaceutical formulations are provided that include particles of a therapeutic agent and at least one dispersing agent or suspending agent for oral administration to a subject. The formulations may be a powder and/or granules for suspension, and upon admixture with water, a substantially uniform suspension is obtained.

In some embodiments, particles formulated for controlled release are incorporated in a gel or a patch or a wound dressing.

In one aspect, liquid formulation dosage forms for oral administration and/or for topical administration as a wash are in the form of aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of a therapeutic agent, the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

In some embodiments, the liquid formulations also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state, as defined in The USP Pharmacists' Pharmacopeia (2005 edition, chapter 905), for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch, or sodium starch glycolate; a cellulose such as methylcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein include, for example, hydrophilic polymers, electrolytes, Tween® 60 or 80, PEG, polyvinylpyrrolidone, and the carbohydrate-based dispersing agents such as, for example, hydroxypropylcellulose and hydroxypropyl cellulose ethers, hydroxypropyl methylcellulose and hydroxypropyl methylcellulose ethers, carboxymethylcellulose sodium, methylcellulose, hydroxy ethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer, 4-(1,1,3,3-tetramethylbutyl)-phenol polymer with ethylene oxide and formaldehyde (also known as tyloxapol), poloxamers; and poloxamines. In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers; hydroxypropyl methylcellulose and hydroxypropyl methylcellulose ethers; carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers; or poloxamines.

Wetting agents suitable for the aqueous suspensions and dispersions described herein include, but are not limited to, cetyl alcohol, glycerol monostearate, polyoxyethylene sorbitan fatty acid esters (e.g., the commercially available Tweens® such as e.g., Tween 20® and Tween 80®, and polyethylene glycols, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, aspartame, chocolate, cinnamon, citrus, cocoa, cyclamate, dextrose, fructose, ginger, glycyrrhetinate, glycyrrhiza (licorice) syrup, monoammonium glyrrhizinate (MagnaSweet®), malitol, mannitol, menthol, neohesperidine DC, neotame, Prosweet® Powder, saccharin, sorbitol, stevia, sucralose, sucrose, sodium saccharin, saccharin, aspartame, acesulfame potassium, mannitol, sucralose, tagatose, thaumatin, vanilla, xylitol, or any combination thereof.

In some embodiments, a therapeutic agent is prepared as transdermal dosage form. In some embodiments, the transdermal formulations described herein include at least three components: (1) a therapeutic agent; (2) a penetration enhancer; and (3) an optional aqueous adjuvant. In some embodiments the transdermal formulations include additional components such as, but not limited to, gelling agents, creams and ointment bases, and the like. In some embodiments, the transdermal formulation is presented as a patch or a wound dressing. In some embodiments, the transdermal formulation further include a woven or non-woven backing material to enhance absorption and prevent the removal of the transdermal formulation from the skin. In other embodiments, the transdermal formulations described herein can maintain a saturated or supersaturated state to promote diffusion into the skin.

In one aspect, formulations suitable for transdermal administration of a therapeutic agent described herein employ transdermal delivery devices and transdermal delivery patches and can be lipophilic emulsions or buffered, aqueous solutions, dissolved and/or dispersed in a polymer or an adhesive. In one aspect, such patches are constructed for continuous, pulsatile, or on demand delivery of pharmaceutical agents. Still further, transdermal delivery of the therapeutic agents described herein can be accomplished by means of iontophoretic patches and the like. In one aspect, transdermal patches provide controlled delivery of a therapeutic agent. In one aspect, transdermal devices are in the form of a bandage comprising a backing member, a reservoir containing the therapeutic agent optionally with carriers, optionally a rate controlling barrier to deliver the therapeutic agent to the skin of the host at a controlled and predetermined rate over a prolonged period of time, and means to secure the device to the skin.

In further embodiments, topical formulations include gel formulations (e.g., gel patches which adhere to the skin). In some of such embodiments, a gel composition includes any polymer that forms a gel upon contact with the body (e.g., gel formulations comprising hyaluronic acid, pluronic polymers, poly(lactic-co-glycolic acid (PLGA)-based polymers or the like). In some forms of the compositions, the formulation comprises a low-melting wax such as, but not limited to, a mixture of fatty acid glycerides, optionally in combination with cocoa butter which is first melted. Optionally, the formulations further comprise a moisturizing agent.

In certain embodiments, delivery systems for pharmaceutical therapeutic agents may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, a therapeutic agent described herein may be administered topically and can be formulated into a variety of topically administrable compositions, such as solutions, suspensions, lotions, gels, pastes, medicated sticks, balms, creams or ointments. Such pharmaceutical therapeutic agents can contain solubilizers, stabilizers, tonicity enhancing agents, buffers and preservatives.

In general, methods disclosed herein comprise administering a therapeutic agent by oral administration. However, In some embodiments, methods comprise administering a therapeutic agent by intraperitoneal injection. In some embodiments, methods comprise administering a therapeutic agent in the form of an anal suppository. In some embodiments, methods comprise administering a therapeutic agent by intravenous (“i.v.”) administration. It is conceivable that one may also administer therapeutic agents disclosed herein by other routes, such as subcutaneous injection, intramuscular injection, intradermal injection, trasndermal injection percutaneous administration, intranasal administration, intralymphatic injection, rectal administration intragastric administration, or any other suitable parenteral administration. In some embodiments, routes for local delivery closer to site of injury or inflammation are preferred over systemic routes. Routes, dosage, time points, and duration of administrating therapeutics may be adjusted. In some embodiments, administration of therapeutics is prior to, or after, onset of either, or both, acute and chronic symptoms of the disease or condition.

An effective dose and dosage of therapeutics to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition (e.g., reduced instances of diarrhea, rectal bleeding, weight loss, and size or number of intestinal lesions or strictures, reduced fibrosis or fibrogenesis, reduced fibrostenosis, reduced inflammation). In some embodiments, the beneficial response may be measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g. individuals who do not suffer from the disease or condition). In instances wherein the therapeutic agent is not therapeutically effective or is not providing a sufficient alleviation of the disease or condition, or symptom of the disease or condition, then the dosage amount and/or route of administration may be changed, or an additional agent may be administered to the subject, along with the therapeutic agent. In some embodiments, as a patient is started on a regimen of a therapeutic agent, the patient is also weaned off (e.g., step-wise decrease in dose) a second treatment regimen.

Suitable dose and dosage administrated to a subject is determined by factors including, but no limited to, the particular therapeutic agent, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated. In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously (or over a short period of time) or at appropriate intervals, for example as two, three, four or more sub-doses per day. Non-limiting examples of effective dosages of for oral delivery of a therapeutic agent include between about 0.1 mg/kg and about 100 mg/kg of body weight per day, and preferably between about 0.5 mg/kg and about 50 mg/kg of body weight per day. In other instances, the oral delivery dosage of effective amount is about 1 mg/kg and about 10 mg/kg of body weight per day of active material. Non-limiting examples of effective dosages for intravenous administration of the therapeutic agent include at a rate between about 0.01 to 100 pmol/kg body weight/min. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the therapeutic agent used, the disease or condition to be treated, the mode of administration, the requirements of the individual subject, the severity of the disease or condition being treated, and the judgment of the practitioner.

In some embodiments, the administration of the therapeutic agent is hourly, once every 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours 22 hours, 23 hours, 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 1 year, 2 years, 3 years, 4 years, or 5 years, or 10 years. The effective dosage ranges may be adjusted based on subject's response to the treatment. Some routes of administration will require higher concentrations of effective amount of therapeutics than other routes.

In certain embodiments wherein the patient's condition does not improve, upon the doctor's discretion the administration of therapeutic agent is administered chronically, that is, for an extended period of time, including throughout the duration of the patient's life in order to ameliorate or otherwise control or limit the symptoms of the patient's disease or condition. In certain embodiments wherein a patient's status does improve, the dose of therapeutic agent being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. In certain embodiments, the dose of drug being administered may be temporarily reduced or temporarily suspended for a certain length of time (i.e., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the therapeutic agent described herein lies within a range of circulating concentrations that include the ED50 with minimal toxicity. In certain embodiments, the daily dosage range and/or the unit dosage amount varies within this range depending upon the dosage form employed and the route of administration utilized.

A therapeutic agent may be used alone or in combination with an additional therapeutic agent. In some cases, an “additional therapeutic agent” as used herein is administered alone. The therapeutic agents may be administered together or sequentially. The combination therapies may be administered within the same day, or may be administered one or more days, weeks, months, or years apart. In some cases, a therapeutic agent provided herein is administered if the subject is determined to be non-responsive to a first line of therapy, e.g., such as TNF inhibitor. Such determination may be made by treatment with the first line therapy and monitoring of disease state and/or diagnostic determination that the subject would be non-responsive to the first line therapy.

In some embodiments, the therapeutic agent or additional therapeutic agent comprises an anti-TNF therapy, e.g., an anti-TNFα therapy. In some embodiments, the additional therapeutic agent or therapeutic agent comprises a second-line treatment to an anti-TNF therapy. In some embodiments, the additional therapeutic agent comprises an immunosuppressant, or a class of drugs that suppress, or reduce, the strength of the immune system. In some embodiments, the immunosuppressant is an antibody. Non-limiting examples of immunosuppressant therapeutic agents include STELARA® (ustekinumab) azathioprine (AZA), 6-mercaptopurine (6-MP), methotrexate, cyclosporin A. (CsA).

In some embodiments, the additional therapeutic agent or therapeutic agent comprises a selective anti-inflammatory drug, or a class of drugs that specifically target pro-inflammatory molecules in the body. In some embodiments, the anti-inflammatory drug comprises an antibody. In some embodiments, the anti-inflammatory drug comprises a small molecule. Non-limiting examples of anti-inflammatory drugs include ENTYVIO (vedolizumab), corticosteroids, aminosalicylates, mesalamine, balsalazide (Colazal) and olsalazine (Dipentum).

In some embodiments, the additional therapeutic agent or therapeutic agent comprises a stem cell therapy. The stem cell therapy may be embryonic or somatic stem cells. The stem cells may be isolated from a donor (allogeneic) or isolated from the subject (autologous). The stem cells may be expanded adipose-derived stem cells (eASCs), hematopoietic stem cells (HSCs), mesenchymal stem (stromal) cells (MSCs), or induced pluripotent stem cells (iPSCs) derived from the cells of the subject. In some embodiments, the therapeutic agent comprises Cx601/Alofisel® (darvadstrocel).

In some embodiments, the additional therapeutic agent comprises a small molecule. The small molecule may be used to treat inflammatory diseases or conditions, or fibrostenonic or fibrotic disease. Non-limiting examples of small molecules include Otezla® (apremilast), alicaforsen, or ozanimod (RPC-1063).

In some embodiments, the additional therapeutic agent or therapeutic agent comprises administering to the subject an antimycotic agent. In some embodiments, the antimycotic agent comprises an active agent that inhibits growth of a fungus. In some embodiments, the antimycotic agent comprises an active agent that kills a fungus. In some embodiments, the antimycotic agent comprises polyene, an azole, an echinocandin, an flucytosine, an allylamine, a tolnaftate, or griseofulvin, or a combination thereof. In other embodiments, the azole comprises triazole, imidazole, clotrimazole, ketoconazole, itraconazole, terconazole, oxiconazole, miconazole, econazole, tioconazole, voriconazole, fluconazole, isavuconazole, itraconazole, pramiconazole, ravuconazole, or posaconazole. In some other embodiments, the polyene comprises amphotericin B, nystatin, or natamycin. In yet other embodiments, the echinocandin comprises caspofungin, anidulafungin, or micafungin. In various other embodiments, the allylamine comprises naftifine or terbinafine.

3. Methods of Monitoring Treatment

Disclosed herein, in some embodiments, are methods of monitoring a treatment regiment of a subject with a disease or a condition described herein. In some embodiments, methods further comprising optimizing the treatment regiment, based at least in part, on the presence/absence or level of expression of the one or more biomarkers provided in Table 1, such a ACE2. In some embodiments, the treatment regimen includes one or more therapeutic agents described herein, such a steroid, and IL-12/23 inhibitor (e.g., ustekinumab), an α4β7 integrin inhibitor (e.g., vedolizumab), or a TNF inhibitor (e.g., infliximab), or a combination thereof. In some embodiments, the treatment regimen includes a targeted therapeutic agent described herein, such as a therapeutic agent that targets activity or expression of ACE2, TMPRSS2, TMPRSS4, SLC6A19, or JAK1, or a combination thereof. In some embodiments, the disease or the condition is IBD, such as CD or UC.

In some embodiments, the treatment regimen is modified based, at least in part, on the presence/absence or level of the one or more biomarkers provided in Table 1 detected in a biological sample obtained from the subject. In some embodiments, methods comprise: (a) providing a biological sample from a subject that was administered a first dosage amount of a therapeutic agent targeting Tumor Necrosis Factor (TNF), interleukin 12 (IL-12), or interleukin 23 (IL-23); (b) measuring an expression level of a biomarker comprising angiotensin-converting enzyme 2 (ACE2), transmembrane serine protease 2 (TMPRSS2), transmembrane serine protease 4 (TMPRSS4), solute carrier family 6 member 19 (SLC6A19), Sigma Non-Opioid Intracellular Receptor 1 (SIGMAR1), or Janus kinase 1 (JAK1), or a combination thereof; (c) comparing the expression level of the biomarker from (b) to an expression level of the biomarker in a control sample obtained from a subject that was not administered the therapeutic agent. In some embodiments, methods further comprise: (d) administering a second dosage amount that is the same as, or higher than, the first dosage amount of the therapeutic agent based, at least in part, on the expression level of the biomarker in the biological sample measured in (b) when the expression level is higher than the expression level of the biomarker in the control sample; or (e) administering a second dosage amount that is lower than the first dosage amount of the therapeutic agent based, at least in part, on the expression level of the biomarker in the biological sample measured in (b) when the expression level is lower than the expression level of the biomarker in the control sample. In some embodiments, the one or more biomarkers are detected using the methods of detection disclosed herein. In some embodiments, the presence/absence or the level of the expression of the one or more biomarkers is indicative that the subject is at high risk for developing a non-response, or loss-of-response to a therapeutic agent in the subject's treatment regimen.

In some embodiments, methods comprise measuring an absolute expression of the one or more biomarkers. In some embodiments, an absolute level of the biomarker is measured, which is calculated by the ratio between the expression of the biomarker and the expression of one or more reference genes (e.g., a house-keeping gene). In some embodiments, the absolute numbers of copies of the biomarker are between about 1,5000 and 6,500, 2,000 and 6,000, 2,500 and 5,500, 3,000 and 5,000, 3,500 and 4,500, or 3,000 and 4,000, copies. In some embodiment, the absolute numbers of copies of the biomarker are between about 150 and 450, 200 and 400, or 250 and 350, copies. In some embodiments, the absolute number of copies of the biomarker is at most or equal to about 2,000, 4,000, 5,000, 6,000, 8,000, 9,000, or 10,000 copies. In some embodiments, the absolute number of copies of the biomarker is at least or equal to about 2,000, 4,000, 5,000, 6,000, 8,000, 9,000, or 10,000 copies.

In some embodiments, methods comprise measuring a relative expression of the one or more biomarkers, for example, as an expression of fold change between two or more samples (e.g., two patient samples at different time points, a control sample and a patient sample at the same time point, and so on). In some embodiments, the expression of the biomarker is about 1-fold, 2-fold, 3-fold, 4-fold, 5-fold, 6-fold, 7-fold, 8-fold, 9-fold, or 10-fold lower than an expression of the biomarker in a control sample. In some embodiments, the expression of the biomarker is about 1-fold, 2-fold, 3-fold, 4-fold, 5-fold, 6-fold, 7-fold, 8-fold, 9-fold, or 10-fold higher than an expression of the biomarker in a control sample. In some embodiments, the expression of the biomarker is about 1-fold, 2-fold, 3-fold, 4-fold, 5-fold, 6-fold, 7-fold, 8-fold, 9-fold, or 10-fold lower than an expression of the biomarker in a biological sample obtained from the subject or patient at a different timepoint (e.g., during treatment course). In some embodiments, the expression of the biomarker is about 1-fold, 2-fold, 3-fold, 4-fold, 5-fold, 6-fold, 7-fold, 8-fold, 9-fold, or 10-fold higher than an expression of the biomarker in a biological sample obtained from the subject or patient at a different timepoint (e.g., during treatment course). In some embodiments, the expression of the biomarker is about 1-fold, 2-fold, 3-fold, 4-fold, 5-fold, 6-fold, 7-fold, 8-fold, 9-fold, or 10-fold lower than an expression of the biomarker in a different biological sample obtained from the same subject, such as a biological sample from the colon of the subject. In some embodiments, the expression of the biomarker is about 1-fold, 2-fold, 3-fold, 4-fold, 5-fold, 6-fold, 7-fold, 8-fold, 9-fold, or 10-fold higher than an expression of the biomarker in a different biological sample obtained from the same subject, such as a biological sample from the colon of the subject. In some embodiments, the expression of the biomarker in a biological sample obtained from the small bowel is at least 10-fold higher than the expression of the biomarker in the colon.

### B. Systems

Provided herein are systems of analyzing gene or gene products (e.g., mRNA, cDNA, protein) in a biological sample obtained from a subject to diagnose, prognose, treat, or monitor a treatment for, a disease or a condition described herein, such as inflammatory bowel disease (IBD). In some embodiments, a biological sample obtained from a subject (directly or indirectly) is analyzed for an expression level of one or more biomarkers provided in Table 1. In some embodiments, the subject is administered a therapeutically effective amount of a therapeutic agent described herein, provided the expression level of the one or more biomarkers is above or below a certain threshold value. In some embodiments, the threshold value is determined based, at least in part, by the expression of the one or more biomarkers in a control sample (e.g., a sample obtained from a non-diseased subject, a different type of sample obtained from the subject, or a sample obtained from the subject at a different type point, such as before or after a treatment course). In some embodiments, the threshold value is an absolute number of copies of the one or more biomarkers. In some embodiments, the threshold is a relative expression (e.g., fold change).

In some embodiments, disclosed herein is a system comprising: (a) a computer processing device, optionally connected to a computer network; and (b) a software module executed by the computer processing device to analyze genes or gene products described above, and provided in Table 1, in a sample obtained from a subject. In some instances, the system comprises a central processing unit (CPU), memory (e.g., random access memory, flash memory), electronic storage unit, computer program, communication interface to communicate with one or more other systems, and any combination thereof. In some instances, the system is coupled to a computer network, for example, the Internet, intranet, and/or extranet that is in communication with the Internet, a telecommunication, or data network. In some embodiments, the system comprises a storage unit to store data and information regarding any aspect of the methods described in this disclosure. Various aspects of the system are a product or article or manufacture.

One feature of a computer program includes a sequence of instructions, executable in the digital processing device's CPU, written to perform a specified task. In some embodiments, computer readable instructions are implemented as program modules, such as functions, features, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions are combined or distributed as desired in various environments. In some instances, a computer program comprises one sequence of instructions or a plurality of sequences of instructions. A computer program may be provided from one location. A computer program may be provided from a plurality of locations. In some embodiment, a computer program includes one or more software modules. In some embodiments, a computer program includes, in part or in whole, one or more web applications, one or more mobile applications, one or more standalone applications, one or more web browser plug-ins, extensions, add-ins, or add-ons, or combinations thereof

4. Web Application

In some embodiments, a computer program includes a web application. In light of the disclosure provided herein, those of skill in the art will recognize that a web application may utilize one or more software frameworks and one or more database systems. A web application, for example, is created upon a software framework such as Microsoft® .NET or Ruby on Rails (RoR). A web application, in some instances, utilizes one or more database systems including, by way of non-limiting examples, relational, non-relational, feature oriented, associative, and XML database systems. Suitable relational database systems include, by way of non-limiting examples, Microsoft® SQL Server, mySQL™, and Oracle®. Those of skill in the art will also recognize that a web application may be written in one or more versions of one or more languages. In some embodiments, a web application is written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™ JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash®, HTML 5, Apple® QuickTime®, Microsoft® Silverlight®, Java™, and Unity®.

5. Mobile Application

In some instances, a computer program includes a mobile application provided to a mobile digital processing device. The mobile application may be provided to a mobile digital processing device at the time it is manufactured. The mobile application may be provided to a mobile digital processing device via the computer network described herein.

A mobile application is created by techniques known to those of skill in the art using hardware, languages, and development environments known to the art. Those of skill in the art will recognize that mobile applications may be written in several languages. Suitable programming languages include, by way of non-limiting examples, C, C++, C#, Featureive-C, Java™, Javascript, Pascal, Feature Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

6. Standalone Application

In some embodiments, a computer program includes a standalone application, which is a program that may be run as an independent computer process, not an add-on to an existing process, e.g., not a plug-in. Those of skill in the art will recognize that standalone applications are sometimes compiled. In some instances, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Python™, Visual Basic, and VB .NET, or combinations thereof. Compilation may be often performed, at least in part, to create an executable program. In some instances, a computer program includes one or more executable complied applications.

7. Web Browser Plug-in

A computer program, in some aspects, includes a web browser plug-in. In computing, a plug-in, in some instances, is one or more software components that add specific functionality to a larger software application. Makers of software applications may support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. The toolbar may comprise one or more web browser extensions, add-ins, or add-ons. The toolbar may comprise one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

In some embodiments, Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. The web browser, in some instances, is a mobile web browser. Mobile web browsers (also called mircrobrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

8. Software Modules

The medium, method, and system disclosed herein comprise one or more softwares, servers, and database modules, or use of the same. In view of the disclosure provided herein, software modules may be created by techniques known to those of skill in the art using machines, software, and languages known to the art. The software modules disclosed herein may be implemented in a multitude of ways. In some embodiments, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprises a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

9. Databases

The medium, method, and system disclosed herein comprise one or more databases, or use of the same. In view of the disclosure provided herein, those of skill in the art will recognize that many databases are suitable for storage and retrieval of geologic profile, operator activities, division of interest, and/or contact information of royalty owners. Suitable databases include, by way of non-limiting examples, relational databases, non-relational databases, feature oriented databases, feature databases, entity-relationship model databases, associative databases, and XML databases. In some embodiments, a database is internet-based. In some embodiments, a database is web-based. In some embodiments, a database is cloud computing-based. A database may be based on one or more local computer storage devices.

10. Data Transmission

The subject matter described herein, are configured to be performed in one or more facilities at one or more locations. Facility locations are not limited by country and include any country or territory. In some instances, one or more steps of a method herein are performed in a different country than another step of the method. In some instances, one or more steps for obtaining a sample are performed in a different country than one or more steps for analyzing a genotype of a sample. In some embodiments, one or more method steps involving a computer system are performed in a different country than another step of the methods provided herein. In some embodiments, data processing and analyses are performed in a different country or location than one or more steps of the methods described herein. In some embodiments, one or more articles, products, or data are transferred from one or more of the facilities to one or more different facilities for analysis or further analysis. An article includes, but is not limited to, one or more components obtained from a sample of a subject and any article or product disclosed herein as an article or product. Data includes, but is not limited to, information regarding genotype and any data produced by the methods disclosed herein. In some embodiments of the methods and systems described herein, the analysis is performed and a subsequent data transmission step will convey or transmit the results of the analysis.

In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, such as cellular material, performed in one facility in a particular location and corresponding data transmitted to another location, or directly to an individual, such as data related to the diagnosis, prognosis, responsiveness to therapy, or the like, in the same or different location or country.

### C. Kits

Disclosed herein, in some embodiments, are kits useful for to detect the biomarkers disclosed herein. In some embodiments, the kits disclosed herein may be used to diagnose and/or treat a disease or condition in a subject; or select a patient for treatment and/or monitor a treatment disclosed herein. In some embodiments, the kit comprises the compositions described herein, which can be used to perform the methods described herein. Kits comprise an assemblage of materials or components, including at least one of the compositions. Thus, in some embodiments the kit contains a composition including of the pharmaceutical composition, for the treatment of IBD. In other embodiments, the kits contains all of the components necessary and/or sufficient to perform an assay for detecting and measuring IBD markers, including all controls, directions for performing assays, and any necessary software for analysis and presentation of results.

In some instances, the kits described herein comprise components for detecting the presence, absence, and/or quantity of a target nucleic acid and/or protein described herein. In some embodiments, the kit comprises the compositions (e.g., primers, probes, antibodies) described herein. The disclosure provides kits suitable for assays such as enzyme-linked immunosorbent assay (ELISA), single-molecular array (Simoa), PCR, and qPCR. The exact nature of the components configured in the kit depends on its intended purpose. For example, some embodiments are configured for the purpose of treating a disease or condition disclosed herein (e.g., IBD, CD, UC) in a subject. In some embodiments, the kit is configured particularly for the purpose of treating mammalian subjects. In some embodiments, the kit is configured particularly for the purpose of treating human subjects. In further embodiments, the kit is configured for veterinary applications, treating subjects such as, but not limited to, farm animals, domestic animals, and laboratory animals. In some embodiments, the kit is configured to select a subject for a therapeutic agent, such as those disclosed herein.

Instructions for use may be included in the kit. In some embodiments, the instructions are for evaluating whether a therapeutic regimen is therapeutically effective to treat a disease or a condition of a subject, based at least in part, on the expression of the one or more biomarkers detected in a biological sample obtained from the subject. In some embodiments, the instructions are for evaluating whether to administer a therapeutic agent disclosed herein to the subject to treat the disease or the condition of a subject, based at least in part, on the expression of the one or more biomarkers detected in a biological sample obtained from the subject. In some embodiments, the instructions are for how to perform the steps described herein for detecting the one or more biomarkers in a biological sample, including preparing the biological sample, isolating the genomic sub-cellular components, and performing one of the assays described herein.

Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of the pharmaceutical composition. The packaging material has an external label which indicates the contents and/or purpose of the kit and its components.

Disclosed herein are methods of contacting a sub-cellular component of a biological sample obtained from a subject with a probe described herein, or using the kit described herein under conditions configured to hybridize the probe to the sub-cellular component. In further embodiments, provided herein are methods of treating the subject with a therapeutic agent disclosed herein, provided that the sub-cellular component from the subject is detected using the kit.

### D. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The term “biomarker” comprises a measurable substance in a subject whose presence, level, or activity, is indicative of a phenomenon (e.g., phenotypic expression or activity; disease, condition, subclinical phenotype of a disease or condition, infection; or environmental stimuli). In some embodiments, a biomarker comprises a gene, gene expression product (e.g., RNA or protein), or a cell-type (e.g., immune cell).

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, the term “about” a number refers to that number plus or minus 10% of that number. The term “about” a range refers to that range minus 10% of its lowest value and plus 10% of its greatest value.

The terms, “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease. Other examples of “decrease” include a decrease of at least 2-fold, at least 5-fold, at least 10-fold, at least 20-fold, at least 50-fold, at least 100-fold, at least 1000-fold or more as compared to a reference level.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence. A “genetic locus” referred to herein, is a particular location within a gene.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J Mol Biol. 1990 Oct. 5; 215(3):403-10; Nucleic Acids Res. 1997 Sep. 1; 25(17):3389-402). Percent homology of sequences can be determined using the most recent version of BLAST, as of the filing date of this application. Percent identity of sequences can be determined using the most recent version of BLAST, as of the filing date of this application.

The terms “increased” or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The term “inflammatory bowel disease” or “IBD” as used herein refers to gastrointestinal disorders of the gastrointestinal tract. Non-limiting examples of IBD include, Crohn's disease (CD), ulcerative colitis (UC), indeterminate colitis (IC), microscopic colitis, diversion colitis, Behcet's disease, and other inconclusive forms of IBD. In some instances, IBD comprises fibrosis, fibrostenosis, stricturing and/or penetrating disease, obstructive disease, or a disease that is refractory (e.g., mrUC, refractory CD), perianal CD, or other complicated forms of IBD.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

The term “in vivo” is used to describe an event that takes place in a subject's body.

The term “medically refractory,” or “refractory,” as used herein, refers to the failure of a standard treatment to induce remission of a disease. In some embodiments, the disease comprises an inflammatory disease disclosed herein. A non-limiting example of refractory inflammatory disease includes refractory Crohn's disease, and refractory ulcerative colitis (e.g., mrUC). Non-limiting examples of standard treatment include glucocorticosteriods, anti-TNF therapy, anti-a4-b7 therapy (vedolizumab), anti-IL12p40 therapy (ustekinumab), Thalidomide, and Cytoxin.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, oral, injection, aerosol, parenteral, and topical administration.

The terms “response,” or “responsive,” as used herein in reference to a subject's reaction to a therapeutic agent, refers to phenomena in which a subject or a patient responds to the induction of a therapy, or a “successful induction” of the therapy, which may in some cases, be an initial therapeutic response or benefit provided by the therapy. By contrast, the terms “non-response,” or “loss-of-response,” as used herein, refer to phenomena in which a subject or a patient does not respond to the induction of a standard treatment (e.g., anti-TNF therapy), or experiences a loss of response to the standard treatment after a successful induction of the therapy. The induction of the standard treatment may include 1, 2, 3, 4, or 5, doses of the therapy. A “successful induction” of the therapy may be an initial therapeutic response or benefit provided by the therapy. The loss of response may be characterized by a reappearance of symptoms consistent with a flare after a successful induction of the therapy.

The terms “subject,” or “individual,” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease. In some embodiments, the subject is a “patient,” who has a disease or a condition disclosed herein.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### E. Examples

The following examples are included for illustrative purposes only and are not intended to limit the scope of the invention.

### Example 1: Methods and Materials

Tissue Samples and Study Subjects

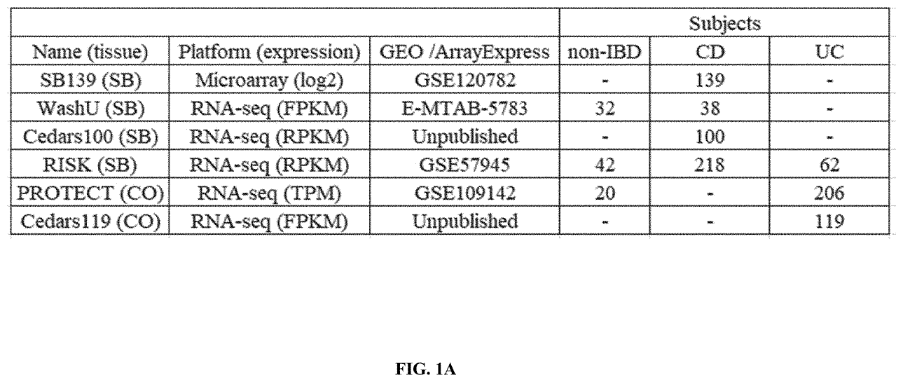

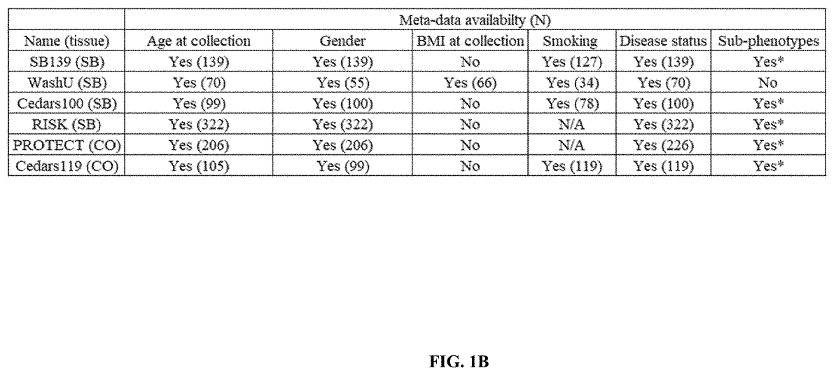

The association of ACE2 mRNA with age at collection, gender, smoking, BMI, diagnosis, disease sub-phenotypes in six independent transcriptomic datasets (FIGS. 1A-1B) of either small bowel gene or colon contingent on cohort-specific meta-data availability.

All specimens from the CD cohorts (SB139, WashU, and Cedars100) cohorts were from macroscopically and microscopically non-inflamed small bowel. All specimens from the UC cohorts (PROTECT, Cedars119) were from macroscopically and microscopically non-inflamed colon.

The ‘SB139’ dataset was generated using whole Human Genome 4×44k Microarrays [Agilent] from formalin fixed paraffin embedded (FFPE) tissue taken from the unaffected margin of SB tissue resected during ileo-cecal or small bowel resection for complicated CD. Median age at time of surgery, which were all performed at Cedars-Sinai Medical Center, Los Angeles, was 32 years. The ‘WashU’ dataset was generated by RNA-seq and similarly was generated from FFPE tissue from the unaffected proximal margin of resected CD tissues and also from FFPE from control (non-IBD) subjects. These subjects had a median age of 51 years at time of surgery which were all performed at the University of Washington, St Louis. The SB139 and WashU samples were all reviewed by a single pathologist (TSS) excluding any samples with microscopic evidence of inflammation. The RISK dataset was generated by RNA-seq from ileal biopsies taken from pediatric subjects in a CD inception cohort from multiple centers across North America (median age at time of biopsy 12 years at the time of biopsy). The age at diagnosis for this cohort is same as the age of subject at specimen collection. CD subjects in RISK cohort had biopsies taken from subjects where the SB/ileum was unaffected (cCD) and others where the ileum was involved (iCD). The Cedars100 dataset has not been previously published but was similarly generated from FFPE from uninvolved proximal resection margins from complicated CD surgeries (performed at Cedars-Sinai Medical Center) and transcriptomics were generated by RNA-seq after review of TSS as described earlier. All study subjects in SB139 and Cedars100 were CD; the WashU cohort consisted of CD and controls (non-IBD) and RISK cohort is a mix of CD, UC, and controls (non-IBD). In three of the four SB cohorts, specimens were taken from macroscopically normal appearing tissue. The RISK cohort had samples from both inflamed (iCD) as well macroscopically normal appearing tissue (cCD)

The PROTECT cohort consisted of pediatric subjects with varying degrees of disease severity in a UC inception cohort from multiple centers across North America (median age at time of biopsy, 13 years). Transcriptomics were used from a sub-cohort of 206 UC subjects with baseline rectal biopsies prior to instigation of any IBD therapy along with 20 non-IBD controls. The Cedars119 cohort has not been previously published and consists of 119 UC subjects with varying disease severity (median age of 42 years, Mayo endoscopy sub score range of 0-3) treated at CSMC. Transcriptomics for Cedars119 cohort was generated from rectal biopsies using RNA-seq.

The effect of drug exposure on small bowel and colonic ACE2 expression was analyzed from three clinical trials investigating biologic therapies used in IBD: Infliximab (IFX cohort), NCT00639821, GSE16879; and ustekinumab (CERTIFI trial), NCT00771667, GSE100833 and ustekinumab (UNITI-2 induction and maintenance) NCT01369342, GSE112366. For the UNITI-2 trial, ileal histologic activity was quantified based on modified global histology activity score (GHAS) and endoscopic activity was quantified by simple endoscopic score for Crohn's Disease (SES-CD).

The transcriptomics for the IFX cohort were generated using Affymetrix Human Genome U133 Plus 2.0 microarray platform using biopsies from inflamed mucosa (n=61 IBD subjects) before and 4-6 weeks after first infliximab infusion and in normal mucosa from 12 control patients (6 colon and 6 ileum). The patients were classified as responders/non-responders for treatment based on endoscopic and histologic findings at 4-6 weeks after Infliximab induction treatment.

The CERTIFI trial consists of microarray (Affymetrix HT HG-U133+PM Array Plate) transcriptomics of human blood and intestinal Biopsy Samples from a Phase 2b, Double-blind, Placebo-controlled Study of Ustekinumab in Crohn's Disease. The cohort contained gene expression on 329 Crohn's biopsies from multiple regions in the intestine of 87 anti-TNFa refractory patients. For consistency, only SB ileal transcriptomics was analyzed for the purpose of this study. Response outcomes to ustekinumab were not available for this cohort.

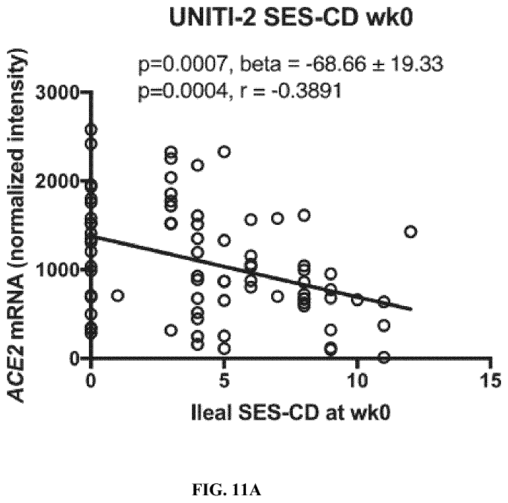

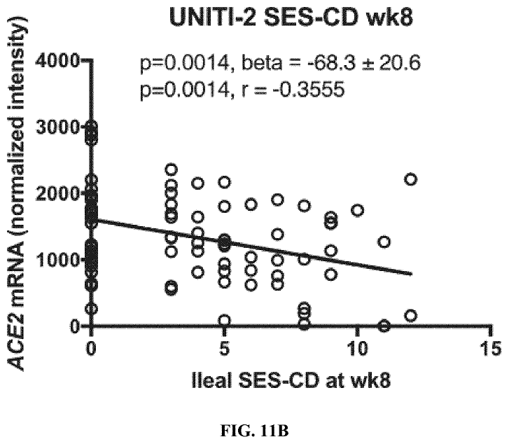

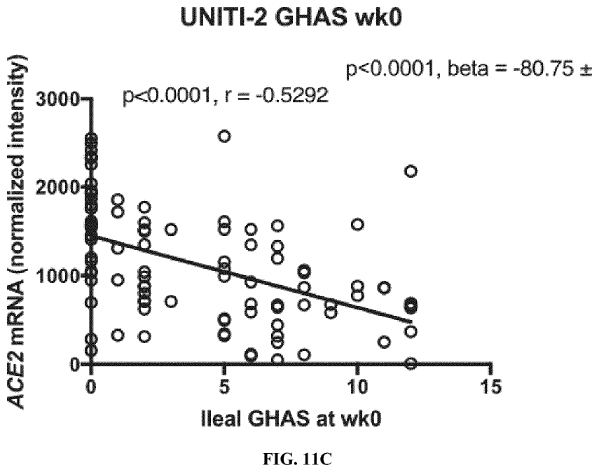

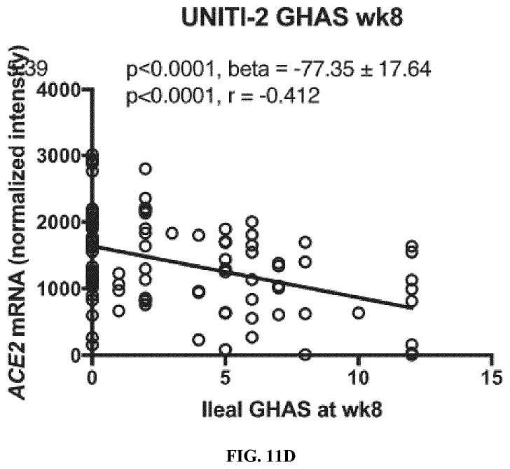

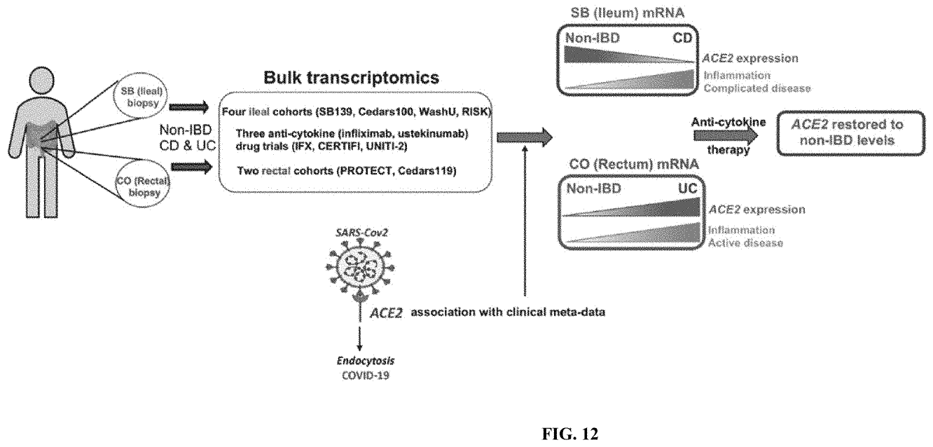

The UNITI-2 induction and maintenance trial consists of microarray (Affymetrix HT HG-U133+PM Array Plate) transcriptomics of terminal ileum biopsy samples collected at baseline, 8 weeks after induction (Ustekinumab or placebo), and 44 weeks after maintenance (Ustekinumab 90 mg SC q12w, Ustekinumab 90 mg SC q8w, or placebo) from patients with moderate-to-severe CD who participated in phase 3 studies. Ileal biopsy specimens were taken from patients with ileal or ileocolonic CD (n=110) as well as non-IBD controls (n=26). Ileal histologic activity was quantified based on modified global histology activity score (GHAS) and endoscopic activity was quantified by simple endoscopic score for Crohn's Disease (SES-CD). FIG. 11A-11D show an inverse correlation between ACE2 expression and increasing severity of inflammation as measured by macroscopic and microscopic criteria (ileal GHAS and SES-CD).

Transcriptomics Data Generation and Processing

The Genome Technology Access Center at Washington University (St Louis, Mo.) generated datasets in the SB139, WashU and Cedars100 cohorts. The methods used to generate and analyze microarray SB139 cohort data is described in Potdar, A. A., et al., Ileal Gene Expression Data from Crohn's Disease Small Bowel Resections Indicate Distinct Clinical Subgroups. Journal of Crohn's and Colitis, 2019. 3: p. 27-12, which is hereby incorporated by reference in its entirety. For the WashU cohort, RNA-seq library preparation, sequencing, and read alignment was described in VanDussen, K. L., et al., Abnormal Small Intestinal Epithelial Microvilli in Patients With Crohn's Disease. Gastroenterology, 2018. 155(3): p. 815-828, which is hereby incorporated by reference in its entirety. Sequencing for WashU was performed on an Illumina HiSeq2000 SR42 (Illumina, San Diego, Calif.) using single reads extending 42 bases.

For the Cedars100 cohort, total RNAs were processed with Sigma Seqplex to create amplified ds-cDNA, followed by traditional Illumina library preparation with unique dual indexing. 100 libraries were run on NovaSeq6000, S2 flow cell, using single-end 100 base reads. The run generated approximately 4.2B reads passing filter, thus an average of 42 million reads per library were generated. The data for the other three cohorts (RISK, IFX, UST) were generating using methods described in Haberman, Y., et al., Pediatric Crohn's disease patients exhibit specific ileal transcriptome and microbiome signature. Journal of Clinical Investigation, 2014. 124(8): p. 3617-3633; Kugathasan, S., et al., Prediction of complicated disease course for children newly diagnosed with Crohn's disease: a multicentre inception cohort study. The Lancet, 2017. 389(10080): p. 1710-1718; Arijs, I., et al., Mucosal Gene Expression of Antimicrobial Peptides in Inflammatory Bowel Disease Before and After First Infliximab Treatment. PloS one, 2009. 4(11): p. e7984-10; and Peters, L. A., et al., A functional genomics predictive network model identifies regulators of inflammatory bowel disease. Nature Genetics, 2017. 49(10): p. 1437-1449, which are hereby incorporated by reference in its entirety.

The Cedars119 RNA-seq dataset was generated by EA genomics, Q² solutions. Briefly, RNA samples were converted into cDNA libraries using the Illumina TruSeq stranded mRNA sample preparation kit and hiSeq-Sequencing-2×50 bp-paired end sequencing performed on an Illumina sequencing platform. Across all samples, the median number of actual reads was 24.8 million with 23.6 million on-target reads, after removal of various sequencing artifacts and normalized data in FPKM generated.

The data generation methods were performed for the other cohorts (RISK, PROTECT, IFX, CERTIFI, UNITI-2) as provided in Arijs I, De Hertogh G, Lemaire K, et al. Mucosal Gene Expression of Antimicrobial Peptides in Inflammatory Bowel Disease Before and After First Infliximab Treatment. PLoS ONE 2009; 4:e7984-10; Peters L A, Perrigoue J, Mortha A, et al. A functional genomics predictive network model identifies regulators of inflammatory bowel disease. Nature Genetics 2017; 49:1437-1449; VanDussen K L, Stojmirovic A, Li K, et al. Abnormal Small Intestinal Epithelial Microvilli in Patients With Crohn's Disease. Gastroenterology 2018; 155:815-828; Haberman Y, Tickle T L, Dexheimer P J, et al. Pediatric Crohn's disease patients exhibit specific ileal transcriptome and microbiome signature. Journal of Clinical Investigation 2014; 124:3617-3633; Kugathasan S, Denson L A, Walters T D, et al. Prediction of complicated disease course for children newly diagnosed with Crohn's disease: a multicentre inception cohort study. The Lancet 2017; 389:1710-1718; Hyams J S, Davis S, Mack D R, et al. Factors associated with early outcomes following standardised therapy in children with ulcerative colitis (PROTECT): a multicentre inception cohort study. The Lancet Gastroenterology and Hepatology 2017; 2:855-868; and Haberman Y, Karns R, Dexheimer P J, et al. Ulcerative colitis mucosal transcriptomes reveal mitochondriopathy and personalized mechanisms underlying disease severity and treatment response. Nature Communications 2018:1-13, each of which is hereby incorporated by reference in its entirety.

The methods used to process microarray data from SB139 cohort have been previously described in Potdar, et al. The pipeline used for RNA-seq data processing and normalizing for the Cedars100 cohort was similar to the one used for the WashU cohort as previously described above. For Cedars100, RNA-seq data was normalized and resultant RPKM values were generated for analysis while for WashU normalized data were generated in FPKM. The methods used to process the RNA-seq data from RISK cohort have also described previously in Haberman et al., and Kugathasan et al., provided above.

Normalized processed data for some cohorts (RISK, PROTECT, IFX and CERTIFI) were downloaded using accession numbers available at GEO in series matrix files which were cleaned and annotated with geneids. Clean, processed data for SB139, Cedars100 and WashU along with respective meta-data was available in-house at Cedars-Sinai. UNITI-2 trial data were analyzed at Janssen.

Clinical and Demographic Data

Meta-data available for the different transcriptomics cohorts used is compiled in FIG. 1A-FIG. 1B. The ‘sub-phenotypes’ meta-data in FIG. 1A-1B includes severe versus mild refractory in SB139, involved versus un-involved SB and subsequent development of disease complication (B1=inflammatory; B2=stricturing, B3=penetrating) in RISK, disease behavior in SB139 and Cedars100, disease recurrence in SB139, meta-data on active disease and Mayo endoscopy subscore for Cedars119 and need for oral steroid or anti-TNF rescue therapy by week 52 in the PROTECT cohort.

The ‘SB139’ and ‘Cedars100’ datasets were generated from ileal biopsies of CD subjects requiring surgery at Cedars-Sinai Medical Center. Subjects in SB139 and Cedars100 have been followed prospectively since surgery. For these cohorts clinical and demographic data were obtained from the prospective database. Clinical phenotype data available for SB139 included age at collection, gender, disease location/severity, disease recurrence after surgery. The Cedars100 cohort included gender, smoking status but did not include age at collection and BMI.

For the ‘WashU cohort, data were extracted from the clinical charts and includes age at collection, gender, disease status, smoking and BMI at collection. Some meta-data for RISK cohort were downloaded from NCBI (GEO/SRA) such as age at collection, gender and disease diagnosis, including information for involved versus unaffected CD but complication data were available from the prospective follow up. Meta-data for IFX, CERTIFI and UNITI-2 trials was downloaded from their respective GEO accession numbers. Some meta-data for PROTECT cohort were downloaded from NCBI (GEO) including age at collection, gender, diagnosis but need for ‘rescue’ medication data were available from the prospective follow up.

Meta-data for IFX (GSE100833) and UST (GSE100833) cohorts was downloaded from their respective GEO accession numbers.

Methods for Datasets Downloaded Via GEO:

Platform annotation, normalized gene expression, and phenotype meta-data were extracted using the R package GEOquery (GEO2R library). The phenotype meta-data table was used to identify categories such as tissue type (non-involved/inflamed terminal ileum biopsy tissue samples), disease status (Control, CD, UC), time points (defined as week 0 and week 6) for treatment, treatment type, etc. as available depending on the cohort.

Univariate and Multivariate Model Fits:

Univariate models were fitted with ACE2 or TMPRSS2 or TMPRSS4 as response and each available demographic data (age, gender, BMI at surgery, smoking status) as a predictor in each cohort. A similar pipeline was followed for clinical predictors such as disease status, CD severity sub-groups, recurrence, and treatment when available in a given cohort. This was followed by fitting multivariate models with ACE2 expression as response and all available predictors within each cohort.

In some cohorts (WashU and RISK), multivariate models were also fitted for other COVID-19 relevant genes such as ACE, TMPRSSS2 and SLC6A19 with response and age, gender and disease status as predictors. The relationship between ACE2 expression and disease recurrence (only available in SB139) was analyzed through a multivariate model with age, gender and first two principal components in genotype data calculated using genetic data published previously in Potdar et al and described above. An association between ACE2 with CD disease behavior B1, B2 and B3 (available in SB139, Cedars100 and RISK) using age and gender as covariates was also performed.

Statistical Tools

Statistical package glm in R (version 3.5.1) was used to perform univariate and multivariate associations with a p<0.05 cutoff as statistical significance. In some cases, GraphPad Prism? (La Jolla, Calif.) was used to perform t or Mann-Whitney test. Kruskal-Wallis test (non-parametric data) was used to compare the differences across multiple groups and adjusted p value (padj) reported for pair-wise comparisons.

ACE2 Gene Co-Expression Analysis

Co-expression analysis of ACE2 with many (˜54) genes of interest involved in either IBD pathogenesis or high probability SARS CoV-2 virus-host protein-protein interaction was performed using the SB139 and Cedars100 cohorts using methods described in Cheng, C., et al., Identification of differentially expressed genes, associated functional terms pathways, and candidate diagnostic biomarkers in inflammatory bowel diseases by bioinformatics analysis. Experimental and Therapeutic Medicine, 2019: p. 1-11 and Gordon, D. E., et al., A SARS-CoV-2-Human Protein-Protein Interaction Map Reveals Drug Targets and Potential Drug-Repurposing. bioRxiv, 2020, which are hereby incorporated by reference in entirety. Genomic annotations for candidate genes of interest were extracted at the probe/transcript level from the platform annotation file for SB139 and Cedars100 [R based GenomicFeatures package in Bioconductor]. The statistical package glm was used to fit a multivariate linear regression model on the gene pairs and included covariates, such as age at collection and gender (when available) with a p<0.05 cutoff as statistical significance. The full list of genes examined in the co-expression analysis are available in Table 1.

TABLE 1
List of candidate genes used for co-expression analysis
with ACE2 from two sources, IBD pathogenesis and high
probability in viral-host protein-protein interaction

| Candidate | |
|---|---|
| Gene | Source |
| ADAM17 | Implicated in IBD Pathogenesis |
| IL6 | Implicated in IBD Pathogenesis |
| IL8 | Implicated in IBD Pathogenesis |
| IL12 | Implicated in IBD Pathogenesis |
| IL17 | Implicated in IBD Pathogenesis |
| IL23 | Implicated in IBD Pathogenesis |
| IL23R | Implicated in IBD Pathogenesis |
| IL12A | Implicated in IBD Pathogenesis |
| IL12B | Implicated in IBD Pathogenesis |
| IL23A | Implicated in IBD Pathogenesis |
| IFNG | Implicated in IBD Pathogenesis |
| JAK1 | Implicated in IBD Pathogenesis |
| JAK3 | Implicated in IBD Pathogenesis |
| TNF | Implicated in IBD Pathogenesis |
| ITGA4 | Implicated in IBD Pathogenesis |
| ITGB7 | Implicated in IBD Pathogenesis |
| AGTR1 | Implicated in IBD Pathogenesis |
| ACE | High Probability in Viral-Host Protein-Protein Interaction |
| TMPRSS2 | High Probability in Viral-Host Protein-Protein Interaction |
| TMPRSS4 | High Probability in Viral-Host Protein-Protein Interaction |
| SLC6A15 | High Probability in Viral-Host Protein-Protein Interaction |
| ABCC1 | High Probability in Viral-Host Protein-Protein Interaction |
| MARK2 | High Probability in Viral-Host Protein-Protein Interaction |
| MARK3 | High Probability in Viral-Host Protein-Protein Interaction |
| RIPK1 | High Probability in Viral-Host Protein-Protein Interaction |
| CSNK2A2 | High Probability in Viral-Host Protein-Protein Interaction |
| CSNK2B | High Probability in Viral-Host Protein-Protein Interaction |
| NEK9 | High Probability in Viral-Host Protein-Protein Interaction |
| HDAC2 | High Probability in Viral-Host Protein-Protein Interaction |
| SIGMAR1 | High Probability in Viral-Host Protein-Protein Interaction |
| TMEM97 | High Probability in Viral-Host Protein-Protein Interaction |
| NDUFs | High Probability in Viral-Host Protein-Protein Interaction |
| GLA | High Probability in Viral-Host Protein-Protein Interaction |
| PLOD1 | High Probability in Viral-Host Protein-Protein Interaction |
| PLOD2 | High Probability in Viral-Host Protein-Protein Interaction |
| PTGES2 | High Probability in Viral-Host Protein-Protein Interaction |
| IMPDH2 | High Probability in Viral-Host Protein-Protein Interaction |
| LARP1 | High Probability in Viral-Host Protein-Protein Interaction |
| FKBP15 | High Probability in Viral-Host Protein-Protein Interaction |
| FKBP7 | High Probability in Viral-Host Protein-Protein Interaction |
| FKBP10 | High Probability in Viral-Host Protein-Protein Interaction |
| COMT | High Probability in Viral-Host Protein-Protein Interaction |
| BRD2 | High Probability in Viral-Host Protein-Protein Interaction |
| BRD4 | High Probability in Viral-Host Protein-Protein Interaction |
| DNMT1 | High Probability in Viral-Host Protein-Protein Interaction |
| VCP | High Probability in Viral-Host Protein-Protein Interaction |
| CUL2 | High Probability in Viral-Host Protein-Protein Interaction |
| CEP250 | High Probability in Viral-Host Protein-Protein Interaction |
| EIF4E2 | High Probability in Viral-Host Protein-Protein Interaction |
| EIF4EH | High Probability in Viral-Host Protein-Protein Interaction |
| F2RL1 | High Probability in Viral-Host Protein-Protein Interaction |
| ATP6AP1 | High Probability in Viral-Host Protein-Protein Interaction |
| LOX | High Probability in Viral-Host Protein-Protein Interaction |
| PRKACA | High Probability in Viral-Host Protein-Protein Interaction |
| SLC1A3 | High Probability in Viral-Host Protein-Protein Interaction |
| DCTPP1 | High Probability in Viral-Host Protein-Protein Interaction |
| TBK1 | High Probability in Viral-Host Protein-Protein Interaction |

ACE2 Whole Exome Sequencing

Paired-end whole exome sequencing (WES) was performed based on Illumina platform with 20× reading depth in 2,712 IBD subjects (CD=1574, UC=1130 and Indeterminate Colitis=8). Read alignment to the human reference genome GRCh37 were performed using BWA and variant calling were performed based on GATK best practices. Individual variants with Genotyping Quality (GQ)<65, depth (DP)<20, Strand Odds Ratio (SOR)>3 or call rate <95% were removed. For SNPs, variants with ReadPosRankSum<−4 or Fisher Strand filter (FS) >60 were also removed. For indels, variants with ReadPosRankSum<−20 or FS>200 were also removed. In total, 3,349,656 variants passed quality control (QC). Samples with a mean genotype quality (GQ)<65, a depth <25, a genotype rate <96.5%, or a transition/transversion (Ti/Tv) ratio <2.5 were removed from further analyses. Individuals of ambiguous imputed sex or of imputed sex inconsistent with reported sex were also removed. A total of 2,590 samples (CD=1463, UC=1119 and Indeterminate=8) passed QC. Allele frequencies (AF) of European population of individual variants were obtained from the Genome Aggregation Database (gnomAD; http://gnomad.broadinstitute.org/), Functional annotations of individual variants were added using ANNOVAR. For deleteriousness prediction, Combined Annotation-Dependent Depletion tool (CADD) was used. Variants located within ACE2 (chrX:15,579,156-15,620,271; GRCh37) were extracted. Among these ACE2 located variants, variants which are rare (MAF<=1% in gnomAD of European), high CADD score (CADD PHRED>10), and functionally meaningful variants (i.e. not synonymous variants) were extracted.

### Example 2: Results

Differences in ACE2 Gene Expression with Age, BMI, Disease, Smoking and Gender

Univariate Associations:

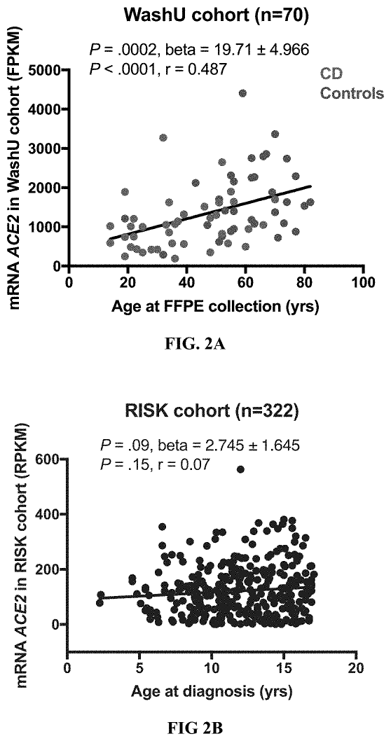

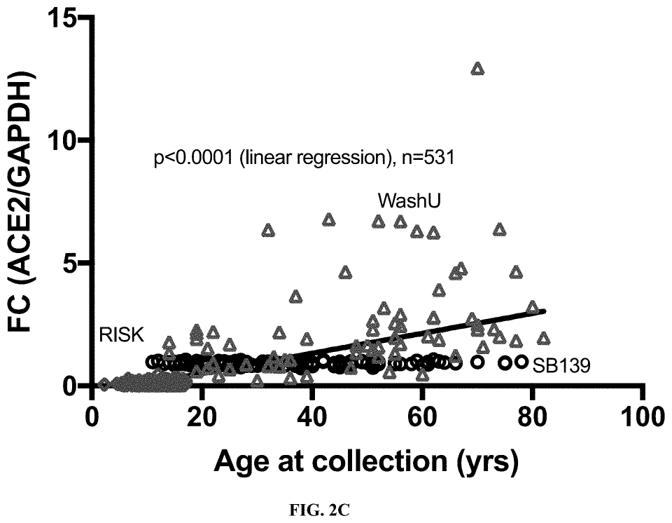

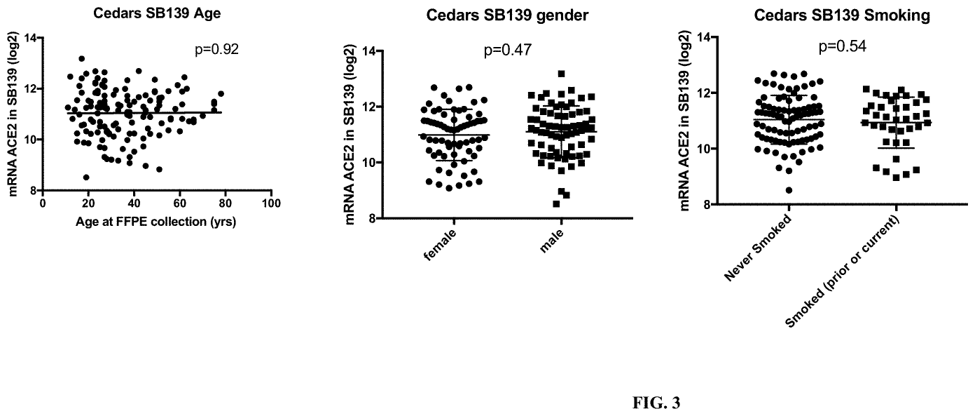

ACE2 mRNA expression by age of the subject at the time of specimen collection was analyzed where this was available. The expression of the most abundantly expressed ACE2 transcript isoform (ENST00000252519) was associated with age at collection in the WashU cohort (FIG. 2A) with higher expression being associated with older age at collection. This was true in CD and controls. The association with age trended towards significance in the pediatric RISK cohort (FIG. 2B). Statistically significant association with age in the microarray platform based SB139 cohort was not observed (FIG. 3, Table 4), and Cedars100 cohort (Table 5) as well as colonic cohorts, PROTECT (Table 6) and Cedars119 (Table 7). Combining SB139, WashU and RISK cohorts to generate fold-change of ACE2 gene expression with respect to the house-keeping gene GAPDH in the respective cohorts, validated the positive correlation of age at specimen collection with ACE2 (FIG. 2C).

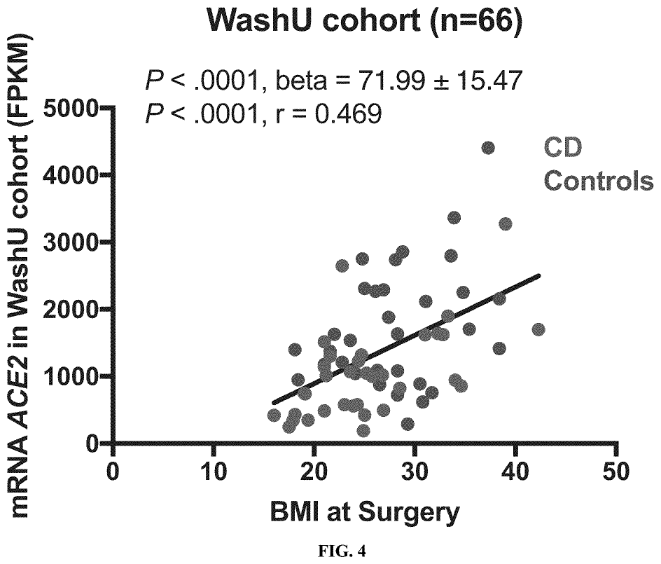

In the WashU cohort, strong association of ACE2 expression with BMI in both CD and controls with higher BMI subjects having elevated ACE2 expression was observed (p<0.0001, linear regression) (FIG. 4).

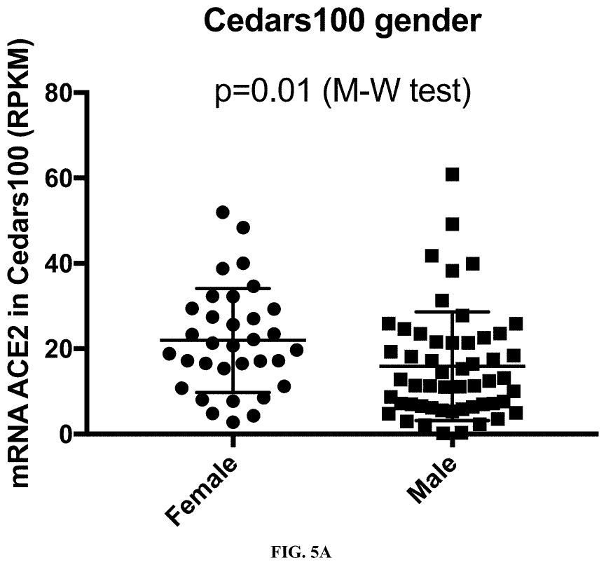

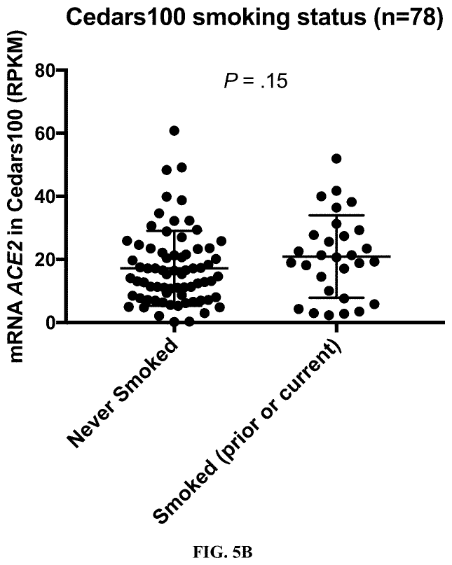

Significant association with gender in SB139, WashU and RISK cohorts was not observed (FIG. 3, Table 2, Table 3, Table 4). However, higher expression of ACE2 in females was observed in the Cedars100 cohort (FIG. 5A).

TABLE 2
Univariate and multivariate models of ACE2 mRNA associations in
the WashU cohort. Tested variables are indicated in parenthesis.

| | | | |
|---|---|---|---|
| Response: ACE2 (FPKM) | Beta | P | N |

Univariate

| | | | |
|---|---|---|---|
| BMI at surgery | 71.99 | 0.000017 | 66 |
| Age at collection | 19.71 | 0.000176 | 70 |
| Disease status (Control) | 684.30 | 0.000515 | 70 |
| Gender (Female) | −5.56 | 0.979007 | 55 |
| Smoking (Yes) | 146.90 | 0.523000 | 35 |

Multivariate

| | | | |
|---|---|---|---|
| BMI at surgery | 51.37 | 0.002 | 51 |
| Age at collection | 5.65 | 0.420 | 51 |
| Disease status (Control) | 487.68 | 0.052 | 51 |
| Gender (Female) | 78.47 | 0.672 | 51 |
| Smoking (Yes) | — | — | — |
| BMI at surgery | — | — | — |
| Age at collection | 9.42 | 0.167 | 55 |
| Disease status (Control) | 550.56 | 0.039 | 55 |
| Gender (Female) | −30.08 | 0.873 | 55 |
| Smoking (Yes) | — | — | — |
| BMI at surgery | — | — | — |
| Age at collection | 13.49 | 0.036 | 70 |
| Disease status (Control) | 369.78 | 0.120 | 70 |
| Gender (Female) | — | — | — |
| Smoking (Yes) | — | — | — |

TABLE 3
Univariate and multivariate models of ACE2 mRNA associations in
the RISK cohort. Tested variables are indicated in parenthesis.

| | Univariate | | Multivariate | |
|---|---|---|---|---|
| ACE2 (RPKM) | Beta | P | Beta | P |
| AU (n = 322) | | | | |
| Age at diagnosis | 2.745 | 0.0963 | 3.368 | 0.023 |
| Disease status (non-IBD) | 109.922 | 9.78E−14 | 113.091 | 2.14E−14 |
| Disease status (UC) | 73.518 | 3.13E−09 | 72.099 | 5.30E−09 |
| Gender(male) | −3.042 | 0.774 | −3.522 | 0.70886 |
| CD only (n = 218) | | | | |
| Age at diagnosis | 1.464 | 0.388 | 1.1361 | 0.494 |
| Gender(male) | −0.196 | 0.985 | 0.9999 | 0.922 |
| CD_type(iCD) | −41.12 | 4.86E−04 | −40.7184 | 5.93E−04 |

TABLE 4
Univariate and multivariate models for predictors
of ACE2, TMPRSS2 and TMPRSS4 expression in SB139.

| | Univariate | | | Multivariate | | |
|---|---|---|---|---|---|---|
| SB139 | Beta | P | N | Beta | P | N |
| Response: ACE2 | | | | | | |
| (log2 expression) | | | | | | |
| Age at collection | 4.77E−04 | 0.925 | 139 | 0.0058 | 0.276 | 125 |
| Gender (female) | −0.112 | 0.475 | 139 | −0.12 | 0.448 | 125 |
| Smoking (Yes) | −0.106 | 0.537 | 127 | −0.16 | 0.381 | 125 |
| Response: TMPRSS2 | | | | | | |
| (log2 expression) | | | | | | |
| Age at collection | 4.90E−04 | 0.116 | 139 | 5.50E−04 | 0.11 | 125 |
| Gender (female) | 0.0061 | 0.53 | 139 | 0.0012 | 0.904 | 125 |
| Smoking (Yes) | 0.008 | 0.49 | 127 | 0.0012 | 0.914 | 125 |
| Response: TMPRSS4 | | | | | | |
| (log2 expression) | | | | | | |
| Age at collection | −3.60E−04 | 0.262 | 139 | −2.20E−04 | 0.52 | 125 |
| Gender (female) | −0.011 | 0.27 | 139 | −0.009 | 0.386 | 125 |
| Smoking (Yes) | −0.009 | 0.43 | 127 | −0.0055 | 0.647 | 125 |

TABLE 5
Univariate and multivariate models for predictors of
ACE2, TMPRSS2 and TMPRSS4 expression in Cedars100.

| | Univariate | | | Multivariate | | |
|---|---|---|---|---|---|---|
| Cedars100 | Beta | P | N | Beta | P | N |
| Response: ACE2 (RPKM) | | | | | | |
| Age at collection | 0.003 | 0.96 | 100 | 0.018 | 0.79 | 97 |
| Gender (female) | 6.08 | 0.017 | 99 | 6.06 | 0.02 | 97 |
| Smoking (Yes) | 3.68 | 0.17 | 100 | 3.17 | 0.25 | 97 |
| Response: TMPRSS2 (RPKM) | | | | | | |
| Age at collection | 0.197 | 0.014 | 100 | 0.189 | 0.015 | 97 |
| Gender (female) | 6.61 | 0.036 | 99 | 7.67 | 0.01 | 97 |
| Smoking (Yes) | 10.96 | 0.00091 | 100 | 9.14 | 0.0045 | 97 |
| Response: TMPRSS4 (RPKM) | | | | | | |
| Age at collection | −0.00037 | 0.98 | 100 | −0.0037 | 0.812 | 97 |
| Gender (female) | −0.055 | 0.924 | 99 | −0.11 | 0.85 | 97 |
| Smoking (Yes) | 0.467 | 0.45 | 100 | 0.55 | 0.398 | 97 |

TABLE 6
Univariate and multivariate models for predictors
of ACE2, TMPRSS2 and TMPRSS4 expression in PROTECT.

| | Univariate (UC) | | | Multivariate | | |
|---|---|---|---|---|---|---|
| PROTECT | Beta | P | N | Beta | P | N |
| Response: ACE2 (TPM) | | | | | | |
| Age at collection | −0.26 | 0.03 | 206 | −0.29 | 0.011 | 226 |
| Gender (female) | −0.05 | 0.949 | 206 | −0.08 | 0.91 | 226 |
| Disease Status (Yes) | | | | 2.93 | 0.023 | 226 |
| Response: TMPRSS2 (TPM) | | | | | | |
| Age at collection | −4.2 | 0.001 | 206 | −4.32 | 3.80E−04 | 226 |
| Gender (female) | −5.75 | 0.49 | 206 | −9.57 | 0.215 | 226 |
| Disease Status (Yes) | | | | 7.813 | 0.5626 | 226 |
| Response: TMPRSS4 (TPM) | | | | | | |
| Age at collection | −2.379 | 2.30E−05 | 206 | −2.36 | 2.90E−05 | 226 |
| Gender (female) | −5.559 | 0.13 | 206 | −4.416 | 0.215 | 226 |
| Disease Status (Yes) | | | | −71.29 | <2E−16 | 226 |

TABLE 7
Univariate and multivariate models for predictors of
ACE2, TMPRSS2 and TMPRSS4 expression in Cedars119.

| | Univariate | | | Multivariate | | |
|---|---|---|---|---|---|---|
| Cedars 119 | Beta | P | N | Beta | P | N |
| Response: ACE2 (FPKM) | | | | | | |
| Age at collection | 0.0072 | 0.9 | 105 | 0.038 | 0.55 | 96 |
| Gender (female) | −1.12 | 0.52 | 99 | −1.42 | 0.43 | 96 |
| Smoking (Yes) | −1.098 | 0.58 | 119 | −1.75 | 0.449 | 96 |
| Response: TMPRSS2 (FPKM) | | | | | | |
| Age at collection | −0.09 | 0.745 | 105 | −0.39 | 0.187 | 96 |
| Gender (female) | −11.009 | 0.18 | 99 | −9.89 | 0.24 | 96 |
| Smoking (Yes) | 16.55 | 0.089 | 119 | 20.16 | 0.062 | 96 |
| Response: TMPRSS4 (FPKM) | | | | | | |
| Age at collection | 0.18 | 0.29 | 105 | 0.017 | 0.93 | 96 |
| Gender (female) | −0.84 | 0.87 | 99 | −0.42 | 0.93 | 96 |
| Smoking (Yes) | 7.36 | 0.19 | 119 | 10.9 | 0.11 | 96 |

TABLE 8
Univariate and multivariate models for predictors
of TMPRSS2 and TMPRSS4 expression in WashU cohort.

| Response: TMPRSS2 (FPKM) | Beta | P | N | Beta | P | N |
|---|---|---|---|---|---|---|
| BMI at surgery | 2.11 | 0.048700 | 66 | 2.29 | 0.114 | 51 |
| Age at collection | 0.30 | 0.365400 | 70 | 0.03 | 0.957 | 51 |
| Disease status (Control) | 7.33 | 0.556000 | 70 | 14.85 | 0.500 | 51 |
| Gender (Female) | 15.5 | 0.314000 | 55 | 19.84 | 0.235 | 51 |
| Smoking (Yes) | 32.69 | 0.891000 | 35 | | | |

| | Univariate | | | Multivariate | | |
|---|---|---|---|---|---|---|
| Response: TMPRSS4 (FPKM) | Beta | P | N | Beta | P | N |
| BMI at surgery | 4.37 | 0.036200 | 66 | 5.935 | 0.024 | 51 |
| Age at collection | 1.12 | 0.080400 | 70 | 0.901 | 0.423 | 51 |
| Disease status (Control) | 1.27 | 0.958000 | 70 | −47.1 | 0.234 | 51 |
| Gender (Female) | −6.99 | 0.801000 | 55 | 7.41 | 0.803 | 51 |
| Smoking (Yes) | 39.95 | 0.293000 | 35 | | | |

In the WashU cohort, a strong positive association of ACE2 expression with BMI in both CD and non-IBD controls (p<0.0001, linear regression) was observed, as shown in FIG. 2D. No significant association of BMI with disease-severity phenotypes within CD (n=34) such as presence of perianal disease, stricturing and penetrating disease was observed.

There was no significant association with gender in SB139, WashU, RISK, PROTECT and Cedars119 cohorts (Tables 2, 3, 6 and 7). However, higher ileal expression of ACE2 was observed in females in the Cedars100 cohort (FIG. 5A, Table 4), consistent with similar observations in GTEx.

A statistical association of smoking with ACE2 expression was not observed in any of the adult cohorts (Table 2 and FIG. 3) although there was a suggestive trend towards higher expression, in the Cedars100 cohort (FIG. 5B) (p=0.15).

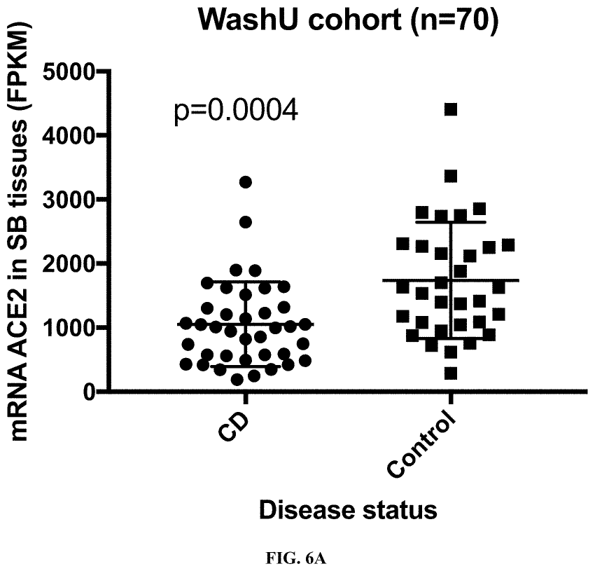

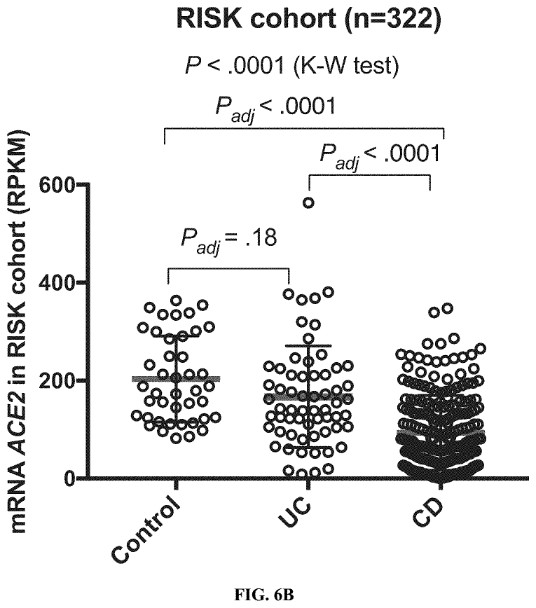

Data from ileal transcriptomics of non-IBD controls for comparison were only available for the WashU and Risk cohorts. In the WashU cohort (FIG. 6A), ileal ACE2 expression was lower in CD compared to controls (p=0.0004). Univariate model with disease status as predictor, was statistically significant for lower ACE2 expression in CD versus control in the WashU cohort (Table 2).

In the RISK cohort, median ACE2 expression in CD, UC and control was statistically different (p<0.0001) (FIG. 6B). Univariate models of ACE2 expression with disease status indicated ACE2 was lower in CD compared to controls (p=9.78e-14) or UC (p=3.13e-09) (Table 3).

Multivariate Associations:

Multivariate models with disease status as predictor, were statistically significant or trending for lower ACE2 expression in CD versus control in the WashU cohort (Table 5). In this cohort, BMI was observed to be the strongest predictor of ACE2 expression after adjusting for age at collection, disease status and gender. In the RISK cohort decreased ACE2 expression was observed in CD compared to controls (p=2.14e-14) or UC (p=5.3e-09) after adjusting for age at diagnosis and gender (Table 2). Age at diagnosis was significantly associated with ACE2 expression after adjusting for disease status and gender in the RISK cohort (Table 2). In contrast to SB, multivariate model of colonic ACE2 with disease status in the PROTECT cohort indicated elevated rectal ACE2 expression in UC compared to non-IBD (Table 6).

Differences in Small Bowel ACE2 Gene Expression in Involved Versus Un-Involved CD

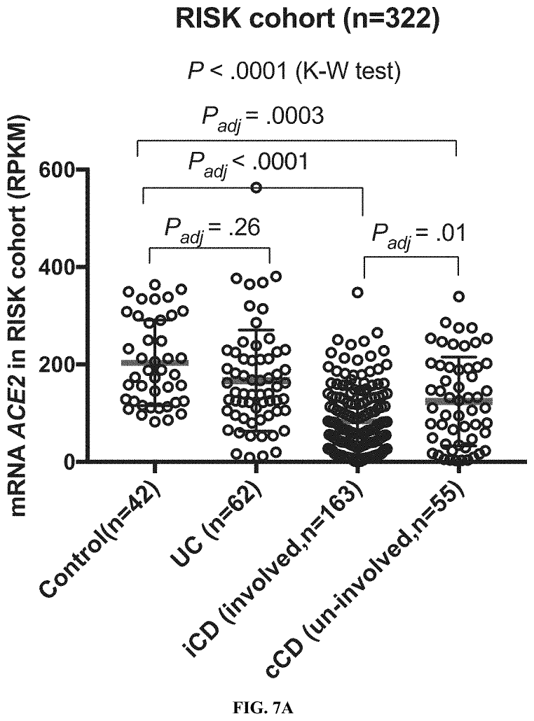

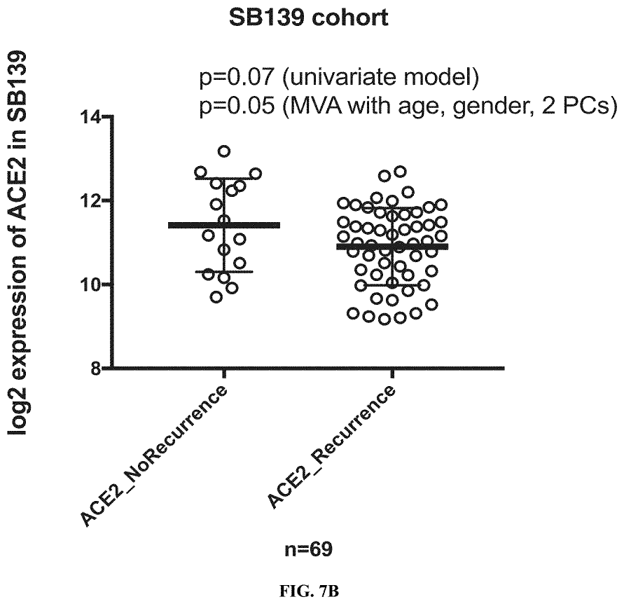

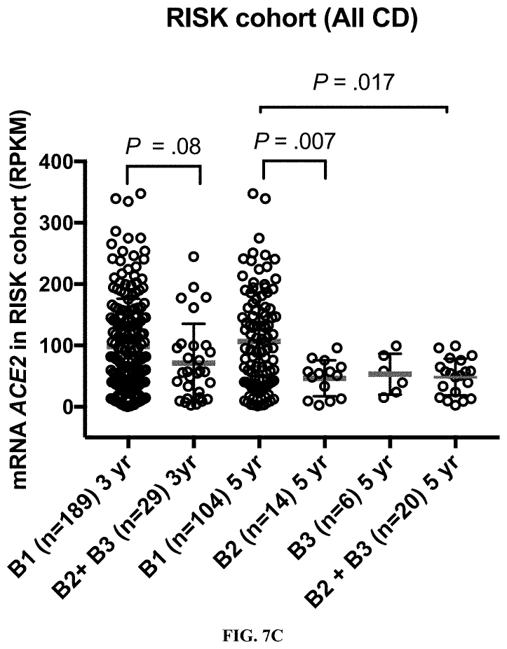

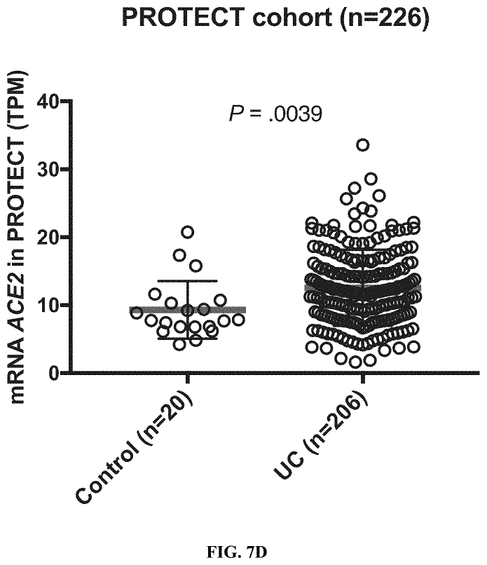

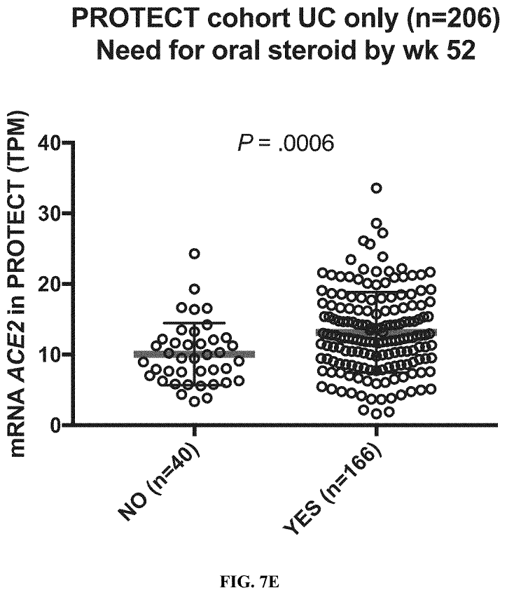

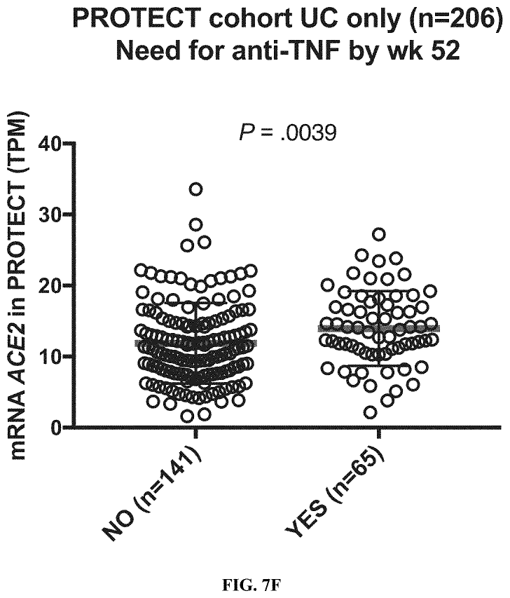

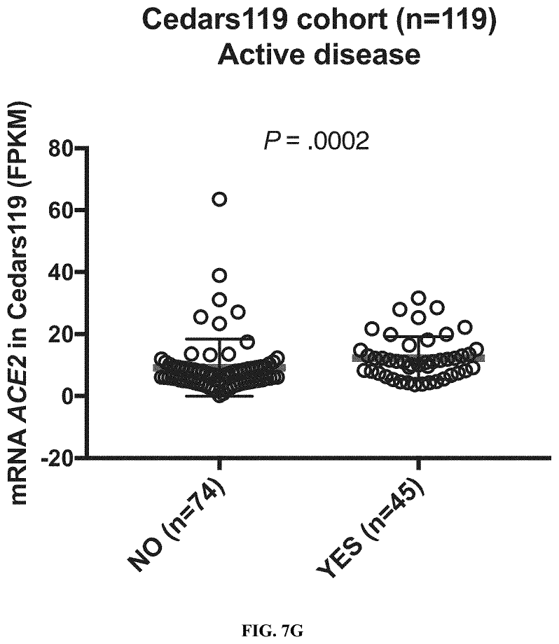

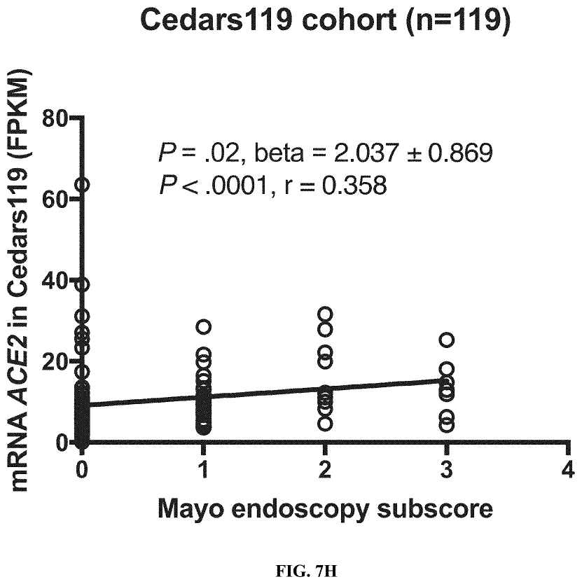

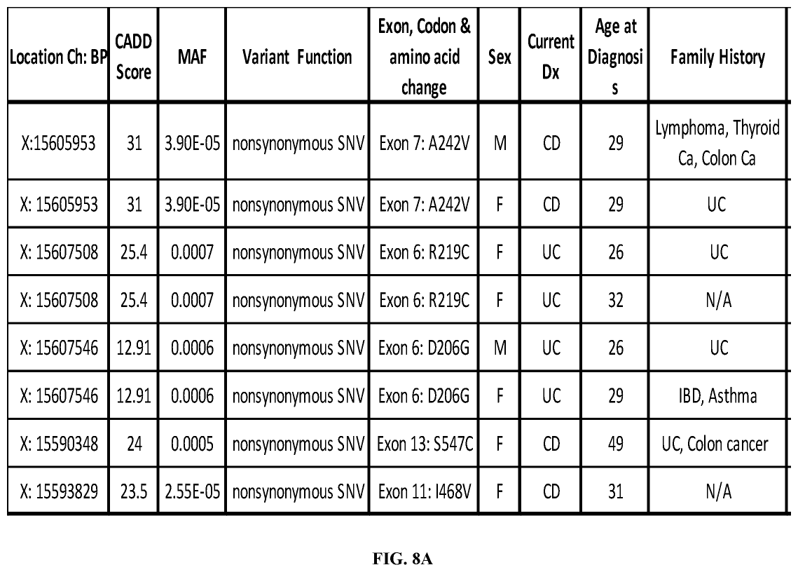

In the RISK cohort, ileal ACE2 expression was lower in CD with small bowel involvement (iCD) compared to uninvolved CD (cCD) (p=0.005, FIG. 7A and Table 3). Median ACE2 expression was statistically different in controls, UC, iCD and cCD (p<0.0001). An association between lower expression of ACE2 at diagnosis with the development of complicated disease by year 3 both without and with adjustment for age and gender (FIG. 7C, p=0.08). This association of ACE2 expression at diagnosis and subsequent development of complicated disease became significant by year 5 of follow-up (FIG. 7C, B2+B3 versus B1, p=0.017 and B2 versus B1, p=0.007; after adjusting for age and gender).

The inventors have previously disclosed a transcriptomics-based sub-groups with varying disease-severity in the SB139 cohort where a severe-refractory sub-group (CD3) was associated with increased recurrence as well as faster time to both recurrence and second surgery compared to the mild-refractory (CD1) sub-group, as reported in WO 2020/010139, which is hereby incorporated by reference in its entirety. In this SB139 cohort, ACE2 was lower in the CD3 versus the CD1 sub-group (FC=−3.23, corrected p<1e-07). Using a multivariate model, lower ACE2 was also observed in subjects with disease recurrence after surgery, when corrected for age, gender and first two PCs in genotype data (FIG. 7D, p=0.05).

ACE2 Expression and Post-Op Recurrence.

Transcriptomics-based sub-groups with varying disease severity in the SB139 cohort have been observed, with a severe refractory sub-group (CD3) to be associated with increased recurrence, faster time to both recurrence and second surgery compared to the ‘mild’ refractory (CD1) sub-group. The gene expression probe for ACE2 was downregulated in CD3 versus CD1 sub-group (FC=−3.23, corrected p<1e-07). In the SB139 cohort, lower ACE2 gene expression was observed in subjects with disease recurrence after surgery after adjusting for age, gender. (FIG. 7B, p=0.05)

Differences in Colonic ACE2 Expression by Disease Sub-Phenotype and Inflammation

In the PROTECT cohort, colonic ACE2 was elevated in biopsies from UC subjects with varying disease severity and associated inflammation compared to controls (p=0.004, FIG. 7E, Table 6). In this cohort, elevated colonic ACE2 observed was predictive of UC patients requiring oral steroid by week 52 (FIG. 7F, p=0.0006) as well as subjects that subsequently developed severe disease requiring the use of anti-TNF rescue therapy by week 52 (p=0.004).

In the Cedars119 cohort, elevated colonic ACE2 was seen in subjects with active disease (FIG. 7G, p=0.0002) and there was positive correlation with ACE2 and increasing Mayo score (FIG. 711, p<0.0001, r=0.358, Spearman correlation).

Expression atlas was queried to determine the impact of complicated CD (stricturing, penetrating or disease recurrence) on colonic ACE2. It was discovered in Peck et al., MicroRNAs Classify DifferentDifferent Disease Behavior Phenotypes of Crohn's Disease and May Have Prognostic Utility. Inflammatory Bowel Diseases 2015; 21:2178-2187, that elevated levels of ACE2 in non-inflamed colon tissue, were associated with stricturing and penetrating disease compared to non-IBD (B2, fold change (FC)=2.1, p_{adj}=0.01; B3, FC=1.5, p_{adj}=0.02) This is in contrast to the observations in non-inflamed ileal tissue (SB139 cohort, lower ACE2 with disease recurrence, FIG. 7D) indicating discordant ACE2 signals (SB versus colon) with complicated disease in macroscopically normal tissue.

ACE2 in Relation to Other COVID-19 Implicated Genes, Inflammatory Cytokines, and Known IBD Targets.

Due to the role of ACE2 in COVID-19, differential expression of COVID-19 related genes ACE, TMPRSS2, TMPRSS4 and SLC6A19 in controls versus CD was analyzed in WashU (Table 9) and RISK cohorts (Table 10). Expression of both ACE and ACE2 was found to be downregulated in CD versus control. Similar trends were observed for SLC6A19 and ACE2. Upregulation of the protease, TMPRSS2, was observed in CD compared to controls in the RISK cohort.

Ileal TMPRSS2 expression was associated with age and positive smoking status in Cedars100. Elevated expression of both TMPRSS2 and TMPRSS4 was associated with BMI in the WashU cohort. Significantly elevated ileal TMPRSS2 in CD compared to controls in the RISK cohort (Table 11) was observed.

The differential expression of ACE and SLC6A19 in non-IBD versus CD in WashU (Table 12) and RISK cohorts (Table 13) were also examined. Similar to ACE2, expression of ACE was lower in CD versus controls in both WashU and RISK. Lower ileal expression of SLC6A19 in CD compared to controls in the RISK cohort (Table 13) and a similar trend in WashU cohort (Table S8) was observed.

In the ACE2 co-expression analysis, several genes that correlated with ACE2 expression in both SB139 and the Cedars100 CD cohorts (Table 14) including SIGMAR1 (r=0.6 to 0.43, p<0.0001) and JAK1 (r=0.34 to 0.25, p<0.05) where r is the Spearman correlation coefficient. JAK3 was inversely correlated with ACE2 (r=−0.39 to −0.38, p<0.0001) in both CD cohorts (Table 14) were observed.

Ileal ACE2 (RISK cohort) was negatively correlated with expression of transcription factor for interferon signaling, STAT1 (p<0.0001, r=−0.6) while in colon ACE2 and STAT1 expression (PROTECT cohort) was positively correlated (p<0.0001, r=0.47). A stronger positive correlation was observed between ACE2 and HNF4A in ileum (p<0.0001, r=0.685) compared to that in colon (p=0.004, r=0.19).

TABLE 9
Univariate and multivariate models for predictors
of TMPRSS2 and TMPRSS4 expression in RISK cohort

| | Univariate | | Multivariate | |
|---|---|---|---|---|
| Response: TMPRSS2 (RPKM) | Beta | P | Beta | P |
| All (n = 322) | | | | |
| Age at diagnosis | −0.125 | 0.769 | −0.2785 | 0.512 |
| Disease status (non-IBD) | −10.5904 | 9.00E−03 | −10.6778 | 8.80E−03 |
| Disease status (UC) | 0.8448 | 8.07E−01 | 0.905 | 7.93E−01 |
| Gender(male) | −4.116 | 0.131 | −3.9613 | 0.1441 |
| CD only (n = 218) | | | | |
| Age at diagnosis | −0.289 | 0.622 | −0.3098 | 0.597 |
| Gender(male) | −5.303 | 0.144 | −5.0829 | 0.162 |
| CD_type(iCD) | −5.236 | 2.04E−01 | −5.1371 | 2.14E−01 |
| | Univariate | | Multivariate | |
| Response: TMPRSS4 (RPKM) | Beta | P | Beta | P |
| All (n = 322) | | | | |
| Age at diagnosis | 0.1 | 0.654 | 0.058 | 0.795 |
| Disease status (non-IBD) | −3.827 | 7.40E−02 | −3.729 | 8.30E−02 |
| Disease status (UC) | −0.786 | 6.67E−01 | −0.825 | 6.52E−01 |
| Gender(male) | −1.203 | 0.402 | −1.121 | 0.4353 |
| CD only (n = 218) | | | | |
| Age at diagnosis | 0.037 | 0.902 | 0.041 | 0.893 |
| Gender(male) | −2.593 | 0.170 | −2.571 | 0.176 |
| CD_type(iCD) | −0.957 | 6.57E−01 | −0.83 | 7.01E−01 |

TABLE 10
Differential expression of other COVID-19 relevant genes,
ACE and SLC6A19 in CD versus control in WashU cohort.

All (n = 55)
Multivariate

| Response: ACE (FPKM) | Beta | P |
|---|---|---|
| Age at collection | 0.361 | 0.918 |
| Disease status (non-IBD) | 498.16 | 6.26E−04 |
| Gender(female) | 38.41 | 0.694 |

TABLE 11
Differential expression of other COVID-19 relevant genes,
ACE, and SLC6A19 in CD versus control in RISK cohort

All (n = 322)
Multivariate

| Response: ACE (RPKM) | Beta | P |
|---|---|---|
| Age at diagnosis | 1.45 | 0.22086 |
| Disease status (non-IBD) | 65.319 | 1.71E−08 |
| Disease status (UC) | 52.337 | 1.02E−07 |
| Gender(male) | −1.72 | 0.8196 |

All (n = 322)
Multivariate

| Response: SLC6A19 (RPKM) | Beta | P |
|---|---|---|
| Age at diagnosis | 1.982 | 0.148693 |
| Disease status (non-IBD) | 79.903 | 2.85E−09 |
| Disease status (UC) | 77.093 | 2.35E−11 |
| Gender(male) | −2.369 | 0.786246 |

All (n = 55)
Multivariate

| Response: SLC6A19 (FPKM) | Beta | P |
|---|---|---|
| Age at collection | 5.205 | 0.049 |
| Disease status (non-IBD) | 160.649 | 0.116 |
| Gender(female) | 56.78 | 0.436 |

TABLE 12
Co-expression of ACE2 with genes of interest in CD cohorts of SB139 and Cedars100.
Beta and P represent slope and pvalue from linear regression model fit.

| | Cohort | | | | | | | |
|---|---|---|---|---|---|---|---|---|
| | SB139 | | | | Cedars100 | | | |
| Gene | Beta | P | Spearman r | Spearman P | Beta | P | Spearman r | Spearman P |
| ACE | 0.685 | 3.66E−29 | 0.769 | <E−12 | 0.228 | 6.19E−12 | 0.699 | 3.14E−13 |
| SIGMAR1 | 1.550 | 4.35E−17 | 0.600 | <E−12 | 0.334 | 6.15E−05 | 0.428 | 1.17E−05 |
| BRD2 | 0.552 | 1.11E−11 | 0.446 | 7.51E−12 | 1.230 | 0.028 | 0.416 | 0.029 |
| EIF4E2 | 0.880 | 7.00E−09 | 0.388 | 4.20E−09 | 4.000 | 0.007 | 0.371 | 0.002 |
| ADAM17 | 1.100 | 1.30E−08 | 0.481 | 9.19E−09 | −0.538 | 0.077 | −0.092 | 0.042 |
| DNMT1 | −2.010 | 1.64E−08 | −0.425 | 3.61E−08 | −0.071 | 0.013 | −0.213 | 0.012 |
| NEK9 | 1.190 | 3.11E−08 | 0.442 | 2.34E−08 | 1.040 | 0.008 | 0.140 | 0.012 |
| PLOD1 | 1.160 | 1.44E−07 | 0.426 | 1.02E−07 | −0.060 | 2.21E−04 | −0.439 | 3.29E−05 |
| CSNK2B | 1.210 | 4.15E−07 | 0.401 | 3.44E−07 | −0.450 | 0.377 | −0.062 | 0.293 |
| TNF | −2.270 | 5.43E−07 | −0.366 | 4.14E−07 | 2.970 | 0.015 | 0.052 | 0.034 |
| JAK3 | −0.671 | 1.34E−06 | −0.389 | 1.48E−06 | −0.917 | 5.81E−04 | −0.382 | 2.58E−04 |
| PLOD2 | 0.900 | 2.46E−06 | 0.450 | 2.47E−06 | 3.810 | 0.010 | 0.219 | 0.004 |
| JAK1 | 1.740 | 2.80E−05 | 0.345 | 2.84E−05 | 0.957 | 0.034 | 0.256 | 0.049 |
| TMPRSS4 | 0.879 | 3.55E−05 | 0.411 | 2.97E−05 | 1.930 | 0.024 | 0.279 | 0.011 |
| IL6 | −1.380 | 3.81E−05 | −0.357 | 9.02E−05 | −1.540 | 0.171 | −0.121 | 0.096 |
| AGTR1 | −1.620 | 5.73E−05 | −0.285 | 3.85E−05 | 3.200 | 0.405 | 0.070 | 0.259 |
| IL23R | −1.420 | 0.008 | −0.258 | 0.006 | −0.829 | 0.056 | −0.346 | 0.022 |
| IL12B | −2.830 | 0.008 | −0.188 | 0.009 | −2.790 | 0.221 | −0.150 | 0.271 |
| TMPRSS2 | −0.501 | 0.014 | −0.221 | 0.014 | 0.198 | 0.020 | 0.318 | 0.005 |
| IFNG | −2.020 | 0.021 | −0.213 | 0.022 | 1.090 | 0.591 | 0.074 | 0.602 |
| IL1 | −0.806 | 0.021 | −0.188 | 0.024 | −0.518 | 7.04E−04 | −0.442 | 1.14E−04 |
| IL17 | −1.790 | 0.194 | −0.139 | 0.163 | −5.570 | 0.064 | −0.140 | 0.112 |
| IL12A | −1.020 | 0.630 | 0.026 | 0.610 | 2.350 | 0.355 | 0.097 | 0.534 |
| IL8 | −0.093 | 0.852 | −0.037 | 0.920 | −0.771 | 0.261 | −0.024 | 0.180 |

In the ACE2 co-expression analysis number of genes that correlated with ACE2 expression was observed in both SB139 and the Cedars100 CD cohorts (Table 8) including SIGMAR1 (coefficient=0.348 to 1.55, p<0.0001), and JAK1 (coefficient=1.51 to 1.74, p<0.05). JAK3 was inversely correlated with ACE2 (coefficient=−0.939 to −0.671, p<0.001) in both CD cohorts (Table 12).

The Effect of Inflammation and Anti-Cytokine Therapy on ACE2 Expression in SB and Colon

Univariate analyses for trials where SB or colonic biopsy samples were collected pre- and post-exposure to anti-TNF (infliximab, IFX trial) and anti-IL12/23 (ustekinumab, CERTIFI and UNITI-2 trials) to query the effect of anti-cytokine monoclonal antibodies used in the treatment of IBD on intestinal ACE2 expression.

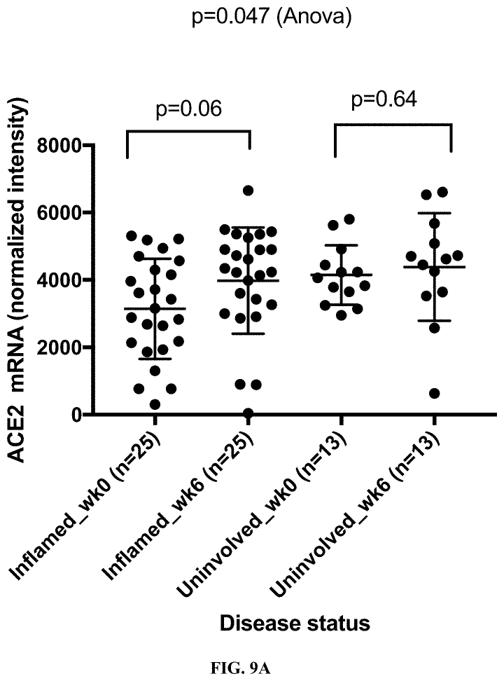

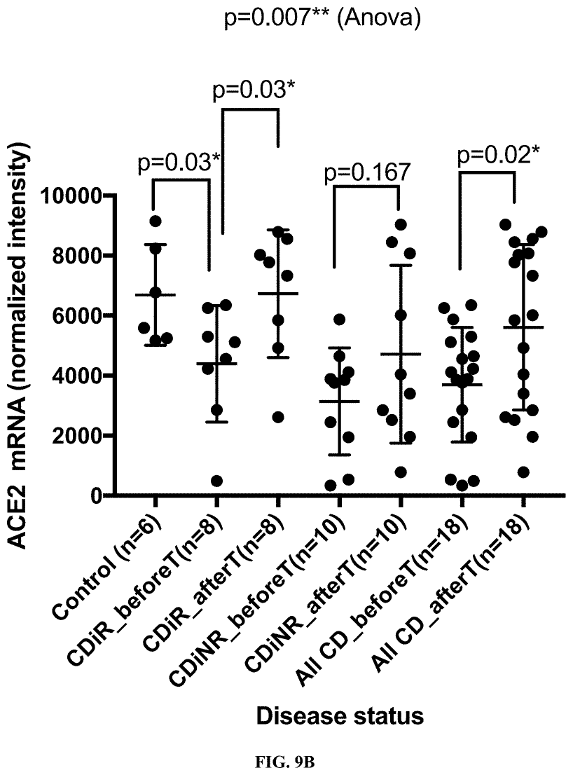

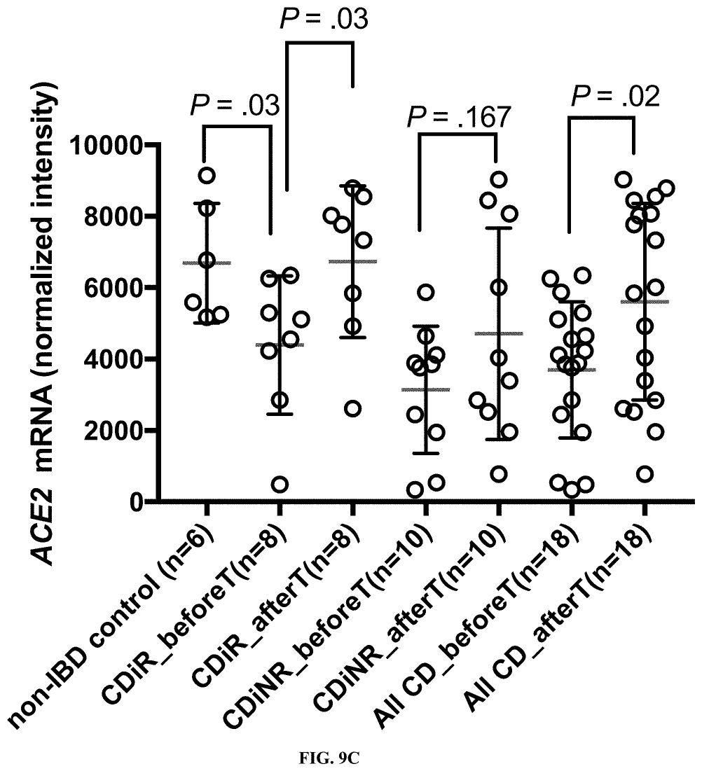

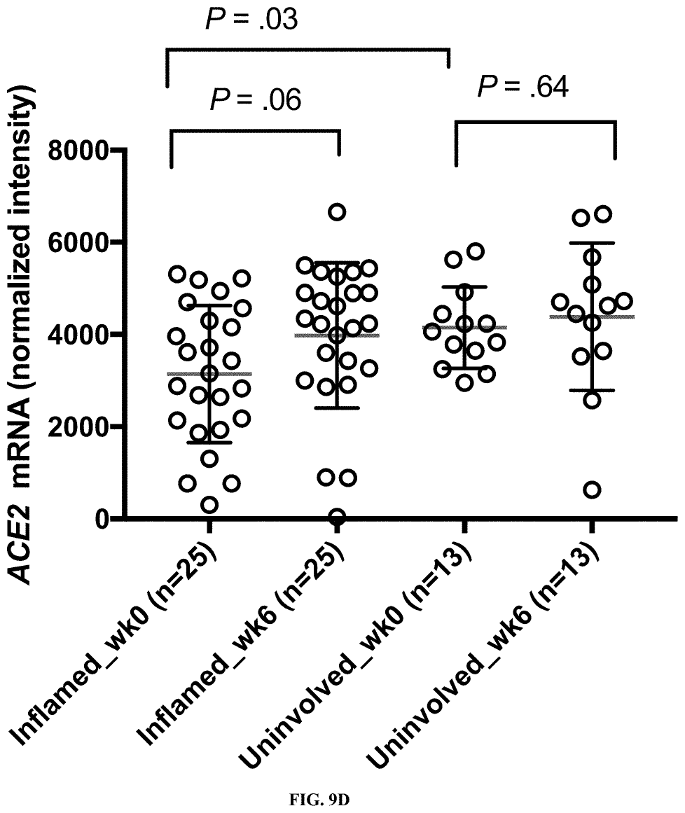

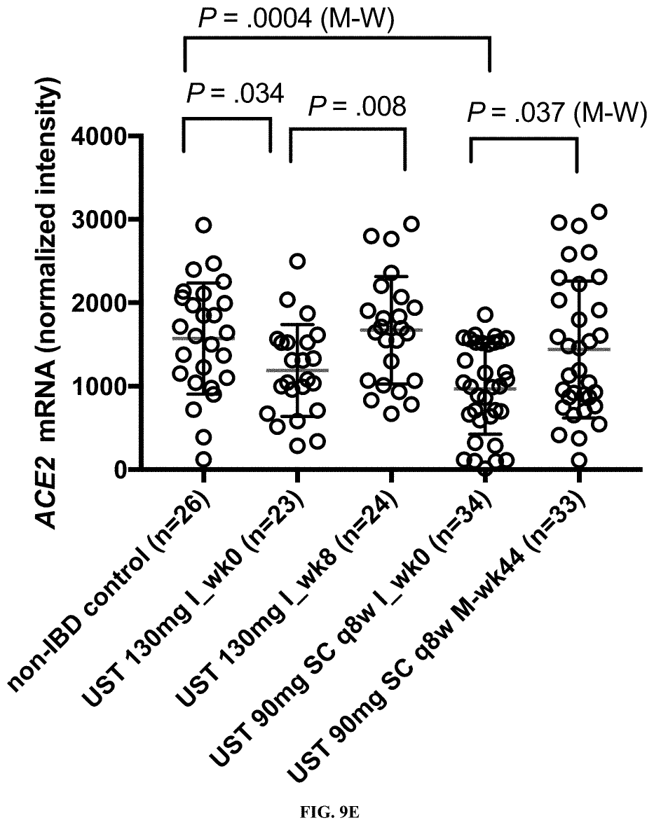

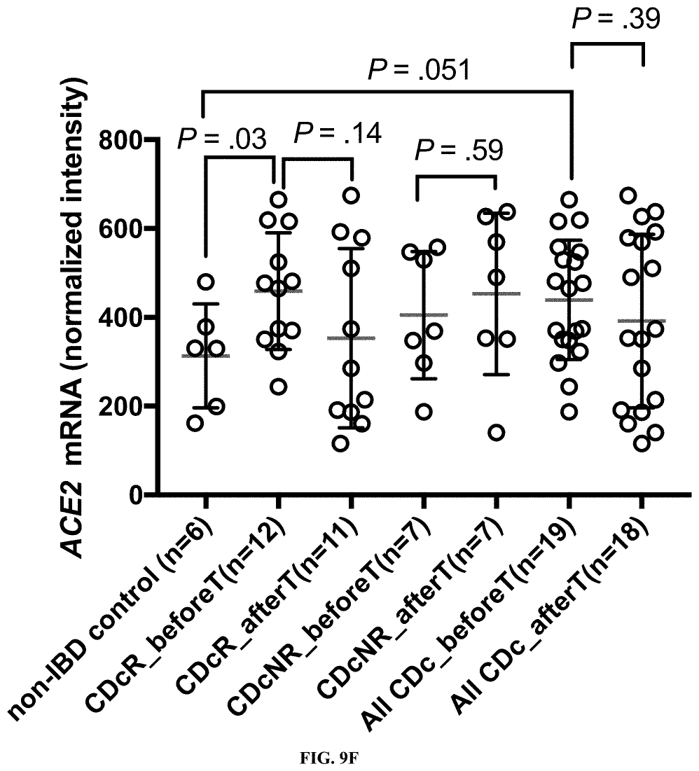

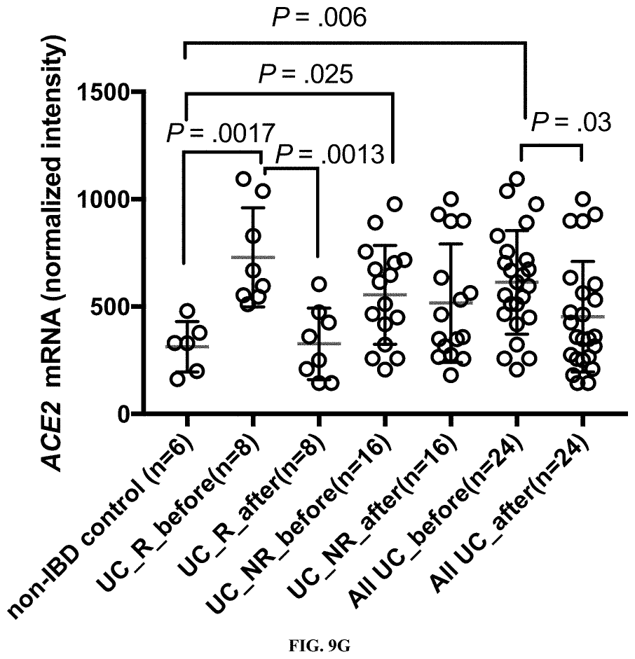

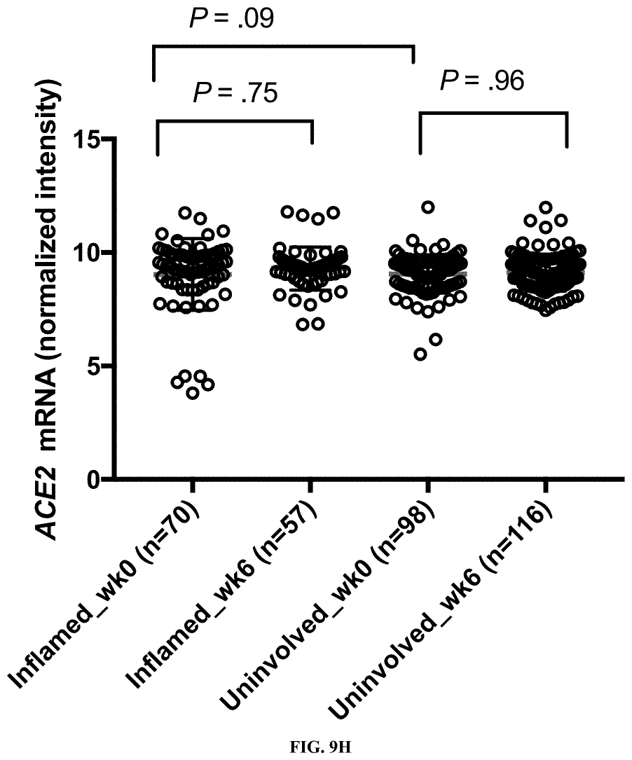

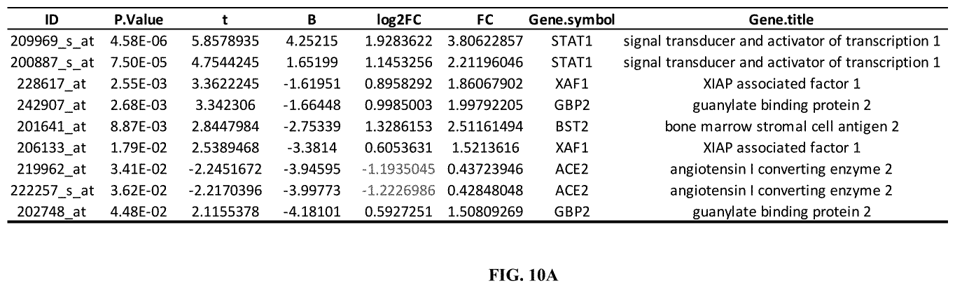

Using the data derived from ileal biopsies from the CERTIFI and UNITI-2 cohorts, a trend towards increased ACE2 expression between pre-treatment and post-treatment (6 week) samples was observed in the inflamed tissues but not non-inflamed (FIG. 9C-9D). In the IFX trial, ileal ACE2 expression significantly increased after infliximab induction in CD subjects (p=0.02). This phenomenon was significant in individuals who responded to treatment (p=0.037) but not in non-responders (FIG. 9C).

Response to treatment was unavailable for CERTIFI trial and a significant association between pre- and post-treatment was not observed (FIG. 9C). The ileal ACE2 levels in UNITI-2 trial (FIG. 9D) were significantly lower at baseline in CD subjects compared to non-IBD controls for the two dosage groups (p=0.034 and p=0.0004). Post-ustekinumab induction, ACE2 levels were significantly restored compared to baseline (p=0.008). In the maintenance-therapy group ACE2 levels were significantly restored after 44 weeks compared to baseline (p=0.037).

SB ACE2 expression was decreased in inflamed SB tissue compared to controls (FIG. 9C and FIG. 9E) and the severity of inflammation as measured by macroscopic and microscopic criteria (ileal SES-CD and GHAS) was negatively correlated with ACE2 expression in UNITI-2 trial dataset (SES-CD: week 0, p=0.0007, beta=−68.66; week 8, p=0.0014, beta=−68.3; GHAS: week 0, p<0.0001, beta=−80.75; week 8, p<0.0001, beta=−77.35) An inverse correlation between ACE2 expression and increasing severity of inflammation as measured by macroscopic and microscopic criteria (ileal GHAS and SES-CD) was also observed, as shown in FIGS. 11A-11D.

In the IFX trial, colonic ACE2 levels (FIG. 9F) at baseline (pre-treatment) were significantly elevated in Crohn's colitis responders (p=0.03). In the same trial, colonic ACE2 was significantly elevated in UC (both responders, p=0.001 and non-responders, p=0.025) at baseline compared to non-IBD (FIG. 9G). After anti-TNF treatment, ACE2 levels were significantly reduced to non-IBD levels in UC responders (p=0.0013) as well as combined UC cohort (p=0.03). A significant impact of treatment on colonic ACE2 levels in the CERTIFI ustekinumab trial (FIG. 911) was not observed.

Modulation was not observed of TMPRSS2 or TMPRSS4 via anti-TNF therapy in ileal or colonic tissue although colonic TMPRSS4 levels were reduced at baseline in both Crohn's colitis as well as UC.

To determine whether the decrease in ACE2 before IFX therapy (FIG. 9B) was simply due to epithelial erosions, the mRNA expression of an epithelial marker, Keratin-8 (KRT-8) was analyzed. KRT8 levels in ileal biopsies pre- and post-treatment was fairly uniform, implying no substantial epithelial erosions were likely present at baseline in CD ileitis samples compared to controls. This indicated that the drop in ACE2 in CD ileum pre-treatment is unlikely to be the result of epithelial cell loss in the areas sampled.

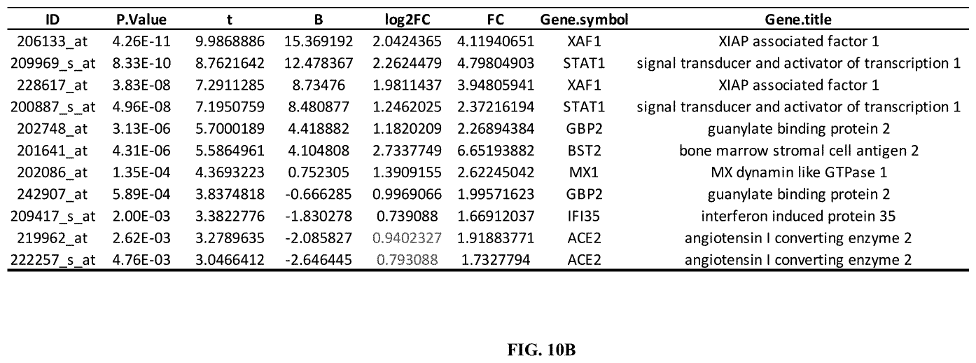

Using the IFX trial colonic and ileal transcriptomics at baseline (pre-treatment), it was observed that the direction of FC in IBD versus non-IBD for some canonical interferon stimulated genes reported in literature (e.g., STAT1, BST2, XAF1, IFI35, MX1, GBP2) is the same as ACE2 in colon but not in ileum (FIG. 10A-10B). The expression of ACE2 itself in ileum was found to be 10 times than that in colon in this dataset (p<0.0001, non-IBD control, ileum versus colon).

Whole Exome Sequencing

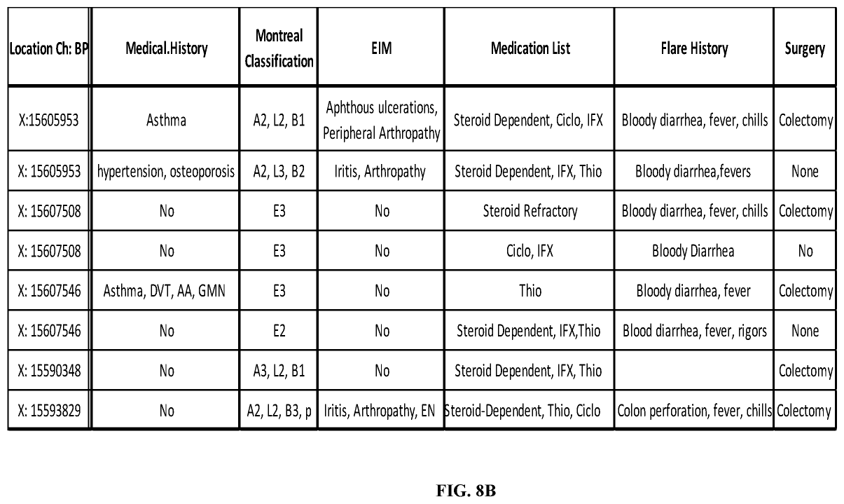

A total of 5 ACE2 variants were observed in 9 subjects which are rare (MAF<=1% in European populations in gnomAD), with a ‘high’ CADD score (CADD PHRED>10) that were also functionally meaningful variants (i.e. not synonymous variants) (Table 4). Clinical data were available for 8 of the subjects (FIG. 8A-8B). These subjects did not develop IBD at a young age but had severe phenotypes with 6 of the 8 being described as having steroid dependent or refractory disease, 5 requiring surgical resection, and 6 of the 8 having fever/chills/rigors documented as predominant symptoms experienced during disease relapse.

Discussion

Robust expression of ACE2 mRNA was observed in SB tissue from both non-IBD controls and subjects with CD and UC. Increased ACE2 mRNA was observed in the ileum with demographic features that have been associated with poor outcomes in COVID-19 including age and raised BMI. This age-related ACE2 expression may be one of the reasons for decreased COVID-19 susceptibility in children versus adults if these data, particularly from the non-IBD subjects, are reflective of ACE2 expression elsewhere in other organs such as the lung. Lower ACE2 expression in uninvolved SB tissue was associated with CD recurrence after surgery in an adult CD cohort. In the ileal biopsies from the RISK pediatric inception cohort, ACE2 levels at diagnosis were negatively associated with inflammation and disease severity (cCD versus iCD and UC versus CD) and remarkably the subsequent development of complicated disease at 5 years after diagnosis.

The demographic associations in non-IBD subjects and also the relationship between ACE2 expression in macroscopically non-inflamed tissue from CD patients point to systemic changes influencing ACE2 mechanisms. In the cases of aging and increased BMI, both conditions are associated with increased immune tone and myeloid skewing, as well as increased ACE2. Higher BMI has been linked with increased risk of infections. Increased ACE2 expression in lung has also been reported to be associated with age. There is speculation that the GI-tract may serve as an alternate route for uptake of SARS-CoV-2 and the findings described herein in the GI-tract may take on increased relevance if this is confirmed. Furthermore, early, but uncontrolled, evaluations of the SECURE-IBD registry suggest that patients with IBD appear to be under-represented in those diagnosed with COVID-19 compared with what has been seen in the general populations in both Northern Italy and China. The data described herein suggest reduced ACE2 expression in subsets of IBD may potentially contribute to this phenomenon.

Recent findings have suggested that men are at risk of higher COVID-19 mortality, however, the inventors of the instant disclosure do not report higher ACE2 expression in men—in fact in one cohort, higher expression in women was observed. This finding is in keeping with ACE2 expression in women (GTEx). However, gender differences in ACE2 may be tissue dependent and reflect tissue-specific escape from X-inactivation. Whether men are more susceptible to COVID-19, or simply more likely to experience worse outcomes, or both, remains unknown. A trend towards increased ACE2 expression in smokers in only one cohort was observed, perhaps reflecting limited power given the relatively low frequency of smokers in our populations, two of which included only children.

In contrast to the ileal tissue in CD, there is elevated ACE2 expression in the colon in UC compared to non-IBD. These findings are consistent with a recent preprint studying tissue specific (SB or colon) patterns of ACE2 expression. Furthermore, these findings suggest this ACE2 ‘compartmentalization’ extends to disease phenotypes including progression to complicated disease and disease recurrence in CD with directionality of association with subsequent development of complicated disease (B2 or B3) dependent on SB (decreased) or colonic (increased) location. Consistent with this effect of location is the finding of increased ACE2 expression with increased Mayo score in UC. Overall, the analyses described herein indicated discordant ACE2 signals in SB versus colon that are enhanced with inflammation but exist even in macroscopically normal tissue where these discordant signals are associated with the development of complicated disease. These observations further emphasize SB/colon ‘compartmentalization’ of ACE2-related immune responses.

In the colon (PROTECT pediatric UC inception cohort), a positive correlation between STAT1 (the reported transcription factor for interferon signaling and a canonical interferon stimulated gene (ISG)³¹) and ACE2 was observed, consistent with recent reported literature of ACE2 being an ISG. However, in the ileum, STAT1 is negatively correlated with ACE2 (RISK pediatric inception cohort of CD subjects). A strong correlation of ACE2 with HNF4A in ileum compared to colon was observed, which is consistent with recent reports that HNF4A is an upstream regulator of ACE2 in ileum. Using the IFX trial colonic and ileal transcriptomics, the findings herein show that the direction of fold change in IBD versus non-IBD for some canonical ISGs reported in literature is similar as ACE2 in colon but not in the ileum, consistent with ACE2 reported as an ISG in colon. Without being bound by any particular theory, the inventors of the instant disclosure have three hypotheses: First, since the expression of ACE2 in ileum is 10 times of that in colon, the local tissue factors, distinct in different intestinal regions, set the homeostatic levels and direction of ACE2 response to inflammation. Second, the threshold of biological control for interferon signaling is surpassed in ileum compared to colon. Third, it is also possible that there are differences in the local RAAS in ileum versus colon as demonstrated by the discordant ACE2 signals in ileal and colonic inflammation shown in this disclosure.

ACE2 may play a paradoxical role in disease progression of COVID-19. Although higher expression of ACE2 increases viral uptake by host, physiologically ACE2 has a significant anti-inflammatory role. ACE2 is required to neutralize the pathological effects of increased Angiotensin-II (Ang-II) in classical RAAS by converting Ang II to Ang1-7. Lung ACE2 expression is protective against diseases such as pulmonary fibrosis, lung injury, and asthma. The inventors of the instant disclosure show that within CD, reduced SB ACE2 expression was associated with inflammation, non-response to anti-cytokine therapy and subsequent relapse of disease and development of complicated disease related to fibrosis.

ACE2 expression in the gut is necessary to maintain amino acid homeostasis, antimicrobial peptide expression, ‘healthy’ intestinal microbiome, and Ace2^{-/-} mice are more prone to developing colitis in induced models. Expression of amino acid transporter SLC6A19 (B(0)AT1) in SB is dependent on presence of ACE2, which acts as a chaperone for membrane trafficking of SLC6A19. Accordingly, expression of SLC6A19 is decreased in SB CD along with that of ACE2. Notably, lower SLC6A19 levels are selectively associated with lower tryptophan levels in SB CD. Dysregulated tryptophan metabolism has been linked to systemic inflammation. The biologic mechanisms that link levels of tryptophan to pathogenic intestinal inflammation and obesity are complex, including host and microbial production of bioactive tryptophan metabolites, the selective roles of these metabolites on molecular processes such as energy checkpoint and transcriptional controls of inflammation pathways. Exploring these mechanisms in the ACE2 deficiency of SB CD may distill how the ACE2 network could serve as a protective pathway for IBD.

Elevated ACE2 levels may promote tissue propagation of virus and, in theory, could promote COVID-19 disease severity. However, the secondary cytokine storm likely promotes tissue injury via mechanisms independent of viral propagation and this process may be independent of ACE2. Alternatively, ACE2, with its anti-inflammatory properties may play a role in protection from the secondary cytokine storm. Due to the SARS-CoV-2/ACE2 interaction, there has been interest in treatments for COVID-19 that modulate ACE2. A study examining ACE2 with TNF-α production found that viral entry modulated TNF-α-converting enzyme via the ACE2 cytoplasmic domain and caused tissue damage through increased TNF-α production ACE2 levels were observed to be restored after infliximab therapy and that this was significant in anti-TNF responders. An increase in ileal ACE2 expression was observed with both ustekinumab induction and maintenance therapies. The inverse relationship of ACE2 with inflammatory cytokines and restoration of enhanced ileal ACE2 levels after response to anti-cytokine therapy point towards the anti-inflammatory function of ACE2 in SB. It has been reported that fecal calprotectin is elevated and correlates with serum IL-6 in COVID-19, linking gut inflammation and systemic cytokines in patients infected with SARS-CoV-2. However, further work will be needed to delineate the anti-inflammatory function of ACE2 in COVID-19 and determine whether anti-cytokine therapies could be effective in modulating the secondary cytokine storm associated with COVID-19.

Consistent with our findings, a recent study by Suarez-Fariñas et. al also reported compartmentalization of intestinal ACE2 in IBD with inflammation and recognized a potential role of anti-cytokine therapy for COVID-19 treatment. Using gene regulatory networks, they also dissected overlapping molecular signals in IBD and COVID-19. Independently, this disclosure reports ACE2 association with other demographics (elevated BMI); significant differences in ileal ACE2 levels in UC and CD subjects in the RISK cohort; and that reduced ileal ACE2 at diagnosis were predictive of development of complicated CD at 5-year follow-up in RISK cohort and also associated with severe refractory CD in the SB139 cohort. The inventors of the instant disclosure also extended the region-specific discordant ACE2 signals in IBD inflammation to both CD and UC disease sub-phenotypes, prognosis and need for therapy.

ACE2 co-expression was analyzed with a set of candidate genes as potential targets for novel or repurposed drugs. SIGMAR1 (candidate target for the drug hydroxychloroquine) to be consistently co-expressed with ACE2. The use of hydroxychloroquine in treating COVID-19 remains controversial. In addition, JAK1 expression was observed to be consistently co-expressed with ACE2 in contrast to JAK3 which shows a consistent but inverse relationship with ACE2. Selective JAK inhibitors are available and in development. Baricitinib (a JAK1/2 inhibitor) is being tested in COVID-19 based on both its anti-inflammatory properties and its possible role in inhibiting endocytosis and viral entry. Our observation of co-occurrence of ileal ACE2 and JAK1 provides some support for the testing of this compound in COVID-19.

To summarize, association of ACE2 with various demographics (associated with worse outcomes from COVID-19) and clinical factors were in multiple IBD transcriptomic datasets. These finds show, for the first time that the discordant ACE2 signals in SB and colonic inflammation related to prognosis and response to therapy. This disclosure also shows that impaired ileal ACE2 expression that leads to worse outcomes in CD and evidence that implicates ACE2 pathway as a protective, tryptophan-dependent anti-inflammatory mechanism in severe IBD. Anti-TNF and anti-IL12/23 may restore ACE2 levels in the context of inflammation reduction, suggesting that restoration of the ACE2 pathway may be a mechanism by which these drugs promote recovery in IBD. Our work supports the potential paradoxical function of ACE2 in inflammation and COVID-19. Individuals with higher ACE2 expression may be at increased risk of infection with SARS-CoV-2 but ACE2 likely has anti-inflammatory and anti-fibrotic functions in SB CD and may play an important role in preventing the secondary cytokine storm seen in COVID-19 as well as preventing the development of complicated disease in IBD.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

SEQUENCES

| SEQ ID | | |
|---|---|---|
| NO | Sequence | Name |
| 1 | AGTCTAGGGAAAGTCATTCAGTGGATGTGATCTTGGCTCACAGGGGACGATGTCAAGCTCTTCCTGGCTC | >NM_001371415.1 |
| | CTTCTCAGCCTTGTTGCTGTAACTGCTGCTCAGTCCACCATTGAGGAACAGGCCAAGACATTTTTGGACA | Homo sapiens |
| | AGTTTAACCACGAAGCCGAAGACCTGTTCTATCAAAGTTCACTTGCTTCTTGGAATTATAACACCAATAT | angiotensin |
| | TACTGAAGAGAATGTCCAAAACATGAATAATGCTGGGGACAAATGGTCTGCCTTTTTAAAGGAACAGTCC | converting |
| | ACACTTGCCCAAATGTATCCACTACAAGAAATTCAGAATCTCACAGTCAAGCTTCAGCTGCAGGCTCTTC | enzyme 2 |
| | AGCAAAATGGGTCTTCAGTGCTCTCAGAAGACAAGAGCAAACGGTTGAACACAATTCTAAATACAATGAG | (ACE2), |
| | CACCATCTACAGTACTGGAAAAGTTTGTAACCCAGATAATCCACAAGAATGCTTATTACTTGAACCAGGT | transcript |
| | TTGAATGAAATAATGGCAAACAGTTTAGACTACAATGAGAGGCTCTGGGCTTGGGAAAGCTGGAGATCTG | variant 1, |
| | AGGTCGGCAAGCAGCTGAGGCCATTATATGAAGAGTATGTGGTCTTGAAAAATGAGATGGCAAGAGCAAA | mRNA |
| | TCATTATGAGGACTATGGGGATTATTGGAGAGGAGACTATGAAGTAAATGGGGTAGATGGCTATGACTAC | |
| | AGCCGCGGCCAGTTGATTGAAGATGTGGAACATACCTTTGAAGAGATTAAACCATTATATGAACATCTTC | |
| | ATGCCTATGTGAGGGCAAAGTTGATGAATGCCTATCCTTCCTATATCAGTCCAATTGGATGCCTCCCTGC | |
| | TCATTTGCTTGGTGATATGTGGGGTAGATTTTGGACAAATCTGTACTCTTTGACAGTTCCCTTTGGACAG | |
| | AAACCAAACATAGATGTTACTGATGCAATGGTGGACCAGGCCTGGGATGCACAGAGAATATTCAAGGAGG | |
| | CCGAGAAGTTCTTTGTATCTGTTGGTCTTCCTAATATGACTCAAGGATTCTGGGAAAATTCCATGCTAAC | |
| | GGACCCAGGAAATGTTCAGAAAGCAGTCTGCCATCCCACAGCTTGGGACCTGGGGAAGGGCGACTTCAGG | |
| | ATCCTTATGTGCACAAAGGTGACAATGGACGACTTCCTGACAGCTCATCATGAGATGGGGCATATCCAGT | |
| | ATGATATGGCATATGCTGCACAACCTTTTCTGCTAAGAAATGGAGCTAATGAAGGATTCCATGAAGCTGT | |
| | TGGGGAAATCATGTCACTTTCTGCAGCCACACCTAAGCATTTAAAATCCATTGGTCTTCTGTCACCCGAT | |
| | TTTCAAGAAGACAATGAAACAGAAATAAACTTCCTGCTCAAACAAGCACTCACGATTGTTGGGACTCTGC | |
| | CATTTACTTACATGTTAGAGAAGTGGAGGTGGATGGTCTTTAAAGGGGAAATTCCCAAAGACCAGTGGAT | |
| | GAAAAAGTGGTGGGAGATGAAGCGAGAGATAGTTGGGGTGGTGGAACCTGTGCCCCATGATGAAACATAC | |
| | TGTGACCCCGCATCTCTGTTCCATGTTTCTAATGATTACTCATTCATTCGATATTACACAAGGACCCTTT | |
| | ACCAATTCCAGTTTCAAGAAGCACTTTGTCAAGCAGCTAAACATGAAGGCCCTCTGCACAAATGTGACAT | |
| | CTCAAACTCTACAGAAGCTGGACAGAAACTGTTCAATATGCTGAGGCTTGGAAAATCAGAACCCTGGACC | |
| | CTAGCATTGGAAAATGTTGTAGGAGCAAAGAACATGAATGTAAGGCCACTGCTCAACTACTTTGAGCCCT | |
| | TATTTACCTGGCTGAAAGACCAGAACAAGAATTCTTTTGTGGGATGGAGTACCGACTGGAGTCCATATGC | |
| | AGACCAAAGCATCAAAGTGAGGATAAGCCTAAAATCAGCTCTTGGAGATAAAGCATATGAATGGAACGAC | |
| | AATGAAATGTACCTGTTCCGATCATCTGTTGCATATGCTATGAGGCAGTACTTTTTAAAAGTAAAAAATC | |
| | AGATGATTCTTTTTGGGGAGGAGGATGTGCGAGTGGCTAATTTGAAACCAAGAATCTCCTTTAATTTCTT | |
| | TGTCACTGCACCTAAAAATGTGTCTGATATCATTCCTAGAACTGAAGTTGAAAAGGCCATCAGGATGTCC | |
| | CGGAGCCGTATCAATGATGCTTTCCGTCTGAATGACAACAGCCTAGAGTTTCTGGGGATACAGCCAACAC | |
| | TTGGACCTCCTAACCAGCCCCCTGTTTCCATATGGCTGATTGTTTTTGGAGTTGTGATGGGAGTGATAGT | |
| | GGTTGGCATTGTCATCCTGATCTTCACTGGGATCAGAGATCGGAAGAAGAAAAATAAAGCAAGAAGTGGA | |
| | GAAAATCCTTATGCCTCCATCGATATTAGCAAAGGAGAAAATAATCCAGGATTCCAAAACACTGATGATG | |
| | TTCAGACCTCCTTTTAGAAAAATCTATGTTTTTCCTCTTGAGGTGATTTTGTTGTATGTAAATGTTAATT | |
| | TCATGGTATAGAAAATATAAGATGATAAAGATATCATTAAATGTCAAAACTATGACTCTGTTCAGAAAAA | |
| | AAATTGTCCAAAGACAACATGGCCAAGGAGAGAGCATCTTCATTGACATTGCTTTCAGTATTTATTTCTG | |
| | TCTCTGGATTTGACTTCTGTTCTGTTTCTTAATAAGGATTTTGTATTAGAGTATATTAGGGAAAGTGTGT | |
| | ATTTGGTCTCACAGGCTGTTCAGGGATAATCTAAATGTAAATGTCTGTTGAATTTCTGAAGTTGAAAACA | |
| | AGGATATATCATTGGAGCAAGTGTTGGATCTTGTATGGAATATGGATGGATCACTTGTAAGGACAGTGCC | |
| | TGGGAACTGGTGTAGCTGCAAGGATTGAGAATGGCATGCATTAGCTCACTTTCATTTAATCCATTGTCAA | |
| | GGATGACATGCTTTCTTCACAGTAACTCAGTTCAAGTACTATGGTGATTTGCCTACAGTGATGTTTGGAA | |
| | TCGATCATGCTTTCTTCAAGGTGACAGGTCTAAAGAGAGAAGAATCCAGGGAACAGGTAGAGGACATTGC | |
| | TTTTTCACTTCCAAGGTGCTTGATCAACATCTCCCTGACAACACAAAACTAGAGCCAGGGGCCTCCGTGA | |
| | ACTCCCAGAGCATGCCTGATAGAAACTCATTTCTACTGTTCTCTAACTGTGGAGTGAATGGAAATTCCAA | |
| | CTGTATGTTCACCCTCTGAAGTGGGTACCCAGTCTCTTAAATCTTTTGTATTTGCTCACAGTGTTTGAGC | |
| | AGTGCTGAGCACAAAGCAGACACTCAATAAATGCTAGATTTACACACTC | |
| 2 | AGTCTAGGGAAAGTCATTCAGTGGATGTGATCTTGGCTCACAGGGGACGATGTCAAGCTCTTCCTGGCTC | >NM_001386259.1 |
| | CTTCTCAGCCTTGTTGCTGTAACTGCTGCTCAGTCCACCATTGAGGAACAGGCCAAGACATTTTTGGACA | Homo sapiens |
| | AGTTTAACCACGAAGCCGAAGACCTGTTCTATCAAAGTTCACTTGCTTCTTGGAATTATAACACCAATAT | angiotensin |
| | TACTGAAGAGAATGTCCAAAACATGAATAATGCTGGGGACAAATGGTCTGCCTTTTTAAAGGAACAGTCC | converting |
| | ACACTTGCCCAAATGTATCCACTACAAGAAATTCAGAATCTCACAGTCAAGCTTCAGCTGCAGGCTCTTC | enzyme 2 |
| | AGCAAAATGGGTCTTCAGTGCTCTCAGAAGACAAGAGCAAACGGTTGAACACAATTCTAAATACAATGAG | (ACE2), |
| | CACCATCTACAGTACTGGAAAAGTTTGTAACCCAGATAATCCACAAGAATGCTTATTACTTGAACCAGGT | transcript |
| | TTGAATGAAATAATGGCAAACAGTTTAGACTACAATGAGAGGCTCTGGGCTTGGGAAAGCTGGAGATCTG | variant 3, |
| | AGGTCGGCAAGCAGCTGAGGCCATTATATGAAGAGTATGTGGTCTTGAAAAATGAGATGGCAAGAGCAAA | mRNA |
| | TCATTATGAGGACTATGGGGATTATTGGAGAGGAGACTATGAAGTAAATGGGGTAGATGGCTATGACTAC | |
| | AGCCGCGGCCAGTTGATTGAAGATGTGGAACATACCTTTGAAGAGATTAAACCATTATATGAACATCTTC | |
| | ATGCCTATGTGAGGGCAAAGTTGATGAATGCCTATCCTTCCTATATCAGTCCAATTGGATGCCTCCCTGC | |
| | TCATTTGCTTGGTGATATGTGGGGTAGATTTTGGACAAATCTGTACTCTTTGACAGTTCCCTTTGGACAG | |
| | AAACCAAACATAGATGTTACTGATGCAATGGTGGACCAGGCCTGGGATGCACAGAGAATATTCAAGGAGG | |
| | CCGAGAAGTTCTTTGTATCTGTTGGTCTTCCTAATATGACTCAAGGATTCTGGGAAAATTCCATGCTAAC | |
| | GGACCCAGGAAATGTTCAGAAAGCAGTCTGCCATCCCACAGCTTGGGACCTGGGGAAGGGCGACTTCAGG | |
| | ATCCTTATGTGCACAAAGGTGACAATGGACGACTTCCTGACAGCTCATCATGAGATGGGGCATATCCAGT | |
| | ATGATATGGCATATGCTGCACAACCTTTTCTGCTAAGAAATGGAGCTAATGAAGGATTCCATGAAGCTGT | |
| | TGGGGAAATCATGTCACTTTCTGCAGCCACACCTAAGCATTTAAAATCCATTGGTCTTCTGTCACCCGAT | |
| | TTTCAAGAAGACAATGAAACAGAAATAAACTTCCTGCTCAAACAAGCACTCACGATTGTTGGGACTCTGC | |
| | CATTTACTTACATGTTAGAGAAGTGGAGGTGGATGGTCTTTAAAGGGGAAATTCCCAAAGACCAGTGGAT | |
| | GAAAAAGTGGTGGGAGATGAAGCGAGAGATAGTTGGGGTGGTGGAACCTGTGCCCCATGATGAAACATAC | |
| | TGTGACCCCGCATCTCTGTTCCATGTTTCTAATGATTACTCATTCATTCGATATTACACAAGGACCCTTT | |
| | ACCAATTCCAGTTTCAAGAAGCACTTTGTCAAGCAGCTAAACATGAAGGCCCTCTGCACAAATGTGACAT | |
| | CTCAAACTCTACAGAAGCTGGACAGAAACTGTTCAATATGCTGAGGCTTGGAAAATCAGAACCCTGGACC | |
| | CTAGCATTGGAAAATGTTGTAGGAGCAAAGAACATGAATGTAAGGCCACTGCTCAACTACTTTGAGCCCT | |
| | TATTTACCTGGCTGAAAGACCAGAACAAGAATTCTTTTGTGGGATGGAGTACCGACTGGAGTCCATATGC | |
| | AGACCAAAGCATCAAAGTGAGGATAAGCCTAAAATCAGCTCTTGGAGATAAAGCATATGAATGGAACGAC | |
| | AATGAAATGTACCTGTTCCGATCATCTGTTGCATATGCTATGAGGCAGTACTTTTTAAAAGTAAAAAATC | |
| | AGATGATTCTTTTTGGGGAGGAGGATGTGCGAGTGGCTAATTTGAAACCAAGAATCTCCTTTAATTTCTT | |
| | TGTCACTGCACCTAAAAATGTGTCTGATATCATTCCTAGAACTGAAGTTGAAAAGGCCATCAGGATGTCC | |
| | CGGAGCCGTATCAATGATGCTTTCCGTCTGAATGACAACAGCCTAGAGTTTCTGGGGATACAGCCAACAC | |
| | TTGGACCTCCTAACCAGCCCCCTGTTTCCATATGGCTGATTGTTTTTGGAGTTGTGATGGGAGTGATAGT | |
| | GGTTGGCATTGTCATCCTGATCTTCACTGGGATCAGAGATCGGAAGAAGCCAACTCCACTCTTGGGAAAA | |
| | AGTTGGCTGACAGCCATCTTGAAAGATTGAGGGCTGAAAATCCAAGAACTGAGGATCAAGATCTCTCCCC | |
| | TGTCATAAAACTACATATGGATCTGCCCTTCAGTAGGAAATTCCTAAAAGTCTCCCATGAGATAAAGAAT | |
| | CAGTGCTGGAAAACTCACTCCGATACCACCACCACCAAATCATGATAGAAACAGCTATGTGTGTCTTTTT | |
| | TTAATTAGACCTCATCTTCCTTGGAACTAACTCTGAAAGGGCCATGAATCTCAGCCCCCCCAAAATCCCT | |
| | CCCCAAAAGCATGCTGCCAGGTGATGCAGGCCCAAGCTAGGTGACAGATGTTTAACTTGGAATGATGTTT | |
| | GCAGTCATGTGATAATAACATTGGATGGAACAATTCAGAGGCTGTTCTTATGATTACAAGTAATGGGGAC | |
| | ATTTTTATCATTTGAGAATGACTGCAAAACTATGGAATTTGGCAAAGACTTTATTTGGAAGCAGGGAAGA | |
| | AAGCCCACTGAATAGCTTTGAAGGGATAATGGAGGGAAAGAATTATGTTGTTTTCTGCTTTTGTCCTATA | |
| | GAGTTTCATTTCAACACCAGGATACTTCCACAAAGCAGTCTTGGCCATGTTGATGGTAAGGAAAGAATGA | |
| | CAGCTAATAACAGCTGCCTGTTATGTGTGATGCCATCTTAAGGACATCTCCCGCATGCACCCATTTTTTC | |
| | TTTTTTTTTTTTTGGTGACTATTTATGGGCTTACTGGCTAGGAAAAGACACAACAATGAAA | |
| 3 | AGTCTAGGGAAAGTCATTCAGTGGATGTGATCTTGGCTCACAGGGGACGATGTCAAGCTCTTCCTGGCTC | >NM_001386260.1 |
| | CTTCTCAGCCTTGTTGCTGTAACTGCTGCTCAGTCCACCATTGAGGAACAGGCCAAGACATTTTTGGACA | Homo sapiens |
| | AGTTTAACCACGAAGCCGAAGACCTGTTCTATCAAAGTTCACTTGCTTCTTGGAATTATAACACCAATAT | angiotensin |
| | TACTGAAGAGAATGTCCAAAACATGAATAATGCTGGGGACAAATGGTCTGCCTTTTTAAAGGAACAGTCC | converting |
| | ACACTTGCCCAAATGTATCCACTACAAGAAATTCAGAATCTCACAGTCAAGCTTCAGCTGCAGGCTCTTC | enzyme 2 |
| | AGCAAAATGGGTCTTCAGTGCTCTCAGAAGACAAGAGCAAACGGTTGAACACAATTCTAAATACAATGAG | (ACE2), |
| | CACCATCTACAGTACTGGAAAAGTTTGTAACCCAGATAATCCACAAGAATGCTTATTACTTGAACCAGGT | transcript |
| | TTGAATGAAATAATGGCAAACAGTTTAGACTACAATGAGAGGCTCTGGGCTTGGGAAAGCTGGAGATCTG | variant 4, |
| | AGGTCGGCAAGCAGCTGAGGCCATTATATGAAGAGTATGTGGTCTTGAAAAATGAGATGGCAAGAGCAAA | mRNA |
| | TCATTATGAGGACTATGGGGATTATTGGAGAGGAGACTATGAAGTAAATGGGGTAGATGGCTATGACTAC | |
| | AGCCGCGGCCAGTTGATTGAAGATGTGGAACATACCTTTGAAGAGATTAAACCATTATATGAACATCTTC | |
| | ATGCCTATGTGAGGGCAAAGTTGATGAATGCCTATCCTTCCTATATCAGTCCAATTGGATGCCTCCCTGC | |
| | TCATTTGCTTGGTGATATGTGGGGTAGATTTTGGACAAATCTGTACTCTTTGACAGTTCCCTTTGGACAG | |
| | AAACCAAACATAGATGTTACTGATGCAATGGTGGACCAGGCCTGGGATGCACAGAGAATATTCAAGGAGG | |
| | CCGAGAAGTTCTTTGTATCTGTTGGTCTTCCTAATATGACTCAAGGATTCTGGGAAAATTCCATGCTAAC | |
| | GGACCCAGGAAATGTTCAGAAAGCAGTCTGCCATCCCACAGCTTGGGACCTGGGGAAGGGCGACTTCAGG | |
| | ATCCTTATGTGCACAAAGGTGACAATGGACGACTTCCTGACAGCTCATCATGAGATGGGGCATATCCAGT | |
| | ATGATATGGCATATGCTGCACAACCTTTTCTGCTAAGAAATGGAGCTAATGAAGGATTCCATGAAGCTGT | |
| | TGGGGAAATCATGTCACTTTCTGCAGCCACACCTAAGCATTTAAAATCCATTGGTCTTCTGTCACCCGAT | |
| | TTTCAAGAAGACAATGAAACAGAAATAAACTTCCTGCTCAAACAAGCACTCACGATTGTTGGGACTCTGC | |
| | CATTTACTTACATGTTAGAGAAGTGGAGGTGGATGGTCTTTAAAGGGGAAATTCCCAAAGACCAGTGGAT | |
| | GAAAAAGTGGTGGGAGATGAAGCGAGAGATAGTTGGGGTGGTGGAACCTGTGCCCCATGATGAAACATAC | |
| | TGTGACCCCGCATCTCTGTTCCATGTTTCTAATGATTACTCATTCATTCGATATTACACAAGGACCCTTT | |
| | ACCAATTCCAGTTTCAAGAAGCACTTTGTCAAGCAGCTAAACATGAAGGCCCTCTGCACAAATGTGACAT | |
| | CTCAAACTCTACAGAAGCTGGACAGAAACTGTTGGAGGAGGATGTGCGAGTGGCTAATTTGAAACCAAGA | |
| | ATCTCCTTTAATTTCTTTGTCACTGCACCTAAAAATGTGTCTGATATCATTCCTAGAACTGAAGTTGAAA | |
| | AGGCCATCAGGATGTCCCGGAGCCGTATCAATGATGCTTTCCGTCTGAATGACAACAGCCTAGAGTTTCT | |
| | GGGGATACAGCCAACACTTGGACCTCCTAACCAGCCCCCTGTTTCCATATGGCTGATTGTTTTTGGAGTT | |
| | GTGATGGGAGTGATAGTGGTTGGCATTGTCATCCTGATCTTCACTGGGATCAGAGATCGGAAGAAGAAAA | |
| | ATAAAGCAAGAAGTGGAGAAAATCCTTATGCCTCCATCGATATTAGCAAAGGAGAAAATAATCCAGGATT | |
| | CCAAAACACTGATGATGTTCAGACCTCCTTTTAGAAAAATCTATGTTTTTCCTCTTGAGGTGATTTTGTT | |
| | GTATGTAAATGTTAATTTCATGGTATAGAAAATATAAGATGATAAAGATATCATTAAATGTCAAAACTAT | |
| | GACTCTGTTCAGAAAAAAAATTGTCCAAAGACAACATGGCCAAGGAGAGAGCATCTTCATTGACATTGCT | |
| | TTCAGTATTTATTTCTGTCTCTGGATTTGACTTCTGTTCTGTTTCTTAATAAGGATTTTGTATTAGAGTA | |
| | TATTAGGGAAAGTGTGTATTTGGTCTCACAGGCTGTTCAGGGATAATCTAAATGTAAATGTCTGTTGAAT | |
| | TTCTGAAGTTGAAAACAAGGATATATCATTGGAGCAAGTGTTGGATCTTGTATGGAATATGGATGGATCA | |
| | CTTGTAAGGACAGTGCCTGGGAACTGGTGTAGCTGCAAGGATTGAGAATGGCATGCATTAGCTCACTTTC | |
| | ATTTAATCCATTGTCAAGGATGACATGCTTTCTTCACAGTAACTCAGTTCAAGTACTATGGTGATTTGCC | |
| | TACAGTGATGTTTGGAATCGATCATGCTTTCTTCAAGGTGACAGGTCTAAAGAGAGAAGAATCCAGGGAA | |
| | CAGGTAGAGGACATTGCTTTTTCACTTCCAAGGTGCTTGATCAACATCTCCCTGACAACACAAAACTAGA | |
| | GCCAGGGGCCTCCGTGAACTCCCAGAGCATGCCTGATAGAAACTCATTTCTACTGTTCTCTAACTGTGGA | |
| | GTGAATGGAAATTCCAACTGTATGTTCACCCTCTGAAGTGGGTACCCAGTCTCTTAAATCTTTTGTATTT | |
| | GCTCACAGTGTTTGAGCAGTGCTGAGCACAAAGCAGACACTCAATAAATGCTAGATTTACACACTC | |
| 4 | GTAATTCCCAGGTTGCAGGCTTGTGAGAGCCTTAGGTTGGATTCCCTAGCTTGAAAAGGAGATCGTTTTA | >NM_001388452.1 |
| | CAAGTGCTTCATTGAGGAGAGCTCTGAGGCAGAGGGGAATGAGGGAAGCAGGCTGGGACAAAGGAGGGAG | Homo sapiens |
| | GATCCTTATGTGCACAAAGGTGACAATGGACGACTTCCTGACAGCTCATCATGAGATGGGGCATATCCAG | angiotensin |
| | TATGATATGGCATATGCTGCACAACCTTTTCTGCTAAGAAATGGAGCTAATGAAGGATTCCATGAAGCTG | converting |
| | TTGGGGAAATCATGTCACTTTCTGCAGCCACACCTAAGCATTTAAAATCCATTGGTCTTCTGTCACCCGA | enzyme 2 |
| | TTTTCAAGAAGACAATGAAACAGAAATAAACTTCCTGCTCAAACAAGCACTCACGATTGTTGGGACTCTG | (ACE2), |
| | CCATTTACTTACATGTTAGAGAAGTGGAGGTGGATGGTCTTTAAAGGGGAAATTCCCAAAGACCAGTGGA | transcript |
| | TGAAAAAGTGGTGGGAGATGAAGCGAGAGATAGTTGGGGTGGTGGAACCTGTGCCCCATGATGAAACATA | variant 5, |
| | CTGTGACCCCGCATCTCTGTTCCATGTTTCTAATGATTACTCATTCATTCGATATTACACAAGGACCCTT | mRNA |
| | TACCAATTCCAGTTTCAAGAAGCACTTTGTCAAGCAGCTAAACATGAAGGCCCTCTGCACAAATGTGACA | |
| | TCTCAAACTCTACAGAAGCTGGACAGAAACTGTTCAATATGCTGAGGCTTGGAAAATCAGAACCCTGGAC | |
| | CCTAGCATTGGAAAATGTTGTAGGAGCAAAGAACATGAATGTAAGGCCACTGCTCAACTACTTTGAGCCC | |
| | TTATTTACCTGGCTGAAAGACCAGAACAAGAATTCTTTTGTGGGATGGAGTACCGACTGGAGTCCATATG | |
| | CAGACCAAAGCATCAAAGTGAGGATAAGCCTAAAATCAGCTCTTGGAGATAAAGCATATGAATGGAACGA | |
| | CAATGAAATGTACCTGTTCCGATCATCTGTTGCATATGCTATGAGGCAGTACTTTTTAAAAGTAAAAAAT | |
| | CAGATGATTCTTTTTGGGGAGGAGGATGTGCGAGTGGCTAATTTGAAACCAAGAATCTCCTTTAATTTCT | |
| | TTGTCACTGCACCTAAAAATGTGTCTGATATCATTCCTAGAACTGAAGTTGAAAAGGCCATCAGGATGTC | |
| | CCGGAGCCGTATCAATGATGCTTTCCGTCTGAATGACAACAGCCTAGAGTTTCTGGGGATACAGCCAACA | |
| | CTTGGACCTCCTAACCAGCCCCCTGTTTCCATATGGCTGATTGTTTTTGGAGTTGTGATGGGAGTGATAG | |
| | TGGTTGGCATTGTCATCCTGATCTTCACTGGGATCAGAGATCGGAAGAAGAAAAATAAAGCAAGAAGTGG | |
| | AGAAAATCCTTATGCCTCCATCGATATTAGCAAAGGAGAAAATAATCCAGGATTCCAAAACACTGATGAT | |
| | GTTCAGACCTCCTTTTAGAAAAATCTATGTTTTTCCTCTTGAGGTGATTTTGTTGTATGTAAATGTTAAT | |
| | TTCATGGTATAGAAAATATAAGATGATAAAGATATCATTAAATGTCAAAACTATGACTCTGTTCAGAAAA | |
| | AAAATTGTCCAAAGACAACATGGCCAAGGAGAGAGCATCTTCATTGACATTGCTTTCAGTATTTATTTCT | |
| | GTCTCTGGATTTGACTTCTGTTCTGTTTCTTAATAAGGATTTTGTATTAGAGTATATTAGGGAAAGTGTG | |
| | TATTTGGTCTCACAGGCTGTTCAGGGATAATCTAAATGTAAATGTCTGTTGAATTTCTGAAGTTGAAAAC | |
| | AAGGATATATCATTGGAGCAAGTGTTGGATCTTGTATGGAATATGGATGGATCACTTGTAAGGACAGTGC | |
| | CTGGGAACTGGTGTAGCTGCAAGGATTGAGAATGGCATGCATTAGCTCACTTTCATTTAATCCATTGTCA | |
| | AGGATGACATGCTTTCTTCACAGTAACTCAGTTCAAGTACTATGGTGATTTGCCTACAGTGATGTTTGGA | |
| | ATCGATCATGCTTTCTTCAAGGTGACAGGTCTAAAGAGAGAAGAATCCAGGGAACAGGTAGAGGACATTG | |
| | CTTTTTCACTTCCAAGGTGCTTGATCAACATCTCCCTGACAACACAAAACTAGAGCCAGGGGCCTCCGTG | |
| | AACTCCCAGAGCATGCCTGATAGAAACTCATTTCTACTGTTCTCTAACTGTGGAGTGAATGGAAATTCCA | |
| | ACTGTATGTTCACCCTCTGAAGTGGGTACCCAGTCTCTTAAATCTTTTGTATTTGCTCACAGTGTTTGAG | |
| | CAGTGCTGAGCACAAAGCAGACACTCAATAAATGCTAGATTTACACACTC | |
| 5 | TTAGAACTTTTTAAAAGAGGCAAAGGCAGAGGAGAACAAAGGAAGGAGGAAGTAACTTGTGGAATGTTGA | >NM_001389402.1 |
| | GAAAGCGCCCAACCCAAGTTCAAAGGCTGATAAGAGAGAAAATCTCATGAGGAGGTTTTAGTCTAGGGAA | Homo sapiens |
| | AGTCATTCAGTGGATGTGATCTTGGCTCACAGGGGACGATGTCAAGCTCTTCCTGGCTCCTTCTCAGCCT | angiotensin |
| | TGTTGCTGTAACTGCTGCTCAGTCCACCATTGAGGAACAGGCCAAGACATTTTTGGACAAGTTTAACCAC | converting |
| | GAAGCCGAAGACCTGTTCTATCAAAGTTCACTTGCTTCTTGGAATTATAACACCAATATTACTGAAGAGA | enzyme 2 |
| | ATGTCCAAAACATGAATAATGCTGGGGACAAATGGTCTGCCTTTTTAAAGGAACAGTCCACACTTGCCCA | (ACE2), |
| | AATGTATCCACTACAAGAAATTCAGAATCTCACAGTCAAGCTTCAGCTGCAGGCTCTTCAGCAAAATGGG | transcript |
| | TCTTCAGTGCTCTCAGAAGACAAGAGCAAACGGTTGAACACAATTCTAAATACAATGAGCACCATCTACA | variant 6, |
| | GTACTGGAAAAGTTTGTAACCCAGATAATCCACAAGAATGCTTATTACTTGAACCAGGTTTGAATGAAAT | mRNA |
| | AATGGCAAACAGTTTAGACTACAATGAGAGGCTCTGGGCTTGGGAAAGCTGGAGATCTGAGGTCGGCAAG | |
| | CAGCTGAGGCCATTATATGAAGAGTATGTGGTCTTGAAAAATGAGATGGCAAGAGCAAATCATTATGAGG | |
| | ACTATGGGGATTATTGGAGAGGAGACTATGAAGTAAATGGGGTAGATGGCTATGACTACAGCCGCGGCCA | |
| | GTTGATTGAAGATGTGGAACATACCTTTGAAGAGATTAAACCATTATATGAACATCTTCATGCCTATGTG | |
| | AGGGCAAAGTTGATGAATGCCTATCCTTCCTATATCAGTCCAATTGGATGCCTCCCTGCTCATTTGCTTG | |
| | GTGATATGTGGGGTAGATTTTGGACAAATCTGTACTCTTTGACAGTTCCCTTTGGACAGAAACCAAACAT | |
| | AGATGTTACTGATGCAATGGTGGACCAGGCCTGGGATGCACAGAGAATATTCAAGGAGGCCGAGAAGTTC | |
| | TTTGTATCTGTTGGTCTTCCTAATATGACTCAAGGATTCTGGGAAAATTCCATGCTAACGGACCCAGGAA | |
| | ATGTTCAGAAAGCAGTCTGCCATCCCACAGCTTGGGACCTGGGGAAGGGCGACTTCAGGATCCTTATGTG | |
| | CACAAAGGTGACAATGGACGACTTCCTGACAGCTCATCATGAGATGGGGCATATCCAGTATGATATGGCA | |
| | TATGCTGCACAACCTTTTCTGCTAAGAAATGGAGCTAATGAAGGATTCCATGAAGCTGTTGGGGAAATCA | |
| | TGTCACTTTCTGCAGCCACACCTAAGCATTTAAAATCCATTGGTCTTCTGTCACCCGATTTTCAAGAAGA | |
| | CAATGAAACAGAAATAAACTTCCTGCTCAAACAAGCACTCACGATTGTTGGGACTCTGCCATTTACTTAC | |
| | ATGTTAGAGAAGTGGAGGTGGATGGTCTTTAAAGGGGAAATTCCCAAAGACCAGTGGATGAAAAAGTGGT | |
| | GGGAGATGAAGCGAGAGATAGTTGGGGTGGTGGAACCTGTGCCCCATGATGAAACATACTGTGACCCCGC | |
| | ATCTCTGTTCCATGTTTCTAATGATTACTCATTCATTCGATATTACACAAGGACCCTTTACCAATTCCAG | |
| | TTTCAAGAAGCACTTTGTCAAGCAGCTAAACATGAAGGCCCTCTGCACAAATGTGACATCTCAAACTCTA | |
| | CAGAAGCTGGACAGAAACTGTTGGAGGAGGATGTGCGAGTGGCTAATTTGAAACCAAGAATCTCCTTTAA | |
| | TTTCTTTGTCACTGCACCTAAAAATGTGTCTGATATCATTCCTAGAACTGAAGTTGAAAAGGCCATCAGG | |
| | ATGTCCCGGAGCCGTATCAATGATGCTTTCCGTCTGAATGACAACAGCCTAGAGTTTCTGGGGATACAGC | |
| | CAACACTTGGACCTCCTAACCAGCCCCCTGTTTCCATATGGCTGATTGTTTTTGGAGTTGTGATGGGAGT | |
| | GATAGTGGTTGGCATTGTCATCCTGATCTTCACTGGGATCAGAGATCGGAAGAAGAAAAATAAAGCAAGA | |
| | AGTGGAGAAAATCCTTATGCCTCCATCGATATTAGCAAAGGAGAAAATAATCCAGGATTCCAAAACACTG | |
| | ATGATGTTCAGACCTCCTTTTAGAAAAATCTATGTTTTTCCTCTTGAGGTGATTTTGTTGTATGTAAATG | |
| | TTAATTTCATGGTATAGAAAATATAAGATGATAAAGATATCATTAAATGTCAAAACTATGACTCTGTTCA | |
| | GAAAAAAAATTGTCCAAAGACAACATGGCCAAGGAGAGAGCATCTTCATTGACATTGCTTTCAGTATTTA | |
| | TTTCTGTCTCTGGATTTGACTTCTGTTCTGTTTCTTAATAAGGATTTTGTATTAGAGTATATTAGGGAAA | |
| | GTGTGTATTTGGTCTCACAGGCTGTTCAGGGATAATCTAAATGTAAATGTCTGTTGAATTTCTGAAGTTG | |
| | AAAACAAGGATATATCATTGGAGCAAGTGTTGGATCTTGTATGGAATATGGATGGATCACTTGTAAGGAC | |
| | AGTGCCTGGGAACTGGTGTAGCTGCAAGGATTGAGAATGGCATGCATTAGCTCACTTTCATTTAATCCAT | |
| | TGTCAAGGATGACATGCTTTCTTCACAGTAACTCAGTTCAAGTACTATGGTGATTTGCCTACAGTGATGT | |
| | TTGGAATCGATCATGCTTTCTTCAAGGTGACAGGTCTAAAGAGAGAAGAATCCAGGGAACAGGTAGAGGA | |
| | CATTGCTTTTTCACTTCCAAGGTGCTTGATCAACATCTCCCTGACAACACAAAACTAGAGCCAGGGGCCT | |
| | CCGTGAACTCCCAGAGCATGCCTGATAGAAACTCATTTCTACTGTTCTCTAACTGTGGAGTGAATGGAAA | |
| | TTCCAACTGTATGTTCACCCTCTGAAGTGGGTACCCAGTCTCTTAAATCTTTTGTATTTGCTCACAGTGT | |
| | TTGAGCAGTGCTGAGCACAAAGCAGACACTCAATAAATGCTAGATTTACACACTC | |
| 6 | GGCACTCATACATACACTCTGGCAATGAGGACACTGAGCTCGCTTCTGAAATTTGACAAGATAACCACTA | >NM_021804.3 |
| | AAATCTCTTTGAATTCTATGTTGTTGTGATCCCATGGCTACAGAGGATCAGGAGTTGACATAGATACTCT | Homo sapiens |
| | TTGGATTTCATACCATGTGGAGGCTTTCTTACTTCCACGTGACCTTGACTGAGTTTTGAATAGCGCCCAA | angiotensin |
| | CCCAAGTTCAAAGGCTGATAAGAGAGAAAATCTCATGAGGAGGTTTTAGTCTAGGGAAAGTCATTCAGTG | converting |
| | GATGTGATCTTGGCTCACAGGGGACGATGTCAAGCTCTTCCTGGCTCCTTCTCAGCCTTGTTGCTGTAAC | enzyme 2 |
| | TGCTGCTCAGTCCACCATTGAGGAACAGGCCAAGACATTTTTGGACAAGTTTAACCACGAAGCCGAAGAC | (ACE2), |
| | CTGTTCTATCAAAGTTCACTTGCTTCTTGGAATTATAACACCAATATTACTGAAGAGAATGTCCAAAACA | transcript |
| | TGAATAATGCTGGGGACAAATGGTCTGCCTTTTTAAAGGAACAGTCCACACTTGCCCAAATGTATCCACT | variant 2, |
| | ACAAGAAATTCAGAATCTCACAGTCAAGCTTCAGCTGCAGGCTCTTCAGCAAAATGGGTCTTCAGTGCTC | mRNA |
| | TCAGAAGACAAGAGCAAACGGTTGAACACAATTCTAAATACAATGAGCACCATCTACAGTACTGGAAAAG | |
| | TTTGTAACCCAGATAATCCACAAGAATGCTTATTACTTGAACCAGGTTTGAATGAAATAATGGCAAACAG | |
| | TTTAGACTACAATGAGAGGCTCTGGGCTTGGGAAAGCTGGAGATCTGAGGTCGGCAAGCAGCTGAGGCCA | |
| | TTATATGAAGAGTATGTGGTCTTGAAAAATGAGATGGCAAGAGCAAATCATTATGAGGACTATGGGGATT | |
| | ATTGGAGAGGAGACTATGAAGTAAATGGGGTAGATGGCTATGACTACAGCCGCGGCCAGTTGATTGAAGA | |
| | TGTGGAACATACCTTTGAAGAGATTAAACCATTATATGAACATCTTCATGCCTATGTGAGGGCAAAGTTG | |
| | ATGAATGCCTATCCTTCCTATATCAGTCCAATTGGATGCCTCCCTGCTCATTTGCTTGGTGATATGTGGG | |
| | GTAGATTTTGGACAAATCTGTACTCTTTGACAGTTCCCTTTGGACAGAAACCAAACATAGATGTTACTGA | |
| | TGCAATGGTGGACCAGGCCTGGGATGCACAGAGAATATTCAAGGAGGCCGAGAAGTTCTTTGTATCTGTT | |
| | GGTCTTCCTAATATGACTCAAGGATTCTGGGAAAATTCCATGCTAACGGACCCAGGAAATGTTCAGAAAG | |
| | CAGTCTGCCATCCCACAGCTTGGGACCTGGGGAAGGGCGACTTCAGGATCCTTATGTGCACAAAGGTGAC | |
| | AATGGACGACTTCCTGACAGCTCATCATGAGATGGGGCATATCCAGTATGATATGGCATATGCTGCACAA | |
| | CCTTTTCTGCTAAGAAATGGAGCTAATGAAGGATTCCATGAAGCTGTTGGGGAAATCATGTCACTTTCTG | |
| | CAGCCACACCTAAGCATTTAAAATCCATTGGTCTTCTGTCACCCGATTTTCAAGAAGACAATGAAACAGA | |
| | AATAAACTTCCTGCTCAAACAAGCACTCACGATTGTTGGGACTCTGCCATTTACTTACATGTTAGAGAAG | |
| | TGGAGGTGGATGGTCTTTAAAGGGGAAATTCCCAAAGACCAGTGGATGAAAAAGTGGTGGGAGATGAAGC | |
| | GAGAGATAGTTGGGGTGGTGGAACCTGTGCCCCATGATGAAACATACTGTGACCCCGCATCTCTGTTCCA | |
| | TGTTTCTAATGATTACTCATTCATTCGATATTACACAAGGACCCTTTACCAATTCCAGTTTCAAGAAGCA | |
| | CTTTGTCAAGCAGCTAAACATGAAGGCCCTCTGCACAAATGTGACATCTCAAACTCTACAGAAGCTGGAC | |
| | AGAAACTGTTCAATATGCTGAGGCTTGGAAAATCAGAACCCTGGACCCTAGCATTGGAAAATGTTGTAGG | |
| | AGCAAAGAACATGAATGTAAGGCCACTGCTCAACTACTTTGAGCCCTTATTTACCTGGCTGAAAGACCAG | |
| | AACAAGAATTCTTTTGTGGGATGGAGTACCGACTGGAGTCCATATGCAGACCAAAGCATCAAAGTGAGGA | |
| | TAAGCCTAAAATCAGCTCTTGGAGATAAAGCATATGAATGGAACGACAATGAAATGTACCTGTTCCGATC | |
| | ATCTGTTGCATATGCTATGAGGCAGTACTTTTTAAAAGTAAAAAATCAGATGATTCTTTTTGGGGAGGAG | |
| | GATGTGCGAGTGGCTAATTTGAAACCAAGAATCTCCTTTAATTTCTTTGTCACTGCACCTAAAAATGTGT | |
| | CTGATATCATTCCTAGAACTGAAGTTGAAAAGGCCATCAGGATGTCCCGGAGCCGTATCAATGATGCTTT | |
| | CCGTCTGAATGACAACAGCCTAGAGTTTCTGGGGATACAGCCAACACTTGGACCTCCTAACCAGCCCCCT | |
| | GTTTCCATATGGCTGATTGTTTTTGGAGTTGTGATGGGAGTGATAGTGGTTGGCATTGTCATCCTGATCT | |
| | TCACTGGGATCAGAGATCGGAAGAAGAAAAATAAAGCAAGAAGTGGAGAAAATCCTTATGCCTCCATCGA | |
| | TATTAGCAAAGGAGAAAATAATCCAGGATTCCAAAACACTGATGATGTTCAGACCTCCTTTTAGAAAAAT | |
| | CTATGTTTTTCCTCTTGAGGTGATTTTGTTGTATGTAAATGTTAATTTCATGGTATAGAAAATATAAGAT | |
| | GATAAAGATATCATTAAATGTCAAAACTATGACTCTGTTCAGAAAAAAAATTGTCCAAAGACAACATGGC | |
| | CAAGGAGAGAGCATCTTCATTGACATTGCTTTCAGTATTTATTTCTGTCTCTGGATTTGACTTCTGTTCT | |
| | GTTTCTTAATAAGGATTTTGTATTAGAGTATATTAGGGAAAGTGTGTATTTGGTCTCACAGGCTGTTCAG | |
| | GGATAATCTAAATGTAAATGTCTGTTGAATTTCTGAAGTTGAAAACAAGGATATATCATTGGAGCAAGTG | |
| | TTGGATCTTGTATGGAATATGGATGGATCACTTGTAAGGACAGTGCCTGGGAACTGGTGTAGCTGCAAGG | |
| | ATTGAGAATGGCATGCATTAGCTCACTTTCATTTAATCCATTGTCAAGGATGACATGCTTTCTTCACAGT | |
| | AACTCAGTTCAAGTACTATGGTGATTTGCCTACAGTGATGTTTGGAATCGATCATGCTTTCTTCAAGGTG | |
| | ACAGGTCTAAAGAGAGAAGAATCCAGGGAACAGGTAGAGGACATTGCTTTTTCACTTCCAAGGTGCTTGA | |
| | TCAACATCTCCCTGACAACACAAAACTAGAGCCAGGGGCCTCCGTGAACTCCCAGAGCATGCCTGATAGA | |
| | AACTCATTTCTACTGTTCTCTAACTGTGGAGTGAATGGAAATTCCAACTGTATGTTCACCCTCTGAAGTG | |
| | GGTACCCAGTCTCTTAAATCTTTTGTATTTGCTCACAGTGTTTGAGCAGTGCTGAGCACAAAGCAGACAC | |
| | TCAATAAATGCTAGATTTACACACTC | |
| 7 | MSSSSWLLLSLVAVTAAQSTIEEQAKTFLDKFNHEAEDLFYQSSLASWNYNTNITEENVQNMNNAGDKWS | >NP_001358344.1 |
| | AFLKEQSTLAQMYPLQEIQNLTVKLQLQALQQNGSSVLSEDKSKRLNTILNTMSTIYSTGKVCNPDNPQE | angiotensin- |
| | CLLLEPGLNEIMANSLDYNERLWAWESWRSEVGKQLRPLYEEYVVLKNEMARANHYEDYGDYWRGDYEVN | converting |
| | GVDGYDYSRGQLIEDVEHTFEEIKPLYEHLHAYVRAKLMNAYPSYISPIGCLPAHLLGDMWGREWTNLYS | enzyme 2 |
| | LTVPFGQKPNIDVTDAMVDQAWDAQRIFKEAEKFFVSVGLPNMTQGFWENSMLTDPGNVQKAVCHPTAWD | isoform 1 |
| | LGKGDFRILMCTKVTMDDFLTAHHEMGHIQYDMAYAAQPFLLRNGANEGFHEAVGEIMSLSAATPKHLKS | precursor |
| | IGLLSPDFQEDNETEINFLLKQALTIVGTLPFTYMLEKWRWMVFKGEIPKDQWMKKWWEMKREIVGVVEP | [Homo |
| | VPHDETYCDPASLFHVSNDYSFIRYYTRTLYQFQFQEALCQAAKHEGPLHKCDISNSTEAGQKLFNMLRL | sapiens] |
| | GKSEPWTLALENVVGAKNMNVRPLLNYFEPLFTWLKDQNKNSFVGWSTDWSPYADQSIKVRISLKSALGD | |
| | KAYEWNDNEMYLERSSVAYAMRQYFLKVKNQMILFGEEDVRVANLKPRISENFFVTAPKNVSDIIPRTEV | |
| | EKAIRMSRSRINDAFRLNDNSLEFLGIQPTLGPPNQPPVSIWLIVFGVVMGVIVVGIVILIFTGIRDRKK | |
| | KNKARSGENPYASIDISKGENNPGFQNTDDVQTSF | |
| 8 | MSSSSWLLLSLVAVTAAQSTIEEQAKTFLDKFNHEAEDLFYQSSLASWNYNTNITEENVQNMNNAGDKWS | >NP_001373189.1 |
| | AFLKEQSTLAQMYPLQEIQNLTVKLQLQALQQNGSSVLSEDKSKRLNTILNTMSTIYSTGKVCNPDNPQE | angiotensin- |
| | CLLLEPGLNEIMANSLDYNERLWAWESWRSEVGKQLRPLYEEYVVLKNEMARANHYEDYGDYWRGDYEVN | converting |
| | GVDGYDYSRGQLIEDVEHTFEEIKPLYEHLHAYVRAKLMNAYPSYISPIGCLPAHLLGDMWGREWTNLYS | enzyme 2 |
| | LTVPFGQKPNIDVTDAMVDQAWDAQRIFKEAEKFFVSVGLPNMTQGFWENSMLTDPGNVQKAVCHPTAWD | isoform 3 |
| | LGKGDFRILMCTKVTMDDFLTAHHEMGHIQYDMAYAAQPFLLRNGANEGFHEAVGEIMSLSAATPKHLKS | precursor |
| | IGLLSPDFQEDNETEINFLLKQALTIVGTLPFTYMLEKWRWMVFKGEIPKDQWMKKWWEMKREIVGVVEP | [Homo |
| | VPHDETYCDPASLFHVSNDYSFIRYYTRTLYQFQFQEALCQAAKHEGPLHKCDISNSTEAGQKLLEEDVR | sapiens] |
| | VANLKPRISENFFVTAPKNVSDIIPRTEVEKAIRMSRSRINDAFRLNDNSLEFLGIQPTLGPPNQPPVSI | |
| | WLIVFGVVMGVIVVGIVILIFTGIRDRKKKNKARSGENPYASIDISKGENNPGFQNTDDVQTSF | |
| 9 | MREAGWDKGGRILMCTKVTMDDFLTAHHEMGHIQYDMAYAAQPFLLRNGANEGFHEAVGEIMSLSAATPK | >NP_001375381.1 |
| | HLKSIGLLSPDFQEDNETEINFLLKQALTIVGTLPFTYMLEKWRWMVFKGEIPKDQWMKKWWEMKREIVG | angiotensin- |
| | VVEPVPHDETYCDPASLFHVSNDYSFIRYYTRTLYQFQFQEALCQAAKHEGPLHKCDISNSTEAGQKLFN | converting |
| | MLRLGKSEPWTLALENVVGAKNMNVRPLLNYFEPLFTWLKDQNKNSFVGWSTDWSPYADQSIKVRISLKS | enzyme 2 |
| | ALGDKAYEWNDNEMYLERSSVAYAMRQYFLKVKNQMILFGEEDVRVANLKPRISENFFVTAPKNVSDIIP | isoform 4 |
| | RTEVEKAIRMSRSRINDAFRLNDNSLEFLGIQPTLGPPNQPPVSIWLIVFGVVMGVIVVGIVILIFTGIR | [Homo |
| | DRKKKNKARSGENPYASIDISKGENNPGFQNTDDVQTSF | sapiens] |
| 10 | MSSSSWLLLSLVAVTAAQSTIEEQAKTFLDKFNHEAEDLFYQSSLASWNYNTNITEENVQNMNNAGDKWS | >NP_001376331.1 |
| | AFLKEQSTLAQMYPLQEIQNLTVKLQLQALQQNGSSVLSEDKSKRLNTILNTMSTIYSTGKVCNPDNPQE | angiotensin- |
| | CLLLEPGLNEIMANSLDYNERLWAWESWRSEVGKQLRPLYEEYVVLKNEMARANHYEDYGDYWRGDYEVN | converting |
| | GVDGYDYSRGQLIEDVEHTFEEIKPLYEHLHAYVRAKLMNAYPSYISPIGCLPAHLLGDMWGREWTNLYS | enzyme 2 |
| | LTVPFGQKPNIDVTDAMVDQAWDAQRIFKEAEKFFVSVGLPNMTQGFWENSMLTDPGNVQKAVCHPTAWD | isoform 3 |
| | LGKGDFRILMCTKVTMDDFLTAHHEMGHIQYDMAYAAQPFLLRNGANEGFHEAVGEIMSLSAATPKHLKS | precursor |
| | IGLLSPDFQEDNETEINFLLKQALTIVGTLPFTYMLEKWRWMVFKGEIPKDQWMKKWWEMKREIVGVVEP | [Homo |
| | VPHDETYCDPASLFHVSNDYSFIRYYTRTLYQFQFQEALCQAAKHEGPLHKCDISNSTEAGQKLLEEDVR | sapiens] |
| | VANLKPRISENFFVTAPKNVSDIIPRTEVEKAIRMSRSRINDAFRLNDNSLEFLGIQPTLGPPNQPPVSI | |
| | WLIVFGVVMGVIVVGIVILIFTGIRDRKKKNKARSGENPYASIDISKGENNPGFQNTDDVQTSF | |
| 11 | MSSSSWLLLSLVAVTAAQSTIEEQAKTFLDKFNHEAEDLFYQSSLASWNYNTNITEENVQNMNNAGDKWS | >NP_068576.1 |
| | AFLKEQSTLAQMYPLQEIQNLTVKLQLQALQQNGSSVLSEDKSKRLNTILNTMSTIYSTGKVCNPDNPQE | angiotensin- |
| | CLLLEPGLNEIMANSLDYNERLWAWESWRSEVGKQLRPLYEEYVVLKNEMARANHYEDYGDYWRGDYEVN | converting |
| | GVDGYDYSRGQLIEDVEHTFEEIKPLYEHLHAYVRAKLMNAYPSYISPIGCLPAHLLGDMWGREWTNLYS | enzyme 2 |
| | LTVPFGQKPNIDVTDAMVDQAWDAQRIFKEAEKFFVSVGLPNMTQGFWENSMLTDPGNVQKAVCHPTAWD | isoform 1 |
| | LGKGDFRILMCTKVTMDDFLTAHHEMGHIQYDMAYAAQPFLLRNGANEGFHEAVGEIMSLSAATPKHLKS | precursor |
| | IGLLSPDFQEDNETEINFLLKQALTIVGTLPFTYMLEKWRWMVFKGEIPKDQWMKKWWEMKREIVGVVEP | [Homo |
| | VPHDETYCDPASLFHVSNDYSFIRYYTRTLYQFQFQEALCQAAKHEGPLHKCDISNSTEAGQKLFNMLRL | sapiens] |
| | GKSEPWTLALENVVGAKNMNVRPLLNYFEPLFTWLKDQNKNSFVGWSTDWSPYADQSIKVRISLKSALGD | |
| | KAYEWNDNEMYLERSSVAYAMRQYFLKVKNQMILFGEEDVRVANLKPRISENFFVTAPKNVSDIIPRTEV | |
| | EKAIRMSRSRINDAFRLNDNSLEFLGIQPTLGPPNQPPVSIWLIVFGVVMGVIVVGIVILIFTGIRDRKK | |
| | KNKARSGENPYASIDISKGENNPGFQNTDDVQTSF | |
| 12 | ACCAGGGTCCCGGCTCGGGGTCCGGGCTGGGGAGGGGAACCTGGGCGCCTGGGACCCGCCGATGCCCCCT | >NM_001135099.1 |
| | GCCCCGCCCGGAGGTGAAAGCGGGTGTGAGGAGCGCGGCGCGGCAGGTCATATTGAACATTCCAGATACC | Homo sapiens |
| | TATCATTACTCGATGCTGTTGATAACAGCAAGATGGCTTTGAACTCAGGGTCACCACCAGCTATTGGACC | transmembrane |
| | TTACTATGAAAACCATGGATACCAACCGGAAAACCCCTATCCCGCACAGCCCACTGTGGTCCCCACTGTC | serine |
| | TACGAGGTGCATCCGGCTCAGTACTACCCGTCCCCCGTGCCCCAGTACGCCCCGAGGGTCCTGACGCAGG | protease 2 |
| | CTTCCAACCCCGTCGTCTGCACGCAGCCCAAATCCCCATCCGGGACAGTGTGCACCTCAAAGACTAAGAA | (TMPRSS2), |
| | AGCACTGTGCATCACCTTGACCCTGGGGACCTTCCTCGTGGGAGCTGCGCTGGCCGCTGGCCTACTCTGG | transcript |
| | AAGTTCATGGGCAGCAAGTGCTCCAACTCTGGGATAGAGTGCGACTCCTCAGGTACCTGCATCAACCCCT | variant 1, |
| | CTAACTGGTGTGATGGCGTGTCACACTGCCCCGGCGGGGAGGACGAGAATCGGTGTGTTCGCCTCTACGG | mRNA |
| | ACCAAACTTCATCCTTCAGGTGTACTCATCTCAGAGGAAGTCCTGGCACCCTGTGTGCCAAGACGACTGG | |
| | AACGAGAACTACGGGCGGGCGGCCTGCAGGGACATGGGCTATAAGAATAATTTTTACTCTAGCCAAGGAA | |
| | TAGTGGATGACAGCGGATCCACCAGCTTTATGAAACTGAACACAAGTGCCGGCAATGTCGATATCTATAA | |
| | AAAACTGTACCACAGTGATGCCTGTTCTTCAAAAGCAGTGGTTTCTTTACGCTGTATAGCCTGCGGGGTC | |
| | AACTTGAACTCAAGCCGCCAGAGCAGGATTGTGGGCGGCGAGAGCGCGCTCCCGGGGGCCTGGCCCTGGC | |
| | AGGTCAGCCTGCACGTCCAGAACGTCCACGTGTGCGGAGGCTCCATCATCACCCCCGAGTGGATCGTGAC | |
| | AGCCGCCCACTGCGTGGAAAAACCTCTTAACAATCCATGGCATTGGACGGCATTTGCGGGGATTTTGAGA | |
| | CAATCTTTCATGTTCTATGGAGCCGGATACCAAGTAGAAAAAGTGATTTCTCATCCAAATTATGACTCCA | |
| | AGACCAAGAACAATGACATTGCGCTGATGAAGCTGCAGAAGCCTCTGACTTTCAACGACCTAGTGAAACC | |
| | AGTGTGTCTGCCCAACCCAGGCATGATGCTGCAGCCAGAACAGCTCTGCTGGATTTCCGGGTGGGGGGCC | |
| | ACCGAGGAGAAAGGGAAGACCTCAGAAGTGCTGAACGCTGCCAAGGTGCTTCTCATTGAGACACAGAGAT | |
| | GCAACAGCAGATATGTCTATGACAACCTGATCACACCAGCCATGATCTGTGCCGGCTTCCTGCAGGGGAA | |
| | CGTCGATTCTTGCCAGGGTGACAGTGGAGGGCCTCTGGTCACTTCGAAGAACAATATCTGGTGGCTGATA | |
| | GGGGATACAAGCTGGGGTTCTGGCTGTGCCAAAGCTTACAGACCAGGAGTGTACGGGAATGTGArGGTAT | |
| | TCACGGACTGGATTTATCGACAAATGAGGGCAGACGGCTAATCCACATGGTCTTCGTCCTTGACGTCGTT | |
| | TTACAAGAAAACAATGGGGCTGGTTTTGCTTCCCCGTGCATGATTTACTCTTAGAGATGATTCAGAGGTC | |
| | ACTTCATTTTTATTAAACAGTGAACTTGTCTGGCTTTGGCACTCTCTGCCATTCTGTGCAGGCTGCAGTG | |
| | GCTCCCCTGCCCAGCCTGCTCTCCCTAACCCCTTGTCCGCAAGGGGTGATGGCCGGCTGGTTGTGGGCAC | |
| | TGGCGGTCAAGTGTGGAGGAGAGGGGTGGAGGCTGCCCCATTGAGATCTTCCTGCTGAGTCCTTTCCAGG | |
| | GGCCAATTTTGGATGAGCATGGAGCTGTCACCTCTCAGCTGCTGGATGACTTGAGATGAAAAAGGAGAGA | |
| | CATGGAAAGGGAGACAGCCAGGTGGCACCTGCAGCGGCTGCCCTCTGGGGCCACTTGGTAGTGTCCCCAG | |
| | CCTACCTCTCCACAAGGGGATTTTGCTGATGGGTTCTTAGAGCCTTAGCAGCCCTGGATGGTGGCCAGAA | |
| | ATAAAGGGACCAGCCCTTCATGGGTGGTGACGTGGTAGTCACTTGTAAGGGGAACAGAAACATTTTTGTT | |
| | CTTATGGGGTGAGAATATAGACAGTGCCCTTGGTGCGAGGGAAGCAATTGAAAAGGAACTTGCCCTGAGC | |
| | ACTCCTGGTGCAGGTCTCCACCTGCACATTGGGTGGGGCTCCTGGGAGGGAGACTCAGCCTTCCTCCTCA | |
| | TCCTCCCTGACCCTGCTCCTAGCACCCTGGAGAGTGCACATGCCCCTTGGTCCTGGCAGGGCGCCAAGTC | |
| | TGGCACCATGTTGGCCTCTTCAGGCCTGCTAGTCACTGGAAATTGAGGTCCATGGGGGAAATCAAGGATG | |
| | CTCAGTTTAAGGTACACTGTTTCCATGTTATGTTTCTACACATTGCTACCTCAGTGCTCCTGGAAACTTA | |
| | GCTTTTGATGTCTCCAAGTAGTCCACCTTCATTTAACTCTTTGAAACTGTATCATCTTTGCCAAGTAAGA | |
| | GTGGTGGCCTATTTCAGCTGCTTTGACAAAATGACTGGCTCCTGACTTAACGTTCTATAAATGAATGTGC | |
| | TGAAGCAAAGTGCCCATGGTGGCGGCGAAGAAGAGAAAGATGTGTTTTGTTTTGGACTCTCTGTGGTCCC | |
| | TTCCAATGCTGTGGGTTTCCAACCAGGGGAAGGGTCCCTTTTGCATTGCCAAGTGCCATAACCATGAGCA | |
| | CTACTCTACCATGGTTCTGCCTCCTGGCCAAGCAGGCTGGTTTGCAAGAATGAAATGAATGATTCTACAG | |
| | CTAGGACTTAACCTTGAAATGGAAAGTCATGCAATCCCATTTGCAGGATCTGTCTGTGCACATGCCTCTG | |
| | TAGAGAGCAGCATTCCCAGGGACCTTGGAAACAGTTGGCACTGTAAGGTGCTTGCTCCCCAAGACACATC | |
| | CTAAAAGGTGTTGTAATGGTGAAAACGTCTTCCTTCTTTATTGCCCCTTCTTATTTATGTGAACAACTGT | |
| | TTGTCTTTTTTTGTATCTTTTTTAAACTGTAAAGTTCAATTGTGAAAATGAATATCATGCAAATAAATTA | |
| | TGCAATTTTTTTTTCAAAGTAAAAAAAAAA | |
| 13 | GAGTAGGCGCGAGCTAAGCAGGAGGCGGAGGCGGAGGCGGAGGGCGAGGGGCGGGGAGCGCCGCCTGGAG | >NM_001382720.1 |
| | CGCGGCAGGTCATATTGAACATTCCAGATACCTATCATTACTCGATGCTGTTGATAACAGCAAGATGGCT | Homo sapiens |
| | TTGAACTCAGGGTCACCACCAGCTATTGGACCTTACTATGAAAACCATGGATACCAACCGGAAAACCCCT | transmembrane |
| | ATCCCGCACAGCCCACTGTGGTCCCCACTGTCTACGAGGTGCATCCGGCTCAGTACTACCCGTCCCCCGT | serine |
| | GCCCCAGTACGCCCCGAGGGTCCTGACGCAGGCTTCCAACCCCGTCGTCTGCACGCAGCCCAAATCCCCA | protease 2 |
| | TCCGGGACAGTGTGCACCTCAAAGACTAAGAAAGCACTGTGCATCACCTTGACCCTGGGGACCTTCCTCG | (TMPRSS2), |
| | TGGGAGCTGCGCTGGCCGCTGGCCTACTCTGGAAGTTCATGGGCAGCAAGTGCTCCAACTCTGGGATAGA | transcript |
| | GTGCGACTCCTCAGGTACCTGCATCAACCCCTCTAACTGGTGTGATGGCGTGTCACACTGCCCCGGCGGG | variant 3, |
| | GAGGACGAGAATCGGTGTGTTCGCCTCTACGGACCAAACTTCATCCTTCAGGTGTACTCATCTCAGAGGA | mRNA |
| | AGTCCTGGCACCCTGTGTGCCAAGACGACTGGAACGAGAACTACGGGCGGGCGGCCTGCAGGGACATGGG | |
| | CTATAAGAATAATTTTTACTCTAGCCAAGGAATAGTGGATGACAGCGGATCCACCAGCTTTATGAAACTG | |
| | AACACAAGTGCCGGCAATGTCGATATCTATAAAAAACTGTACCACAGTGATGCCTGTTCTTCAAAAGCAG | |
| | TGGTTTCTTTACGCTGTATAGCCTGCGGGGTCAACTTGAACTCAAGCCGCCAGAGCAGGATTGTGGGCGG | |
| | CGAGAGCGCGCTCCCGGGGGCCTGGCCCTGGCAGGTCAGCCTGCACGTCCAGAACGTCCACGTGTGCGGA | |
| | GGCTCCATCATCACCCCCGAGTGGATCGTGACAGCCGCCCACTGCGTGGAAAAACCTCTTAACAATCCAT | |
| | GGCATTGGACGGCATTTGCGGGGATTTTGAGACAATCTTTCATGTTCTATGGAGCCGGATACCAAGTAGA | |
| | AAAAGTGATTTCTCATCCAAATTATGACTCCAAGACCAAGAACAATGACATTGCGCTGATGAAGCTGCAG | |
| | AAGCCTCTGACTTTCAACGACCTAGTGAAACCAGTGTGTCTGCCCAACCCAGGCATGATGCTGCAGCCAG | |
| | AACAGCTCTGCTGGATTTCCGGGTGGGGGGCCACCGAGGAGAAAGGGAAGACCTCAGAAGTGCTGAACGC | |
| | TGCCAAGGTGCTTCTCATTGAGACACAGAGATGCAACAGCAGATATGTCTATGACAACCTGATCACACCA | |
| | GCCATGATCTGTGCCGGCTTCCTGCAGGGGAACGTCGATTCTTGCCAGGGTGACAGTGGAGGGCCTCTGG | |
| | TCACTTCGAAGAACAATATCTGGTGGCTGATAGGGGATACAAGCTGGGGTTCTGGCTGTGCCAAAGCTTA | |
| | CAGACCAGGAGTGTACGGGAATGTGATGGTATTCACGGACTGGATTTATCGACAAATGAGGACGGCTAAT | |
| | CCACATGGTCTTCGTCCTTGACGTCGTTTTACAAGAAAACAATGGGGCTGGTTTTGCTTCCCCGTGCATG | |
| | ATTTACTCTTAGAGATGATTCAGAGGTCACTTCATTTTTATTAAACAGTGAACTTGTCTGGCTTTGGCAC | |
| | TCTCTGCCATTCTGTGCAGGCTGCAGTGGCTCCCCTGCCCAGCCTGCTCTCCCTAACCCCTTGTCCGCAA | |
| | GGGGTGATGGCCGGCTGGTTGTGGGCACTGGCGGTCAAGTGTGGAGGAGAGGGGTGGAGGCTGCCCCATT | |
| | GAGATCTTCCTGCTGAGTCCTTTCCAGGGGCCAATTTTGGATGAGCATGGAGCTGTCACCTCTCAGCTGC | |
| | TGGATGACTTGAGATGAAAAAGGAGAGACATGGAAAGGGAGACAGCCAGGTGGCACCTGCAGCGGCTGCC | |
| | CTCTGGGGCCACTTGGTAGTGTCCCCAGCCTACCTCTCCACAAGGGGATTTTGCTGATGGGTTCTTAGAG | |
| | CCTTAGCAGCCCTGGATGGTGGCCAGAAATAAAGGGACCAGCCCTTCATGGGTGGTGACGTGGTAGTCAC | |
| | TTGTAAGGGGAACAGAAACATTTTTGTTCTTATGGGGTGAGAATATAGACAGTGCCCTTGGTGCGAGGGA | |
| | AGCAATTGAAAAGGAACTTGCCCTGAGCACTCCTGGTGCAGGTCTCCACCTGCACATTGGGTGGGGCTCC | |
| | TGGGAGGGAGACTCAGCCTTCCTCCTCATCCTCCCTGACCCTGCTCCTAGCACCCTGGAGAGTGCACATG | |
| | CCCCTTGGTCCTGGCAGGGCGCCAAGTCTGGCACCATGTTGGCCTCTTCAGGCCTGCTAGTCACTGGAAA | |
| | TTGAGGTCCATGGGGGAAATCAAGGATGCTCAGTTTAAGGTACACTGTTTCCATGTTATGTTTCTACACA | |
| | TTGCTACCTCAGTGCTCCTGGAAACTTAGCTTTTGATGTCTCCAAGTAGTCCACCTTCATTTAACTCTTT | |
| | GAAACTGTATCATCTTTGCCAAGTAAGAGTGGTGGCCTATTTCAGCTGCTTTGACAAAATGACTGGCTCC | |
| | TGACTTAACGTTCTATAAATGAATGTGCTGAAGCAAAGTGCCCATGGTGGCGGCGAAGAAGAGAAAGATG | |
| | TGTTTTGTTTTGGACTCTCTGTGGTCCCTTCCAATGCTGTGGGTTTCCAACCAGGGGAAGGGTCCCTTTT | |
| | GCATTGCCAAGTGCCATAACCATGAGCACTACTCTACCATGGTTCTGCCTCCTGGCCAAGCAGGCTGGTT | |
| | TGCAAGAATGAAATGAATGATTCTACAGCTAGGACTTAACCTTGAAATGGAAAGTCATGCAATCCCATTT | |
| | GCAGGATCTGTCTGTGCACATGCCTCTGTAGAGAGCAGCATTCCCAGGGACCTTGGAAACAGTTGGCACT | |
| | GTAAGGTGCTTGCTCCCCAAGACACATCCTAAAAGGTGTTGTAATGGTGAAAACGTCTTCCTTCTTTATT | |
| | GCCCCTTCTTATTTATGTGAACAACTGTTTGTCTTTTTTTGTATCTTTTTTAAACTGTAAAGTTCAATTG | |
| | TGAAAATGAATATCATGCAAATAAATTATGCAATTTTTTTTTCAAAGTAA | |
| 14 | GAGTAGGCGCGAGCTAAGCAGGAGGCGGAGGCGGAGGCGGAGGGCGAGGGGCGGGGAGCGCCGCCTGGAG | >NM_005656.4 |
| | CGCGGCAGGTCATATTGAACATTCCAGATACCTATCATTACTCGATGCTGTTGATAACAGCAAGATGGCT | Homo sapiens |
| | TTGAACTCAGGGTCACCACCAGCTATTGGACCTTACTATGAAAACCATGGATACCAACCGGAAAACCCCT | transmembrane |
| | ATCCCGCACAGCCCACTGTGGTCCCCACTGTCTACGAGGTGCATCCGGCTCAGTACTACCCGTCCCCCGT | serine |
| | GCCCCAGTACGCCCCGAGGGTCCTGACGCAGGCTTCCAACCCCGTCGTCTGCACGCAGCCCAAATCCCCA | protease 2 |
| | TCCGGGACAGTGTGCACCTCAAAGACTAAGAAAGCACTGTGCATCACCTTGACCCTGGGGACCTTCCTCG | (TMPRSS2), |
| | TGGGAGCTGCGCTGGCCGCTGGCCTACTCTGGAAGTTCATGGGCAGCAAGTGCTCCAACTCTGGGATAGA | transcript |
| | GTGCGACTCCTCAGGTACCTGCATCAACCCCTCTAACTGGTGTGATGGCGTGTCACACTGCCCCGGCGGG | variant 2, |
| | GAGGACGAGAATCGGTGTGTTCGCCTCTACGGACCAAACTTCATCCTTCAGGTGTACTCATCTCAGAGGA | mRNA |
| | AGTCCTGGCACCCTGTGTGCCAAGACGACTGGAACGAGAACTACGGGCGGGCGGCCTGCAGGGACATGGG | |
| | CTATAAGAATAATTTTTACTCTAGCCAAGGAATAGTGGATGACAGCGGATCCACCAGCTTTATGAAACTG | |
| | AACACAAGTGCCGGCAATGTCGATATCTATAAAAAACTGTACCACAGTGATGCCTGTTCTTCAAAAGCAG | |
| | TGGTTTCTTTACGCTGTATAGCCTGCGGGGTCAACTTGAACTCAAGCCGCCAGAGCAGGATTGTGGGCGG | |
| | CGAGAGCGCGCTCCCGGGGGCCTGGCCCTGGCAGGTCAGCCTGCACGTCCAGAACGTCCACGTGTGCGGA | |
| | GGCTCCATCATCACCCCCGAGTGGATCGTGACAGCCGCCCACTGCGTGGAAAAACCTCTTAACAATCCAT | |
| | GGCATTGGACGGCATTTGCGGGGATTTTGAGACAATCTTTCATGTTCTATGGAGCCGGATACCAAGTAGA | |
| | AAAAGTGATTTCTCATCCAAATTATGACTCCAAGACCAAGAACAATGACATTGCGCTGATGAAGCTGCAG | |
| | AAGCCTCTGACTTTCAACGACCTAGTGAAACCAGTGTGTCTGCCCAACCCAGGCATGATGCTGCAGCCAG | |
| | AACAGCTCTGCTGGATTTCCGGGTGGGGGGCCACCGAGGAGAAAGGGAAGACCTCAGAAGTGCTGAACGC | |
| | TGCCAAGGTGCTTCTCATTGAGACACAGAGATGCAACAGCAGATATGTCTATGACAACCTGATCACACCA | |
| | GCCATGATCTGTGCCGGCTTCCTGCAGGGGAACGTCGATTCTTGCCAGGGTGACAGTGGAGGGCCTCTGG | |
| | TCACTTCGAAGAACAATATCTGGTGGCTGATAGGGGATACAAGCTGGGGTTCTGGCTGTGCCAAAGCTTA | |
| | CAGACCAGGAGTGTACGGGAATGTGATGGTATTCACGGACTGGATTTATCGACAAATGAGGGCAGACGGC | |
| | TAATCCACATGGTCTTCGTCCTTGACGTCGTTTTACAAGAAAACAATGGGGCTGGTTTTGCTTCCCCGTG | |
| | CATGATTTACTCTTAGAGATGATTCAGAGGTCACTTCATTTTTATTAAACAGTGAACTTGTCTGGCTTTG | |
| | GCACTCTCTGCCATTCTGTGCAGGCTGCAGTGGCTCCCCTGCCCAGCCTGCTCTCCCTAACCCCTTGTCC | |
| | GCAAGGGGTGATGGCCGGCTGGTTGTGGGCACTGGCGGTCAAGTGTGGAGGAGAGGGGTGGAGGCTGCCC | |
| | CATTGAGATCTTCCTGCTGAGTCCTTTCCAGGGGCCAATTTTGGATGAGCATGGAGCTGTCACCTCTCAG | |
| | CTGCTGGATGACTTGAGATGAAAAAGGAGAGACATGGAAAGGGAGACAGCCAGGTGGCACCTGCAGCGGC | |
| | TGCCCTCTGGGGCCACTTGGTAGTGTCCCCAGCCTACCTCTCCACAAGGGGATTTTGCTGATGGGTTCTT | |
| | AGAGCCTTAGCAGCCCTGGATGGTGGCCAGAAATAAAGGGACCAGCCCTTCATGGGTGGTGACGTGGTAG | |
| | TCACTTGTAAGGGGAACAGAAACATTTTTGTTCTTATGGGGTGAGAATATAGACAGTGCCCTTGGTGCGA | |
| | GGGAAGCAATTGAAAAGGAACTTGCCCTGAGCACTCCTGGTGCAGGTCTCCACCTGCACATTGGGTGGGG | |
| | CTCCTGGGAGGGAGACTCAGCCTTCCTCCTCATCCTCCCTGACCCTGCTCCTAGCACCCTGGAGAGTGCA | |
| | CATGCCCCTTGGTCCTGGCAGGGCGCCAAGTCTGGCACCATGTTGGCCTCTTCAGGCCTGCTAGTCACTG | |
| | GAAATTGAGGTCCATGGGGGAAATCAAGGATGCTCAGTTTAAGGTACACTGTTTCCATGTTATGTTTCTA | |
| | CACATTGCTACCTCAGTGCTCCTGGAAACTTAGCTTTTGATGTCTCCAAGTAGTCCACCTTCATTTAACT | |
| | CTTTGAAACTGTATCATCTTTGCCAAGTAAGAGTGGTGGCCTATTTCAGCTGCTTTGACAAAATGACTGG | |
| | CTCCTGACTTAACGTTCTATAAATGAATGTGCTGAAGCAAAGTGCCCATGGTGGCGGCGAAGAAGAGAAA | |
| | GATGTGTTTTGTTTTGGACTCTCTGTGGTCCCTTCCAATGCTGTGGGTTTCCAACCAGGGGAAGGGTCCC | |
| | TTTTGCATTGCCAAGTGCCATAACCATGAGCACTACTCTACCATGGTTCTGCCTCCTGGCCAAGCAGGCT | |
| | GGTTTGCAAGAATGAAATGAATGATTCTACAGCTAGGACTTAACCTTGAAATGGAAAGTCATGCAATCCC | |
| | ATTTGCAGGATCTGTCTGTGCACATGCCTCTGTAGAGAGCAGCATTCCCAGGGACCTTGGAAACAGTTGG | |
| | CACTGTAAGGTGCTTGCTCCCCAAGACACATCCTAAAAGGTGTTGTAATGGTGAAAACGTCTTCCTTCTT | |
| | TATTGCCCCTTCTTATTTATGTGAACAACTGTTTGTCTTTTTTTGTATCTTTTTTAAACTGTAAAGTTCA | |
| | ATTGTGAAAATGAATATCATGCAAATAAATTATGCAATTTTTTTTTCAAAGTAACTACTGCATCTTTGAA | |
| | GTTCTGCCTGGTGAGTAGGACCAGCCTCCATTTCCTTATAAGGGGGTGATGTTGAGGCTGCTGGTCAGAG | |
| | GACCAAAGGTGAGGCAAGGCCAGACTTGGTGCTCCTGTGGTTGGTGCCCTCAGTTCCTGCAGCCTGTCCT | |
| | GTTGGAGAGGTCCCTCAAATGACTCCTTCTTATTATTCTATTAGTCTGTTTCCATGCTCCTAATAAAGAC | |
| | ATACCCAAGACTGCAATTTA | |
| 15 | MPPAPPGGESGCEERGAAGHIEHSRYLSLLDAVDNSKMALNSGSPPAIGPYYENHGYQPENPYPAQPTVV | >NP_001128571.1 |
| | PTVYEVHPAQYYPSPVPQYAPRVLTQASNPVVCTQPKSPSGTVCTSKTKKALCITLTLGTFLVGAALAAG | transmembrane |
| | LLWKFMGSKCSNSGIECDSSGTCINPSNWCDGVSHCPGGEDENRCVRLYGPNFILQVYSSQRKSWHPVCQ | protease |
| | DDWNENYGRAACRDMGYKNNFYSSQGIVDDSGSTSFMKLNTSAGNVDIYKKLYHSDACSSKAVVSLRCIA | serine 2 |
| | CGVNLNSSRQSRIVGGESALPGAWPWQVSLHVQNVHVCGGSIITPEWIVTAAHCVEKPLNNPWHWTAFAG | isoform 1 |
| | ILRQSFMFYGAGYQVEKVISHPNYDSKTKNNDIALMKLQKPLTENDLVKPVCLPNPGMMLQPEQLCWISG | [Homo |
| | WGATEEKGKTSEVLNAAKVLLIETQRCNSRYVYDNLITPAMICAGFLQGNVDSCQGDSGGPLVTSKNNIW | sapiens] |
| | WLIGDTSWGSGCAKAYRPGVYGNVMVFTDWIYRQMRADG | |
| 16 | MALNSGSPPAIGPYYENHGYQPENPYPAQPTVVPTVYEVHPAQYYPSPVPQYAPRVLTQASNPVVCTQPK | >NP_001369649.1 |
| | SPSGTVCTSKTKKALCITLTLGTFLVGAALAAGLLWKFMGSKCSNSGIECDSSGTCINPSNWCDGVSHCP | transmembrane |
| | GGEDENRCVRLYGPNFILQVYSSQRKSWHPVCQDDWNENYGRAACRDMGYKNNFYSSQGIVDDSGSTSFM | protease |
| | KLNTSAGNVDIYKKLYHSDACSSKAVVSLRCIACGVNLNSSRQSRIVGGESALPGAWPWQVSLHVQNVHV | serine 2 |
| | CGGSIITPEWIVTAAHCVEKPLNNPWHWTAFAGILRQSFMFYGAGYQVEKVISHPNYDSKTKNNDIALMK | isoform 3 |
| | LQKPLTENDLVKPVCLPNPGMMLQPEQLCWISGWGATEEKGKTSEVLNAAKVLLIETQRCNSRYVYDNLI | [Homo |
| | TPAMICAGFLQGNVDSCQGDSGGPLVTSKNNIWWLIGDTSWGSGCAKAYRPGVYGNVMVFTDWIYRQMRT | sapiens] |
| | ANPHGLRP | |
| 17 | MALNSGSPPAIGPYYENHGYQPENPYPAQPTVVPTVYEVHPAQYYPSPVPQYAPRVLTQASNPVVCTQPK | >NP_005647.3 |
| | SPSGTVCTSKTKKALCITLTLGTFLVGAALAAGLLWKFMGSKCSNSGIECDSSGTCINPSNWCDGVSHCP | transmembrane |
| | GGEDENRCVRLYGPNFILQVYSSQRKSWHPVCQDDWNENYGRAACRDMGYKNNFYSSQGIVDDSGSTSFM | protease |
| | KLNTSAGNVDIYKKLYHSDACSSKAVVSLRCIACGVNLNSSRQSRIVGGESALPGAWPWQVSLHVQNVHV | serine 2 |
| | CGGSIITPEWIVTAAHCVEKPLNNPWHWTAFAGILRQSFMFYGAGYQVEKVISHPNYDSKTKNNDIALMK | isoform 2 |
| | LQKPLTENDLVKPVCLPNPGMMLQPEQLCWISGWGATEEKGKTSEVLNAAKVLLIETQRCNSRYVYDNLI | [Homo |
| | TPAMICAGFLQGNVDSCQGDSGGPLVTSKNNIWWLIGDTSWGSGCAKAYRPGVYGNVMVFTDWIYRQMRA | sapiens] |
| | DG | |
| 18 | ACTCCTGGAATACACAGAGAGAGGCAGCAGCTTGCTCAGCGGACAAGGATGCTGGGCGTGAGGGACCAAG | >NM_001083947.2 |
| | GCCTGCCCTGCACTCGGGCCTCCTCCAGCCAGTGCTGACCAGGGACTTCTGACCTGCTGGCCAGCCAGGA | Homo sapiens |
| | CCTGTGTGGGGAGGCCCTCCTGCTGCCTTGGGGTGACAATCTCAGCTCCAGGCTACAGGGAGACCGGGAG | transmembrane |
| | GATCACAGAGCCAGCATGTTACAGGATCCTGACAGTGATCAACCTCTGAACAGCCTCGATGTCAAACCCC | serine |
| | TGCGCAAACCCCGTATCCCCATGGAGACCTTCAGAAAGGTGGGGATCCCCATCATCATAGCACTACTGAG | protease 4 |
| | CCTGGCGAGTATCATCATTGTGGTTGTCCTCATCAAGGTGATTCTGGATAAATACTACTTCCTCTGCGGG | (TMPRSS4), |
| | CAGCCTCTCCACTTCATCCCGAGGAAGCAGCTGTGTGACGGAGAGCTGGACTGTCCCTTGGGGGAGGACG | transcript |
| | AGGAGCACTGTGTCAAGAGCTTCCCCGAAGGGCCTGCAGTGGCAGTCCGCCTCTCCAAGGACCGATCCAC | variant 3, |
| | ACTGCAGGTGCTGGACTCGGCCACAGGGAACTGGTTCTCTGCCTGTTTCGACAACTTCACAGAAGCTCTC | mRNA |
| | GCTGAGACAGCCTGTAGGCAGATGGGCTACAGCAGAGCTGTGGAGATTGGCCCAGACCAGGATCTGGATG | |
| | TTGTTGAAATCACAGAAAACAGCCAGGAGCTTCGCATGCGGAACTCAAGTGGGCCCTGTCTCTCAGGCTC | |
| | CCTGGTCTCCCTGCACTGTCTTGCCTGTGGGAAGAGCCTGAAGACCCCCCGTGTGGTGGGTGTGGAGGAG | |
| | GCCTCTGTGGATTCTTGGCCTTGGCAGGTCAGCATCCAGTACGACAAACAGCACGTCTGTGGAGGGAGCA | |
| | TCCTGGACCCCCACTGGGTCCTCACGGCAGCCCACTGCTTCAGGAAACATACCGATGTGTTCAACTGGAA | |
| | GGTGCGGGCAGGCTCAGACAAACTGGGCAGCTTCCCATCCCTGGCTGTGGCCAAGATCATCATCATTGAA | |
| | TTCAACCCCATGTACCCCAAAGACAATGACATCGCCCTCATGAAGCTGCAGTTCCCACTCACTTTCTCAG | |
| | GCACAGTCAGGCCCATCTGTCTGCCCTTCTTTGATGAGGAGCTCACTCCAGCCACCCCACTCTGGATCAT | |
| | TGGATGGGGCTTTACGAAGCAGAATGGAGGGAAGATGTCTGACATACTGCTGCAGGCGTCAGTCCAGGTC | |
| | ATTGACAGCACACGGTGCAATGCAGACGATGCGTACCAGGGGGAAGTCACCGAGAAGATGATGTGTGCAG | |
| | GCATCCCGGAAGGGGGTGTGGACACCTGCCAGGGTGACAGTGGTGGGCCCCTGATGTACCAATCTGACCA | |
| | GTGGCATGTGGTGGGCATCGTTAGTTGGGGCTATGGCTGCGGGGGCCCGAGCACCCCAGGAGTATACACC | |
| | AAGGTCTCAGCCTATCTCAACTGGATCTACAATGTCTGGAAGGCTGAGCTGTAATGCTGCTGCCCCTTTG | |
| | CAGTGCTGGGAGCCGCTTCCTTCCTGCCCTGCCCACCTGGGGATCCCCCAAAGTCAGACACAGAGCAAGA | |
| | GTCCCCTTGGGTACACCCCTCTGCCCACAGCCTCAGCATTTCTTGGAGCAGCAAAGGGCCTCAATTCCTA | |
| | TAAGAGACCCTCGCAGCCCAGAGGCGCCCAGAGGAAGTCAGCAGCCCTAGCTCGGCCACACTTGGTGCTC | |
| | CCAGCATCCCAGGGAGAGACACAGCCCACTGAACAAGGTCTCAGGGGTATTGCTAAGCCAAGAAGGAACT | |
| | TTCCCACACTACTGAATGGAAGCAGGCTGTCTTGTAAAAGCCCAGATCACTGTGGGCTGGAGAGGAGAAG | |
| | GAAAGGGTCTGCGCCAGCCCTGTCCGTCTTCACCCATCCCCAAGCCTACTAGAGCAAGAAACCAGTTGTA | |
| | ATATAAAATGCACTGCCCTACTGTTGGTATGACTACCGTTACCTACTGTTGTCATTGTTATTACAGCTAT | |
| | GGCCACTATTATTAAAGAGCTGTGTAACATCTCTGGCATAGGCTAGCTGGAATGCTTGATAAGAACTGAG | |
| | CTGGGATGATTGAACTTTCATTCTTTGGCTTGGGGAGAAAAGAAGTCCTGGGGAAGCAATTGAGTCTCAA | |
| | AGTAGAGGCAGGGGAAAAAAGAGTTAGGGAGACCAGATCTGCTGAGTGGCAGCAAGAGTGAGCTGCAGAT | |
| | TACAGAAACCAGGGTGAGCAAGTTTGAGTCCCACACAGGGCCTTCTCCCTTTGCCTCTTTCCCTCCCTCC | |
| | CTGCCTGTGATAATCAGCCAGGAGCCAGGGATAACCTATGACTTGGGAAAGAGATGAGTTAGGCAGTCAA | |
| | GGGTGACATTCAATCAGGGATCCACAAGTGGCTGGAAAGAAATGCTGGTCCTGTGTCCTAACTTTTTCCG | |
| | CCTGGAGAGCCCTCAGTGTGGCTTCTTACATTTAAAAAACAAAAAGGATCAGCTGCCAGGTGTGAGGCAG | |
| | TCCCCAAGCTGAGTTGTGAGGATGTAAGCATGAATAAGTCCCTGCACTCAAAATGGTCAAAGAATTAAAC | |
| | CCCATGGACTTTTTTGGCATCTGTATGAAAGCTTGGGTTTTCTGAGGACTGTCTTGCTATAGTTAAGTCA | |
| | GATCCTAGATGAAATATACTTGTTCATACTGTACTAGGTTCTTAGGAAACAACAGAATTCCTCAAATGCC | |
| | AAAAACAAAGAAAATAGAAACCCAGAAAACAAAACAAAATAAAACAAAACCATCAGAACTGTGAGTGGAA | |
| | ACTAAGGTGATGATCTGGGAGCAATACACTAAAATCTTGGGTCGAGACCTATATGAAGGCTGGCAGTGGA | |
| | GCTAAACCTGGACACACTGAAGACAAGGGAGCTGAACCAGGGCTCCTACATGAAGCAGGGATAACTGATG | |
| | GCAGTAAATGTGGTCTCAAATTGCAGATGGTCTGGAGGAAAATTTCCCAAATTTAGAGCCTCAGGATTCC | |
| | CAAAGATCCTCCAAATATGAGCTCACAATCAAAGATCAGAGACGTTGAAAAATAAAAAACACCTTAAGTG | |
| | GGCAGCATAAAAAACAGCTAATTTAGAACCCCAAAGGCTTCAGATGTCAGAATATTAGAGACTTATGATA | |
| | ATAAGCAATATTTGCAGAGTATTTGTATGTGCCAGACACTATTGTAAGTGCTTCATCATGTACTGATTCA | |
| | TTTAATACTCACAGAAATCTGTGAGATGGGTATTATTCTTATCCTCACTCTATGGATTAAAAAAACTAAG | |
| | GCACAAAGTGGTTAAGCTCCTTGCCTGAGATTATAGACTGTAAGTTGAACGTGAGCACTTGGAATACAGA | |
| | GTTCATGCTGTAAACTACCACACTATAGGGCCTCCAATATGATAATTTATAAAATATTTGAATAAAAAAT | |
| | GAATACTAGTTCCACATTTTAAAATCATGTTTAACTGTGGTCAAATGCACATAACACAAGTTGCCATCTT | |
| | CACCATTTTTAGGTGTATAGTTCAGTGGTGTTATGTACATTCACACTATTGTGCAGTCATCACCACCATC | |
| | CATCTCCAGAACAGAAACTCAGTACCCATCAAACAACTCTCCATTTCCCCCTCCTCCCAATCTCTGGCAA | |
| | CCACCATTGTGCTTTCAGTCTCTGTGAACTGGATTACTCTGGGTACCTCATTTAAGTGAAGTCATGCAGT | |
| | ATTGGTCTTTTTGTACTTGTTTTATTTCACTTCACATTGTGTCTTCAAGTTTCACCCATGTTGTAGCATG | |
| | TGTCAGAATTTCTTCCCTTTTTAGACTAAATAATATTCTATTGTTTATACGAACATTCAGGTTACTTCTA | |
| | TCTTTTGGCTATTGTGAATTATGCTGCTGTGAACATGGGTGTACAAGTATCTCTTTGAGGCCCTGCTTTC | |
| | AATTCTCTTGGGTATATTCCCAGAAGTGGAATTGCTGGATCATATGGTAATTCTATTTTGAATTTTTTGA | |
| | GGAACTGATATATTGCTTTCCATAGAGACTGCACCATTTTACATTCCCATCAACAGTTTGCAGGAGTTAC | |
| | TATTTCTCCATATCCCCCCTAACACTTGCTATTTTCTGTTAAAAATGGATATCTTAATAATCAAGCAAAA | |
| | ATAACAGGCAGATTTGAAAAAGAACTGAATACAGCTTTTAGAAATAAAAACTATAATTATAAAAATAAAA | |
| | AACTAAGTGGATGGGGTAAATAACAATTAAAACACCAATTAAGAGAGAACAAATGAACTGGAAGATAAAT | |
| | TGAAGAAGTGACTAGGCTTAACAGCAGAGAGAGATAAGGAGATTAAAAATATGAAAACAAGGCCAGGAGC | |
| | AATGAAGCCTAGAATGGTAAATTCTAACATATCCAGAATCCCAGAAAGAGAGAATCAAGACAATGAGAGA | |
| | GAGACAGTACCAAAGAGATAAGAGCTGAGAATGTTCCAGAATTGATAAAAGGTGTGAATCCACAGAACAT | |
| | ACACCACCATAGTGTACACGCATACAACCAAGGTGGAAAAATTAGAATAAATCCACACCTATGTACATTA | |
| | TAATGAAACTGCAGAACACCAAAGACAAAAAGAAACTCCTTATAGCAGCAGAGAGAAAACCCAGACCACC | |
| | CACAGTACCACAAATCTACCACAATTAGACTGACAACAGGCTTTCCCACAGCAATAAAGGAGCTAGAAGT | |
| | CAGTGGAAGTATATCTCCAGCATGCCAAAAGATAACAATCAATCAGGGATTGTGAACCCTACAAAACTAT | |
| | CTTTCAAGAATAAAGGCATTTTCAAGAAAACAAAAACAGACTTTACCATCAACAAACCTTCTCTAAAAGA | |
| | ATATATAAAGCATTTACTTTAGGAAGAAGGAAAATGATCCTAAAAGGAAGAACCAAGAAGCAAGTAGCAA | |
| | TAGTGAGGCAATTGTGAAAATGTAGGTAAGTCTAAACACACTCTGTCTACTTCTTCTTCTTCTTCTTCTT | |
| | CTTCTTCTTCTTATTTTGAGACTGAGTCTTGCCCTGTCACCCAGACTGGAGTGCAGTGGCAGGATCTTGG | |
| | CTCACTGCTATCTCCACCTCCCAGGTTCAAGTGATTCTTCTGCCTCAGCCTCCCGAGTAGCTGGGATTAC | |
| | ATGCACATGCCACCATATCCGGCTAATTTTTGAATTTTTAGTAGAGATGGGGTTTCACTGTGTTGGCCAG | |
| | GCCGGTCTCAAACTCCCGACCTCAAGTGATCCCCCCGCCTCGGCCTCCCAAAGTGCTGGGATTACAGGCG | |
| | TGTCTACATATTATTAAAATAACAATAATATTTATTTTGTGGGTTAATTTTTTTTGAAACAGATATTGAA | |
| | TTTATTGGTTGGCTATGAGTAGAAAAATACATCAGTAAAGAAAAAAGACCCTGTATATAAATATAATACT | |
| | AGCTAGTTAAAATTTGACCAAGAAGTTTCCATTGTGGGTTAATTTTTAAAGGCCTAACTGAAATATGGAG | |
| | TAACCACAGCATGCAGCATGTAAATTAAAGGGGATAGCTGG | |
| 19 | ACTCCTGGAATACACAGAGAGAGGCAGCAGCTTGCTCAGCGGACAAGGATGCTGGGCGTGAGGGACCAAG | >NM_001173551.2 |
| | GCCTGCCCTGCACTCGGGCCTCCTCCAGCCAGTGCTGACCAGGGACTTCTGACCTGCTGGCCAGCCAGGA | Homo sapiens |
| | CCTGTGTGGGGAGGCCCTCCTGCTGCCTTGGGGTGACAATCTCAGCTCCAGGCTACAGGGAGACCGGGAG | transmembrane |
| | GATCACAGAGCCAGCATGGATCCTGACAGTGATCAACCTCTGAACAGCCTCGATGTCAAACCCCTGCGCA | serine |
| | AACCCCGTATCCCCATGGAGACCTTCAGAAAGGTGGGGATCCCCATCATCATAGCACTACTGAGCCTGGC | protease 4 |
| | GAGTATCATCATTGTGGTTGTCCTCATCAAGGTGATTCTGGATAAATACTACTTCCTCTGCGGGCAGCCT | (TMPRSS4), |
| | CTCCACTTCATCCCGAGGAAGCAGCTGTGTGACGGAGAGCTGGACTGTCCCTTGGGGGAGGACGAGGAGC | transcript |
| | ACTGTGTCAAGAGCTTCCCCGAAGGGCCTGCAGTGGCAGTCCGCCTCTCCAAGGACCGATCCACACTGCA | variant 4, |
| | GGTGCTGGACTCGGCCACAGGGAACTGGTTCTCTGCCTGTTTCGACAACTTCACAGAAGCTCTCGCTGAG | mRNA |
| | ACAGCCTGTAGGCAGATGGGCTACAGCAGCAAACCCACTTTCAGAGCTGTGGAGATTGGCCCAGACCAGG | |
| | ATCTGGATGTTGTTGAAATCACAGAAAACAGCCAGGAGCTTCGCATGCGGAACTCAAGTGGGCCCTGTCT | |
| | CTCAGGCTCCCTGGTCTCCCTGCACTGTCTTGCCTGTGGGAAGAGCCTGAAGACCCCCCGTGTGGTGGGT | |
| | GTGGAGGAGGCCTCTGTGGATTCTTGGCCTTGGCAGGTCAGCATCCAGTACGACAAACAGCACGTCTGTG | |
| | GAGGGAGCATCCTGGACCCCCACTGGGTCCTCACGGCAGCCCACTGCTTCAGGAAACATACCGATGTGTT | |
| | CAACTGGAAGGTGCGGGCAGGCTCAGACAAACTGGGCAGCTTCCCATCCCTGGCTGTGGCCAAGATCATC | |
| | ATCATTGAATTCAACCCCATGTACCCCAAAGACAATGACATCGCCCTCATGAAGCTGCAGTTCCCACTCA | |
| | CTTTCTCAGGCACAGTCAGGCCCATCTGTCTGCCCTTCTTTGATGAGGAGCTCACTCCAGCCACCCCACT | |
| | CTGGATCATTGGATGGGGCTTTACGAAGCAGAATGGAGGGAAGATGTCTGACATACTGCTGCAGGCGTCA | |
| | GTCCAGGTCATTGACAGCACACGGTGCAATGCAGACGATGCGTACCAGGGGGAAGTCACCGAGAAGATGA | |
| | TGTGTGCAGGCATCCCGGAAGGGGGTGTGGACACCTGCCAGGGTGACAGTGGTGGGCCCCTGATGTACCA | |
| | ATCTGACCAGTGGCATGTGGTGGGCATCGTTAGTTGGGGCTATGGCTGCGGGGGCCCGAGCACCCCAGGA | |
| | GTATACACCAAGGTCTCAGCCTATCTCAACTGGATCTACAATGTCTGGAAGGCTGAGCTGTAATGCTGCT | |
| | GCCCCTTTGCAGTGCTGGGAGCCGCTTCCTTCCTGCCCTGCCCACCTGGGGATCCCCCAAAGTCAGACAC | |
| | AGAGCAAGAGTCCCCTTGGGTACACCCCTCTGCCCACAGCCTCAGCATTTCTTGGAGCAGCAAAGGGCCT | |
| | CAATTCCTATAAGAGACCCTCGCAGCCCAGAGGCGCCCAGAGGAAGTCAGCAGCCCTAGCTCGGCCACAC | |
| | TTGGTGCTCCCAGCATCCCAGGGAGAGACACAGCCCACTGAACAAGGTCTCAGGGGTATTGCTAAGCCAA | |
| | GAAGGAACTTTCCCACACTACTGAATGGAAGCAGGCTGTCTTGTAAAAGCCCAGATCACTGTGGGCTGGA | |
| | GAGGAGAAGGAAAGGGTCTGCGCCAGCCCTGTCCGTCTTCACCCATCCCCAAGCCTACTAGAGCAAGAAA | |
| | CCAGTTGTAATATAAAATGCACTGCCCTACTGTTGGTATGACTACCGTTACCTACTGTTGTCATTGTTAT | |
| | TACAGCTATGGCCACTATTATTAAAGAGCTGTGTAACATCTCTGGCATAGGCTAGCTGGAATGCTTGATA | |
| | AGAACTGAGCTGGGATGATTGAACTTTCATTCTTTGGCTTGGGGAGAAAAGAAGTCCTGGGGAAGCAATT | |
| | GAGTCTCAAAGTAGAGGCAGGGGAAAAAAGAGTTAGGGAGACCAGATCTGCTGAGTGGCAGCAAGAGTGA | |
| | GCTGCAGATTACAGAAACCAGGGTGAGCAAGTTTGAGTCCCACACAGGGCCTTCTCCCTTTGCCTCTTTC | |
| | CCTCCCTCCCTGCCTGTGATAATCAGCCAGGAGCCAGGGATAACCTATGACTTGGGAAAGAGATGAGTTA | |
| | GGCAGTCAAGGGTGACATTCAATCAGGGATCCACAAGTGGCTGGAAAGAAATGCTGGTCCTGTGTCCTAA | |
| | CTTTTTCCGCCTGGAGAGCCCTCAGTGTGGCTTCTTACATTTAAAAAACAAAAAGGATCAGCTGCCAGGT | |
| | GTGAGGCAGTCCCCAAGCTGAGTTGTGAGGATGTAAGCATGAATAAGTCCCTGCACTCAAAATGGTCAAA | |
| | GAATTAAACCCCATGGACTTTTTTGGCATCTGTATGAAAGCTTGGGTTTTCTGAGGACTGTCTTGCTATA | |
| | GTTAAGTCAGATCCTAGATGAAATATACTTGTTCATACTGTACTAGGTTCTTAGGAAACAACAGAATTCC | |
| | TCAAATGCCAAAAACAAAGAAAATAGAAACCCAGAAAACAAAACAAAATAAAACAAAACCATCAGAACTG | |
| | TGAGTGGAAACTAAGGTGATGATCTGGGAGCAATACACTAAAATCTTGGGTCGAGACCTATATGAAGGCT | |
| | GGCAGTGGAGCTAAACCTGGACACACTGAAGACAAGGGAGCTGAACCAGGGCTCCTACATGAAGCAGGGA | |
| | TAACTGATGGCAGTAAATGTGGTCTCAAATTGCAGATGGTCTGGAGGAAAATTTCCCAAATTTAGAGCCT | |
| | CAGGATTCCCAAAGATCCTCCAAATATGAGCTCACAATCAAAGATCAGAGACGTTGAAAAATAAAAAACA | |
| | CCTTAAGTGGGCAGCATAAAAAACAGCTAATTTAGAACCCCAAAGGCTTCAGATGTCAGAATATTAGAGA | |
| | CTTATGATAATAAGCAATATTTGCAGAGTATTTGTATGTGCCAGACACTATTGTAAGTGCTTCATCATGT | |
| | ACTGATTCATTTAATACTCACAGAAATCTGTGAGATGGGTATTATTCTTATCCTCACTCTATGGATTAAA | |
| | AAAACTAAGGCACAAAGTGGTTAAGCTCCTTGCCTGAGATTATAGACTGTAAGTTGAACGTGAGCACTTG | |
| | GAATACAGAGTTCATGCTGTAAACTACCACACTATAGGGCCTCCAATATGATAATTTATAAAATATTTGA | |
| | ATAAAAAATGAATACTAGTTCCACATTTTAAAATCATGTTTAACTGTGGTCAAATGCACATAACACAAGT | |
| | TGCCATCTTCACCATTTTTAGGTGTATAGTTCAGTGGTGTTATGTACATTCACACTATTGTGCAGTCATC | |
| | ACCACCATCCATCTCCAGAACAGAAACTCAGTACCCATCAAACAACTCTCCATTTCCCCCTCCTCCCAAT | |
| | CTCTGGCAACCACCATTGTGCTTTCAGTCTCTGTGAACTGGATTACTCTGGGTACCTCATTTAAGTGAAG | |
| | TCATGCAGTATTGGTCTTTTTGTACTTGTTTTATTTCACTTCACATTGTGTCTTCAAGTTTCACCCATGT | |
| | TGTAGCATGTGTCAGAATTTCTTCCCTTTTTAGACTAAATAATATTCTATTGTTTATACGAACATTCAGG | |
| | TTACTTCTATCTTTTGGCTATTGTGAATTATGCTGCTGTGAACATGGGTGTACAAGTATCTCTTTGAGGC | |
| | CCTGCTTTCAATTCTCTTGGGTATATTCCCAGAAGTGGAATTGCTGGATCATATGGTAATTCTATTTTGA | |
| | ATTTTTTGAGGAACTGATATATTGCTTTCCATAGAGACTGCACCATTTTACATTCCCATCAACAGTTTGC | |
| | AGGAGTTACTATTTCTCCATATCCCCCCTAACACTTGCTATTTTCTGTTAAAAATGGATATCTTAATAAT | |
| | CAAGCAAAAATAACAGGCAGATTTGAAAAAGAACTGAATACAGCTTTTAGAAATAAAAACTATAATTATA | |
| | AAAATAAAAAACTAAGTGGATGGGGTAAATAACAATTAAAACACCAATTAAGAGAGAACAAATGAACTGG | |
| | AAGATAAATTGAAGAAGTGACTAGGCTTAACAGCAGAGAGAGATAAGGAGATTAAAAATATGAAAACAAG | |
| | GCCAGGAGCAATGAAGCCTAGAATGGTAAATTCTAACATATCCAGAATCCCAGAAAGAGAGAATCAAGAC | |
| | AATGAGAGAGAGACAGTACCAAAGAGATAAGAGCTGAGAATGTTCCAGAATTGATAAAAGGTGTGAATCC | |
| | ACAGAACATACACCACCATAGTGTACACGCATACAACCAAGGTGGAAAAATTAGAATAAATCCACACCTA | |
| | TGTACATTATAATGAAACTGCAGAACACCAAAGACAAAAAGAAACTCCTTATAGCAGCAGAGAGAAAACC | |
| | CAGACCACCCACAGTACCACAAATCTACCACAATTAGACTGACAACAGGCTTTCCCACAGCAATAAAGGA | |
| | GCTAGAAGTCAGTGGAAGTATATCTCCAGCATGCCAAAAGATAACAATCAATCAGGGATTGTGAACCCTA | |
| | CAAAACTATCTTTCAAGAATAAAGGCATTTTCAAGAAAACAAAAACAGACTTTACCATCAACAAACCTTC | |
| | TCTAAAAGAATATATAAAGCATTTACTTTAGGAAGAAGGAAAATGATCCTAAAAGGAAGAACCAAGAAGC | |
| | AAGTAGCAATAGTGAGGCAATTGTGAAAATGTAGGTAAGTCTAAACACACTCTGTCTACTTCTTCTTCTT | |
| | CTTCTTCTTCTTCTTCTTCTTATTTTGAGACTGAGTCTTGCCCTGTCACCCAGACTGGAGTGCAGTGGCA | |
| | GGATCTTGGCTCACTGCTATCTCCACCTCCCAGGTTCAAGTGATTCTTCTGCCTCAGCCTCCCGAGTAGC | |
| | TGGGATTACATGCACATGCCACCATATCCGGCTAATTTTTGAATTTTTAGTAGAGATGGGGTTTCACTGT | |
| | GTTGGCCAGGCCGGTCTCAAACTCCCGACCTCAAGTGATCCCCCCGCCTCGGCCTCCCAAAGTGCTGGGA | |
| | TTACAGGCGTGTCTACATATTATTAAAATAACAATAATATTTATTTTGTGGGTTAATTTTTTTTGAAACA | |
| | GATATTGAATTTATTGGTTGGCTATGAGTAGAAAAATACATCAGTAAAGAAAAAAGACCCTGTATATAAA | |
| | TATAATACTAGCTAGTTAAAATTTGACCAAGAAGTTTCCATTGTGGGTTAATTTTTAAAGGCCTAACTGA | |
| | AATATGGAGTAACCACAGCATGCAGCATGTAAATTAAAGGGGATAGCTGG | |
| 20 | ACTCCTGGAATACACAGAGAGAGGCAGCAGCTTGCTCAGCGGACAAGGATGCTGGGCGTGAGGGACCAAG | >NM_001173552.2 |
| | GCCTGCCCTGCACTCGGGCCTCCTCCAGCCAGTGCTGACCAGGGACTTCTGACCTGCTGGCCAGCCAGGA | Homo sapiens |
| | CCTGTGTGGGGAGGCCCTCCTGCTGCCTTGGGGTGACAATCTCAGCTCCAGGCTACAGGGAGACCGGGAG | transmembrane |
| | GATCACAGAGCCAGCATGGATCCTGACAGTGATCAACCTCTGAACAGCCTCGTCAAGGTGATTCTGGATA | serine |
| | AATACTACTTCCTCTGCGGGCAGCCTCTCCACTTCATCCCGAGGAAGCAGCTGTGTGACGGAGAGCTGGA | protease 4 |
| | CTGTCCCTTGGGGGAGGACGAGGAGCACTGTGTCAAGAGCTTCCCCGAAGGGCCTGCAGTGGCAGTCCGC | (TMPRSS4), |
| | CTCTCCAAGGACCGATCCACACTGCAGGTGCTGGACTCGGCCACAGGGAACTGGTTCTCTGCCTGTTTCG | transcript |
| | ACAACTTCACAGAAGCTCTCGCTGAGACAGCCTGTAGGCAGATGGGCTACAGCAGCAAACCCACTTTCAG | variant 5, |
| | AGCTGTGGAGATTGGCCCAGACCAGGATCTGGATGTTGTTGAAATCACAGAAAACAGCCAGGAGCTTCGC | mRNA |
| | ATGCGGAACTCAAGTGGGCCCTGTCTCTCAGGCTCCCTGGTCTCCCTGCACTGTCTTGCCTGTGGGAAGA | |
| | GCCTGAAGACCCCCCGTGTGGTGGGTGTGGAGGAGGCCTCTGTGGATTCTTGGCCTTGGCAGGTCAGCAT | |
| | CCAGTACGACAAACAGCACGTCTGTGGAGGGAGCATCCTGGACCCCCACTGGGTCCTCACGGCAGCCCAC | |
| | TGCTTCAGGAAACATACCGATGTGTTCAACTGGAAGGTGCGGGCAGGCTCAGACAAACTGGGCAGCTTCC | |
| | CATCCCTGGCTGTGGCCAAGATCATCATCATTGAATTCAACCCCATGTACCCCAAAGACAATGACATCGC | |
| | CCTCATGAAGCTGCAGTTCCCACTCACTTTCTCAGGCACAGTCAGGCCCATCTGTCTGCCCTTCTTTGAT | |
| | GAGGAGCTCACTCCAGCCACCCCACTCTGGATCATTGGATGGGGCTTTACGAAGCAGAATGGAGGGAAGA | |
| | TGTCTGACATACTGCTGCAGGCGTCAGTCCAGGTCATTGACAGCACACGGTGCAATGCAGACGATGCGTA | |
| | CCAGGGGGAAGTCACCGAGAAGATGATGTGTGCAGGCATCCCGGAAGGGGGTGTGGACACCTGCCAGGGT | |
| | GACAGTGGTGGGCCCCTGATGTACCAATCTGACCAGTGGCATGTGGTGGGCATCGTTAGTTGGGGCTATG | |
| | GCTGCGGGGGCCCGAGCACCCCAGGAGTATACACCAAGGTCTCAGCCTATCTCAACTGGATCTACAATGT | |
| | CTGGAAGGCTGAGCTGTAATGCTGCTGCCCCTTTGCAGTGCTGGGAGCCGCTTCCTTCCTGCCCTGCCCA | |
| | CCTGGGGATCCCCCAAAGTCAGACACAGAGCAAGAGTCCCCTTGGGTACACCCCTCTGCCCACAGCCTCA | |
| | GCATTTCTTGGAGCAGCAAAGGGCCTCAATTCCTATAAGAGACCCTCGCAGCCCAGAGGCGCCCAGAGGA | |
| | AGTCAGCAGCCCTAGCTCGGCCACACTTGGTGCTCCCAGCATCCCAGGGAGAGACACAGCCCACTGAACA | |
| | AGGTCTCAGGGGTATTGCTAAGCCAAGAAGGAACTTTCCCACACTACTGAATGGAAGCAGGCTGTCTTGT | |
| | AAAAGCCCAGATCACTGTGGGCTGGAGAGGAGAAGGAAAGGGTCTGCGCCAGCCCTGTCCGTCTTCACCC | |
| | ATCCCCAAGCCTACTAGAGCAAGAAACCAGTTGTAATATAAAATGCACTGCCCTACTGTTGGTATGACTA | |
| | CCGTTACCTACTGTTGTCATTGTTATTACAGCTATGGCCACTATTATTAAAGAGCTGTGTAACATCTCTG | |
| | GCATAGGCTAGCTGGAATGCTTGATAAGAACTGAGCTGGGATGATTGAACTTTCATTCTTTGGCTTGGGG | |
| | AGAAAAGAAGTCCTGGGGAAGCAATTGAGTCTCAAAGTAGAGGCAGGGGAAAAAAGAGTTAGGGAGACCA | |
| | GATCTGCTGAGTGGCAGCAAGAGTGAGCTGCAGATTACAGAAACCAGGGTGAGCAAGTTTGAGTCCCACA | |
| | CAGGGCCTTCTCCCTTTGCCTCTTTCCCTCCCTCCCTGCCTGTGATAATCAGCCAGGAGCCAGGGATAAC | |
| | CTATGACTTGGGAAAGAGATGAGTTAGGCAGTCAAGGGTGACATTCAATCAGGGATCCACAAGTGGCTGG | |
| | AAAGAAATGCTGGTCCTGTGTCCTAACTTTTTCCGCCTGGAGAGCCCTCAGTGTGGCTTCTTACATTTAA | |
| | AAAACAAAAAGGATCAGCTGCCAGGTGTGAGGCAGTCCCCAAGCTGAGTTGTGAGGATGTAAGCATGAAT | |
| | AAGTCCCTGCACTCAAAATGGTCAAAGAATTAAACCCCATGGACTTTTTTGGCATCTGTATGAAAGCTTG | |
| | GGTTTTCTGAGGACTGTCTTGCTATAGTTAAGTCAGATCCTAGATGAAATATACTTGTTCATACTGTACT | |
| | AGGTTCTTAGGAAACAACAGAATTCCTCAAATGCCAAAAACAAAGAAAATAGAAACCCAGAAAACAAAAC | |
| | AAAATAAAACAAAACCATCAGAACTGTGAGTGGAAACTAAGGTGATGATCTGGGAGCAATACACTAAAAT | |
| | CTTGGGTCGAGACCTATATGAAGGCTGGCAGTGGAGCTAAACCTGGACACACTGAAGACAAGGGAGCTGA | |
| | ACCAGGGCTCCTACATGAAGCAGGGATAACTGATGGCAGTAAATGTGGTCTCAAATTGCAGATGGTCTGG | |
| | AGGAAAATTTCCCAAATTTAGAGCCTCAGGATTCCCAAAGATCCTCCAAATATGAGCTCACAATCAAAGA | |
| | TCAGAGACGTTGAAAAATAAAAAACACCTTAAGTGGGCAGCATAAAAAACAGCTAATTTAGAACCCCAAA | |
| | GGCTTCAGATGTCAGAATATTAGAGACTTATGATAATAAGCAATATTTGCAGAGTATTTGTATGTGCCAG | |
| | ACACTATTGTAAGTGCTTCATCATGTACTGATTCATTTAATACTCACAGAAATCTGTGAGATGGGTATTA | |
| | TTCTTATCCTCACTCTATGGATTAAAAAAACTAAGGCACAAAGTGGTTAAGCTCCTTGCCTGAGATTATA | |
| | GACTGTAAGTTGAACGTGAGCACTTGGAATACAGAGTTCATGCTGTAAACTACCACACTATAGGGCCTCC | |
| | AATATGATAATTTATAAAATATTTGAATAAAAAATGAATACTAGTTCCACATTTTAAAATCATGTTTAAC | |
| | TGTGGTCAAATGCACATAACACAAGTTGCCATCTTCACCATTTTTAGGTGTATAGTTCAGTGGTGTTATG | |
| | TACATTCACACTATTGTGCAGTCATCACCACCATCCATCTCCAGAACAGAAACTCAGTACCCATCAAACA | |
| | ACTCTCCATTTCCCCCTCCTCCCAATCTCTGGCAACCACCATTGTGCTTTCAGTCTCTGTGAACTGGATT | |
| | ACTCTGGGTACCTCATTTAAGTGAAGTCATGCAGTATTGGTCTTTTTGTACTTGTTTTATTTCACTTCAC | |
| | ATTGTGTCTTCAAGTTTCACCCATGTTGTAGCATGTGTCAGAATTTCTTCCCTTTTTAGACTAAATAATA | |
| | TTCTATTGTTTATACGAACATTCAGGTTACTTCTATCTTTTGGCTATTGTGAATTATGCTGCTGTGAACA | |
| | TGGGTGTACAAGTATCTCTTTGAGGCCCTGCTTTCAATTCTCTTGGGTATATTCCCAGAAGTGGAATTGC | |
| | TGGATCATATGGTAATTCTATTTTGAATTTTTTGAGGAACTGATATATTGCTTTCCATAGAGACTGCACC | |
| | ATTTTACATTCCCATCAACAGTTTGCAGGAGTTACTATTTCTCCATATCCCCCCTAACACTTGCTATTTT | |
| | CTGTTAAAAATGGATATCTTAATAATCAAGCAAAAAAACAGGCAGATTTGAAAAAGAACTGAATTACAGC | |
| | TTTTAGAAATAAAAACTATAATTATAAAAATAAAAAACTAAGTGGATGGGGTAAATAACAATTAAAACAC | |
| | CAATTAAGAGAGAACAAATGAACTGGAAGATAAATTGAAGAAGTGACTAGGCTTAACAGCAGAGAGAGAT | |
| | AAGGAGATTAAAAATATGAAAACAAGGCCAGGAGCAATGAAGCCTAGAATGGTAAATTCTAACATATCCA | |
| | GAATCCCAGAAAGAGAGAATCAAGACAATGAGAGAGAGACAGTACCAAAGAGATAAGAGCTGAGAATGTT | |
| | CCAGAATTGATAAAAGGTGTGAATCCACAGAACATACACCACCATAGTGTACACGCATACAACCAAGGTG | |
| | GAAAAATTAGAATAAATCCACACCTATGTACATTATAATGAAACTGCAGAACACCAAAGACAAAAAGAAA | |
| | CTCCTTATAGCAGCAGAGAGAAAACCCAGACCACCCACAGTACCACAAATCTACCACAATTAGACTGACA | |
| | ACAGGCTTTCCCACAGCAATAAAGGAGCTAGAAGTCAGTGGAAGTATATCTCCAGCATGCCAAAAGATAA | |
| | CAATCAATCAGGGATTGTGAACCCTACAAAACTATCTTTCAAGAATAAAGGCATTTTCAAGAAAACAAAA | |
| | ACAGACTTTACCATCAACAAACCTTCTCTAAAAGAATATATAAAGCATTTACTTTAGGAAGAAGGAAAAT | |
| | GATCCTAAAAGGAAGAACCAAGAAGCAAGTAGCAATAGTGAGGCAATTGTGAAAATGTAGGTAAGTCTAA | |
| | ACACACTCTGTCTACTTCTTCTTCTTCTTCTTCTTCTTCTTCTTCTTATTTTGAGACTGAGTCTTGCCCT | |
| | GTCACCCAGACTGGAGTGCAGTGGCAGGATCTTGGCTCACTGCTATCTCCACCTCCCAGGTTCAAGTGAT | |
| | TCTTCTGCCTCAGCCTCCCGAGTAGCTGGGATTACATGCACATGCCACCATATCCGGCTAATTTTTGAAT | |
| | TTTTAGTAGAGATGGGGTTTCACTGTGTTGGCCAGGCCGGTCTCAAACTCCCGACCTCAAGTGATCCCCC | |
| | CGCCTCGGCCTCCCAAAGTGCTGGGATTACAGGCGTGTCTACATATTATTAAAATAACAATAATATTTAT | |
| | TTTGTGGGTTAATTTTTTTTGAAACAGATATTGAATTTATTGGTTGGCTATGAGTAGAAAAATACATCAG | |
| | TAAAGAAAAAAGACCCTGTATATAAATATAATACTAGCTAGTTAAAATTTGACCAAGAAGTTTCCATTGT | |
| | GGGTTAATTTTTAAAGGCCTAACTGAAATATGGAGTAACCACAGCATGCAGCATGTAAATTAAAGGGGAT | |
| | AGCTGG | |
| 21 | ACTCCTGGAATACACAGAGAGAGGCAGCAGCTTGCTCAGCGGACAAGGATGCTGGGCGTGAGGGACCAAG | >NM_001290094.2 |
| | GCCTGCCCTGCACTCGGGCCTCCTCCAGCCAGTGCTGACCAGGGACTTCTGACCTGCTGGCCAGCCAGGA | Homo sapiens |
| | CCTGTGTGGGGAGGCCCTCCTGCTGCCTTGGGGTGACAATCTCAGCTCCAGGCTACAGGGAGACCGGGAG | transmembrane |
| | GATCACAGAGCCAGCATGGATCCTGACAGTGATCAACCTCTGAACAGCCTCGGTAAGTTCAGATGTCAAA | serine |
| | CCCCTGCGCAAACCCCGTATCCCCATGGAGACCTTCAGAAAGGTGGGGATCCCCATCATCATAGCACTAC | protease 4 |
| | TGAGCCTGGCGAGTATCATCATTGTGGTTGTCCTCATCAAGGTGATTCTGGATAAATACTACTTCCTCTG | (TMPRSS4), |
| | CGGGCAGCCTCTCCACTTCATCCCGAGGAAGCAGCTGTGTGACGGAGAGCTGGACTGTCCCTTGGGGGAG | transcript |
| | GACGAGGAGCACTGTGTCAAGAGCTTCCCCGAAGGGCCTGCAGTGGCAGTCCGCCTCTCCAAGGACCGAT | variant 6, |
| | CCACACTGCAGGTGCTGGACTCGGCCACAGGGAACTGGTTCTCTGCCTGTTTCGACAACTTCACAGAAGC | mRNA |
| | TCTCGCTGAGACAGCCTGTAGGCAGATGGGCTACAGCAGCAAACCCACTTTCAGAGCTGTGGAGATTGGC | |
| | CCAGACCAGGATCTGGATGTTGTTGAAATCACAGAAAACAGCCAGGAGCTTCGCATGCGGAACTCAAGTG | |
| | GGCCCTGTCTCTCAGGCTCCCTGGTCTCCCTGCACTGTCTTGCCTGTGGGAAGAGCCTGAAGACCCCCCG | |
| | TGTGGTGGGTGTGGAGGAGGCCTCTGTGGATTCTTGGCCTTGGCAGGTCAGCATCCAGTACGACAAACAG | |
| | CACGTCTGTGGAGGGAGCATCCTGGACCCCCACTGGGTCCTCACGGCAGCCCACTGCTTCAGGAAACATA | |
| | CCGATGTGTTCAACTGGAAGGTGCGGGCAGGCTCAGACAAACTGGGCAGCTTCCCATCCCTGGCTGTGGC | |
| | CAAGATCATCATCATTGAATTCAACCCCATGTACCCCAAAGACAATGACATCGCCCTCATGAAGCTGCAG | |
| | TTCCCACTCACTTTCTCAGGCACAGTCAGGCCCATCTGTCTGCCCTTCTTTGATGAGGAGCTCACTCCAG | |
| | CCACCCCACTCTGGATCATTGGATGGGGCTTTACGAAGCAGAATGGAGGGAAGATGTCTGACATACTGCT | |
| | GCAGGCGTCAGTCCAGGTCATTGACAGCACACGGTGCAATGCAGACGATGCGTACCAGGGGGAAGTCACC | |
| | GAGAAGATGATGTGTGCAGGCATCCCGGAAGGGGGTGTGGACACCTGCCAGGGTGACAGTGGTGGGCCCC | |
| | TGATGTACCAATCTGACCAGTGGCATGTGGTGGGCATCGTTAGTTGGGGCTATGGCTGCGGGGGCCCGAG | |
| | CACCCCAGGAGTATACACCAAGGTCTCAGCCTATCTCAACTGGATCTACAATGTCTGGAAGGCTGAGCTG | |
| | TAATGCTGCTGCCCCTTTGCAGTGCTGGGAGCCGCTTCCTTCCTGCCCTGCCCACCTGGGGATCCCCCAA | |
| | AGTCAGACACAGAGCAAGAGTCCCCTTGGGTACACCCCTCTGCCCACAGCCTCAGCATTTCTTGGAGCAG | |
| | CAAAGGGCCTCAATTCCTATAAGAGACCCTCGCAGCCCAGAGGCGCCCAGAGGAAGTCAGCAGCCCTAGC | |
| | TCGGCCACACTTGGTGCTCCCAGCATCCCAGGGAGAGACACAGCCCACTGAACAAGGTCTCAGGGGTATT | |
| | GCTAAGCCAAGAAGGAACTTTCCCACACTACTGAATGGAAGCAGGCTGTCTTGTAAAAGCCCAGATCACT | |
| | GTGGGCTGGAGAGGAGAAGGAAAGGGTCTGCGCCAGCCCTGTCCGTCTTCACCCATCCCCAAGCCTACTA | |
| | GAGCAAGAAACCAGTTGTAATATAAAATGCACTGCCCTACTGTTGGTATGACTACCGTTACCTACTGTTG | |
| | TCATTGTTATTACAGCTATGGCCACTATTATTAAAGAGCTGTGTAACATCTCTGGCATAGGCTAGCTGGA | |
| | ATGCTTGATAAGAACTGAGCTGGGATGATTGAACTTTCATTCTTTGGCTTGGGGAGAAAAGAAGTCCTGG | |
| | GGAAGCAATTGAGTCTCAAAGTAGAGGCAGGGGAAAAAAGAGTTAGGGAGACCAGATCTGCTGAGTGGCA | |
| | GCAAGAGTGAGCTGCAGATTACAGAAACCAGGGTGAGCAAGTTTGAGTCCCACACAGGGCCTTCTCCCTT | |
| | TGCCTCTTTCCCTCCCTCCCTGCCTGTGATAATCAGCCAGGAGCCAGGGATAACCTATGACTTGGGAAAG | |
| | AGATGAGTTAGGCAGTCAAGGGTGACATTCAATCAGGGATCCACAAGTGGCTGGAAAGAAATGCTGGTCC | |
| | TGTGTCCTAACTTTTTCCGCCTGGAGAGCCCTCAGTGTGGCTTCTTACATTTAAAAAACAAAAAGGATCA | |
| | GCTGCCAGGTGTGAGGCAGTCCCCAAGCTGAGTTGTGAGGATGTAAGCATGAATAAGTCCCTGCACTCAA | |
| | AATGGTCAAAGAATTAAACCCCATGGACTTTTTTGGCATCTGTATGAAAGCTTGGGTTTTCTGAGGACTG | |
| | TCTTGCTATAGTTAAGTCAGATCCTAGATGAAATATACTTGTTCATACTGTACTAGGTTCTTAGGAAACA | |
| | ACAGAATTCCTCAAATGCCAAAAACAAAGAAAATAGAAACCCAGAAAACAAAACAAAATAAAACAAAACC | |
| | ATCAGAACTGTGAGTGGAAACTAAGGTGATGATCTGGGAGCAATACACTAAAATCTTGGGTCGAGACCTA | |
| | TATGAAGGCTGGCAGTGGAGCTAAACCTGGACACACTGAAGACAAGGGAGCTGAACCAGGGCTCCTACAT | |
| | GAAGCAGGGATAACTGATGGCAGTAAATGTGGTCTCAAATTGCAGATGGTCTGGAGGAAAATTTCCCAAA | |
| | TTTAGAGCCTCAGGATTCCCAAAGATCCTCCAAATATGAGCTCACAATCAAAGATCAGAGACGTTGAAAA | |
| | ATAAAAAACACCTTAAGTGGGCAGCATAAAAAACAGCTAATTTAGAACCCCAAAGGCTTCAGATGTCAGA | |
| | ATATTAGAGACTTATGATAATAAGCAATATTTGCAGAGTATTTGTATGTGCCAGACACTATTGTAAGTGC | |
| | TTCATCATGTACTGATTCATTTAATACTCACAGAAATCTGTGAGATGGGTATTATTCTTATCCTCACTCT | |
| | ATGGATTAAAAAAACTAAGGCACAAAGTGGTTAAGCTCCTTGCCTGAGATTATAGACTGTAAGTTGAACG | |
| | TGAGCACTTGGAATACAGAGTTCATGCTGTAAACTACCACACTATAGGGCCTCCAATATGATAATTTATA | |
| | AAATATTTGAATAAAAAATGAATACTAGTTCCACATTTTAAAATCATGTTTAACTGTGGTCAAATGCACA | |
| | TAACACAAGTTGCCATCTTCACCATTTTTAGGTGTATAGTTCAGTGGTGTTATGTACATTCACACTATTG | |
| | TGCAGTCATCACCACCATCCATCTCCAGAACAGAAACTCAGTACCCATCAAACAACTCTCCATTTCCCCC | |
| | TCCTCCCAATCTCTGGCAACCACCATTGTGCTTTCAGTCTCTGTGAACTGGATTACTCTGGGTACCTCAT | |
| | TTAAGTGAAGTCATGCAGTATTGGTCTTTTTGTACTTGTTTTATTTCACTTCACATTGTGTCTTCAAGTT | |
| | TCACCCATGTTGTAGCATGTGTCAGAATTTCTTCCCTTTTTAGACTAAATAATATTCTATTGTTTATACG | |
| | AACATTCAGGTTACTTCTATCTTTTGGCTATTGTGAATTATGCTGCTGTGAACATGGGTGTACAAGTATC | |
| | TCTTTGAGGCCCTGCTTTCAATTCTCTTGGGTATATTCCCAGAAGTGGAATTGCTGGATCATATGGTAAT | |
| | TCTATTTTGAATTTTTTGAGGAACTGATATATTGCTTTCCATAGAGACTGCACCATTTTACATTCCCATC | |
| | AACAGTTTGCAGGAGTTACTATTTCTCCATATCCCCCCTAACACTTGCTATTTTCTGTTAAAAATGGATA | |
| | TCTTAATAATCAAGCAAAAATAACAGGCAGATTTGAAAAAGAACTGAATACAGCTTTTAGAAATAAAAAC | |
| | TATAATTATAAAAATAAAAAACTAAGTGGATGGGGTAAATAACAATTAAAACACCAATTAAGAGAGAACA | |
| | AATGAACTGGAAGATAAATTGAAGAAGTGACTAGGCTTAACAGCAGAGAGAGATAAGGAGATTAAAAATA | |
| | TGAAAACAAGGCCAGGAGCAATGAAGCCTAGAATGGTAAATTCTAACATATCCAGAATCCCAGAAAGAGA | |
| | GAATCAAGACAATGAGAGAGAGACAGTACCAAAGAGATAAGAGCTGAGAATGTTCCAGAATTGATAAAAG | |
| | GTGTGAATCCACAGAACATACACCACCATAGTGTACACGCATACAACCAAGGTGGAAAAATTAGAATAAA | |
| | TCCACACCTATGTACATTATAATGAAACTGCAGAACACCAAAGACAAAAAGAAACTCCTTATAGCAGCAG | |
| | AGAGAAAACCCAGACCACCCACAGTACCACAAATCTACCACAATTAGACTGACAACAGGCTTTCCCACAG | |
| | CAATAAAGGAGCTAGAAGTCAGTGGAAGTATATCTCCAGCATGCCAAAAGATAACAATCAATCAGGGATT | |
| | GTGAACCCTACAAAACTATCTTTCAAGAATAAAGGCATTTTCAAGAAAACAAAAACAGACTTTACCATCA | |
| | ACAAACCTTCTCTAAAAGAATATATAAAGCATTTACTTTAGGAAGAAGGAAAATGATCCTAAAAGGAAGA | |
| | ACCAAGAAGCAAGTAGCAATAGTGAGGCAATTGTGAAAATGTAGGTAAGTCTAAACACACTCTGTCTACT | |
| | TCTTCTTCTTCTTCTTCTTCTTCTTCTTCTTATTTTGAGACTGAGTCTTGCCCTGTCACCCAGACTGGAG | |
| | TGCAGTGGCAGGATCTTGGCTCACTGCTATCTCCACCTCCCAGGTTCAAGTGATTCTTCTGCCTCAGCCT | |
| | CCCGAGTAGCTGGGATTACATGCACATGCCACCATATCCGGCTAATTTTTGAATTTTTAGTAGAGATGGG | |
| | GTTTCACTGTGTTGGCCAGGCCGGTCTCAAACTCCCGACCTCAAGTGATCCCCCCGCCTCGGCCTCCCAA | |
| | AGTGCTGGGATTACAGGCGTGTCTACATATTATTAAAATAACAATAATATTTATTTTGTGGGTTAATTTT | |
| | TTTTGAAACAGATATTGAATTTATTGGTTGGCTATGAGTAGAAAAATACATCAGTAAAGAAAAAAGACCC | |
| | TGTATATAAATATAATACTAGCTAGTTAAAATTTGACCAAGAAGTTTCCATTGTGGGTTAATTTTTAAAG | |
| | GCCTAACTGAAATATGGAGTAACCACAGCATGCAGCATGTAAATTAAAGGGGATAGCTGG | |
| 22 | ACTCCTGGAATACACAGAGAGAGGCAGCAGCTTGCTCAGCGGACAAGGATGCTGGGCGTGAGGGACCAAG | >NM_001290096.2 |
| | GCCTGCCCTGCACTCGGGCCTCCTCCAGCCAGTGCTGACCAGGGACTTCTGACCTGCTGGCCAGCCAGGA | Homo sapiens |
| | CCTGTGTGGGGAGGCCCTCCTGCTGCCTTGGGGTGACAATCTCAGCTCCAGGCTACAGGGAGACCGGGAG | transmembrane |
| | GATCACAGAGCCAGCATGGATCCTGACAGTGATCAACCTCTGAACAGCCTCGATGTCAAACCCCTGCGCA | serine |
| | AACCCCGTATCCCCATGGAGACCTTCAGAAAGTCAAGGTGATTCTGGATAAATACTACTTCCTCTGCGGG | protease 4 |
| | CAGCCTCTCCACTTCATCCCGAGGAAGCAGCTGTGTGACGGAGAGCTGGACTGTCCCTTGGGGGAGGACG | (TMPRSS4), |
| | AGGAGCACTGTGTCAAGAGCTTCCCCGAAGGGCCTGCAGTGGCAGTCCGCCTCTCCAAGGACCGATCCAC | transcript |
| | ACTGCAGGTGCTGGACTCGGCCACAGGGAACTGGTTCTCTGCCTGTTTCGACAACTTCACAGAAGCTCTC | variant 7, |
| | GCTGAGACAGCCTGTAGGCAGATGGGCTACAGCAGAGCTGTGGAGATTGGCCCAGACCAGGATCTGGATG | mRNA |
| | TTGTTGAAATCACAGAAAACAGCCAGGAGCTTCGCATGCGGAACTCAAGTGGGCCCTGTCTCTCAGGCTC | |
| | CCTGGTCTCCCTGCACTGTCTTGCCTGTGGGAAGAGCCTGAAGACCCCCCGTGTGGTGGGTGTGGAGGAG | |
| | GCCTCTGTGGATTCTTGGCCTTGGCAGGTCAGCATCCAGTACGACAAACAGCACGTCTGTGGAGGGAGCA | |
| | TCCTGGACCCCCACTGGGTCCTCACGGCAGCCCACTGCTTCAGGAAACATACCGATGTGTTCAACTGGAA | |
| | GGTGCGGGCAGGCTCAGACAAACTGGGCAGCTTCCCATCCCTGGCTGTGGCCAAGATCATCATCATTGAA | |
| | TTCAACCCCATGTACCCCAAAGACAATGACATCGCCCTCATGAAGCTGCAGTTCCCACTCACTTTCTCAG | |
| | GCACAGTCAGGCCCATCTGTCTGCCCTTCTTTGATGAGGAGCTCACTCCAGCCACCCCACTCTGGATCAT | |
| | TGGATGGGGCTTTACGAAGCAGAATGGAGGGAAGATGTCTGACATACTGCTGCAGGCGTCAGTCCAGGTC | |
| | ATTGACAGCACACGGTGCAATGCAGACGATGCGTACCAGGGGGAAGTCACCGAGAAGATGATGTGTGCAG | |
| | GCATCCCGGAAGGGGGTGTGGACACCTGCCAGGGTGACAGTGGTGGGCCCCTGATGTACCAATCTGACCA | |
| | GTGGCATGTGGTGGGCATCGTTAGTTGGGGCTATGGCTGCGGGGGCCCGAGCACCCCAGGAGTATACACC | |
| | AAGGTCTCAGCCTATCTCAACTGGATCTACAATGTCTGGAAGGCTGAGCTGTAATGCTGCTGCCCCTTTG | |
| | CAGTGCTGGGAGCCGCTTCCTTCCTGCCCTGCCCACCTGGGGATCCCCCAAAGTCAGACACAGAGCAAGA | |
| | GTCCCCTTGGGTACACCCCTCTGCCCACAGCCTCAGCATTTCTTGGAGCAGCAAAGGGCCTCAATTCCTA | |
| | TAAGAGACCCTCGCAGCCCAGAGGCGCCCAGAGGAAGTCAGCAGCCCTAGCTCGGCCACACTTGGTGCTC | |
| | CCAGCATCCCAGGGAGAGACACAGCCCACTGAACAAGGTCTCAGGGGTATTGCTAAGCCAAGAAGGAACT | |
| | TTCCCACACTACTGAATGGAAGCAGGCTGTCTTGTAAAAGCCCAGATCACTGTGGGCTGGAGAGGAGAAG | |
| | GAAAGGGTCTGCGCCAGCCCTGTCCGTCTTCACCCATCCCCAAGCCTACTAGAGCAAGAAACCAGTTGTA | |
| | ATATAAAATGCACTGCCCTACTGTTGGTATGACTACCGTTACCTACTGTTGTCATTGTTATTACAGCTAT | |
| | GGCCACTATTATTAAAGAGCTGTGTAACATCTCTGGCATAGGCTAGCTGGAATGCTTGATAAGAACTGAG | |
| | CTGGGATGATTGAACTTTCATTCTTTGGCTTGGGGAGAAAAGAAGTCCTGGGGAAGCAATTGAGTCTCAA | |
| | AGTAGAGGCAGGGGAAAAAAGAGTTAGGGAGACCAGATCTGCTGAGTGGCAGCAAGAGTGAGCTGCAGAT | |
| | TACAGAAACCAGGGTGAGCAAGTTTGAGTCCCACACAGGGCCTTCTCCCTTTGCCTCTTTCCCTCCCTCC | |
| | CTGCCTGTGATAATCAGCCAGGAGCCAGGGATAACCTATGACTTGGGAAAGAGATGAGTTAGGCAGTCAA | |
| | GGGTGACATTCAATCAGGGATCCACAAGTGGCTGGAAAGAAATGCTGGTCCTGTGTCCTAACTTTTTCCG | |
| | CCTGGAGAGCCCTCAGTGTGGCTTCTTACATTTAAAAAACAAAAAGGATCAGCTGCCAGGTGTGAGGCAG | |
| | TCCCCAAGCTGAGTTGTGAGGATGTAAGCATGAATAAGTCCCTGCACTCAAAATGGTCAAAGAATTAAAC | |
| | CCCATGGACTTTTTTGGCATCTGTATGAAAGCTTGGGTTTTCTGAGGACTGTCTTGCTATAGTTAAGTCA | |
| | GATCCTAGATGAAATATACTTGTTCATACTGTACTAGGTTCTTAGGAAACAACAGAATTCCTCAAATGCC | |
| | AAAAACAAAGAAAATAGAAACCCAGAAAACAAAACAAAATAAAACAAAACCATCAGAACTGTGAGTGGAA | |
| | ACTAAGGTGATGATCTGGGAGCAATACACTAAAATCTTGGGTCGAGACCTATATGAAGGCTGGCAGTGGA | |
| | GCTAAACCTGGACACACTGAAGACAAGGGAGCTGAACCAGGGCTCCTACATGAAGCAGGGATAACTGATG | |
| | GCAGTAAATGTGGTCTCAAATTGCAGATGGTCTGGAGGAAAATTTCCCAAATTTAGAGCCTCAGGATTCC | |
| | CAAAGATCCTCCAAATATGAGCTCACAATCAAAGATCAGAGACGTTGAAAAATAAAAAACACCTTAAGTG | |
| | GGCAGCATAAAAAACAGCTAATTTAGAACCCCAAAGGCTTCAGATGTCAGAATATTAGAGACTTATGATA | |
| | ATAAGCAATATTTGCAGAGTATTTGTATGTGCCAGACACTATTGTAAGTGCTTCATCATGTACTGATTCA | |
| | TTTAATACTCACAGAAATCTGTGAGATGGGTATTATTCTTATCCTCACTCTATGGATTAAAAAAACTAAG | |
| | GCACAAAGTGGTTAAGCTCCTTGCCTGAGATTATAGACTGTAAGTTGAACGTGAGCACTTGGAATACAGA | |
| | GTTCATGCTGTAAACTACCACACTATAGGGCCTCCAATATGATAATTTATAAAATATTTGAATAAAAAAT | |
| | GAATACTAGTTCCACATTTTAAAATCATGTTTAACTGTGGTCAAATGCACATAACACAAGTTGCCATCTT | |
| | CACCATTTTTAGGTGTATAGTTCAGTGGTGTTATGTACATTCACACTATTGTGCAGTCATCACCACCATC | |
| | CATCTCCAGAACAGAAACTCAGTACCCATCAAACAACTCTCCATTTCCCCCTCCTCCCAATCTCTGGCAA | |
| | CCACCATTGTGCTTTCAGTCTCTGTGAACTGGATTACTCTGGGTACCTCATTTAAGTGAAGTCATGCAGT | |
| | ATTGGTCTTTTTGTACTTGTTTTATTTCACTTCACATTGTGTCTTCAAGTTTCACCCATGTTGTAGCATG | |
| | TGTCAGAATTTCTTCCCTTTTTAGACTAAATAATATTCTATTGTTTATACGAACATTCAGGTTACTTCTA | |
| | TCTTTTGGCTATTGTGAATTATGCTGCTGTGAACATGGGTGTACAAGTATCTCTTTGAGGCCCTGCTTTC | |
| | AATTCTCTTGGGTATATTCCCAGAAGTGGAATTGCTGGATCATATGGTAATTCTATTTTGAATTTTTTGA | |
| | GGAACTGATATATTGCTTTCCATAGAGACTGCACCATTTTACATTCCCATCAACAGTTTGCAGGAGTTAC | |
| | TATTTCTCCATATCCCCCCTAACACTTGCTATTTTCTGTTAAAAATGGATATCTTAATAATCAAGCAAAA | |
| | ATAACAGGCAGATTTGAAAAAGAACTGAATACAGCTTTTAGAAATAAAAACTATAATTATAAAAATAAAA | |
| | AACTAAGTGGATGGGGTAAATAACAATTAAAACACCAATTAAGAGAGAACAAATGAACTGGAAGATAAAT | |
| | TGAAGAAGTGACTAGGCTTAACAGCAGAGAGAGATAAGGAGATTAAAAATATGAAAACAAGGCCAGGAGC | |
| | AATGAAGCCTAGAATGGTAAATTCTAACATATCCAGAATCCCAGAAAGAGAGAATCAAGACAATGAGAGA | |
| | GAGACAGTACCAAAGAGATAAGAGCTGAGAATGTTCCAGAATTGATAAAAGGTGTGAATCCACAGAACAT | |
| | ACACCACCATAGTGTACACGCATACAACCAAGGTGGAAAAATTAGAATAAATCCACACCTATGTACATTA | |
| | TAATGAAACTGCAGAACACCAAAGACAAAAAGAAACTCCTTATAGCAGCAGAGAGAAAACCCAGACCACC | |
| | CACAGTACCACAAATCTACCACAATTAGACTGACAACAGGCTTTCCCACAGCAATAAAGGAGCTAGAAGT | |
| | CAGTGGAAGTATATCTCCAGCATGCCAAAAGATAACAATCAATCAGGGATTGTGAACCCTACAAAACTAT | |
| | CTTTCAAGAATAAAGGCATTTTCAAGAAAACAAAAACAGACTTTACCATCAACAAACCTTCTCTAAAAGA | |
| | ATATATAAAGCATTTACTTTAGGAAGAAGGAAAATGATCCTAAAAGGAAGAACCAAGAAGCAAGTAGCAA | |
| | TAGTGAGGCAATTGTGAAAATGTAGGTAAGTCTAAACACACTCTGTCTACTTCTTCTTCTTCTTCTTCTT | |
| | CTTCTTCTTCTTATTTTGAGACTGAGTCTTGCCCTGTCACCCAGACTGGAGTGCAGTGGCAGGATCTTGG | |
| | CTCACTGCTATCTCCACCTCCCAGGTTCAAGTGATTCTTCTGCCTCAGCCTCCCGAGTAGCTGGGATTAC | |
| | ATGCACATGCCACCATATCCGGCTAATTTTTGAATTTTTAGTAGAGATGGGGTTTCACTGTGTTGGCCAG | |
| | GCCGGTCTCAAACTCCCGACCTCAAGTGATCCCCCCGCCTCGGCCTCCCAAAGTGCTGGGATTACAGGCG | |
| | TGTCTACATATTATTAAAATAACAATAATATTTATTTTGTGGGTTAATTTTTTTTGAAACAGATATTGAA | |
| | TTTATTGGTTGGCTATGAGTAGAAAAATACATCAGTAAAGAAAAAAGACCCTGTATATAAATATAATACT | |
| | AGCTAGTTAAAATTTGACCAAGAAGTTTCCATTGTGGGTTAATTTTTAAAGGCCTAACTGAAATATGGAG | |
| | TAACCACAGCATGCAGCATGTAAATTAAAGGGGATAGCTGG | |
| 23 | ACTCCTGGAATACACAGAGAGAGGCAGCAGCTTGCTCAGCGGACAAGGATGCTGGGCGTGAGGGACCAAG | >NM_019894.4 |
| | GCCTGCCCTGCACTCGGGCCTCCTCCAGCCAGTGCTGACCAGGGACTTCTGACCTGCTGGCCAGCCAGGA | Homo sapiens |
| | CCTGTGTGGGGAGGCCCTCCTGCTGCCTTGGGGTGACAATCTCAGCTCCAGGCTACAGGGAGACCGGGAG | transmembrane |
| | GATCACAGAGCCAGCATGTTACAGGATCCTGACAGTGATCAACCTCTGAACAGCCTCGATGTCAAACCCC | serine |
| | TGCGCAAACCCCGTATCCCCATGGAGACCTTCAGAAAGGTGGGGATCCCCATCATCATAGCACTACTGAG | protease 4 |
| | CCTGGCGAGTATCATCATTGTGGTTGTCCTCATCAAGGTGATTCTGGATAAATACTACTTCCTCTGCGGG | (TMPRSS4), |
| | CAGCCTCTCCACTTCATCCCGAGGAAGCAGCTGTGTGACGGAGAGCTGGACTGTCCCTTGGGGGAGGACG | transcript |
| | AGGAGCACTGTGTCAAGAGCTTCCCCGAAGGGCCTGCAGTGGCAGTCCGCCTCTCCAAGGACCGATCCAC | variant 1, |
| | ACTGCAGGTGCTGGACTCGGCCACAGGGAACTGGTTCTCTGCCTGTTTCGACAACTTCACAGAAGCTCTC | mRNA |
| | GCTGAGACAGCCTGTAGGCAGATGGGCTACAGCAGCAAACCCACTTTCAGAGCTGTGGAGATTGGCCCAG | |
| | ACCAGGATCTGGATGTTGTTGAAATCACAGAAAACAGCCAGGAGCTTCGCATGCGGAACTCAAGTGGGCC | |
| | CTGTCTCTCAGGCTCCCTGGTCTCCCTGCACTGTCTTGCCTGTGGGAAGAGCCTGAAGACCCCCCGTGTG | |
| | GTGGGTGTGGAGGAGGCCTCTGTGGATTCTTGGCCTTGGCAGGTCAGCATCCAGTACGACAAACAGCACG | |
| | TCTGTGGAGGGAGCATCCTGGACCCCCACTGGGTCCTCACGGCAGCCCACTGCTTCAGGAAACATACCGA | |
| | TGTGTTCAACTGGAAGGTGCGGGCAGGCTCAGACAAACTGGGCAGCTTCCCATCCCTGGCTGTGGCCAAG | |
| | ATCATCATCATTGAATTCAACCCCATGTACCCCAAAGACAATGACATCGCCCTCATGAAGCTGCAGTTCC | |
| | CACTCACTTTCTCAGGCACAGTCAGGCCCATCTGTCTGCCCTTCTTTGATGAGGAGCTCACTCCAGCCAC | |
| | CCCACTCTGGATCATTGGATGGGGCTTTACGAAGCAGAATGGAGGGAAGATGTCTGACATACTGCTGCAG | |
| | GCGTCAGTCCAGGTCATTGACAGCACACGGTGCAATGCAGACGATGCGTACCAGGGGGAAGTCACCGAGA | |
| | AGATGATGTGTGCAGGCATCCCGGAAGGGGGTGTGGACACCTGCCAGGGTGACAGTGGTGGGCCCCTGAT | |
| | GTACCAATCTGACCAGTGGCATGTGGTGGGCATCGTTAGTTGGGGCTATGGCTGCGGGGGCCCGAGCACC | |
| | CCAGGAGTATACACCAAGGTCTCAGCCTATCTCAACTGGATCTACAATGTCTGGAAGGCTGAGCTGTAAT | |
| | GCTGCTGCCCCTTTGCAGTGCTGGGAGCCGCTTCCTTCCTGCCCTGCCCACCTGGGGATCCCCCAAAGTC | |
| | AGACACAGAGCAAGAGTCCCCTTGGGTACACCCCTCTGCCCACAGCCTCAGCATTTCTTGGAGCAGCAAA | |
| | GGGCCTCAATTCCTATAAGAGACCCTCGCAGCCCAGAGGCGCCCAGAGGAAGTCAGCAGCCCTAGCTCGG | |
| | CCACACTTGGTGCTCCCAGCATCCCAGGGAGAGACACAGCCCACTGAACAAGGTCTCAGGGGTATTGCTA | |
| | AGCCAAGAAGGAACTTTCCCACACTACTGAATGGAAGCAGGCTGTCTTGTAAAAGCCCAGATCACTGTGG | |
| | GCTGGAGAGGAGAAGGAAAGGGTCTGCGCCAGCCCTGTCCGTCTTCACCCATCCCCAAGCCTACTAGAGC | |
| | AAGAAACCAGTTGTAATATAAAATGCACTGCCCTACTGTTGGTATGACTACCGTTACCTACTGTTGTCAT | |
| | TGTTATTACAGCTATGGCCACTATTATTAAAGAGCTGTGTAACATCTCTGGCATAGGCTAGCTGGAATGC | |
| | TTGATAAGAACTGAGCTGGGATGATTGAACTTTCATTCTTTGGCTTGGGGAGAAAAGAAGTCCTGGGGAA | |
| | GCAATTGAGTCTCAAAGTAGAGGCAGGGGAAAAAAGAGTTAGGGAGACCAGATCTGCTGAGTGGCAGCAA | |
| | GAGTGAGCTGCAGATTACAGAAACCAGGGTGAGCAAGTTTGAGTCCCACACAGGGCCTTCTCCCTTTGCC | |
| | TCTTTCCCTCCCTCCCTGCCTGTGATAATCAGCCAGGAGCCAGGGATAACCTATGACTTGGGAAAGAGAT | |
| | GAGTTAGGCAGTCAAGGGTGACATTCAATCAGGGATCCACAAGTGGCTGGAAAGAAATGCTGGTCCTGTG | |
| | TCCTAACTTTTTCCGCCTGGAGAGCCCTCAGTGTGGCTTCTTACATTTAAAAAACAAAAAGGATCAGCTG | |
| | CCAGGTGTGAGGCAGTCCCCAAGCTGAGTTGTGAGGATGTAAGCATGAATAAGTCCCTGCACTCAAAATG | |
| | GTCAAAGAATTAAACCCCATGGACTTTTTTGGCATCTGTATGAAAGCTTGGGTTTTCTGAGGACTGTCTT | |
| | GCTATAGTTAAGTCAGATCCTAGATGAAATATACTTGTTCATACTGTACTAGGTTCTTAGGAAACAACAG | |
| | AATTCCTCAAATGCCAAAAACAAAGAAAATAGAAACCCAGAAAACAAAACAAAATAAAACAAAACCATCA | |
| | GAACTGTGAGTGGAAACTAAGGTGATGATCTGGGAGCAATACACTAAAATCTTGGGTCGAGACCTATATG | |
| | AAGGCTGGCAGTGGAGCTAAACCTGGACACACTGAAGACAAGGGAGCTGAACCAGGGCTCCTACATGAAG | |
| | CAGGGATAACTGATGGCAGTAAATGTGGTCTCAAATTGCAGATGGTCTGGAGGAAAATTTCCCAAATTTA | |
| | GAGCCTCAGGATTCCCAAAGATCCTCCAAATATGAGCTCACAATCAAAGATCAGAGACGTTGAAAAATAA | |
| | AAAACACCTTAAGTGGGCAGCATAAAAAACAGCTAATTTAGAACCCCAAAGGCTTCAGATGTCAGAATAT | |
| | TAGAGACTTATGATAATAAGCAATATTTGCAGAGTATTTGTATGTGCCAGACACTATTGTAAGTGCTTCA | |
| | TCATGTACTGATTCATTTAATACTCACAGAAATCTGTGAGATGGGTATTATTCTTATCCTCACTCTATGG | |
| | ATTAAAAAAACTAAGGCACAAAGTGGTTAAGCTCCTTGCCTGAGATTATAGACTGTAAGTTGAACGTGAG | |
| | CACTTGGAATACAGAGTTCATGCTGTAAACTACCACACTATAGGGCCTCCAATATGATAATTTATAAAAT | |
| | ATTTGAATAAAAAATGAATACTAGTTCCACATTTTAAAATCATGTTTAACTGTGGTCAAATGCACATAAC | |
| | ACAAGTTGCCATCTTCACCATTTTTAGGTGTATAGTTCAGTGGTGTTATGTACATTCACACTATTGTGCA | |
| | GTCATCACCACCATCCATCTCCAGAACAGAAACTCAGTACCCATCAAACAACTCTCCATTTCCCCCTCCT | |
| | CCCAATCTCTGGCAACCACCATTGTGCTTTCAGTCTCTGTGAACTGGATTACTCTGGGTACCTCATTTAA | |
| | GTGAAGTCATGCAGTATTGGTCTTTTTGTACTTGTTTTATTTCACTTCACATTGTGTCTTCAAGTTTCAC | |
| | CCATGTTGTAGCATGTGTCAGAATTTCTTCCCTTTTTAGACTAAATAATATTCTATTGTTTATACGAACA | |
| | TTCAGGTTACTTCTATCTTTTGGCTATTGTGAATTATGCTGCTGTGAACATGGGTGTACAAGTATCTCTT | |
| | TGAGGCCCTGCTTTCAATTCTCTTGGGTATATTCCCAGAAGTGGAATTGCTGGATCATATGGTAATTCTA | |
| | TTTTGAATTTTTTGAGGAACTGATATATTGCTTTCCATAGAGACTGCACCATTTTACATTCCCATCAACA | |
| | GTTTGCAGGAGTTACTATTTCTCCATATCCCCCCTAACACTTGCTATTTTCTGTTAAAAATGGATATCTT | |
| | AATAATCAAGCAAAAATAACAGGCAGATTTGAAAAAGAACTGAATACAGCTTTTAGAAATAAAAACTATA | |
| | ATTATAAAAATAAAAAACTAAGTGGATGGGGTAAATAACAATTAAAACACCAATTAAGAGAGAACAAATG | |
| | AACTGGAAGATAAATTGAAGAAGTGACTAGGCTTAACAGCAGAGAGAGATAAGGAGATTAAAAATATGAA | |
| | AACAAGGCCAGGAGCAATGAAGCCTAGAATGGTAAATTCTAACATATCCAGAATCCCAGAAAGAGAGAAT | |
| | CAAGACAATGAGAGAGAGACAGTACCAAAGAGATAAGAGCTGAGAATGTTCCAGAATTGATAAAAGGTGT | |
| | GAATCCACAGAACATACACCACCATAGTGTACACGCATACAACCAAGGTGGAAAAATTAGAATAAATCCA | |
| | CACCTATGTACATTATAATGAAACTGCAGAACACCAAAGACAAAAAGAAACTCCTTATAGCAGCAGAGAG | |
| | AAAACCCAGACCACCCACAGTACCACAAATCTACCACAATTAGACTGACAACAGGCTTTCCCACAGCAAT | |
| | AAAGGAGCTAGAAGTCAGTGGAAGTATATCTCCAGCATGCCAAAAGATAACAATCAATCAGGGATTGTGA | |
| | ACCCTACAAAACTATCTTTCAAGAATAAAGGCATTTTCAAGAAAACAAAAACAGACTTTACCATCAACAA | |
| | ACCTTCTCTAAAAGAATATATAAAGCATTTACTTTAGGAAGAAGGAAAATGATCCTAAAAGGAAGAACCA | |
| | AGAAGCAAGTAGCAATAGTGAGGCAATTGTGAAAATGTAGGTAAGTCTAAACACACTCTGTCTACTTCTT | |
| | CTTCTTCTTCTTCTTCTTCTTCTTCTTATTTTGAGACTGAGTCTTGCCCTGTCACCCAGACTGGAGTGCA | |
| | GTGGCAGGATCTTGGCTCACTGCTATCTCCACCTCCCAGGTTCAAGTGATTCTTCTGCCTCAGCCTCCCG | |
| | AGTAGCTGGGATTACATGCACATGCCACCATATCCGGCTAATTTTTGAATTTTTAGTAGAGATGGGGTTT | |
| | CACTGTGTTGGCCAGGCCGGTCTCAAACTCCCGACCTCAAGTGATCCCCCCGCCTCGGCCTCCCAAAGTG | |
| | CTGGGATTACAGGCGTGTCTACATATTATTAAAATAACAATAATATTTATTTTGTGGGTTAATTTTTTTT | |
| | GAAACAGATATTGAATTTATTGGTTGGCTATGAGTAGAAAAATACATCAGTAAAGAAAAAAGACCCTGTA | |
| | TATAAATATAATACTAGCTAGTTAAAATTTGACCAAGAAGTTTCCATTGTGGGTTAATTTTTAAAGGCCT | |
| | AACTGAAATATGGAGTAACCACAGCATGCAGCATGTAAATTAAAGGGGATAGCTGG | |
| 24 | MLQDPDSDQPLNSLDVKPLRKPRIPMETFRKVGIPIIIALLSLASIIIVVVLIKVILDKYYFLCGQPLHF | >NP_001077416.2 |
| | IPRKQLCDGELDCPLGEDEEHCVKSFPEGPAVAVRLSKDRSTLQVLDSATGNWFSACFDNFTEALAETAC | transmembrane |
| | RQMGYSRAVEIGPDQDLDVVEITENSQELRMRNSSGPCLSGSLVSLHCLACGKSLKTPRVVGVEEASVDS | protease |
| | WPWQVSIQYDKQHVCGGSILDPHWVLTAAHCFRKHTDVFNWKVRAGSDKLGSFPSLAVAKIIIIEFNPMY | serine 4 |
| | PKDNDIALMKLQFPLTFSGTVRPICLPFFDEELTPATPLWIIGWGFTKQNGGKMSDILLQASVQVIDSTR | isoform 3 |
| | CNADDAYQGEVTEKMMCAGIPEGGVDTCQGDSGGPLMYQSDQWHVVGIVSWGYGCGGPSTPGVYTKVSAY | [Homo |
| | LNWIYNVWKAEL | sapiens] |
| 25 | MDPDSDQPLNSLDVKPLRKPRIPMETFRKVGIPIIIALLSLASIIIVVVLIKVILDKYYFLCGQPLHFIP | >NP_001167022.2 |
| | RKQLCDGELDCPLGEDEEHCVKSFPEGPAVAVRLSKDRSTLQVLDSATGNWFSACFDNFTEALAETACRQ | transmembrane |
| | MGYSSKPTFRAVEIGPDQDLDVVEITENSQELRMRNSSGPCLSGSLVSLHCLACGKSLKTPRVVGVEEAS | protease |
| | VDSWPWQVSIQYDKQHVCGGSILDPHWVLTAAHCFRKHTDVFNWKVRAGSDKLGSFPSLAVAKIIIIEFN | serine 4 |
| | PMYPKDNDIALMKLQFPLTFSGTVRPICLPFFDEELTPATPLWIIGWGFTKQNGGKMSDILLQASVQVID | isoform 4 |
| | STRCNADDAYQGEVTEKMMCAGIPEGGVDTCQGDSGGPLMYQSDQWHVVGIVSWGYGCGGPSTPGVYTKV | [Homo |
| | SAYLNWIYNVWKAEL | sapiens] |
| 26 | MDPDSDQPLNSLVKVILDKYYFLCGQPLHFIPRKQLCDGELDCPLGEDEEHCVKSFPEGPAVAVRLSKDR | >NP_001167023.2 |
| | STLQVLDSATGNWFSACFDNFTEALAETACRQMGYSSKPTFRAVEIGPDQDLDVVEITENSQELRMRNSS | transmembrane |
| | GPCLSGSLVSLHCLACGKSLKTPRVVGVEEASVDSWPWQVSIQYDKQHVCGGSILDPHWVLTAAHCFRKH | protease |
| | TDVFNWKVRAGSDKLGSFPSLAVAKIIIIEFNPMYPKDNDIALMKLQFPLTFSGTVRPICLPFFDEELTP | serine 4 |
| | ATPLWIIGWGFTKQNGGKMSDILLQASVQVIDSTRCNADDAYQGEVTEKMMCAGIPEGGVDTCQGDSGGP | isoform 5 |
| | LMYQSDQWHVVGIVSWGYGCGGPSTPGVYTKVSAYLNWIYNVWKAEL | [Homo sapiens] |
| 27 | METFRKVGIPIIIALLSLASIIIVVVLIKVILDKYYFLCGQPLHFIPRKQLCDGELDCPLGEDEEHCVKS | >NP_001277023.2 |
| | FPEGPAVAVRLSKDRSTLQVLDSATGNWFSACFDNFTEALAETACRQMGYSSKPTFRAVEIGPDQDLDVV | transmembrane |
| | EITENSQELRMRNSSGPCLSGSLVSLHCLACGKSLKTPRVVGVEEASVDSWPWQVSIQYDKQHVCGGSIL | protease |
| | DPHWVLTAAHCFRKHTDVFNWKVRAGSDKLGSFPSLAVAKIIIIEFNPMYPKDNDIALMKLQFPLTFSGT | serine 4 |
| | VRPICLPFFDEELTPATPLWIIGWGFTKQNGGKMSDILLQASVQVIDSTRCNADDAYQGEVTEKMMCAGI | isoform 6 |
| | PEGGVDTCQGDSGGPLMYQSDQWHVVGIVSWGYGCGGPSTPGVYTKVSAYLNWIYNVWKAEL | [Homo sapiens] |
| 28 | MGYSRAVEIGPDQDLDVVEITENSQELRMRNSSGPCLSGSLVSLHCLACGKSLKTPRVVGVEEASVDSWP | >NP_001277025.2 |
| | WQVSIQYDKQHVCGGSILDPHWVLTAAHCFRKHTDVFNWKVRAGSDKLGSFPSLAVAKIIIIEFNPMYPK | transmembrane |
| | DNDIALMKLQFPLTFSGTVRPICLPFFDEELTPATPLWIIGWGFTKQNGGKMSDILLQASVQVIDSTRCN | protease |
| | ADDAYQGEVTEKMMCAGIPEGGVDTCQGDSGGPLMYQSDQWHVVGIVSWGYGCGGPSTPGVYTKVSAYLN | serine 4 |
| | WIYNVWKAEL | isoform 7 |
| | | [Homo |
| | | sapiens] |
| 29 | MLQDPDSDQPLNSLDVKPLRKPRIPMETFRKVGIPIIIALLSLASIIIVVVLIKVILDKYYFLCGQPLHF | >NP_063947.2 |
| | IPRKQLCDGELDCPLGEDEEHCVKSFPEGPAVAVRLSKDRSTLQVLDSATGNWFSACFDNFTEALAETAC | transmembrane |
| | RQMGYSSKPTFRAVEIGPDQDLDVVEITENSQELRMRNSSGPCLSGSLVSLHCLACGKSLKTPRVVGVEE | protease |
| | ASVDSWPWQVSIQYDKQHVCGGSILDPHWVLTAAHCFRKHTDVFNWKVRAGSDKLGSFPSLAVAKIIIIE | serine 4 |
| | FNPMYPKDNDIALMKLQFPLTFSGTVRPICLPFFDEELTPATPLWIIGWGFTKQNGGKMSDILLQASVQV | isoform 1 |
| | IDSTRCNADDAYQGEVTEKMMCAGIPEGGVDTCQGDSGGPLMYQSDQWHVVGIVSWGYGCGGPSTPGVYT | [Homo |
| | KVSAYLNWIYNVWKAEL | sapiens] |
| 30 | ACTCGCCCTCCAGCTTCTGCCCTGCCTGCTGTGTGCGGAGCCGTCCAGCGACCACCATGGTGAGGCTCGT | >NM_001003841.3 |
| | GCTGCCCAACCCCGGCCTAGACGCCCGGATCCCGTCCCTGGCTGAGCTGGAGACCATCGAGCAGGAGGAG | Homo sapiens |
| | GCCAGCTCCCGGCCGAAGTGGGACAACAAGGCGCAGTACATGCTCACCTGCCTGGGCTTCTGCGTGGGCC | solute |
| | TCGGCAACGTGTGGCGCTTCCCCTACCTGTGTCAGAGCCACGGAGGAGGAGCCTTCATGATCCCGTTCCT | carrier |
| | CATCCTGCTGGTCCTGGAGGGCATCCCCCTGCTGTACCTGGAGTTCGCCATCGGGCAGCGGCTGCGGCGG | family 6 |
| | GGCAGCCTGGGTGTGTGGAGCTCCATCCACCCGGCCCTGAAGGGCCTAGGCCTGGCCTCCATGCTCACGT | member 19 |
| | CCTTCATGGTGGGACTGTATTACAACACCATCATCTCCTGGATCATGTGGTACTTATTCAACTCCTTCCA | (SLC6A19), |
| | GGAGCCTCTGCCCTGGAGCGACTGCCCGCTCAACGAGAACCAGACAGGGTATGTGGACGAGTGCGCCAGG | mRNA |
| | AGCTCCCCTGTGGACTACTTCTGGTACCGAGAGACGCTCAACATCTCCACGTCCATCAGCGACTCGGGCT | |
| | CCATCCAGTGGTGGATGCTGCTGTGCCTGGCCTGCGCATGGAGCGTCCTGTACATGTGCACCATCCGCGG | |
| | CATCGAGACCACCGGGAAGGCCGTGTACATCACCTCCACGCTGCCCTATGTCGTCCTGACCATCTTCCTC | |
| | ATCCGAGGGCTGACGCTGAAGGGCGCCACCAATGGCATCGTCTTCCTCTTCACGCCCAACGTCACGGAGC | |
| | TGGCCCAGCCGGACACCTGGCTGGACGCGGGCGCACAGGTCTTCTTCTCCTTCTCCCTGGCCTTCGGGGG | |
| | CCTCATCTCCTTCTCCAGCTACAACTCTCTCCACAACAACTCCGACAASCACTCCCTGATTCTCTCCATC | |
| | ATCAACGGCTTCACATCGGTGTATGTGGCCATCGTGGTCTACTCCGTCATTGGGTTCCGCGCCACACAGC | |
| | GCTACGACGACTGCTTCAGCACGAACATCCTGACCCTCATCAACGGGTTCGACCTGCCTGAAGGCAACGT | |
| | GACCCAGGAGAACTTTGTGGACATGCAGCAGCGGTGCAACGCCTCCGACCCCGCGGCCTACGCGCAGCTG | |
| | GTGTTCCAGACCTGCGACATCAACGCCTTCCTCTCAGAGGCCGTGGAGGGCACAGCCCTGGCCTTCATCG | |
| | TCTTCACCGAGGCCATCACCAAGATGCCGTTGTCCCCACTGTGGTCTGTGCTCTTCTTCATTATGCTCTT | |
| | CTGCCTGGGGCTGTCATCTATGTTTGGGAACATGGAGGGCGTCGTTGTGCCCCTGCAGGACCTCAGAGTC | |
| | ATCCCCCCGAAGTGGCCCAAGGAGGTGCTCACAGGCCTCATCTGCCTGGGGACATTCCTCATTGGCTTCA | |
| | TCTTCACGCTGAACTCCGGCCAGTACTGGCTCTCCCTGCTGGACAGCTATGCCGGCTCCATTCCCCTGCT | |
| | CATCATCGCCTTCTGCGAGATGTTCTCTGTGGTCTACGTGTACGGTGTGGACAGGTTCAATAAGGACATC | |
| | GAGTTCATGATCGGCCACAAGCCCAACATCTTCTGGCAAGTCACGTGGCGCGTGGTCAGCCCCCTGCTCA | |
| | TGCTGATCATCTTCCTCTTCTTCTTCGTGGTAGAGGTCAGTCAGGAGCTGACCTACAGCATCTGGGACCC | |
| | TGGCTACGAGGAATTTCCCAAATCCCAGAAGATCTCCTACCCGAACTGGGTGTATGTGGTGGTGGTGATT | |
| | GTGGCTGGAGTGCCCTCCCTCACCATCCCTGGCTATGCCATCTACAAGCTCATCAGGAACCACTGCCAGA | |
| | AGCCAGGGGACCATCAGGGGCTGGTGAGCACACTGTCCACAGCCTCCATGAACGGGGACCTGAAGTACTG | |
| | AGAAGGCCCATCCCACGGCGTGCCATACACTGGTGTCAGGGAAGGAGGAACCAGCAAGACCTGTGGGGTG | |
| | GGGGCCGGGCTGCACCTGCATGTGTGTAAGCGTGAGTGTATGCTCGTGTGTGAGTGTGTGTATTGTACAC | |
| | GCATGTGCCATGTGTGCAGATATGTATCGTGTGTGCATGTACATGCATGGGCACTGTGTGAGTGTGCACG | |
| | TGTATGCACACATATACATGTGTGTGGGTGTGTGTATTGTATGTGCATGTGCCATGTGTGCAGATGTGTC | |
| | ATGTTGTGTGTGTGCATGTACATGTATGGACATTGTGTGAGTGTGCAAGTGTGCATGCATATACATGTGT | |
| | GCGATATTTGCTGCCCGTGTGTGTGCATGTATATATAGACATACATGCCTATGTTGTGTGTGGTGTGCAT | |
| | ATGTGTGAACACACACGTGTATACATGCATGCACATGTGCTCGTACAATGGGTGTCCACATGCACGTGTA | |
| | TATGTATATCTGTGAGTGTATATACATGCATGCAATTGTGTGTATGTGTGTTCTGTGTGTGCGTTTGCAA | |
| | GTATATATGCACATGTGTATATGTACATGTATGCCTGTGTGACGTGTGTATATGTGAGCATGTGTACGTG | |
| | TGTGTATACGTGTGTTGTGTATATGTGTGTGTCTGTACCTGTTTGTGTATATGTGTGTGATGTGTGCTCG | |
| | TGTGTGTGCATATTCAGGCAGGTGTGCATTTGTGCATGCCAGTGTGTATGTATGTGCGCATATGGACACG | |
| | CATGGACACGCATATGGACACATATGGACACACATATGGACACGTGTGGATATGTGTGCGTACACGTCGC | |
| | TGGGACACATGCCTGGCACTCGGGGCCCAGCTGCCCTCTGTGTTTGTCCTTGCCACAGTCACGGGGTGCA | |
| | TGTGCAGAGGGGAGCAGACCACTGGGGACGTGCTGTGCCCTGCACGTGCCCGGGGGAAGCGGAAGCTGCA | |
| | GCTGGGGTGGGGGCAGCACCTCTATGCTTCATCTCTGTGGGTGGCAGGAGACAAAAGCACAGGGTACTAT | |
| | CTTGGCTCCTGGGAGCGACTCTTGCTACCCACCCCCACCCATCCCCTTCCCCTTGGTGTTGACCTTTGAC | |
| | CTGGGGGTTCCCAGAGCCCTGTAGCCCTCGACCCGGAGCAGCCTCTCGGAAGCCGGAGTGGGCAGTTGCT | |
| | GGCGATTCTGAGAAAACTTGGCCGCATCCACCGGGGCCCTGCCTCCAGTCGGCCGCTGCCGAGTCTCTGC | |
| | GTTCTGGCCGCTTCCCGGCTTAATGAATGCCAGCCATTTAATCATTGCTCCTGCCACCACAAATAGATGA | |
| | GCAGTTAAATAAAACTCAACTTGGCATAATTCAAGGCAAATACCACTCTGTGCATTTTCTTAAGAGGACA | |
| | TGAGCTGTGTGAATTTTTAGCCAGCCTTTGGAAAAGATGGGTTACAGGGTAACTCAACCCTGGCTGCCAT | |
| | CCTTGGGCACTGTGTGTGTCCAGGGCACCTTGGAGGACCGTGCAGCCCCCAGAAGCTTCCAGCTCCCGCA | |
| | CCACTCAGTGAAGCCCAGCCTGGCGCCTGCCCTGCCCCCGTCACGGGATGGGCCCCCATTGGGGTTCAAC | |
| | ATTCCATCGCAGCCAAAGGCAGTCGGCACTTGGGACATCTGCTTCCACGGACAGGTCACCTCCGCTTTGC | |
| | ACGGAAGAATCTGGATGCTTACATTAAACTGGTGTTCTGAGAGTTCCTACGGACAGGTCACCTCCGCTTT | |
| | GCATGGAAGAATCTGGATGCTTACATTAAACTGGTGTTCTGAGAGTTCCTACGGACAGGTCACCTCTGCT | |
| | TTCCATAGAAGAATCTGGACGCTTACATTAAACTGATGTTCTGAGAATTCCTACAGGCAGGACTGAAAGC | |
| | CTGGTGTGTGCCAGTATGATGTTCCACCCACAGAAACCTGGTCACAATCGTCCCTTCCAGCACCCCATCC | |
| | AGCAGTGACTGCACACACTGAGTCCCCTACCAGCCCCTTTCACCCTGCTGACTGTCACTGGGCCCTGGGA | |
| | TGCGCAAGACTCCACAGCAGCAGAGGTGGGGGGACATATCACAGCCTCTGCCCCCGGCTGTGATGCCACC | |
| | GAGGGGCTCGCCTGCTGATGGCTTCAACAGGGTCTCACCTCATCTTTTCCTGCTCTTTGGCCCTGGATCG | |
| | AGAAAATTTCCATCAGTGCCCCATTAATATGCTGCCCTGTGGCATCTGCCCAGGAGGCCCTGCCAGGCGT | |
| | GCACAGGTGTGCATTGGTGTACCCTGGCATGCACAGGTGTGCACTGATGTGCCCTGGCATCCATTGGTGT | |
| | ACCCTGGTGTGCCTGCCATAGGACCCTGGGCGGGAGCTCCCATCTCATCTACATCTCCTGATTCATGCGT | |
| | TGTTTCATAGGTTTCAATGTCTCTGTAAATGTGGTAGAAATGCAGGCTTTATGGGCATAAAGTGTACATT | |
| | TCTAAATAAATCCCTTCTATTGAGTATGCTCACCCTAGAAGTTACTGTTGTCCAGACGTAGAGGGATGAG | |
| | TGAGCCAGTGACCTCAGACGGGATGGTGGGGACGGCAGGTCCAGCTCCTGCCTCCTCCTGGGGGGTCTGG | |
| | CTTTGGGGGCTTGCTCCGAAGAGGCCATGGCCCAGGCCTGTGGCCTCACAATGGGGACCAACCAGCTCTT | |
| | CTCATCTTCTTCCCTCACACTTCCTCTCACTCAAATAAGAACCTTCCAAAAATGTGTCCACCTGGGCCCC | |
| | TGCCCTGGGACTCATGGATTTGGAGTTGTGGCCACACGGTTGAGGGGTGCAGTGTCCAGTGGAATGGGGC | |
| | AATTGCGGGCCTGGGGGCCCTTGGCCTGTCCGTGGCGGGAGCATCTGCAAGGAGGAGCCCCAGAGTCCAG | |
| | GGAGCACTGTGGGGAGCTCCTTAGAGCTGAACTCACCCGGCGTCAACTCATCAACCCTCCACCCATGGAC | |
| | AGGGGTGCCCCCAGCACAGGAGAGGACTCAGCCCTCTGCCCCCACGCACGGTGGGTGCCTGTCACCCTGT | |
| | CCTGCCCAGCGGCCCGAGGGCAGCAGTGGGTGTGAGGGCAGCCCCCGGCCTCCCAAGAGCAGCTGAGAGG | |
| | ATCCCTGCGGGAATCCGGGCTTCGGGTGCATGCGATCTGATCTGAGTTGTTTCTGACAGTGACAGAGTGA | |
| | CAATCTATAAGTATCTCAAGATCAAATGGTTAAATAAAACATAAGAAATTTAAAACGA | |
| 31 | MVRLVLPNPGLDARIPSLAELETIEQEEASSRPKWDNKAQYMLTCLGFCVGLGNVWRFPYLCQSHGGGAF | >NP_001003841.1 |
| | MIPFLILLVLEGIPLLYLEFAIGQRLRRGSLGVWSSIHPALKGLGLASMLTSFMVGLYYNTIISWIMWYL | sodium- |
| | FNSFQEPLPWSDCPLNENQTGYVDECARSSPVDYFWYRETLNISTSISDSGSIQWWMLLCLACAWSVLYM | dependent |
| | CTIRGIETTGKAVYITSTLPYVVLTIFLIRGLTLKGATNGIVFLFTPNVTELAQPDTWLDAGAQVFFSFS | neutral amino |
| | LAFGGLISFSSYNSVHNNCEKDSVIVSIINGFTSVYVAIVVYSVIGFRATQRYDDCFSTNILTLINGFDL | acid |
| | PEGNVTQENFVDMQQRCNASDPAAYAQLVFQTCDINAFLSEAVEGTGLAFIVFTEAITKMPLSPLWSVLF | transporter |
| | FIMLFCLGLSSMFGNMEGVVVPLQDLRVIPPKWPKEVLTGLICLGTFLIGFIFTLNSGQYWLSLLDSYAG | B(0)AT1 |
| | SIPLLITAFCEMFSVVYVYGVDRFNKDIEFMIGHKPNIFWQVTWRVVSPLLMLIIFLFFFVVEVSQELTY | [Homo sapiens] |
| | SIWDPGYEEFPKSQKISYPNWVYVVVVIVAGVPSLTIPGYAIYKLIRNHCQKPGDHQGLVSTLSTASMNG | |
| | DLKY | |
| 32 | AGAAGCGGAGCGTATACGGAGGAGGCGGGATGCATTTCTGCATCGAGCGCACAAAGTTATCTAAAACAGT | >NM_001320923.2 |
| | TCATGCTGCTGAAAACCTCCTTCCTGGCAGATGTCCCTCAACCCTACTGGTGCCTGGCTTCTGAGACACA | Homo sapiens |
| | CGCTTCTCTGAAGTAGCTTTGGAAAGTAGAGAAGAAAATCCAGTTTGCTTCTTGGAGAACACTGGACAGC | Janus |
| | TGAATAAATGCAGTATCTAAATATAAAAGAGGACTGCAATGCCATGGCTTTCTGTGCTAAAATGAGGAGC | kinase 1 |
| | TCCAAGAAGACTGAGGTGAACCTGGAGGCCCCTGAGCCAGGGGTGGAAGTGATCTTCTATCTGTCGGACA | (JAK1), |
| | GGGAGCCCCTCCGGCTGGGCAGTGGAGAGTACACAGCAGAGGAACTGTGCATCAGGGCTGCACAGGCATG | transcript |
| | CCGTATCTCTCCTCTTTGTCACAACCTCTTTGCCCTGTATGACGAGAACACCAAGCTCTGGTATGCTCCA | variant 2, |
| | AATCGCACCATCACCGTTGATGACAAGATGTCCCTCCGGCTCCACTACCGGATGAGGTTCTATTTCACCA | mRNA |
| | ATTGGCATGGAACCAACGACAATGAGCAGTCAGTGTGGCGTCATTCTCCAAAGAAGCAGAAAAATGGCTA | |
| | CGAGAAAAAAAAGATTCCAGATGCAACCCCTCTCCTTGATGCCAGCTCACTGGAGTATCTGTTTGCTCAG | |
| | GGACAGTATGATTTGGTGAAATGCCTGGCTCCTATTCGAGACCCCAAGACCGAGCAGGATGGACATGATA | |
| | TTGAGAACGAGTGTCTAGGGATGGCTGTCCTGGCCATCTCACACTATGCCATGATGAAGAAGATGCAGTT | |
| | GCCAGAACTGCCCAAGGACATCAGCTACAAGCGATATATTCCAGAAACATTGAATAAGTCCATCAGACAG | |
| | AGGAACCTTCTCACCAGGATGCGGATAAATAATGTTTTCAAGGATTTCCTAAAGGAATTTAACAACAAGA | |
| | CCATTTGTGACAGCAGCGTGTCCACGCATGACCTGAAGGTGAAATACTTGGCTACCTTGGAAACTTTGAC | |
| | AAAACATTACGGTGCTGAAATATTTGAGACTTCCATGTTACTGATTTCATCAGAAAATGAGATGAATTGG | |
| | TTTCATTCGAATGACGGTGGAAACGTTCTCTACTACGAAGTGATGGTGACTGGGAATCTTGGAATCCAGT | |
| | GGAGGCATAAACCAAATGTTGTTTCTGTTGAAAAGGAAAAAAATAAACTGAAGCGGAAAAAACTGGAAAA | |
| | TAAACACAAGAAGGATGAGGAGAAAAACAAGATCCGGGAAGAGTGGAACAATTTTTCTTACTTCCCTGAA | |
| | ATCACTCACATTGTAATAAAGGAGTCTGTGGTCAGCATTAACAAGCAGGACAACAAGAAAATGGAACTGA | |
| | AGCTCTCTTCCCACGAGGAGGCCTTGTCCTTTGTGTCCCTGGTAGATGGCTACTTCCGGCTCACAGCAGA | |
| | TGCCCATCATTACCTCTGCACCGACGTGGCCCCCCCGTTGATCGTCCACAACATACAGAATGGCTGTCAT | |
| | GGTCCAATCTGTACAGAATACGCCATCAATAAATTGCGGCAAGAAGGAAGCGAGGAGGGGATGTACGTGC | |
| | TGAGGTGGAGCTGCACCGACTTTGACAACATCCTCATGACCGTCACCTGCTTTGAGAAGTCTGAGCAGGT | |
| | GCAGGGTGCCCAGAAGCAGTTCAAGAACTTTCAGATCGAGGTGCAGAAGGGCCGCTACAGTCTGCACGGT | |
| | TCGGACCGCAGCTTCCCCAGCTTGGGAGACCTCATGAGCCACCTCAAGAAGCAGATCCTGCGCACGGATA | |
| | ACATCAGCTTCATGCTAAAACGCTGCTGCCAGCCCAAGCCCCGAGAAATCTCCAACCTGCTGGTGGCTAC | |
| | TAAGAAAGCCCAGGAGTGGCAGCCCGTCTACCCCATGAGCCAGCTGAGTTTCGATCGGATCCTCAAGAAG | |
| | GATCTGGTGCAGGGCGAGCACCTTGGGAGAGGCACGAGAACACACATCTATTCTGGGACCCTGATGGATT | |
| | ACAAGGATGACGAAGGAACTTCTGAAGAGAAGAAGATAAAAGTGATCCTCAAAGTCTTAGACCCCAGCCA | |
| | CAGGGATATTTCCCTGGCCTTCTTCGAGGCAGCCAGCATGATGAGACAGGTCTCCCACAAACACATCGTG | |
| | TACCTCTATGGCGTCTGTGTCCGCGACGTGGAGAATATCATGGTGGAAGAGTTTGTGGAAGGGGGTCCTC | |
| | TGGATCTCTTCATGCACCGGAAAAGCGATGTCCTTACCACACCATGGAAATTCAAAGTTGCCAAACAGCT | |
| | GGCCAGTGCCCTGAGCTACTTGGAGGATAAAGACCTGGTCCATGGAAATGTGTGTACTAAAAACCTCCTC | |
| | CTGGCCCGTGAGGGCATCGACAGTGAGTGTGGCCCATTCATCAAGCTCAGTGACCCCGGCATCCCCATTA | |
| | CGGTGCTGTCTAGGCAAGAATGCATTGAACGAATCCCATGGATTGCTCCTGAGTGTGTTGAGGACTCCAA | |
| | GAACCTGAGTGTGGCTGCTGACAAGTGGAGCTTTGGAACCACGCTCTGGGAAATCTGCTACAATGGCGAG | |
| | ATCCCCTTGAAAGACAAGACGCTGATTGAGAAAGAGAGATTCTATGAAAGCCGGTGCAGGCCAGTGACAC | |
| | CATCATGTAAGGAGCTGGCTGACCTCATGACCCGCTGCATGAACTATGACCCCAATCAGAGGCCTTTCTT | |
| | CCGAGCCATCATGAGAGACATTAATAAGCTTGAAGAGCAGAATCCAGATATTGTTTCAGAAAAAAAACCA | |
| | GCAACTGAAGTGGACCCCACACATTTTGAAAAGCGCTTCCTAAAGAGGATCCGTGACTTGGGAGAGGGCC | |
| | ACTTTGGGAAGGTTGAGCTCTGCAGGTATGACCCCGAAGGGGACAATACAGGGGAGCAGGTGGCTGTTAA | |
| | ATCTCTGAAGCCTGAGAGTGGAGGTAACCACATAGCTGATCTGAAAAAGGAAATCGAGATCTTAAGGAAC | |
| | CTCTATCATGAGAACATTGTGAAGTACAAAGGAATCTGCACAGAAGACGGAGGAAATGGTATTAAGCTCA | |
| | TCATGGAATTTCTGCCTTCGGGAAGCCTTAAGGAATATCTTCCAAAGAATAAGAACAAAATAAACCTCAA | |
| | ACAGCAGCTAAAATATGCCGTTCAGATTTGTAAGGGGATGGACTATTTGGGTTCTCGGCAATACGTTCAC | |
| | CGGGACTTGGCAGCAAGAAATGTCCTTGTTGAGAGTGAACACCAAGTGAAAATTGGAGACTTCGGTTTAA | |
| | CCAAAGCAATTGAAACCGATAAGGAGTATTACACCGTCAAGGATGACCGGGACAGCCCTGTGTTTTGGTA | |
| | TGCTCCAGAATGTTTAATGCAATCTAAATTTTATATTGCCTCTGACGTCTGGTCTTTTGGAGTCACTCTG | |
| | CATGAGCTGCTGACTTACTGTGATTCAGATTCTAGTCCCATGGCTTTGTTCCTGAAAATGATAGGCCCAA | |
| | CCCATGGCCAGATGACAGTCACAAGACTTGTGAATACGTTAAAAGAAGGAAAACGCCTGCCGTGCCCACC | |
| | TAACTGTCCAGATGAGGTTTATCAACTTATGAGGAAATGCTGGGAATTCCAACCATCCAATCGGACAAGC | |
| | TTTCAGAACCTTATTGAAGGATTTGAAGCACTTTTAAAATAAGAAGCATGAATAACATTTAAATTCCACA | |
| | GATTATCAAGTCCTTCTCCTGCAACAAATGCCCAAGTCATTTTTTAAAAATTTCTAATGAAAGAAGTTTG | |
| | TGTTCTGTCCAAAAAGTCACTGAACTCATACTTCAGTACATATACATGTATAAGGCACACTGTAGTGCTT | |
| | AATATGTGTAAGGACTTCCTCTTTAAATTTGGTACCAGTAACTTAGTGACACATAATGACAACCAAAATA | |
| | TTTGAAAGCACTTAAGCACTCCTCCTTGTGGAAAGAATATACCACCATTTCATCTGGCTAGTTCACCATC | |
| | ACAACTGCATTACCAAAAGGGGATTTTTGAAAACGAGGAGTTGACCAAAATAATATCTGAAGATGATTGC | |
| | TTTTCCCTGCTGCCAGCTGATCTGAAATGTTTTGCTGGCACATTAATCATAGATAAAGAAAGATTGATGG | |
| | ACTTAGCCCTCAAATTTCAGTATCTATACAGTACTAGACCATGCATTCTTAAAATATTAGATACCAGGTA | |
| | GTATATATTGTTTCTGTACAAAAATGACTGTATTCTCTCACCAGTAGGACTTAAACTTTGTTTCTCCAGT | |
| | GGCTTAGCTCCTGTTCCTTTGGGTGATCACTAGCACCCATTTTTGAGAAAGCTGGTTCTACATGGGGGGA | |
| | TAGCTGTGGAATAGATAATTTGCTGCATGTTAATTCTCAAGAACTAAGCCTGTGCCAGTGCTTTCCTAAG | |
| | CAGTATACCTTTAATCAGAACTCATTCCCAGAACCTGGATGCTATTACACATGCTTTTAAGAAACGTCAA | |
| | TGTATATCCTTTTATAACTCTACCACTTTGGGGCAAGCTATTCCAGCACTGGTTTTGAATGCTGTATGCA | |
| | ACCAGTCTGAATACCACATACGCTGCACTGTTCTTAGAGGGTTTCCATACTTACCACCGATCTACAAGGG | |
| | TTGATCCCTGTTTTTACCATCAATCATCACCCTGTGGTGCAACACTTGAAAGACCCGGCTAGAGGCACTA | |
| | TGGACTTCAGGATCCACTAGACAGTTTTCAGTTTGCTTGGAGGTAGCTGGGTAATCAAAAATGTTTAGTC | |
| | ATTGATTCAATGTGAACGATTACGGTCTTTATGACCAAGAGTCTGAAAATCTTTTTGTTATGCTGTTTAG | |
| | TATTCGTTTGATATTGTTACTTTTCACCTGTTGAGCCCAAATTCAGGATTGGTTCAGTGGCAGCAATGAA | |
| | GTTGCCATTTAAATTTGTTCATAGCCTACATCACCAAGGTCTCTGTGTCAAACCTGTGGCCACTCTATAT | |
| | GCACTTTGTTTACTCTTTATACAAATAAATATACTAAAGACTTTA | |
| 33 | ATCTATCACATGGCAGAGATAGAATAAAAACAGAAAAATGGCGACGGTCACGTTGTGGCGAGCCTTGCTG | >NM_001321852.2 |
| | CGTCATTAGATAATCCTCATGCAAATAGCGGGAAGAACAAAGGAAGGGGAGCCCGGGACCCCCGGGGGCG | Homo sapiens |
| | CAGCGCTTCTCTGAAGTAGCTTTGGAAAGTAGAGAAGAAAATCCAGTTTGCTTCTTGGAGAACACTGGAC | Janus |
| | AGCTGAATAAATGCAGTATCTAAATATAAAAGAGGACTGCAATGCCATGGCTTTCTGTGCTAAAATGAGG | kinase 1 |
| | AGCTCCAAGAAGACTGAGGTGAACCTGGAGGCCCCTGAGCCAGGGGTGGAAGTGATCTTCTATCTGTCGG | (JAK1), |
| | ACAGGGAGCCCCTCCGGCTGGGCAGTGGAGAGTACACAGCAGAGGAACTGTGCATCAGGGCTGCACAGGC | transcript |
| | ATGCCGTATCTCTCCTCTTTGTCACAACCTCTTTGCCCTGTATGACGAGAACACCAAGCTCTGGTATGCT | variant 3, |
| | CCAAATCGCACCATCACCGTTGATGACAAGATGTCCCTCCGGCTCCACTACCGGATGAGGTTCTATTTCA | mRNA |
| | CCAATTGGCATGGAACCAACGACAATGAGCAGTCAGTGTGGCGTCATTCTCCAAAGAAGCAGAAAAATGG | |
| | CTACGAGAAAAAAAAGATTCCAGATGCAACCCCTCTCCTTGATGCCAGCTCACTGGAGTATCTGTTTGCT | |
| | CAGGGACAGTATGATTTGGTGAAATGCCTGGCTCCTATTCGAGACCCCAAGACCGAGCAGGATGGACATG | |
| | ATATTGAGAACGAGTGTCTAGGGATGGCTGTCCTGGCCATCTCACACTATGCCATGATGAAGAAGATGCA | |
| | GTTGCCAGAACTGCCCAAGGACATCAGCTACAAGCGATATATTCCAGAAACATTGAATAAGTCCATCAGA | |
| | CAGAGGAACCTTCTCACCAGGATGCGGATAAATAATGTTTTCAAGGATTTCCTAAAGGAATTTAACAACA | |
| | AGACCATTTGTGACAGCAGCGTGTCCACGCATGACCTGAAGGTGAAATACTTGGCTACCTTGGAAACTTT | |
| | GACAAAACATTACGGTGCTGAAATATTTGAGACTTCCATGTTACTGATTTCATCAGAAAATGAGATGAAT | |
| | TGGTTTCATTCGAATGACGGTGGAAACGTTCTCTACTACGAAGTGATGGTGACTGGGAATCTTGGAATCC | |
| | AGTGGAGGCATAAACCAAATGTTGTTTCTGTTGAAAAGGAAAAAAATAAACTGAAGCGGAAAAAACTGGA | |
| | AAATAAACACAAGAAGGATGAGGAGAAAAACAAGATCCGGGAAGAGTGGAACAATTTTTCTTACTTCCCT | |
| | GAAATCACTCACATTGTAATAAAGGAGTCTGTGGTCAGCATTAACAAGCAGGACAACAAGAAAATGGAAC | |
| | TGAAGCTCTCTTCCCACGAGGAGGCCTTGTCCTTTGTGTCCCTGGTAGATGGCTACTTCCGGCTCACAGC | |
| | AGATGCCCATCATTACCTCTGCACCGACGTGGCCCCCCCGTTGATCGTCCACAACATACAGAATGGCTGT | |
| | CATGGTCCAATCTGTACAGAATACGCCATCAATAAATTGCGGCAAGAAGGAAGCGAGGAGGGGATGTACG | |
| | TGCTGAGGTGGAGCTGCACCGACTTTGACAACATCCTCATGACCGTCACCTGCTTTGAGAAGTCTGAGCA | |
| | GGTGCAGGGTGCCCAGAAGCAGTTCAAGAACTTTCAGATCGAGGTGCAGAAGGGCCGCTACAGTCTGCAC | |
| | GGTTCGGACCGCAGCTTCCCCAGCTTGGGAGACCTCATGAGCCACCTCAAGAAGCAGATCCTGCGCACGG | |
| | ATAACATCAGCTTCATGCTAAAACGCTGCTGCCAGCCCAAGCCCCGAGAAATCTCCAACCTGCTGGTGGC | |
| | TACTAAGAAAGCCCAGGAGTGGCAGCCCGTCTACCCCATGAGCCAGCTGAGTTTCGATCGGATCCTCAAG | |
| | AAGGATCTGGTGCAGGGCGAGCACCTTGGGAGAGGCACGAGAACACACATCTATTCTGGGACCCTGATGG | |
| | ATTACAAGGATGACGAAGGAACTTCTGAAGAGAAGAAGATAAAAGTGATCCTCAAAGTCTTAGACCCCAG | |
| | CCACAGGGATATTTCCCTGGCCTTCTTCGAGGCAGCCAGCATGATGAGACAGGTCTCCCACAAACACATC | |
| | GTGTACCTCTATGGCGTCTGTGTCCGCGACGTGGAGAATATCATGGTGGAAGAGTTTGTGGAAGGGGGTC | |
| | CTCTGGATCTCTTCATGCACCGGAAAAGCGATGTCCTTACCACACCATGGAAATTCAAAGTTGCCAAACA | |
| | GCTGGCCAGTGCCCTGAGCTACTTGGAGGATAAAGACCTGGTCCATGGAAATGTGTGTACTAAAAACCTC | |
| | CTCCTGGCCCGTGAGGGCATCGACAGTGAGTGTGGCCCATTCATCAAGCTCAGTGACCCCGGCATCCCCA | |
| | TTACGGTGCTGTCTAGGCAAGAATGCATTGAACGAATCCCATGGATTGCTCCTGAGTGTGTTGAGGACTC | |
| | CAAGAACCTGAGTGTGGCTGCTGACAAGTGGAGCTTTGGAACCACGCTCTGGGAAATCTGCTACAATGGC | |
| | GAGATCCCCTTGAAAGACAAGACGCTGATTGAGAAAGAGAGATTCTATGAAAGCCGGTGCAGGCCAGTGA | |
| | CACCATCATGTAAGGAGCTGGCTGACCTCATGACCCGCTGCATGAACTATGACCCCAATCAGAGGCCTTT | |
| | CTTCCGAGCCATCATGAGAGACATTAATAAGCTTGAAGAGCAGAATCCAGATATTGTTTCAGAAAAAAAA | |
| | CCAGCAACTGAAGTGGACCCCACACATTTTGAAAAGCGCTTCCTAAAGAGGATCCGTGACTTGGGAGAGG | |
| | GCCACTTTGGGAAGGTTGAGCTCTGCAGGTATGACCCCGAAGGGGACAATACAGGGGAGCAGGTGGCTGT | |
| | TAAATCTCTGAAGCCTGAGAGTGGAGGTAACCACATAGCTGATCTGAAAAAGGAAATCGAGATCTTAAGG | |
| | AACCTCTATCATGAGAACATTGTGAAGTACAAAGGAATCTGCACAGAAGACGGAGGAAATGGTATTAAGC | |
| | TCATCATGGAATTTCTGCCTTCGGGAAGCCTTAAGGAATATCTTCCAAAGAATAAGAACAAAATAAACCT | |
| | CAAACAGCAGCTAAAATATGCCGTTCAGATTTGTAAGGGGATGGACTATTTGGGTTCTCGGCAATACGTT | |
| | CACCGGGACTTGGCAGCAAGAAATGTCCTTGTTGAGAGTGAACACCAAGTGAAAATTGGAGACTTCGGTT | |
| | TAACCAAAGCAATTGAAACCGATAAGGAGTATTACACCGTCAAGGATGACCGGGACAGCCCTGTGTTTTG | |
| | GTATGCTCCAGAATGTTTAATGCAATCTAAATTTTATATTGCCTCTGACGTCTGGTCTTTTGGAGTCACT | |
| | CTGCATGAGCTGCTGACTTACTGTGATTCAGATTCTAGTCCCATGGCTTTGTTCCTGAAAATGATAGGCC | |
| | CAACCCATGGCCAGATGACAGTCACAAGACTTGTGAATACGTTAAAAGAAGGAAAACGCCTGCCGTGCCC | |
| | ACCTAACTGTCCAGATGAGGTTTATCAACTTATGAGGAAATGCTGGGAATTCCAACCATCCAATCGGACA | |
| | AGCTTTCAGAACCTTATTGAAGGATTTGAAGCACTTTTAAAATAAGAAGCATGAATAACATTTAAATTCC | |
| | ACAGATTATCAAGTCCTTCTCCTGCAACAAATGCCCAAGTCATTTTTTAAAAATTTCTAATGAAAGAAGT | |
| | TTGTGTTCTGTCCAAAAAGTCACTGAACTCATACTTCAGTACATATACATGTATAAGGCACACTGTAGTG | |
| | CTTAATATGTGTAAGGACTTCCTCTTTAAATTTGGTACCAGTAACTTAGTGACACATAATGACAACCAAA | |
| | ATATTTGAAAGCACTTAAGCACTCCTCCTTGTGGAAAGAATATACCACCATTTCATCTGGCTAGTTCACC | |
| | ATCACAACTGCATTACCAAAAGGGGATTTTTGAAAACGAGGAGTTGACCAAAATAATATCTGAAGATGAT | |
| | TGCTTTTCCCTGCTGCCAGCTGATCTGAAATGTTTTGCTGGCACATTAATCATAGATAAAGAAAGATTGA | |
| | TGGACTTAGCCCTCAAATTTCAGTATCTATACAGTACTAGACCATGCATTCTTAAAATATTAGATACCAG | |
| | GTAGTATATATTGTTTCTGTACAAAAATGACTGTATTCTCTCACCAGTAGGACTTAAACTTTGTTTCTCC | |
| | AGTGGCTTAGCTCCTGTTCCTTTGGGTGATCACTAGCACCCATTTTTGAGAAAGCTGGTTCTACATGGGG | |
| | GGATAGCTGTGGAATAGATAATTTGCTGCATGTTAATTCTCAAGAACTAAGCCTGTGCCAGTGCTTTCCT | |
| | AAGCAGTATACCTTTAATCAGAACTCATTCCCAGAACCTGGATGCTATTACACATGCTTTTAAGAAACGT | |
| | CAATGTATATCCTTTTATAACTCTACCACTTTGGGGCAAGCTATTCCAGCACTGGTTTTGAATGCTGTAT | |
| | GCAACCAGTCTGAATACCACATACGCTGCACTGTTCTTAGAGGGTTTCCATACTTACCACCGATCTACAA | |
| | GGGTTGATCCCTGTTTTTACCATCAATCATCACCCTGTGGTGCAACACTTGAAAGACCCGGCTAGAGGCA | |
| | CTATGGACTTCAGGATCCACTAGACAGTTTTCAGTTTGCTTGGAGGTAGCTGGGTAATCAAAAATGTTTA | |
| | GTCATTGATTCAATGTGAACGATTACGGTCTTTATGACCAAGAGTCTGAAAATCTTTTTGTTATGCTGTT | |
| | TAGTATTCGTTTGATATTGTTACTTTTCACCTGTTGAGCCCAAATTCAGGATTGGTTCAGTGGCAGCAAT | |
| | GAAGTTGCCATTTAAATTTGTTCATAGCCTACATCACCAAGGTCTCTGTGTCAAACCTGTGGCCACTCTA | |
| | TATGCACTTTGTTTACTCTTTATACAAATAAATATACTAAAGACTTTA | |
| 34 | ATCTATCACATGGCAGAGATAGAATAAAAACAGAAAAATGGCGACGGTCACGTTGTGGCGAGCCTTGCTG | >NM_001321853.2 |
| | CGTCATTAGATAATCCTCATGCAAATAGCGGGAAGAACAAAGGAAGGGGAGCCCGGGACCCCCGGGGGCG | Homo sapiens |
| | CAGGATCCGGCGGGAGGAGTCTAAGAGGAGGAGGCGGCGGTGCCGGAGGAGGAGGAGGAGGGAGGGAGAA | Janus |
| | GAGAGGAAGACCGGAGTCCCCGCGGCGGCGGCGGTCCGGAGAGAGGGCGAGCCCCGCGCGGCGCCGGGGA | kinase 1 |
| | CCGGGCGCTACCACGAGGCCGGGACGCTGGAGTCTGGGTTATCTAAAACAGTTCATGCTGCTGAAAACCT | (JAK1), |
| | CCTTCCTGGCAGATGTCCCTCAACCCTACTGGTGCCTGGCTTCTGAGACACACGCTTCTCTGAAGTAGCT | transcript |
| | TTGGAAAGTAGAGAAGAAAATCCAGTTTGCTTCTTGGAGAACACTGGACAGCTGAATAAATGCAGTATCT | variant 4, |
| | AAATATAAAAGAGGACTGCAATGCCATGGCTTTCTGTGCTAAAATGAGGAGCTCCAAGAAGACTGAGGTG | mRNA |
| | AACCTGGAGGCCCCTGAGCCAGGGGTGGAAGTGATCTTCTATCTGTCGGACAGGGAGCCCCTCCGGCTGG | |
| | GCAGTGGAGAGTACACAGCAGAGGAACTGTGCATCAGGGCTGCACAGGCATGCCGTATCTCTCCTCTTTG | |
| | TCACAACCTCTTTGCCCTGTATGACGAGAACACCAAGCTCTGGTATGCTCCAAATCGCACCATCACCGTT | |
| | GATGACAAGATGTCCCTCCGGCTCCACTACCGGATGAGGTTCTATTTCACCAATTGGCATGGAACCAACG | |
| | ACAATGAGCAGTCAGTGTGGCGTCATTCTCCAAAGAAGCAGAAAAATGGCTACGAGAAAAAAAAGATTCC | |
| | AGATGCAACCCCTCTCCTTGATGCCAGCTCACTGGAGTATCTGTTTGCTCAGGGACAGTATGATTTGGTG | |
| | AAATGCCTGGCTCCTATTCGAGACCCCAAGACCGAGCAGGATGGACATGATATTGAGAACGAGTGTCTAG | |
| | GGATGGCTGTCCTGGCCATCTCACACTATGCCATGATGAAGAAGATGCAGTTGCCAGAACTGCCCAAGGA | |
| | CATCAGCTACAAGCGATATATTCCAGAAACATTGAATAAGTCCATCAGACAGAGGAACCTTCTCACCAGG | |
| | ATGCGGATAAATAATGTTTTCAAGGATTTCCTAAAGGAATTTAACAACAAGACCATTTGTGACAGCAGCG | |
| | TGTCCACGCATGACCTGAAGGTGAAATACTTGGCTACCTTGGAAACTTTGACAAAACATTACGGTGCTGA | |
| | AATATTTGAGACTTCCATGTTACTGATTTCATCAGAAAATGAGATGAATTGGTTTCATTCGAATGACGGT | |
| | GGAAACGTTCTCTACTACGAAGTGATGGTGACTGGGAATCTTGGAATCCAGTGGAGGCATAAACCAAATG | |
| | TTGTTTCTGTTGAAAAGGAAAAAAATAAACTGAAGCGGAAAAAACTGGAAAATAAACACAAGAAGGATGA | |
| | GGAGAAAAACAAGATCCGGGAAGAGTGGAACAATTTTTCTTACTTCCCTGAAATCACTCACATTGTAATA | |
| | AAGGAGTCTGTGGTCAGCATTAACAAGCAGGACAACAAGAAAATGGAACTGAAGCTCTCTTCCCACGAGG | |
| | AGGCCTTGTCCTTTGTGTCCCTGGTAGATGGCTACTTCCGGCTCACAGCAGATGCCCATCATTACCTCTG | |
| | CACCGACGTGGCCCCCCCGTTGATCGTCCACAACATACAGAATGGCTGTCATGGTCCAATCTGTACAGAA | |
| | TACGCCATCAATAAATTGCGGCAAGAAGGAAGCGAGGAGGGGATGTACGTGCTGAGGTGGAGCTGCACCG | |
| | ACTTTGACAACATCCTCATGACCGTCACCTGCTTTGAGAAGTCTGAGCAGGTGCAGGGTGCCCAGAAGCA | |
| | GTTCAAGAACTTTCAGATCGAGGTGCAGAAGGGCCGCTACAGTCTGCACGGTTCGGACCGCAGCTTCCCC | |
| | AGCTTGGGAGACCTCATGAGCCACCTCAAGAAGCAGATCCTGCGCACGGATAACATCAGCTTCATGCTAA | |
| | AACGCTGCTGCCAGCCCAAGCCCCGAGAAATCTCCAACCTGCTGGTGGCTACTAAGAAAGCCCAGGAGTG | |
| | GCAGCCCGTCTACCCCATGAGCCAGCTGAGTTTCGATCGGATCCTCAAGAAGGATCTGGTGCAGGGCGAG | |
| | CACCTTGGGAGAGGCACGAGAACACACATCTATTCTGGGACCCTGATGGATTACAAGGATGACGAAGGAA | |
| | CTTCTGAAGAGAAGAAGATAAAAGTGATCCTCAAAGTCTTAGACCCCAGCCACAGGGATATTTCCCTGGC | |
| | CTTCTTCGAGGCAGCCAGCATGATGAGACAGGTCTCCCACAAACACATCGTGTACCTCTATGGCGTCTGT | |
| | GTCCGCGACGTGGAGAATATCATGGTGGAAGAGTTTGTGGAAGGGGGTCCTCTGGATCTCTTCATGCACC | |
| | GGAAAAGCGATGTCCTTACCACACCATGGAAATTCAAAGTTGCCAAACAGCTGGCCAGTGCCCTGAGCTA | |
| | CTTGGAGGATAAAGACCTGGTCCATGGAAATGTGTGTACTAAAAACCTCCTCCTGGCCCGTGAGGGCATC | |
| | GACAGTGAGTGTGGCCCATTCATCAAGCTCAGTGACCCCGGCATCCCCATTACGGTGCTGTCTAGGCAAG | |
| | AATGCATTGAACGAATCCCATGGATTGCTCCTGAGTGTGTTGAGGACTCCAAGAACCTGAGTGTGGCTGC | |
| | TGACAAGTGGAGCTTTGGAACCACGCTCTGGGAAATCTGCTACAATGGCGAGATCCCCTTGAAAGACAAG | |
| | ACGCTGATTGAGAAAGAGAGATTCTATGAAAGCCGGTGCAGGCCAGTGACACCATCATGTAAGGAGCTGG | |
| | CTGACCTCATGACCCGCTGCATGAACTATGACCCCAATCAGAGGCCTTTCTTCCGAGCCATCATGAGAGA | |
| | CATTAATAAGCTTGAAGAGCAGAATCCAGATATTGTTTCAGAAAAAAAACCAGCAACTGAAGTGGACCCC | |
| | ACACATTTTGAAAAGCGCTTCCTAAAGAGGATCCGTGACTTGGGAGAGGGCCACTTTGGGAAGGTTGAGC | |
| | TCTGCAGGTATGACCCCGAAGGGGACAATACAGGGGAGCAGGTGGCTGTTAAATCTCTGAAGCCTGAGAG | |
| | TGGAGGTAACCACATAGCTGATCTGAAAAAGGAAATCGAGATCTTAAGGAACCTCTATCATGAGAACATT | |
| | GTGAAGTACAAAGGAATCTGCACAGAAGACGGAGGAAATGGTATTAAGCTCATCATGGAATTTCTGCCTT | |
| | CGGGAAGCCTTAAGGAATATCTTCCAAAGAATAAGAACAAAATAAACCTCAAACAGCAGCTAAAATATGC | |
| | CGTTCAGATTTGTAAGGGGATGGACTATTTGGGTTCTCGGCAATACGTTCACCGGGACTTGGCAGCAAGA | |
| | AATGTCCTTGTTGAGAGTGAACACCAAGTGAAAATTGGAGACTTCGGTTTAACCAAAGCAATTGAAACCG | |
| | ATAAGGAGTATTACACCGTCAAGGATGACCGGGACAGCCCTGTGTTTTGGTATGCTCCAGAATGTTTAAT | |
| | GCAATCTAAATTTTATATTGCCTCTGACGTCTGGTCTTTTGGAGTCACTCTGCATGAGCTGCTGACTTAC | |
| | TGTGATTCAGATTCTAGTCCCATGGCTTTGTTCCTGAAAATGATAGGCCCAACCCATGGCCAGATGACAG | |
| | TCACAAGACTTGTGAATACGTTAAAAGAAGGAAAACGCCTGCCGTGCCCACCTAACTGTCCAGATGAGGT | |
| | TTATCAACTTATGAGGAAATGCTGGGAATTCCAACCATCCAATCGGACAAGCTTTCAGAACCTTATTGAA | |
| | GGATTTGAAGCACTTTTAAAATAAGAAGCATGAATAACATTTAAATTCCACAGATTATCAAGTCCTTCTC | |
| | CTGCAACAAATGCCCAAGTCATTTTTTAAAAATTTCTAATGAAAGAAGTTTGTGTTCTGTCCAAAAAGTC | |
| | ACTGAACTCATACTTCAGTACATATACATGTATAAGGCACACTGTAGTGCTTAATATGTGTAAGGACTTC | |
| | CTCTTTAAATTTGGTACCAGTAACTTAGTGACACATAATGACAACCAAAATATTTGAAAGCACTTAAGCA | |
| | CTCCTCCTTGTGGAAAGAATATACCACCATTTCATCTGGCTAGTTCACCATCACAACTGCATTACCAAAA | |
| | GGGGATTTTTGAAAACGAGGAGTTGACCAAAATAATATCTGAAGATGATTGCTTTTCCCTGCTGCCAGCT | |
| | GATCTGAAATGTTTTGCTGGCACATTAATCATAGATAAAGAAAGATTGATGGACTTAGCCCTCAAATTTC | |
| | AGTATCTATACAGTACTAGACCATGCATTCTTAAAATATTAGATACCAGGTAGTATATATTGTTTCTGTA | |
| | CAAAAATGACTGTATTCTCTCACCAGTAGGACTTAAACTTTGTTTCTCCAGTGGCTTAGCTCCTGTTCCT | |
| | TTGGGTGATCACTAGCACCCATTTTTGAGAAAGCTGGTTCTACATGGGGGGATAGCTGTGGAATAGATAA | |
| | TTTGCTGCATGTTAATTCTCAAGAACTAAGCCTGTGCCAGTGCTTTCCTAAGCAGTATACCTTTAATCAG | |
| | AACTCATTCCCAGAACCTGGATGCTATTACACATGCTTTTAAGAAACGTCAATGTATATCCTTTTATAAC | |
| | TCTACCACTTTGGGGCAAGCTATTCCAGCACTGGTTTTGAATGCTGTATGCAACCAGTCTGAATACCACA | |
| | TACGCTGCACTGTTCTTAGAGGGTTTCCATACTTACCACCGATCTACAAGGGTTGATCCCTGTTTTTACC | |
| | ATCAATCATCACCCTGTGGTGCAACACTTGAAAGACCCGGCTAGAGGCACTATGGACTTCAGGATCCACT | |
| | AGACAGTTTTCAGTTTGCTTGGAGGTAGCTGGGTAATCAAAAATGTTTAGTCATTGATTCAATGTGAACG | |
| | ATTACGGTCTTTATGACCAAGAGTCTGAAAATCTTTTTGTTATGCTGTTTAGTATTCGTTTGATATTGTT | |
| | ACTTTTCACCTGTTGAGCCCAAATTCAGGATTGGTTCAGTGGCAGCAATGAAGTTGCCATTTAAATTTGT | |
| | TCATAGCCTACATCACCAAGGTCTCTGTGTCAAACCTGTGGCCACTCTATATGCACTTTGTTTACTCTTT | |
| | ATACAAATAAATATACTAAAGACTTTA | |
| 35 | ATCTATCACATGGCAGAGATAGAATAAAAACAGAAAAATGGCGACGGTCACGTTGTGGCGAGCCTTGCTG | >NM_001321854.2 |
| | CGTCATTAGATAATCCTCATGCAAATAGCGGGAAGAACAAAGGAAGGGGAGCCCGGGACCCCCGGGGGCG | Homo sapiens |
| | CAGGATCCGGCGGGAGGAGTCTAAGAGGAGGAGGCGGCGGTGCCGGAGGAGGAGGAGGAGGGAGGGAGAA | Janus |
| | GAGAGGAAGACCGGAGTCCCCGCGGCGGCGGCGGTCCGGAGAGAGGGCGAGCCCCGCGCGGCGCCGGGGA | kinase 1 |
| | CCGGGCGCTACCACGAGGCCGGGACGCTGGAGTCTGGGCGCTTCTCTGAAGTAGCTTTGGAAAGTAGAGA | (JAK1), |
| | AGAAAATCCAGTTTGCTTCTTGGAGAACACTGGACAGCTGAATAAATGCAGTATCTAAATATAAAAGAGG | transcript |
| | ACTGCAATGCCATGGCTTTCTGTGCTAAAATGAGGAGCTCCAAGAAGACTGAGGTGAACCTGGAGGCCCC | variant 5, |
| | TGAGCCAGGGGTGGAAGTGATCTTCTATCTGTCGGACAGGGAGCCCCTCCGGCTGGGCAGTGGAGAGTAC | mRNA |
| | ACAGCAGAGGAACTGTGCATCAGGGCTGCACAGGCATGCCGTATCTCTCCTCTTTGTCACAACCTCTTTG | |
| | CCCTGTATGACGAGAACACCAAGCTCTGGTATGCTCCAAATCGCACCATCACCGTTGATGACAAGATGTC | |
| | CCTCCGGCTCCACTACCGGATGAGGTTCTATTTCACCAATTGGCATGGAACCAACGACAATGAGCAGTCA | |
| | GTGTGGCGTCATTCTCCAAAGAAGCAGAAAAATGGCTACGAGAAAAAAAAGATTCCAGATGCAACCCCTC | |
| | TCCTTGATGCCAGCTCACTGGAGTATCTGTTTGCTCAGGGACAGTATGATTTGGTGAAATGCCTGGCTCC | |
| | TATTCGAGACCCCAAGACCGAGCAGGATGGACATGATATTGAGAACGAGTGTCTAGGGATGGCTGTCCTG | |
| | GCCATCTCACACTATGCCATGATGAAGAAGATGCAGTTGCCAGAACTGCCCAAGGACATCAGCTACAAGC | |
| | GATATATTCCAGAAACATTGAATAAGTCCATCAGACAGAGGAACCTTCTCACCAGGATGCGGATAAATAA | |
| | TGTTTTCAAGGATTTCCTAAAGGAATTTAACAACAAGACCATTTGTGACAGCAGCGTGTCCACGCATGAC | |
| | CTGAAGGTGAAATACTTGGCTACCTTGGAAACTTTGACAAAACATTACGGTGCTGAAATATTTGAGACTT | |
| | CCATGTTACTGATTTCATCAGAAAATGAGATGAATTGGTTTCATTCGAATGACGGTGGAAACGTTCTCTA | |
| | CTACGAAGTGATGGTGACTGGGAATCTTGGAATCCAGTGGAGGCATAAACCAAATGTTGTTTCTGTTGAA | |
| | AAGGAAAAAAATAAACTGAAGCGGAAAAAACTGGAAAATAAACACAAGAAGGATGAGGAGAAAAACAAGA | |
| | TCCGGGAAGAGTGGAACAATTTTTCTTACTTCCCTGAAATCACTCACATTGTAATAAAGGAGTCTGTGGT | |
| | CAGCATTAACAAGCAGGACAACAAGAAAATGGAACTGAAGCTCTCTTCCCACGAGGAGGCCTTGTCCTTT | |
| | GTGTCCCTGGTAGATGGCTACTTCCGGCTCACAGCAGATGCCCATCATTACCTCTGCACCGACGTGGCCC | |
| | CCCCGTTGATCGTCCACAACATACAGAATGGCTGTCATGGTCCAATCTGTACAGAATACGCCATCAATAA | |
| | ATTGCGGCAAGAAGGAAGCGAGGAGGGGATGTACGTGCTGAGGTGGAGCTGCACCGACTTTGACAACATC | |
| | CTCATGACCGTCACCTGCTTTGAGAAGTCTGAGCAGGTGCAGGGTGCCCAGAAGCAGTTCAAGAACTTTC | |
| | AGATCGAGGTGCAGAAGGGCCGCTACAGTCTGCACGGTTCGGACCGCAGCTTCCCCAGCTTGGGAGACCT | |
| | CATGAGCCACCTCAAGAAGCAGATCCTGCGCACGGATAACATCAGCTTCATGCTAAAACGCTGCTGCCAG | |
| | CCCAAGCCCCGAGAAATCTCCAACCTGCTGGTGGCTACTAAGAAAGCCCAGGAGTGGCAGCCCGTCTACC | |
| | CCATGAGCCAGCTGAGTTTCGATCGGATCCTCAAGAAGGATCTGGTGCAGGGCGAGCACCTTGGGAGAGG | |
| | CACGAGAACACACATCTATTCTGGGACCCTGATGGATTACAAGGATGACGAAGGAACTTCTGAAGAGAAG | |
| | AAGATAAAAGTGATCCTCAAAGTCTTAGACCCCAGCCACAGGGATATTTCCCTGGCCTTCTTCGAGGCAG | |
| | CCAGCATGATGAGACAGGTCTCCCACAAACACATCGTGTACCTCTATGGCGTCTGTGTCCGCGACGTGGA | |
| | GAATATCATGGTGGAAGAGTTTGTGGAAGGGGGTCCTCTGGATCTCTTCATGCACCGGAAAAGCGATGTC | |
| | CTTACCACACCATGGAAATTCAAAGTTGCCAAACAGCTGGCCAGTGCCCTGAGCTACTTGGAGGATAAAG | |
| | ACCTGGTCCATGGAAATGTGTGTACTAAAAACCTCCTCCTGGCCCGTGAGGGCATCGACAGTGAGTGTGG | |
| | CCCATTCATCAAGCTCAGTGACCCCGGCATCCCCATTACGGTGCTGTCTAGGCAAGAATGCATTGAACGA | |
| | ATCCCATGGATTGCTCCTGAGTGTGTTGAGGACTCCAAGAACCTGAGTGTGGCTGCTGACAAGTGGAGCT | |
| | TTGGAACCACGCTCTGGGAAATCTGCTACAATGGCGAGATCCCCTTGAAAGACAAGACGCTGATTGAGAA | |
| | AGAGAGATTCTATGAAAGCCGGTGCAGGCCAGTGACACCATCATGTAAGGAGCTGGCTGACCTCATGACC | |
| | CGCTGCATGAACTATGACCCCAATCAGAGGCCTTTCTTCCGAGCCATCATGAGAGACATTAATAAGCTTG | |
| | AAGAGCAGAATCCAGATATTGTTTCAGAAAAAAAACCAGCAACTGAAGTGGACCCCACACATTTTGAAAA | |
| | GCGCTTCCTAAAGAGGATCCGTGACTTGGGAGAGGGCCACTTTGGGAAGGTTGAGCTCTGCAGGTATGAC | |
| | CCCGAAGGGGACAATACAGGGGAGCAGGTGGCTGTTAAATCTCTGAAGCCTGAGAGTGGAGGTAACCACA | |
| | TAGCTGATCTGAAAAAGGAAATCGAGATCTTAAGGAACCTCTATCATGAGAACATTGTGAAGTACAAAGG | |
| | AATCTGCACAGAAGACGGAGGAAATGGTATTAAGCTCATCATGGAATTTCTGCCTTCGGGAAGCCTTAAG | |
| | GAATATCTTCCAAAGAATAAGAACAAAATAAACCTCAAACAGCAGCTAAAATATGCCGTTCAGATTTGTA | |
| | AGGGGATGGACTATTTGGGTTCTCGGCAATACGTTCACCGGGACTTGGCAGCAAGAAATGTCCTTGTTGA | |
| | GAGTGAACACCAAGTGAAAATTGGAGACTTCGGTTTAACCAAAGCAATTGAAACCGATAAGGAGTATTAC | |
| | ACCGTCAAGGATGACCGGGACAGCCCTGTGTTTTGGTATGCTCCAGAATGTTTAATGCAATCTAAATTTT | |
| | ATATTGCCTCTGACGTCTGGTCTTTTGGAGTCACTCTGCATGAGCTGCTGACTTACTGTGATTCAGATTC | |
| | TAGTCCCATGGCTTTGTTCCTGAAAATGATAGGCCCAACCCATGGCCAGATGACAGTCACAAGACTTGTG | |
| | AATACGTTAAAAGAAGGAAAACGCCTGCCGTGCCCACCTAACTGTCCAGATGAGGTTTATCAACTTATGA | |
| | GGAAATGCTGGGAATTCCAACCATCCAATCGGACAAGCTTTCAGAACCTTATTGAAGGATTTGAAGCACT | |
| | TTTAAAATAAGAAGCATGAATAACATTTAAATTCCACAGATTATCAAGTCCTTCTCCTGCAACAAATGCC | |
| | CAAGTCATTTTTTAAAAATTTCTAATGAAAGAAGTTTGTGTTCTGTCCAAAAAGTCACTGAACTCATACT | |
| | TCAGTACATATACATGTATAAGGCACACTGTAGTGCTTAATATGTGTAAGGACTTCCTCTTTAAATTTGG | |
| | TACCAGTAACTTAGTGACACATAATGACAACCAAAATATTTGAAAGCACTTAAGCACTCCTCCTTGTGGA | |
| | AAGAATATACCACCATTTCATCTGGCTAGTTCACCATCACAACTGCATTACCAAAAGGGGATTTTTGAAA | |
| | ACGAGGAGTTGACCAAAATAATATCTGAAGATGATTGCTTTTCCCTGCTGCCAGCTGATCTGAAATGTTT | |
| | TGCTGGCACATTAATCATAGATAAAGAAAGATTGATGGACTTAGCCCTCAAATTTCAGTATCTATACAGT | |
| | ACTAGACCATGCATTCTTAAAATATTAGATACCAGGTAGTATATATTGTTTCTGTACAAAAATGACTGTA | |
| | TTCTCTCACCAGTAGGACTTAAACTTTGTTTCTCCAGTGGCTTAGCTCCTGTTCCTTTGGGTGATCACTA | |
| | GCACCCATTTTTGAGAAAGCTGGTTCTACATGGGGGGATAGCTGTGGAATAGATAATTTGCTGCATGTTA | |
| | ATTCTCAAGAACTAAGCCTGTGCCAGTGCTTTCCTAAGCAGTATACCTTTAATCAGAACTCATTCCCAGA | |
| | ACCTGGATGCTATTACACATGCTTTTAAGAAACGTCAATGTATATCCTTTTATAACTCTACCACTTTGGG | |
| | GCAAGCTATTCCAGCACTGGTTTTGAATGCTGTATGCAACCAGTCTGAATACCACATACGCTGCACTGTT | |
| | CTTAGAGGGTTTCCATACTTACCACCGATCTACAAGGGTTGATCCCTGTTTTTACCATCAATCATCACCC | |
| | TGTGGTGCAACACTTGAAAGACCCGGCTAGAGGCACTATGGACTTCAGGATCCACTAGACAGTTTTCAGT | |
| | TTGCTTGGAGGTAGCTGGGTAATCAAAAATGTTTAGTCATTGATTCAATGTGAACGATTACGGTCTTTAT | |
| | GACCAAGAGTCTGAAAATCTTTTTGTTATGCTGTTTAGTATTCGTTTGATATTGTTACTTTTCACCTGTT | |
| | GAGCCCAAATTCAGGATTGGTTCAGTGGCAGCAATGAAGTTGCCATTTAAATTTGTTCATAGCCTACATC | |
| | ACCAAGGTCTCTGTGTCAAACCTGTGGCCACTCTATATGCACTTTGTTTACTCTTTATACAAATAAATAT | |
| | ACTAAAGACTTTA | |
| 36 | GCGTCGCTGAGCGCAGGCCGCGGCGGCCGCGGAGTATCCTGGAGCTGCAGACAGTGCGGGCCTGCGCCCA | >NM_001321855.2 |
| | GTCCCGGCTGTCCTCGCCGCGACCCCTCCTCAGCCCTGGGCGCGCGCACGCTGGGGCCCCGCGGGGCTGG | Homo sapiens |
| | CCGCCTAGCGAGCCTGCCGGTCGACCCCAGCCAGCGCAGCGACGGGGCGCTGCCTGGCCCAGGCGCACAC | Janus |
| | GGAAGTGTTATCTAAAACAGTTCATGCTGCTGAAAACCTCCTTCCTGGCAGATGTCCCTCAACCCTACTG | kinase 1 |
| | GTGCCTGGCTTCTGAGACACACGCTTCTCTGAAGTAGCTTTGGAAAGTAGAGAAGAAAATCCAGTTTGCT | (JAK1), |
| | TCTTGGAGAACACTGGACAGCTGAATAAATGCAGTATCTAAATATAAAAGAGGACTGCAATGCCATGGCT | transcript |
| | TTCTGTGCTAAAATGAGGAGCTCCAAGAAGACTGAGGTGAACCTGGAGGCCCCTGAGCCAGGGGTGGAAG | variant 6, |
| | TGATCTTCTATCTGTCGGACAGGGAGCCCCTCCGGCTGGGCAGTGGAGAGTACACAGCAGAGGAACTGTG | mRNA |
| | CATCAGGGCTGCACAGGCATGCCGTATCTCTCCTCTTTGTCACAACCTCTTTGCCCTGTATGACGAGAAC | |
| | ACCAAGCTCTGGTATGCTCCAAATCGCACCATCACCGTTGATGACAAGATGTCCCTCCGGCTCCACTACC | |
| | GGATGAGGTTCTATTTCACCAATTGGCATGGAACCAACGACAATGAGCAGTCAGTGTGGCGTCATTCTCC | |
| | AAAGAAGCAGAAAAATGGCTACGAGAAAAAAAAGATTCCAGATGCAACCCCTCTCCTTGATGCCAGCTCA | |
| | CTGGAGTATCTGTTTGCTCAGGGACAGTATGATTTGGTGAAATGCCTGGCTCCTATTCGAGACCCCAAGA | |
| | CCGAGCAGGATGGACATGATATTGAGAACGAGTGTCTAGGGATGGCTGTCCTGGCCATCTCACACTATGC | |
| | CATGATGAAGAAGATGCAGTTGCCAGAACTGCCCAAGGACATCAGCTACAAGCGATATATTCCAGAAACA | |
| | TTGAATAAGTCCATCAGACAGAGGAACCTTCTCACCAGGATGCGGATAAATAATGTTTTCAAGGATTTCC | |
| | TAAAGGAATTTAACAACAAGACCATTTGTGACAGCAGCGTGTCCACGCATGACCTGAAGGTGAAATACTT | |
| | GGCTACCTTGGAAACTTTGACAAAACATTACGGTGCTGAAATATTTGAGACTTCCATGTTACTGATTTCA | |
| | TCAGAAAATGAGATGAATTGGTTTCATTCGAATGACGGTGGAAACGTTCTCTACTACGAAGTGATGGTGA | |
| | CTGGGAATCTTGGAATCCAGTGGAGGCATAAACCAAATGTTGTTTCTGTTGAAAAGGAAAAAAATAAACT | |
| | GAAGCGGAAAAAACTGGAAAATAAACACAAGAAGGATGAGGAGAAAAACAAGATCCGGGAAGAGTGGAAC | |
| | AATTTTTCTTACTTCCCTGAAATCACTCACATTGTAATAAAGGAGTCTGTGGTCAGCATTAACAAGCAGG | |
| | ACAACAAGAAAATGGAACTGAAGCTCTCTTCCCACGAGGAGGCCTTGTCCTTTGTGTCCCTGGTAGATGG | |
| | CTACTTCCGGCTCACAGCAGATGCCCATCATTACCTCTGCACCGACGTGGCCCCCCCGTTGATCGTCCAC | |
| | AACATACAGAATGGCTGTCATGGTCCAATCTGTACAGAATACGCCATCAATAAATTGCGGCAAGAAGGAA | |
| | GCGAGGAGGGGATGTACGTGCTGAGGTGGAGCTGCACCGACTTTGACAACATCCTCATGACCGTCACCTG | |
| | CTTTGAGAAGTCTGAGCAGGTGCAGGGTGCCCAGAAGCAGTTCAAGAACTTTCAGATCGAGGTGCAGAAG | |
| | GGCCGCTACAGTCTGCACGGTTCGGACCGCAGCTTCCCCAGCTTGGGAGACCTCATGAGCCACCTCAAGA | |
| | AGCAGATCCTGCGCACGGATAACATCAGCTTCATGCTAAAACGCTGCTGCCAGCCCAAGCCCCGAGAAAT | |
| | CTCCAACCTGCTGGTGGCTACTAAGAAAGCCCAGGAGTGGCAGCCCGTCTACCCCATGAGCCAGCTGAGT | |
| | TTCGATCGGATCCTCAAGAAGGATCTGGTGCAGGGCGAGCACCTTGGGAGAGGCACGAGAACACACATCT | |
| | ATTCTGGGACCCTGATGGATTACAAGGATGACGAAGGAACTTCTGAAGAGAAGAAGATAAAAGTGATCCT | |
| | CAAAGTCTTAGACCCCAGCCACAGGGATATTTCCCTGGCCTTCTTCGAGGCAGCCAGCATGATGAGACAG | |
| | GTCTCCCACAAACACATCGTGTACCTCTATGGCGTCTGTGTCCGCGACGTGGAGAATATCATGGTGGAAG | |
| | AGTTTGTGGAAGGGGGTCCTCTGGATCTCTTCATGCACCGGAAAAGCGATGTCCTTACCACACCATGGAA | |
| | ATTCAAAGTTGCCAAACAGCTGGCCAGTGCCCTGAGCTACTTGGAGGATAAAGACCTGGTCCATGGAAAT | |
| | GTGTGTACTAAAAACCTCCTCCTGGCCCGTGAGGGCATCGACAGTGAGTGTGGCCCATTCATCAAGCTCA | |
| | GTGACCCCGGCATCCCCATTACGGTGCTGTCTAGGCAAGAATGCATTGAACGAATCCCATGGATTGCTCC | |
| | TGAGTGTGTTGAGGACTCCAAGAACCTGAGTGTGGCTGCTGACAAGTGGAGCTTTGGAACCACGCTCTGG | |
| | GAAATCTGCTACAATGGCGAGATCCCCTTGAAAGACAAGACGCTGATTGAGAAAGAGAGATTCTATGAAA | |
| | GCCGGTGCAGGCCAGTGACACCATCATGTAAGGAGCTGGCTGACCTCATGACCCGCTGCATGAACTATGA | |
| | CCCCAATCAGAGGCCTTTCTTCCGAGCCATCATGAGAGACATTAATAAGCTTGAAGAGCAGAATCCAGAT | |
| | ATTGTTTCAGAAAAAAAACCAGCAACTGAAGTGGACCCCACACATTTTGAAAAGCGCTTCCTAAAGAGGA | |
| | TCCGTGACTTGGGAGAGGGCCACTTTGGGAAGGTTGAGCTCTGCAGGTATGACCCCGAAGGGGACAATAC | |
| | AGGGGAGCAGGTGGCTGTTAAATCTCTGAAGCCTGAGAGTGGAGGTAACCACATAGCTGATCTGAAAAAG | |
| | GAAATCGAGATCTTAAGGAACCTCTATCATGAGAACATTGTGAAGTACAAAGGAATCTGCACAGAAGACG | |
| | GAGGAAATGGTATTAAGCTCATCATGGAATTTCTGCCTTCGGGAAGCCTTAAGGAATATCTTCCAAAGAA | |
| | TAAGAACAAAATAAACCTCAAACAGCAGCTAAAATATGCCGTTCAGATTTGTAAGGGGATGGACTATTTG | |
| | GGTTCTCGGCAATACGTTCACCGGGACTTGGCAGCAAGAAATGTCCTTGTTGAGAGTGAACACCAAGTGA | |
| | AAATTGGAGACTTCGGTTTAACCAAAGCAATTGAAACCGATAAGGAGTATTACACCGTCAAGGATGACCG | |
| | GGACAGCCCTGTGTTTTGGTATGCTCCAGAATGTTTAATGCAATCTAAATTTTATATTGCCTCTGACGTC | |
| | TGGTCTTTTGGAGTCACTCTGCATGAGCTGCTGACTTACTGTGATTCAGATTCTAGTCCCATGGCTTTGT | |
| | TCCTGAAAATGATAGGCCCAACCCATGGCCAGATGACAGTCACAAGACTTGTGAATACGTTAAAAGAAGG | |
| | AAAACGCCTGCCGTGCCCACCTAACTGTCCAGATGAGGTTTATCAACTTATGAGGAAATGCTGGGAATTC | |
| | CAACCATCCAATCGGACAAGCTTTCAGAACCTTATTGAAGGATTTGAAGCACTTTTAAAATAAGAAGCAT | |
| | GAATAACATTTAAATTCCACAGATTATCAAGTCCTTCTCCTGCAACAAATGCCCAAGTCATTTTTTAAAA | |
| | ATTTCTAATGAAAGAAGTTTGTGTTCTGTCCAAAAAGTCACTGAACTCATACTTCAGTACATATACATGT | |
| | ATAAGGCACACTGTAGTGCTTAATATGTGTAAGGACTTCCTCTTTAAATTTGGTACCAGTAACTTAGTGA | |
| | CACATAATGACAACCAAAATATTTGAAAGCACTTAAGCACTCCTCCTTGTGGAAAGAATATACCACCATT | |
| | TCATCTGGCTAGTTCACCATCACAACTGCATTACCAAAAGGGGATTTTTGAAAACGAGGAGTTGACCAAA | |
| | ATAATATCTGAAGATGATTGCTTTTCCCTGCTGCCAGCTGATCTGAAATGTTTTGCTGGCACATTAATCA | |
| | TAGATAAAGAAAGATTGATGGACTTAGCCCTCAAATTTCAGTATCTATACAGTACTAGACCATGCATTCT | |
| | TAAAATATTAGATACCAGGTAGTATATATTGTTTCTGTACAAAAATGACTGTATTCTCTCACCAGTAGGA | |
| | CTTAAACTTTGTTTCTCCAGTGGCTTAGCTCCTGTTCCTTTGGGTGATCACTAGCACCCATTTTTGAGAA | |
| | AGCTGGTTCTACATGGGGGGATAGCTGTGGAATAGATAATTTGCTGCATGTTAATTCTCAAGAACTAAGC | |
| | CTGTGCCAGTGCTTTCCTAAGCAGTATACCTTTAATCAGAACTCATTCCCAGAACCTGGATGCTATTACA | |
| | CATGCTTTTAAGAAACGTCAATGTATATCCTTTTATAACTCTACCACTTTGGGGCAAGCTATTCCAGCAC | |
| | TGGTTTTGAATGCTGTATGCAACCAGTCTGAATACCACATACGCTGCACTGTTCTTAGAGGGTTTCCATA | |
| | CTTACCACCGATCTACAAGGGTTGATCCCTGTTTTTACCATCAATCATCACCCTGTGGTGCAACACTTGA | |
| | AAGACCCGGCTAGAGGCACTATGGACTTCAGGATCCACTAGACAGTTTTCAGTTTGCTTGGAGGTAGCTG | |
| | GGTAATCAAAAATGTTTAGTCATTGATTCAATGTGAACGATTACGGTCTTTATGACCAAGAGTCTGAAAA | |
| | TCTTTTTGTTATGCTGTTTAGTATTCGTTTGATATTGTTACTTTTCACCTGTTGAGCCCAAATTCAGGAT | |
| | TGGTTCAGTGGCAGCAATGAAGTTGCCATTTAAATTTGTTCATAGCCTACATCACCAAGGTCTCTGTGTC | |
| | AAACCTGTGGCCACTCTATATGCACTTTGTTTACTCTTTATACAAATAAATATACTAAAGACTTTA | |
| 37 | AGAAGCGGAGCGTATACGGAGGAGGCGGGATGCATTTCTGCATCGAGCGCACAAAGCGCTTCTCTGAAGT | >NM_001321856.2 |
| | AGCTTTGGAAAGTAGAGAAGAAAATCCAGTTTGCTTCTTGGAGAACACTGGACAGCTGAATAAATGCAGT | Homo sapiens |
| | ATCTAAATATAAAAGAGGACTGCAATGCCATGGCTTTCTGTGCTAAAATGAGGAGCTCCAAGAAGACTGA | Janus |
| | GGTGAACCTGGAGGCCCCTGAGCCAGGGGTGGAAGTGATCTTCTATCTGTCGGACAGGGAGCCCCTCCGG | kinase 1 |
| | CTGGGCAGTGGAGAGTACACAGCAGAGGAACTGTGCATCAGGGCTGCACAGGCATGCCGTATCTCTCCTC | (JAK1), |
| | TTTGTCACAACCTCTTTGCCCTGTATGACGAGAACACCAAGCTCTGGTATGCTCCAAATCGCACCATCAC | transcript |
| | CGTTGATGACAAGATGTCCCTCCGGCTCCACTACCGGATGAGGTTCTATTTCACCAATTGGCATGGAACC | variant 7, |
| | AACGACAATGAGCAGTCAGTGTGGCGTCATTCTCCAAAGAAGCAGAAAAATGGCTACGAGAAAAAAAAGA | mRNA |
| | TTCCAGATGCAACCCCTCTCCTTGATGCCAGCTCACTGGAGTATCTGTTTGCTCAGGGACAGTATGATTT | |
| | GGTGAAATGCCTGGCTCCTATTCGAGACCCCAAGACCGAGCAGGATGGACATGATATTGAGAACGAGTGT | |
| | CTAGGGATGGCTGTCCTGGCCATCTCACACTATGCCATGATGAAGAAGATGCAGTTGCCAGAACTGCCCA | |
| | AGGACATCAGCTACAAGCGATATATTCCAGAAACATTGAATAAGTCCATCAGACAGAGGAACCTTCTCAC | |
| | CAGGATGCGGATAAATAATGTTTTCAAGGATTTCCTAAAGGAATTTAACAACAAGACCATTTGTGACAGC | |
| | AGCGTGTCCACGCATGACCTGAAGGTGAAATACTTGGCTACCTTGGAAACTTTGACAAAACATTACGGTG | |
| | CTGAAATATTTGAGACTTCCATGTTACTGATTTCATCAGAAAATGAGATGAATTGGTTTCATTCGAATGA | |
| | CGGTGGAAACGTTCTCTACTACGAAGTGATGGTGACTGGGAATCTTGGAATCCAGTGGAGGCATAAACCA | |
| | AATGTTGTTTCTGTTGAAAAGGAAAAAAATAAACTGAAGCGGAAAAAACTGGAAAATAAACACAAGAAGG | |
| | ATGAGGAGAAAAACAAGATCCGGGAAGAGTGGAACAATTTTTCTTACTTCCCTGAAATCACTCACATTGT | |
| | AATAAAGGAGTCTGTGGTCAGCATTAACAAGCAGGACAACAAGAAAATGGAACTGAAGCTCTCTTCCCAC | |
| | GAGGAGGCCTTGTCCTTTGTGTCCCTGGTAGATGGCTACTTCCGGCTCACAGCAGATGCCCATCATTACC | |
| | TCTGCACCGACGTGGCCCCCCCGTTGATCGTCCACAACATACAGAATGGCTGTCATGGTCCAATCTGTAC | |
| | AGAATACGCCATCAATAAATTGCGGCAAGAAGGAAGCGAGGAGGGGATGTACGTGCTGAGGTGGAGCTGC | |
| | ACCGACTTTGACAACATCCTCATGACCGTCACCTGCTTTGAGAAGTCTGAGCAGGTGCAGGGTGCCCAGA | |
| | AGCAGTTCAAGAACTTTCAGATCGAGGTGCAGAAGGGCCGCTACAGTCTGCACGGTTCGGACCGCAGCTT | |
| | CCCCAGCTTGGGAGACCTCATGAGCCACCTCAAGAAGCAGATCCTGCGCACGGATAACATCAGCTTCATG | |
| | CTAAAACGCTGCTGCCAGCCCAAGCCCCGAGAAATCTCCAACCTGCTGGTGGCTACTAAGAAAGCCCAGG | |
| | AGTGGCAGCCCGTCTACCCCATGAGCCAGCTGAGTTTCGATCGGATCCTCAAGAAGGATCTGGTGCAGGG | |
| | CGAGCACCTTGGGAGAGGCACGAGAACACACATCTATTCTGGGACCCTGATGGATTACAAGGATGACGAA | |
| | GGAACTTCTGAAGAGAAGAAGATAAAAGTGATCCTCAAAGTCTTAGACCCCAGCCACAGGGATATTTCCC | |
| | TGGCCTTCTTCGAGGCAGCCAGCATGATGAGACAGGTCTCCCACAAACACATCGTGTACCTCTATGGCGT | |
| | CTGTGTCCGCGACGTGGAGAATATCATGGTGGAAGAGTTTGTGGAAGGGGGTCCTCTGGATCTCTTCATG | |
| | CACCGGAAAAGCGATGTCCTTACCACACCATGGAAATTCAAAGTTGCCAAACAGCTGGCCAGTGCCCTGA | |
| | GCTACTTGGAGGATAAAGACCTGGTCCATGGAAATGTGTGTACTAAAAACCTCCTCCTGGCCCGTGAGGG | |
| | CATCGACAGTGAGTGTGGCCCATTCATCAAGCTCAGTGACCCCGGCATCCCCATTACGGTGCTGTCTAGG | |
| | CAAGAATGCATTGAACGAATCCCATGGATTGCTCCTGAGTGTGTTGAGGACTCCAAGAACCTGAGTGTGG | |
| | CTGCTGACAAGTGGAGCTTTGGAACCACGCTCTGGGAAATCTGCTACAATGGCGAGATCCCCTTGAAAGA | |
| | CAAGACGCTGATTGAGAAAGAGAGATTCTATGAAAGCCGGTGCAGGCCAGTGACACCATCATGTAAGGAG | |
| | CTGGCTGACCTCATGACCCGCTGCATGAACTATGACCCCAATCAGAGGCCTTTCTTCCGAGCCATCATGA | |
| | GAGACATTAATAAGCTTGAAGAGCAGAATCCAGATATTGTTTCAGAAAAAAAACCAGCAACTGAAGTGGA | |
| | CCCCACACATTTTGAAAAGCGCTTCCTAAAGAGGATCCGTGACTTGGGAGAGGGCCACTTTGGGAAGGTT | |
| | GAGCTCTGCAGGTATGACCCCGAAGGGGACAATACAGGGGAGCAGGTGGCTGTTAAATCTCTGAAGCCTG | |
| | AGAGTGGAGGTAACCACATAGCTGATCTGAAAAAGGAAATCGAGATCTTAAGGAACCTCTATCATGAGAA | |
| | CATTGTGAAGTACAAAGGAATCTGCACAGAAGACGGAGGAAATGGTATTAAGCTCATCATGGAATTTCTG | |
| | CCTTCGGGAAGCCTTAAGGAATATCTTCCAAAGAATAAGAACAAAATAAACCTCAAACAGCAGCTAAAAT | |
| | ATGCCGTTCAGATTTGTAAGGGGATGGACTATTTGGGTTCTCGGCAATACGTTCACCGGGACTTGGCAGC | |
| | AAGAAATGTCCTTGTTGAGAGTGAACACCAAGTGAAAATTGGAGACTTCGGTTTAACCAAAGCAATTGAA | |
| | ACCGATAAGGAGTATTACACCGTCAAGGATGACCGGGACAGCCCTGTGTTTTGGTATGCTCCAGAATGTT | |
| | TAATGCAATCTAAATTTTATATTGCCTCTGACGTCTGGTCTTTTGGAGTCACTCTGCATGAGCTGCTGAC | |
| | TTACTGTGATTCAGATTCTAGTCCCATGGCTTTGTTCCTGAAAATGATAGGCCCAACCCATGGCCAGATG | |
| | ACAGTCACAAGACTTGTGAATACGTTAAAAGAAGGAAAACGCCTGCCGTGCCCACCTAACTGTCCAGATG | |
| | AGGTTTATCAACTTATGAGGAAATGCTGGGAATTCCAACCATCCAATCGGACAAGCTTTCAGAACCTTAT | |
| | TGAAGGATTTGAAGCACTTTTAAAATAAGAAGCATGAATAACATTTAAATTCCACAGATTATCAAGTCCT | |
| | TCTCCTGCAACAAATGCCCAAGTCATTTTTTAAAAATTTCTAATGAAAGAAGTTTGTGTTCTGTCCAAAA | |
| | AGTCACTGAACTCATACTTCAGTACATATACATGTATAAGGCACACTGTAGTGCTTAATATGTGTAAGGA | |
| | CTTCCTCTTTAAATTTGGTACCAGTAACTTAGTGACACATAATGACAACCAAAATATTTGAAAGCACTTA | |
| | AGCACTCCTCCTTGTGGAAAGAATATACCACCATTTCATCTGGCTAGTTCACCATCACAACTGCATTACC | |
| | AAAAGGGGATTTTTGAAAACGAGGAGTTGACCAAAATAATATCTGAAGATGATTGCTTTTCCCTGCTGCC | |
| | AGCTGATCTGAAATGTTTTGCTGGCACATTAATCATAGATAAAGAAAGATTGATGGACTTAGCCCTCAAA | |
| | TTTCAGTATCTATACAGTACTAGACCATGCATTCTTAAAATATTAGATACCAGGTAGTATATATTGTTTC | |
| | TGTACAAAAATGACTGTATTCTCTCACCAGTAGGACTTAAACTTTGTTTCTCCAGTGGCTTAGCTCCTGT | |
| | TCCTTTGGGTGATCACTAGCACCCATTTTTGAGAAAGCTGGTTCTACATGGGGGGATAGCTGTGGAATAG | |
| | ATAATTTGCTGCATGTTAATTCTCAAGAACTAAGCCTGTGCCAGTGCTTTCCTAAGCAGTATACCTTTAA | |
| | TCAGAACTCATTCCCAGAACCTGGATGCTATTACACATGCTTTTAAGAAACGTCAATGTATATCCTTTTA | |
| | TAACTCTACCACTTTGGGGCAAGCTATTCCAGCACTGGTTTTGAATGCTGTATGCAACCAGTCTGAATAC | |
| | CACATACGCTGCACTGTTCTTAGAGGGTTTCCATACTTACCACCGATCTACAAGGGTTGATCCCTGTTTT | |
| | TACCATCAATCATCACCCTGTGGTGCAACACTTGAAAGACCCGGCTAGAGGCACTATGGACTTCAGGATC | |
| | CACTAGACAGTTTTCAGTTTGCTTGGAGGTAGCTGGGTAATCAAAAATGTTTAGTCATTGATTCAATGTG | |
| | AACGATTACGGTCTTTATGACCAAGAGTCTGAAAATCTTTTTGTTATGCTGTTTAGTATTCGTTTGATAT | |
| | TGTTACTTTTCACCTGTTGAGCCCAAATTCAGGATTGGTTCAGTGGCAGCAATGAAGTTGCCATTTAAAT | |
| | TTGTTCATAGCCTACATCACCAAGGTCTCTGTGTCAAACCTGTGGCCACTCTATATGCACTTTGTTTACT | |
| | CTTTATACAAATAAATATACTAAAGACTTTA | |
| 38 | GCGTCGCTGAGCGCAGGCCGCGGCGGCCGCGGAGTATCCTGGAGCTGCAGACAGTGCGGGCCTGCGCCCA | >NM_001321857.2 |
| | GTCCCGGCTGTCCTCGCCGCGACCCCTCCTCAGCCCTGGGCGCGCGCACGCTGGGGCCCCGCGGGGCTGG | Homo sapiens |
| | CCGCCTAGCGAGCCTGCCGGTCGACCCCAGCCAGCGCAGCGACGGGGCGCTGCCTGGCCCAGGCGCACAC | Janus |
| | GGAAGTGCGCTTCTCTGAAGTAGCTTTGGAAAGTAGAGAAGAAAATCCAGTTTGCTTCTTGGAGAACACT | kinase 1 |
| | GGACAGCTGAATAAATGCAGTATCTAAATATAAAAGAGGACTGCAATGCCATGGCTTTCTGTGCTAAAAT | (JAK1), |
| | GAGGAGCTCCAAGAAGACTGAGGTGAACCTGGAGGCCCCTGAGCCAGGGGTGGAAGTGATCTTCTATCTG | transcript |
| | TCGGACAGGGAGCCCCTCCGGCTGGGCAGTGGAGAGTACACAGCAGAGGAACTGTGCATCAGGGCTGCAC | variant 8, |
| | AGGCATGCCGTATCTCTCCTCTTTGTCACAACCTCTTTGCCCTGTATGACGAGAACACCAAGCTCTGGTA | mRNA |
| | TGCTCCAAATCGCACCATCACCGTTGATGACAAGATGTCCCTCCGGCTCCACTACCGGATGAGGTTCTAT | |
| | TTCACCAATTGGCATGGAACCAACGACAATGAGCAGTCAGTGTGGCGTCATTCTCCAAAGAAGCAGAAAA | |
| | ATGGCTACGAGAAAAAAAAGATTCCAGATGCAACCCCTCTCCTTGATGCCAGCTCACTGGAGTATCTGTT | |
| | TGCTCAGGGACAGTATGATTTGGTGAAATGCCTGGCTCCTATTCGAGACCCCAAGACCGAGCAGGATGGA | |
| | CATGATATTGAGAACGAGTGTCTAGGGATGGCTGTCCTGGCCATCTCACACTATGCCATGATGAAGAAGA | |
| | TGCAGTTGCCAGAACTGCCCAAGGACATCAGCTACAAGCGATATATTCCAGAAACATTGAATAAGTCCAT | |
| | CAGACAGAGGAACCTTCTCACCAGGATGCGGATAAATAATGTTTTCAAGGATTTCCTAAAGGAATTTAAC | |
| | AACAAGACCATTTGTGACAGCAGCGTGTCCACGCATGACCTGAAGGTGAAATACTTGGCTACCTTGGAAA | |
| | CTTTGACAAAACATTACGGTGCTGAAATATTTGAGACTTCCATGTTACTGATTTCATCAGAAAATGAGAT | |
| | GAATTGGTTTCATTCGAATGACGGTGGAAACGTTCTCTACTACGAAGTGATGGTGACTGGGAATCTTGGA | |
| | ATCCAGTGGAGGCATAAACCAAATGTTGTTTCTGTTGAAAAGGAAAAAAATAAACTGAAGCGGAAAAAAC | |
| | TGGAAAATAAACACAAGAAGGATGAGGAGAAAAACAAGATCCGGGAAGAGTGGAACAATTTTTCTTACTT | |
| | CCCTGAAATCACTCACATTGTAATAAAGGAGTCTGTGGTCAGCATTAACAAGCAGGACAACAAGAAAATG | |
| | GAACTGAAGCTCTCTTCCCACGAGGAGGCCTTGTCCTTTGTGTCCCTGGTAGATGGCTACTTCCGGCTCA | |
| | CAGCAGATGCCCATCATTACCTCTGCACCGACGTGGCCCCCCCGTTGATCGTCCACAACATACAGAATGG | |
| | CTGTCATGGTCCAATCTGTACAGAATACGCCATCAATAAATTGCGGCAAGAAGGAAGCGAGGAGGGGATG | |
| | TACGTGCTGAGGTGGAGCTGCACCGACTTTGACAACATCCTCATGACCGTCACCTGCTTTGAGAAGTCTG | |
| | AGGTGCAGGGTGCCCAGAAGCAGTTCAAGAACTTTCAGATCGAGGTGCAGAAGGGCCGCTACAGTCTGCA | |
| | CGGTTCGGACCGCAGCTTCCCCAGCTTGGGAGACCTCATGAGCCACCTCAAGAAGCAGATCCTGCGCACG | |
| | GATAACATCAGCTTCATGCTAAAACGCTGCTGCCAGCCCAAGCCCCGAGAAATCTCCAACCTGCTGGTGG | |
| | CTACTAAGAAAGCCCAGGAGTGGCAGCCCGTCTACCCCATGAGCCAGCTGAGTTTCGATCGGATCCTCAA | |
| | GAAGGATCTGGTGCAGGGCGAGCACCTTGGGAGAGGCACGAGAACACACATCTATTCTGGGACCCTGATG | |
| | GATTACAAGGATGACGAAGGAACTTCTGAAGAGAAGAAGATAAAAGTGATCCTCAAAGTCTTAGACCCCA | |
| | GCCACAGGGATATTTCCCTGGCCTTCTTCGAGGCAGCCAGCATGATGAGACAGGTCTCCCACAAACACAT | |
| | CGTGTACCTCTATGGCGTCTGTGTCCGCGACGTGGAGAATATCATGGTGGAAGAGTTTGTGGAAGGGGGT | |
| | CCTCTGGATCTCTTCATGCACCGGAAAAGCGATGTCCTTACCACACCATGGAAATTCAAAGTTGCCAAAC | |
| | AGCTGGCCAGTGCCCTGAGCTACTTGGAGGATAAAGACCTGGTCCATGGAAATGTGTGTACTAAAAACCT | |
| | CCTCCTGGCCCGTGAGGGCATCGACAGTGAGTGTGGCCCATTCATCAAGCTCAGTGACCCCGGCATCCCC | |
| | ATTACGGTGCTGTCTAGGCAAGAATGCATTGAACGAATCCCATGGATTGCTCCTGAGTGTGTTGAGGACT | |
| | CCAAGAACCTGAGTGTGGCTGCTGACAAGTGGAGCTTTGGAACCACGCTCTGGGAAATCTGCTACAATGG | |
| | CGAGATCCCCTTGAAAGACAAGACGCTGATTGAGAAAGAGAGATTCTATGAAAGCCGGTGCAGGCCAGTG | |
| | ACACCATCATGTAAGGAGCTGGCTGACCTCATGACCCGCTGCATGAACTATGACCCCAATCAGAGGCCTT | |
| | TCTTCCGAGCCATCATGAGAGACATTAATAAGCTTGAAGAGCAGAATCCAGATATTGTTTCAGAAAAAAA | |
| | ACCAGCAACTGAAGTGGACCCCACACATTTTGAAAAGCGCTTCCTAAAGAGGATCCGTGACTTGGGAGAG | |
| | GGCCACTTTGGGAAGGTTGAGCTCTGCAGGTATGACCCCGAAGGGGACAATACAGGGGAGCAGGTGGCTG | |
| | TTAAATCTCTGAAGCCTGAGAGTGGAGGTAACCACATAGCTGATCTGAAAAAGGAAATCGAGATCTTAAG | |
| | GAACCTCTATCATGAGAACATTGTGAAGTACAAAGGAATCTGCACAGAAGACGGAGGAAATGGTATTAAG | |
| | CTCATCATGGAATTTCTGCCTTCGGGAAGCCTTAAGGAATATCTTCCAAAGAATAAGAACAAAATAAACC | |
| | TCAAACAGCAGCTAAAATATGCCGTTCAGATTTGTAAGGGGATGGACTATTTGGGTTCTCGGCAATACGT | |
| | TCACCGGGACTTGGCAGCAAGAAATGTCCTTGTTGAGAGTGAACACCAAGTGAAAATTGGAGACTTCGGT | |
| | TTAACCAAAGCAATTGAAACCGATAAGGAGTATTACACCGTCAAGGATGACCGGGACAGCCCTGTGTTTT | |
| | GGTATGCTCCAGAATGTTTAATGCAATCTAAATTTTATATTGCCTCTGACGTCTGGTCTTTTGGAGTCAC | |
| | TCTGCATGAGCTGCTGACTTACTGTGATTCAGATTCTAGTCCCATGGCTTTGTTCCTGAAAATGATAGGC | |
| | CCAACCCATGGCCAGATGACAGTCACAAGACTTGTGAATACGTTAAAAGAAGGAAAACGCCTGCCGTGCC | |
| | CACCTAACTGTCCAGATGAGGTTTATCAACTTATGAGGAAATGCTGGGAATTCCAACCATCCAATCGGAC | |
| | AAGCTTTCAGAACCTTATTGAAGGATTTGAAGCACTTTTAAAATAAGAAGCATGAATAACATTTAAATTC | |
| | CACAGATTATCAAGTCCTTCTCCTGCAACAAATGCCCAAGTCATTTTTTAAAAATTTCTAATGAAAGAAG | |
| | TTTGTGTTCTGTCCAAAAAGTCACTGAACTCATACTTCAGTACATATACATGTATAAGGCACACTGTAGT | |
| | GCTTAATATGTGTAAGGACTTCCTCTTTAAATTTGGTACCAGTAACTTAGTGACACATAATGACAACCAA | |
| | AATATTTGAAAGCACTTAAGCACTCCTCCTTGTGGAAAGAATATACCACCATTTCATCTGGCTAGTTCAC | |
| | CATCACAACTGCATTACCAAAAGGGGATTTTTGAAAACGAGGAGTTGACCAAAATAATATCTGAAGATGA | |
| | TTGCTTTTCCCTGCTGCCAGCTGATCTGAAATGTTTTGCTGGCACATTAATCATAGATAAAGAAAGATTG | |
| | ATGGACTTAGCCCTCAAATTTCAGTATCTATACAGTACTAGACCATGCATTCTTAAAATATTAGATACCA | |
| | GGTAGTATATATTGTTTCTGTACAAAAATGACTGTATTCTCTCACCAGTAGGACTTAAACTTTGTTTCTC | |
| | CAGTGGCTTAGCTCCTGTTCCTTTGGGTGATCACTAGCACCCATTTTTGAGAAAGCTGGTTCTACATGGG | |
| | GGGATAGCTGTGGAATAGATAATTTGCTGCATGTTAATTCTCAAGAACTAAGCCTGTGCCAGTGCTTTCC | |
| | TAAGCAGTATACCTTTAATCAGAACTCATTCCCAGAACCTGGATGCTATTACACATGCTTTTAAGAAACG | |
| | TCAATGTATATCCTTTTATAACTCTACCACTTTGGGGCAAGCTATTCCAGCACTGGTTTTGAATGCTGTA | |
| | TGCAACCAGTCTGAATACCACATACGCTGCACTGTTCTTAGAGGGTTTCCATACTTACCACCGATCTACA | |
| | AGGGTTGATCCCTGTTTTTACCATCAATCATCACCCTGTGGTGCAACACTTGAAAGACCCGGCTAGAGGC | |
| | ACTATGGACTTCAGGATCCACTAGACAGTTTTCAGTTTGCTTGGAGGTAGCTGGGTAATCAAAAATGTTT | |
| | AGTCATTGATTCAATGTGAACGATTACGGTCTTTATGACCAAGAGTCTGAAAATCTTTTTGTTATGCTGT | |
| | TTAGTATTCGTTTGATATTGTTACTTTTCACCTGTTGAGCCCAAATTCAGGATTGGTTCAGTGGCAGCAA | |
| | TGAAGTTGCCATTTAAATTTGTTCATAGCCTACATCACCAAGGTCTCTGTGTCAAACCTGTGGCCACTCT | |
| | ATATGCACTTTGTTTACTCTTTATACAAATAAATATACTAAAGACTTTA | |
| 39 | GCGTCGCTGAGCGCAGGCCGCGGCGGCCGCGGAGTATCCTGGAGCTGCAGACAGTGCGGGCCTGCGCCCA | >NM_002227.4 |
| | GTCCCGGCTGTCCTCGCCGCGACCCCTCCTCAGCCCTGGGCGCGCGCACGCTGGGGCCCCGCGGGGCTGG | Homo sapiens |
| | CCGCCTAGCGAGCCTGCCGGTCGACCCCAGCCAGCGCAGCGACGGGGCGCTGCCTGGCCCAGGCGCACAC | Janus kinase 1 |
| | GGAAGTGCGCTTCTCTGAAGTAGCTTTGGAAAGTAGAGAAGAAAATCCAGTTTGCTTCTTGGAGAACACT | (JAK1), |
| | GGACAGCTGAATAAATGCAGTATCTAAATATAAAAGAGGACTGCAATGCCATGGCTTTCTGTGCTAAAAT | transcript |
| | GAGGAGCTCCAAGAAGACTGAGGTGAACCTGGAGGCCCCTGAGCCAGGGGTGGAAGTGATCTTCTATCTG | variant 1, |
| | TCGGACAGGGAGCCCCTCCGGCTGGGCAGTGGAGAGTACACAGCAGAGGAACTGTGCATCAGGGCTGCAC | mRNA |
| | AGGCATGCCGTATCTCTCCTCTTTGTCACAACCTCTTTGCCCTGTATGACGAGAACACCAAGCTCTGGTA | |
| | TGCTCCAAATCGCACCATCACCGTTGATGACAAGATGTCCCTCCGGCTCCACTACCGGATGAGGTTCTAT | |
| | TTCACCAATTGGCATGGAACCAACGACAATGAGCAGTCAGTGTGGCGTCATTCTCCAAAGAAGCAGAAAA | |
| | ATGGCTACGAGAAAAAAAAGATTCCAGATGCAACCCCTCTCCTTGATGCCAGCTCACTGGAGTATCTGTT | |
| | TGCTCAGGGACAGTATGATTTGGTGAAATGCCTGGCTCCTATTCGAGACCCCAAGACCGAGCAGGATGGA | |
| | CATGATATTGAGAACGAGTGTCTAGGGATGGCTGTCCTGGCCATCTCACACTATGCCATGATGAAGAAGA | |
| | TGCAGTTGCCAGAACTGCCCAAGGACATCAGCTACAAGCGATATATTCCAGAAACATTGAATAAGTCCAT | |
| | CAGACAGAGGAACCTTCTCACCAGGATGCGGATAAATAATGTTTTCAAGGATTTCCTAAAGGAATTTAAC | |
| | AACAAGACCATTTGTGACAGCAGCGTGTCCACGCATGACCTGAAGGTGAAATACTTGGCTACCTTGGAAA | |
| | CTTTGACAAAACATTACGGTGCTGAAATATTTGAGACTTCCATGTTACTGATTTCATCAGAAAATGAGAT | |
| | GAATTGGTTTCATTCGAATGACGGTGGAAACGTTCTCTACTACGAAGTGATGGTGACTGGGAATCTTGGA | |
| | ATCCAGTGGAGGCATAAACCAAATGTTGTTTCTGTTGAAAAGGAAAAAAATAAACTGAAGCGGAAAAAAC | |
| | TGGAAAATAAACACAAGAAGGATGAGGAGAAAAACAAGATCCGGGAAGAGTGGAACAATTTTTCTTACTT | |
| | CCCTGAAATCACTCACATTGTAATAAAGGAGTCTGTGGTCAGCATTAACAAGCAGGACAACAAGAAAATG | |
| | GAACTGAAGCTCTCTTCCCACGAGGAGGCCTTGTCCTTTGTGTCCCTGGTAGATGGCTACTTCCGGCTCA | |
| | CAGCAGATGCCCATCATTACCTCTGCACCGACGTGGCCCCCCCGTTGATCGTCCACAACATACAGAATGG | |
| | CTGTCATGGTCCAATCTGTACAGAATACGCCATCAATAAATTGCGGCAAGAAGGAAGCGAGGAGGGGATG | |
| | TACGTGCTGAGGTGGAGCTGCACCGACTTTGACAACATCCTCATGACCGTCACCTGCTTTGAGAAGTCTG | |
| | AGCAGGTGCAGGGTGCCCAGAAGCAGTTCAAGAACTTTCAGATCGAGGTGCAGAAGGGCCGCTACAGTCT | |
| | GCACGGTTCGGACCGCAGCTTCCCCAGCTTGGGAGACCTCATGAGCCACCTCAAGAAGCAGATCCTGCGC | |
| | ACGGATAACATCAGCTTCATGCTAAAACGCTGCTGCCAGCCCAAGCCCCGAGAAATCTCCAACCTGCTGG | |
| | TGGCTACTAAGAAAGCCCAGGAGTGGCAGCCCGTCTACCCCATGAGCCAGCTGAGTTTCGATCGGATCCT | |
| | CAAGAAGGATCTGGTGCAGGGCGAGCACCTTGGGAGAGGCACGAGAACACACATCTATTCTGGGACCCTG | |
| | ATGGATTACAAGGATGACGAAGGAACTTCTGAAGAGAAGAAGATAAAAGTGATCCTCAAAGTCTTAGACC | |
| | CCAGCCACAGGGATATTTCCCTGGCCTTCTTCGAGGCAGCCAGCATGATGAGACAGGTCTCCCACAAACA | |
| | CATCGTGTACCTCTATGGCGTCTGTGTCCGCGACGTGGAGAATATCATGGTGGAAGAGTTTGTGGAAGGG | |
| | GGTCCTCTGGATCTCTTCATGCACCGGAAAAGCGATGTCCTTACCACACCATGGAAATTCAAAGTTGCCA | |
| | AACAGCTGGCCAGTGCCCTGAGCTACTTGGAGGATAAAGACCTGGTCCATGGAAATGTGTGTACTAAAAA | |
| | CCTCCTCCTGGCCCGTGAGGGCATCGACAGTGAGTGTGGCCCATTCATCAAGCTCAGTGACCCCGGCATC | |
| | CCCATTACGGTGCTGTCTAGGCAAGAATGCATTGAACGAATCCCATGGATTGCTCCTGAGTGTGTTGAGG | |
| | ACTCCAAGAACCTGAGTGTGGCTGCTGACAAGTGGAGCTTTGGAACCACGCTCTGGGAAATCTGCTACAA | |
| | TGGCGAGATCCCCTTGAAAGACAAGACGCTGATTGAGAAAGAGAGATTCTATGAAAGCCGGTGCAGGCCA | |
| | GTGACACCATCATGTAAGGAGCTGGCTGACCTCATGACCCGCTGCATGAACTATGACCCCAATCAGAGGC | |
| | CTTTCTTCCGAGCCATCATGAGAGACATTAATAAGCTTGAAGAGCAGAATCCAGATATTGTTTCAGAAAA | |
| | AAAACCAGCAACTGAAGTGGACCCCACACATTTTGAAAAGCGCTTCCTAAAGAGGATCCGTGACTTGGGA | |
| | GAGGGCCACTTTGGGAAGGTTGAGCTCTGCAGGTATGACCCCGAAGGGGACAATACAGGGGAGCAGGTGG | |
| | CTGTTAAATCTCTGAAGCCTGAGAGTGGAGGTAACCACATAGCTGATCTGAAAAAGGAAATCGAGATCTT | |
| | AAGGAACCTCTATCATGAGAACATTGTGAAGTACAAAGGAATCTGCACAGAAGACGGAGGAAATGGTATT | |
| | AAGCTCATCATGGAATTTCTGCCTTCGGGAAGCCTTAAGGAATATCTTCCAAAGAATAAGAACAAAATAA | |
| | ACCTCAAACAGCAGCTAAAATATGCCGTTCAGATTTGTAAGGGGATGGACTATTTGGGTTCTCGGCAATA | |
| | CGTTCACCGGGACTTGGCAGCAAGAAATGTCCTTGTTGAGAGTGAACACCAAGTGAAAATTGGAGACTTC | |
| | GGTTTAACCAAAGCAATTGAAACCGATAAGGAGTATTACACCGTCAAGGATGACCGGGACAGCCCTGTGT | |
| | TTTGGTATGCTCCAGAATGTTTAATGCAATCTAAATTTTATATTGCCTCTGACGTCTGGTCTTTTGGAGT | |
| | CACTCTGCATGAGCTGCTGACTTACTGTGATTCAGATTCTAGTCCCATGGCTTTGTTCCTGAAAATGATA | |
| | GGCCCAACCCATGGCCAGATGACAGTCACAAGACTTGTGAATACGTTAAAAGAAGGAAAACGCCTGCCGT | |
| | GCCCACCTAACTGTCCAGATGAGGTTTATCAACTTATGAGGAAATGCTGGGAATTCCAACCATCCAATCG | |
| | GACAAGCTTTCAGAACCTTATTGAAGGATTTGAAGCACTTTTAAAATAAGAAGCATGAATAACATTTAAA | |
| | TTCCACAGATTATCAAGTCCTTCTCCTGCAACAAATGCCCAAGTCATTTTTTAAAAATTTCTAATGAAAG | |
| | AAGTTTGTGTTCTGTCCAAAAAGTCACTGAACTCATACTTCAGTACATATACATGTATAAGGCACACTGT | |
| | AGTGCTTAATATGTGTAAGGACTTCCTCTTTAAATTTGGTACCAGTAACTTAGTGACACATAATGACAAC | |
| | CAAAATATTTGAAAGCACTTAAGCACTCCTCCTTGTGGAAAGAATATACCACCATTTCATCTGGCTAGTT | |
| | CACCATCACAACTGCATTACCAAAAGGGGATTTTTGAAAACGAGGAGTTGACCAAAATAATATCTGAAGA | |
| | TGATTGCTTTTCCCTGCTGCCAGCTGATCTGAAATGTTTTGCTGGCACATTAATCATAGATAAAGAAAGA | |
| | TTGATGGACTTAGCCCTCAAATTTCAGTATCTATACAGTACTAGACCATGCATTCTTAAAATATTAGATA | |
| | CCAGGTAGTATATATTGTTTCTGTACAAAAATGACTGTATTCTCTCACCAGTAGGACTTAAACTTTGTTT | |
| | CTCCAGTGGCTTAGCTCCTGTTCCTTTGGGTGATCACTAGCACCCATTTTTGAGAAAGCTGGTTCTACAT | |
| | GGGGGGATAGCTGTGGAATAGATAATTTGCTGCATGTTAATTCTCAAGAACTAAGCCTGTGCCAGTGCTT | |
| | TCCTAAGCAGTATACCTTTAATCAGAACTCATTCCCAGAACCTGGATGCTATTACACATGCTTTTAAGAA | |
| | ACGTCAATGTATATCCTTTTATAACTCTACCACTTTGGGGCAAGCTATTCCAGCACTGGTTTTGAATGCT | |
| | GTATGCAACCAGTCTGAATACCACATACGCTGCACTGTTCTTAGAGGGTTTCCATACTTACCACCGATCT | |
| | ACAAGGGTTGATCCCTGTTTTTACCATCAATCATCACCCTGTGGTGCAACACTTGAAAGACCCGGCTAGA | |
| | GGCACTATGGACTTCAGGATCCACTAGACAGTTTTCAGTTTGCTTGGAGGTAGCTGGGTAATCAAAAATG | |
| | TTTAGTCATTGATTCAATGTGAACGATTACGGTCTTTATGACCAAGAGTCTGAAAATCTTTTTGTTATGC | |
| | TGTTTAGTATTCGTTTGATATTGTTACTTTTCACCTGTTGAGCCCAAATTCAGGATTGGTTCAGTGGCAG | |
| | CAATGAAGTTGCCATTTAAATTTGTTCATAGCCTACATCACCAAGGTCTCTGTGTCAAACCTGTGGCCAC | |
| | TCTATATGCACTTTGTTTACTCTTTATACAAATAAATATACTAAAGACTTTA | |
| 40 | MQYLNIKEDCNAMAFCAKMRSSKKTEVNLEAPEPGVEVIFYLSDREPLRLGSGEYTAEELCIRAAQACRI | >NP_001307852.1 |
| | SPLCHNLFALYDENTKLWYAPNRTITVDDKMSLRLHYRMRFYFTNWHGTNDNEQSVWRHSPKKQKNGYEK | tyrosine- |
| | KKIPDATPLLDASSLEYLFAQGQYDLVKCLAPIRDPKTEQDGHDIENECLGMAVLAISHYAMMKKMQLPE | protein |
| | LPKDISYKRYIPETLNKSIRQRNLLTRMRINNVEKDFLKEENNKTICDSSVSTHDLKVKYLATLETLTKH | kinase JAK1 |
| | YGAEIFETSMLLISSENEMNWFHSNDGGNVLYYEVMVTGNLGIQWRHKPNVVSVEKEKNKLKRKKLENKH | isoform 1 |
| | KKDEEKNKIREEWNNFSYFPEITHIVIKESVVSINKQDNKKMELKLSSHEEALSFVSLVDGYFRLTADAH | [Homo |
| | HYLCTDVAPPLIVHNIQNGCHGPICTEYAINKLRQEGSEEGMYVLRWSCTDFDNILMTVTCFEKSEQVQG | sapiens] |
| | AQKQFKNFQIEVQKGRYSLHGSDRSFPSLGDLMSHLKKQILRTDNISFMLKRCCQPKPREISNLLVATKK | |
| | AQEWQPVYPMSQLSFDRILKKDLVQGEHLGRGTRTHIYSGTLMDYKDDEGTSEEKKIKVILKVLDPSHRD | |
| | ISLAFFEAASMMRQVSHKHIVYLYGVCVRDVENIMVEEFVEGGPLDLFMHRKSDVLTTPWKFKVAKQLAS | |
| | ALSYLEDKDLVHGNVCTKNLLLAREGIDSECGPFIKLSDPGIPITVLSRQECIERIPWIAPECVEDSKNL | |
| | SVAADKWSEGTTLWEICYNGEIPLKDKTLIEKERFYESRCRPVTPSCKELADLMTRCMNYDPNQRPFFRA | |
| | IMRDINKLEEQNPDIVSEKKPATEVDPTHFEKRFLKRIRDLGEGHFGKVELCRYDPEGDNTGEQVAVKSL | |
| | KPESGGNHIADLKKEIEILRNLYHENIVKYKGICTEDGGNGIKLIMEFLPSGSLKEYLPKNKNKINLKQQ | |
| | LKYAVQICKGMDYLGSRQYVHRDLAARNVLVESEHQVKIGDFGLTKAIETDKEYYTVKDDRDSPVFWYAP | |
| | ECLMQSKFYIASDVWSEGVTLHELLTYCDSDSSPMALFLKMIGPTHGQMTVTRLVNTLKEGKRLPCPPNC | |
| | PDEVYQLMRKCWEFQPSNRTSFQNLIEGFEALLK | |
| 41 | MQYLNIKEDCNAMAFCAKMRSSKKTEVNLEAPEPGVEVIFYLSDREPLRLGSGEYTAEELCIRAAQACRI | >NP_001308781.1 |
| | SPLCHNLFALYDENTKLWYAPNRTITVDDKMSLRLHYRMRFYFTNWHGTNDNEQSVWRHSPKKQKNGYEK | tyrosine- |
| | KKIPDATPLLDASSLEYLFAQGQYDLVKCLAPIRDPKTEQDGHDIENECLGMAVLAISHYAMMKKMQLPE | protein |
| | LPKDISYKRYIPETLNKSIRQRNLLTRMRINNVEKDFLKEENNKTICDSSVSTHDLKVKYLATLETLTKH | kinase JAK1 |
| | YGAEIFETSMLLISSENEMNWFHSNDGGNVLYYEVMVTGNLGIQWRHKPNVVSVEKEKNKLKRKKLENKH | isoform 1 |
| | KKDEEKNKIREEWNNFSYFPEITHIVIKESVVSINKQDNKKMELKLSSHEEALSFVSLVDGYFRLTADAH | [Homo |
| | HYLCTDVAPPLIVHNIQNGCHGPICTEYAINKLRQEGSEEGMYVLRWSCTDFDNILMTVTCFEKSEQVQG | sapiens] |
| | AQKQFKNFQIEVQKGRYSLHGSDRSFPSLGDLMSHLKKQILRTDNISFMLKRCCQPKPREISNLLVATKK | |
| | AQEWQPVYPMSQLSFDRILKKDLVQGEHLGRGTRTHIYSGTLMDYKDDEGTSEEKKIKVILKVLDPSHRD | |
| | ISLAFFEAASMMRQVSHKHIVYLYGVCVRDVENIMVEEFVEGGPLDLFMHRKSDVLTTPWKFKVAKQLAS | |
| | ALSYLEDKDLVHGNVCTKNLLLAREGIDSECGPFIKLSDPGIPITVLSRQECIERIPWIAPECVEDSKNL | |
| | SVAADKWSEGTTLWEICYNGEIPLKDKTLIEKERFYESRCRPVTPSCKELADLMTRCMNYDPNQRPFFRA | |
| | IMRDINKLEEQNPDIVSEKKPATEVDPTHFEKRFLKRIRDLGEGHFGKVELCRYDPEGDNTGEQVAVKSL | |
| | KPESGGNHIADLKKEIEILRNLYHENIVKYKGICTEDGGNGIKLIMEFLPSGSLKEYLPKNKNKINLKQQ | |
| | LKYAVQICKGMDYLGSRQYVHRDLAARNVLVESEHQVKIGDFGLTKAIETDKEYYTVKDDRDSPVFWYAP | |
| | ECLMQSKFYIASDVWSEGVTLHELLTYCDSDSSPMALFLKMIGPTHGQMTVTRLVNTLKEGKRLPCPPNC | |
| | PDEVYQLMRKCWEFQPSNRTSFQNLIEGFEALLK | |
| 42 | MQYLNIKEDCNAMAFCAKMRSSKKTEVNLEAPEPGVEVIFYLSDREPLRLGSGEYTAEELCIRAAQACRI | >NP_001308782.1 |
| | SPLCHNLFALYDENTKLWYAPNRTITVDDKMSLRLHYRMRFYFTNWHGTNDNEQSVWRHSPKKQKNGYEK | tyrosine- |
| | KKIPDATPLLDASSLEYLFAQGQYDLVKCLAPIRDPKTEQDGHDIENECLGMAVLAISHYAMMKKMQLPE | protein |
| | LPKDISYKRYIPETLNKSIRQRNLLTRMRINNVEKDFLKEENNKTICDSSVSTHDLKVKYLATLETLTKH | kinase JAK1 |
| | YGAEIFETSMLLISSENEMNWFHSNDGGNVLYYEVMVTGNLGIQWRHKPNVVSVEKEKNKLKRKKLENKH | isoform 1 |
| | KKDEEKNKIREEWNNFSYFPEITHIVIKESVVSINKQDNKKMELKLSSHEEALSFVSLVDGYFRLTADAH | [Homo |
| | HYLCTDVAPPLIVHNIQNGCHGPICTEYAINKLRQEGSEEGMYVLRWSCTDFDNILMTVTCFEKSEQVQG | sapiens] |
| | AQKQFKNFQIEVQKGRYSLHGSDRSFPSLGDLMSHLKKQILRTDNISFMLKRCCQPKPREISNLLVATKK | |
| | AQEWQPVYPMSQLSFDRILKKDLVQGEHLGRGTRTHIYSGTLMDYKDDEGTSEEKKIKVILKVLDPSHRD | |
| | ISLAFFEAASMMRQVSHKHIVYLYGVCVRDVENIMVEEFVEGGPLDLFMHRKSDVLTTPWKFKVAKQLAS | |
| | ALSYLEDKDLVHGNVCTKNLLLAREGIDSECGPFIKLSDPGIPITVLSRQECIERIPWIAPECVEDSKNL | |
| | SVAADKWSEGTTLWEICYNGEIPLKDKTLIEKERFYESRCRPVTPSCKELADLMTRCMNYDPNQRPFFRA | |
| | IMRDINKLEEQNPDIVSEKKPATEVDPTHFEKRFLKRIRDLGEGHFGKVELCRYDPEGDNTGEQVAVKSL | |
| | KPESGGNHIADLKKEIEILRNLYHENIVKYKGICTEDGGNGIKLIMEFLPSGSLKEYLPKNKNKINLKQQ | |
| | LKYAVQICKGMDYLGSRQYVHRDLAARNVLVESEHQVKIGDFGLTKAIETDKEYYTVKDDRDSPVFWYAP | |
| | ECLMQSKFYIASDVWSEGVTLHELLTYCDSDSSPMALFLKMIGPTHGQMTVTRLVNTLKEGKRLPCPPNC | |
| | PDEVYQLMRKCWEFQPSNRTSFQNLIEGFEALLK | |
| | MQYLNIKEDCNAMAFCAKMRSSKKTEVNLEAPEPGVEVIFYLSDREPLRLGSGEYTAEELCIRAAQACRI | >NP_001308783.1 |
| | SPLCHNLFALYDENTKLWYAPNRTITVDDKMSLRLHYRMRFYFTNWHGTNDNEQSVWRHSPKKQKNGYEK | tyrosine- |
| | KKIPDATPLLDASSLEYLFAQGQYDLVKCLAPIRDPKTEQDGHDIENECLGMAVLAISHYAMMKKMQLPE | protein |
| | LPKDISYKRYIPETLNKSIRQRNLLTRMRINNVEKDFLKEENNKTICDSSVSTHDLKVKYLATLETLTKH | kinase JAK1 |
| | YGAEIFETSMLLISSENEMNWFHSNDGGNVLYYEVMVTGNLGIQWRHKPNVVSVEKEKNKLKRKKLENKH | isoform 1 |
| | KKDEEKNKIREEWNNFSYFPEITHIVIKESVVSINKQDNKKMELKLSSHEEALSFVSLVDGYFRLTADAH | [Homo |
| | HYLCTDVAPPLIVHNIQNGCHGPICTEYAINKLRQEGSEEGMYVLRWSCTDFDNILMTVTCFEKSEQVQG | sapiens] |
| | AQKQFKNFQIEVQKGRYSLHGSDRSFPSLGDLMSHLKKQILRTDNISFMLKRCCQPKPREISNLLVATKK | |
| | AQEWQPVYPMSQLSFDRILKKDLVQGEHLGRGTRTHIYSGTLMDYKDDEGTSEEKKIKVILKVLDPSHRD | |
| | ISLAFFEAASMMRQVSHKHIVYLYGVCVRDVENIMVEEFVEGGPLDLFMHRKSDVLTTPWKFKVAKQLAS | |
| | ALSYLEDKDLVHGNVCTKNLLLAREGIDSECGPFIKLSDPGIPITVLSRQECIERIPWIAPECVEDSKNL | |
| | SVAADKWSEGTTLWEICYNGEIPLKDKTLIEKERFYESRCRPVTPSCKELADLMTRCMNYDPNQRPFFRA | |
| | IMRDINKLEEQNPDIVSEKKPATEVDPTHFEKRFLKRIRDLGEGHFGKVELCRYDPEGDNTGEQVAVKSL | |
| | KPESGGNHIADLKKEIEILRNLYHENIVKYKGICTEDGGNGIKLIMEFLPSGSLKEYLPKNKNKINLKQQ | |
| | LKYAVQICKGMDYLGSRQYVHRDLAARNVLVESEHQVKIGDFGLTKAIETDKEYYTVKDDRDSPVFWYAP | |
| | ECLMQSKFYIASDVWSEGVTLHELLTYCDSDSSPMALFLKMIGPTHGQMTVTRLVNTLKEGKRLPCPPNC | |
| | PDEVYQLMRKCWEFQPSNRTSFQNLIEGFEALLK | |
| 43 | MQYLNIKEDCNAMAFCAKMRSSKKTEVNLEAPEPGVEVIFYLSDREPLRLGSGEYTAEELCIRAAQACRI | >NP_001308784.1 |
| | SPLCHNLFALYDENTKLWYAPNRTITVDDKMSLRLHYRMRFYFTNWHGTNDNEQSVWRHSPKKQKNGYEK | tyrosine- |
| | KKIPDATPLLDASSLEYLFAQGQYDLVKCLAPIRDPKTEQDGHDIENECLGMAVLAISHYAMMKKMQLPE | protein |
| | LPKDISYKRYIPETLNKSIRQRNLLTRMRINNVEKDFLKEENNKTICDSSVSTHDLKVKYLATLETLTKH | kinase JAK1 |
| | YGAEIFETSMLLISSENEMNWFHSNDGGNVLYYEVMVTGNLGIQWRHKPNVVSVEKEKNKLKRKKLENKH | isoform 1 |
| | KKDEEKNKIREEWNNFSYFPEITHIVIKESVVSINKQDNKKMELKLSSHEEALSFVSLVDGYFRLTADAH | [Homo |
| | HYLCTDVAPPLIVHNIQNGCHGPICTEYAINKLRQEGSEEGMYVLRWSCTDFDNILMTVTCFEKSEQVQG | sapiens] |
| | AQKQFKNFQIEVQKGRYSLHGSDRSFPSLGDLMSHLKKQILRTDNISFMLKRCCQPKPREISNLLVATKK | |
| | AQEWQPVYPMSQLSFDRILKKDLVQGEHLGRGTRTHIYSGTLMDYKDDEGTSEEKKIKVILKVLDPSHRD | |
| | ISLAFFEAASMMRQVSHKHIVYLYGVCVRDVENIMVEEFVEGGPLDLFMHRKSDVLTTPWKFKVAKQLAS | |
| | ALSYLEDKDLVHGNVCTKNLLLAREGIDSECGPFIKLSDPGIPITVLSRQECIERIPWIAPECVEDSKNL | |
| | SVAADKWSEGTTLWEICYNGEIPLKDKTLIEKERFYESRCRPVTPSCKELADLMTRCMNYDPNQRPFFRA | |
| | IMRDINKLEEQNPDIVSEKKPATEVDPTHFEKRFLKRIRDLGEGHFGKVELCRYDPEGDNTGEQVAVKSL | |
| | KPESGGNHIADLKKEIEILRNLYHENIVKYKGICTEDGGNGIKLIMEFLPSGSLKEYLPKNKNKINLKQQ | |
| | LKYAVQICKGMDYLGSRQYVHRDLAARNVLVESEHQVKIGDFGLTKAIETDKEYYTVKDDRDSPVFWYAP | |
| | ECLMQSKFYIASDVWSEGVTLHELLTYCDSDSSPMALFLKMIGPTHGQMTVTRLVNTLKEGKRLPCPPNC | |
| | PDEVYQLMRKCWEFQPSNRTSFQNLIEGFEALLK | |
| 44 | MQYLNIKEDCNAMAFCAKMRSSKKTEVNLEAPEPGVEVIFYLSDREPLRLGSGEYTAEELCIRAAQACRI | >NP_001308785.1 |
| | SPLCHNLFALYDENTKLWYAPNRTITVDDKMSLRLHYRMRFYFTNWHGTNDNEQSVWRHSPKKQKNGYEK | tyrosine- |
| | KKIPDATPLLDASSLEYLFAQGQYDLVKCLAPIRDPKTEQDGHDIENECLGMAVLAISHYAMMKKMQLPE | protein |
| | LPKDISYKRYIPETLNKSIRQRNLLTRMRINNVEKDFLKEENNKTICDSSVSTHDLKVKYLATLETLTKH | kinase JAK1 |
| | YGAEIFETSMLLISSENEMNWFHSNDGGNVLYYEVMVTGNLGIQWRHKPNVVSVEKEKNKLKRKKLENKH | isoform 1 |
| | KKDEEKNKIREEWNNFSYFPEITHIVIKESVVSINKQDNKKMELKLSSHEEALSFVSLVDGYFRLTADAH | [Homo |
| | HYLCTDVAPPLIVHNIQNGCHGPICTEYAINKLRQEGSEEGMYVLRWSCTDFDNILMTVTCFEKSEQVQG | sapiens] |
| | AQKQFKNFQIEVQKGRYSLHGSDRSFPSLGDLMSHLKKQILRTDNISFMLKRCCQPKPREISNLLVATKK | |
| | AQEWQPVYPMSQLSFDRILKKDLVQGEHLGRGTRTHIYSGTLMDYKDDEGTSEEKKIKVILKVLDPSHRD | |
| | ISLAFFEAASMMRQVSHKHIVYLYGVCVRDVENIMVEEFVEGGPLDLFMHRKSDVLTTPWKFKVAKQLAS | |
| | ALSYLEDKDLVHGNVCTKNLLLAREGIDSECGPFIKLSDPGIPITVLSRQECIERIPWIAPECVEDSKNL | |
| | SVAADKWSEGTTLWEICYNGEIPLKDKTLIEKERFYESRCRPVTPSCKELADLMTRCMNYDPNQRPFFRA | |
| | IMRDINKLEEQNPDIVSEKKPATEVDPTHFEKRFLKRIRDLGEGHFGKVELCRYDPEGDNTGEQVAVKSL | |
| | KPESGGNHIADLKKEIEILRNLYHENIVKYKGICTEDGGNGIKLIMEFLPSGSLKEYLPKNKNKINLKQQ | |
| | LKYAVQICKGMDYLGSRQYVHRDLAARNVLVESEHQVKIGDFGLTKAIETDKEYYTVKDDRDSPVFWYAP | |
| | ECLMQSKFYIASDVWSEGVTLHELLTYCDSDSSPMALFLKMIGPTHGQMTVTRLVNTLKEGKRLPCPPNC | |
| | PDEVYQLMRKCWEFQPSNRTSFQNLIEGFEALLK | |
| 45 | MQYLNIKEDCNAMAFCAKMRSSKKTEVNLEAPEPGVEVIFYLSDREPLRLGSGEYTAEELCIRAAQACRI | >NP_001308786.1 |
| | SPLCHNLFALYDENTKLWYAPNRTITVDDKMSLRLHYRMRFYFTNWHGTNDNEQSVWRHSPKKQKNGYEK | tyrosine- |
| | KKIPDATPLLDASSLEYLFAQGQYDLVKCLAPIRDPKTEQDGHDIENECLGMAVLAISHYAMMKKMQLPE | protein |
| | LPKDISYKRYIPETLNKSIRQRNLLTRMRINNVEKDFLKEENNKTICDSSVSTHDLKVKYLATLETLTKH | kinase JAK1 |
| | YGAEIFETSMLLISSENEMNWFHSNDGGNVLYYEVMVTGNLGIQWRHKPNVVSVEKEKNKLKRKKLENKH | isoform 2 |
| | KKDEEKNKIREEWNNFSYFPEITHIVIKESVVSINKQDNKKMELKLSSHEEALSFVSLVDGYFRLTADAH | [Homo |
| | HYLCTDVAPPLIVHNIQNGCHGPICTEYAINKLRQEGSEEGMYVLRWSCTDFDNILMTVTCFEKSEVQGA | sapiens] |
| | QKQEKNFQIEVQKGRYSLHGSDRSEPSLGDLMSHLKKQILRTDNISFMLKRCCQPKPREISNLLVATKKA | |
| | QEWQPVYPMSQLSFDRILKKDLVQGEHLGRGTRTHIYSGTLMDYKDDEGTSEEKKIKVILKVLDPSHRDI | |
| | SLAFFEAASMMRQVSHKHIVYLYGVCVRDVENIMVEEFVEGGPLDLFMHRKSDVLTTPWKFKVAKQLASA | |
| | LSYLEDKDLVHGNVCTKNLLLAREGIDSECGPFIKLSDPGIPITVLSRQECIERIPWIAPECVEDSKNLS | |
| | VAADKWSEGTTLWEICYNGEIPLKDKTLIEKERFYESRCRPVTPSCKELADLMTRCMNYDPNQRPFFRAI | |
| | MRDINKLEEQNPDIVSEKKPATEVDPTHFEKRFLKRIRDLGEGHFGKVELCRYDPEGDNTGEQVAVKSLK | |
| | PESGGNHIADLKKEIEILRNLYHENIVKYKGICTEDGGNGIKLIMEFLPSGSLKEYLPKNKNKINLKQQL | |
| | KYAVQICKGMDYLGSRQYVHRDLAARNVLVESEHQVKIGDFGLTKAIETDKEYYTVKDDRDSPVFWYAPE | |
| | CLMQSKFYIASDVWSEGVTLHELLTYCDSDSSPMALFLKMIGPTHGQMTVTRLVNTLKEGKRLPCPPNCP | |
| | DEVYQLMRKCWEFQPSNRTSFQNLIEGFEALLK | |
| 46 | MQYLNIKEDCNAMAFCAKMRSSKKTEVNLEAPEPGVEVIFYLSDREPLRLGSGEYTAEELCIRAAQACRI | >NP_002218.2 |
| | SPLCHNLFALYDENTKLWYAPNRTITVDDKMSLRLHYRMRFYFTNWHGTNDNEQSVWRHSPKKQKNGYEK | tyrosine- |
| | KKIPDATPLLDASSLEYLFAQGQYDLVKCLAPIRDPKTEQDGHDIENECLGMAVLAISHYAMMKKMQLPE | protein |
| | LPKDISYKRYIPETLNKSIRQRNLLTRMRINNVEKDFLKEENNKTICDSSVSTHDLKVKYLATLETLTKH | kinase JAK1 |
| | YGAEIFETSMLLISSENEMNWFHSNDGGNVLYYEVMVTGNLGIQWRHKPNVVSVEKEKNKLKRKKLENKH | isoform 1 |
| | KKDEEKNKIREEWNNFSYFPEITHIVIKESVVSINKQDNKKMELKLSSHEEALSFVSLVDGYFRLTADAH | [Homo |
| | HYLCTDVAPPLIVHNIQNGCHGPICTEYAINKLRQEGSEEGMYVLRWSCTDFDNILMTVTCFEKSEQVQG | sapiens] |
| | AQKQFKNFQIEVQKGRYSLHGSDRSFPSLGDLMSHLKKQILRTDNISFMLKRCCQPKPREISNLLVATKK | |
| | AQEWQPVYPMSQLSFDRILKKDLVQGEHLGRGTRTHIYSGTLMDYKDDEGTSEEKKIKVILKVLDPSHRD | |
| | ISLAFFEAASMMRQVSHKHIVYLYGVCVRDVENIMVEEFVEGGPLDLFMHRKSDVLTTPWKFKVAKQLAS | |
| | ALSYLEDKDLVHGNVCTKNLLLAREGIDSECGPFIKLSDPGIPITVLSRQECIERIPWIAPECVEDSKNL | |
| | SVAADKWSEGTTLWEICYNGEIPLKDKTLIEKERFYESRCRPVTPSCKELADLMTRCMNYDPNQRPFFRA | |
| | IMRDINKLEEQNPDIVSEKKPATEVDPTHFEKRFLKRIRDLGEGHFGKVELCRYDPEGDNTGEQVAVKSL | |
| | KPESGGNHIADLKKEIEILRNLYHENIVKYKGICTEDGGNGIKLIMEFLPSGSLKEYLPKNKNKINLKQQ | |
| | LKYAVQICKGMDYLGSRQYVHRDLAARNVLVESEHQVKIGDFGLTKAIETDKEYYTVKDDRDSPVFWYAP | |
| | ECLMQSKFYIASDVWSEGVTLHELLTYCDSDSSPMALFLKMIGPTHGQMTVTRLVNTLKEGKRLPCPPNC | |
| | PDEVYQLMRKCWEFQPSNRTSFQNLIEGFEALLK | |
| 47 | GTGGGCAGCCGGCGGGCTCCGAGGCCGTGAGCGCAAAGCCTCAGGCCCCGGCTCCCTCCTGAGCTGCGCC | >NM_005866.4 |
| | GTGCCAGGCCGCCCGCCGGGATGCAGTGGGCCGTGGGCCGGCGGTGGGCGTGGGCCGCGCTGCTCCTGGC | Homo sapiens |
| | TGTCGCAGCGGTGCTGACCCAGGTCGTCTGGCTCTGGCTGGGTACGCAGAGCTTCGTCTTCCAGCGCGAA | sigma non- |
| | GAGATAGCGCAGTTGGCGCGGCAGTACGCTGGGCTGGACCACGAGCTGGCCTTCTCTCGTCTGATCGTGG | opioid |
| | AGCTGCGGCGGCTGCACCCAGGCCACGTGCTGCCCGACGAGGAGCTGCAGTGGGTGTTCGTGAATGCGGG | intracellular |
| | TGGCTGGATGGGCGCCATGTGCCTTCTGCACGCCTCGCTGTCCGAGTATGTGCTGCTCTTCGGCACCGCC | receptor 1 |
| | TTGGGCTCCCGCGGCCACTCGGGGCGCTACTGGGCTGAGATCTCGGATACCATCATCTCTGGCACCTTCC | (SIGMAR1), |
| | ACCAGTGGAGAGAGGGCACCACCAAAAGTGAGGTCTTCTACCCAGGGGAGACGGTAGTACACGGGCCTGG | transcript |
| | TGAGGCAACAGCTGTGGAGTGGGGGCCAAACACATGGATGGTGGAGTACGGCCGGGGCGTCATCCCATCC | variant 1, |
| | ACCCTGGCCTTCGCGCTGGCCGACACTGTCTTCAGCACCCAGGACTTCCTCACCCTCTTCTATACTCTTC | mRNA |
| | GCTCCTATGCTCGGGGCCTCCGGCTTGAGCTCACCACCTACCTCTTTGGCCAGGACCCTTGACCAGCCAG | |
| | GCCTGAAGGAAGACCTGCGGATAGACAGGAGCGGGCAGGCCCGCACATATCCACTTGCTGGAGCCCATGT | |
| | TTACAGACAGGGACATACACCATGCAGATCCTGAGTTCCTGCTGTATGAGCAGGGATATCCATGCTTATG | |
| | TATCCAAACACAGAGACCCATGGGAACAAATGAGACACATATAGATACTGAGACCTGTGTGTACAGTAGG | |
| | ACCATGCACTCACACCCATCTGGAGAGGGAGCCCCCGGTATACCAAGGGAGCCAGTTGTGTTCAGACACA | |
| | CACATCACAGCTTGACTCACTAACTGAGGCCTTTCCATAGCTCCACAGCTTCCCACCTCCTCCCCACCAA | |
| | ACCGGGGTTCTAGAGTTAAGGATGGGGGAGGGTATTATACTGCCTCAGTCTGACTCCTCAACCCAGCAGC | |
| | AATTTGAGGGGATGAGGGGGAAGAGGAGCTGCCTTTTGGAGGCCCCCTTCACCTGCAGCTATGATGCCCT | |
| | TCCCCTTCTCCCCTGTCCTCACCATATGCCTTATCCCCATTCTACTCCCCTGCTATGCAAGTGCCCCTGT | |
| | GGCTTGTCCCCAACCCCCTCAGCAACAAAGCTCAGCTGGGGAACGAGAGTAATTTGAAGAATGCTTGAAG | |
| | TCAGCGTCTTCCATTCCAGAAAGACCCCCATTCTTCCTTTGGGGGTATGATGTGGAAGCTGGTTTCAGCC | |
| | CAGGACCCACCACTGAGGAGAGGATCTAGACAGGTGGGCCTAATTCCAAGGGGCCCTTCCTGGCCTGGAG | |
| | AAGGCCTTTTACACACACACAACACATACACACACACACACACACACACATATCACAGTTTTCACACAGC | |
| | CCCTGCTGCATTCTCTGTCCATCTGTCTGTTTCTATTAATAAAGATTTGTTGATCTGTTCCA | |
| 48 | MQWAVGRRWANAALLLAVAAVLTQVVWLWLGTQSFVFQREEIAQLARQYAGLDHELAFSRLIVELRRLHP | >NP_005857.1 |
| | GHVLPDEELQWVEVNAGGWMGAMCLLHASLSEYVLLEGTALGSRGHSGRYWAEISDTIISGTFHQWREGT | sigma non- |
| | TKSEVFYPGETVVHGPGEATAVEWGPNTWMVEYGRGVIPSTLAFALADTVFSTQDFLTLFYTLRSYARGL | opioid |
| | RLELTTYLFGQDP | intracellular |
| | | receptor 1 |
| | | isoform 1 |
| | | [Homo sapiens] |

What is claimed:

1. A method of treating an inflammatory, fibrostenotic, or fibrotic disease or condition in a subject, the method comprising: administering a therapeutic agent to the subject based, at least in part, on an expression level of a biomarker comprising angiotensin-converting enzyme 2 (ACE2), transmembrane serine protease 2 (TMPRSS2), transmembrane serine protease 4 (TMPRSS4), solute carrier family 6 member 19 (SLC6A19), Sigma Non-Opioid Intracellular Receptor 1 (SIGMAR1), or Janus kinase 1 (JAK1), or a combination thereof, as compared to an expression level of the biomarker in a control sample obtained from a subject that does not have the inflammatory, fibrostenotic, or fibrotic disease or condition.

2. The method of claim 1, wherein the expression level of the biomarker in the biological sample is lower than the expression level of the biomarker in the control sample when the inflammatory, fibrostenotic, or fibrotic disease or condition is Crohn's disease; and wherein the expression level of the biomarker in the biological sample is higher than the expression level of the biomarker in the control sample when the inflammatory, fibrostenotic, or fibrotic disease or condition is ulcerative colitis.

3. The method of claim 1, wherein the biomarker comprises two or more biomarkers.

4. The method of claim 1, wherein the biomarker is RNA.

5. The method of claim 1, wherein the biomarker is encoded by a nucleic acid sequence that is at least 90% identical to:
(a) any one of SEQ ID NOS: 1-6 when the biomarker comprises ACE2;
(b) any one of SEQ ID NOS: 12-14 when the biomarker comprises TMPRSS2;
(c) any one of SEQ ID NOS: 18-23 when the biomarker comprises TMPRSS4;
(d) SEQ ID NO: 30 when the biomarker comprises SLC6A19;
(e) any one of SEQ ID NOS: 32-39 when the biomarker comprises JAK1; or
(f) SEQ ID NO: 47 when the biomarker comprises SIGMAR1.

6. The method of claim 1, wherein the inflammatory, fibrostenotic, or fibrotic disease or condition comprises inflammatory bowel disease (IBD), Crohn's disease (CD), or ulcerative colitis (UC), or a combination thereof.

7. The method of claim 1, wherein the expression level of the biomarker in the biological sample that is lower than the expression level of the biomarker in the control sample is indicative of the subject having a high risk of a non-response to an inhibitor of Tumor Necrosis Factor (TNF), interleukin 12 (IL-12), or interleukin 23 (IL-23) when the inflammatory, fibrostenotic, or fibrotic disease or condition is Crohn's disease; and wherein the expression level of the biomarker in the biological sample that is higher than the expression level of the biomarker in the control sample is indicative of the subject having a high risk of a non-response to an inhibitor of TNF, IL-12, or IL-23 when the inflammatory, fibrostenotic, or fibrotic disease or condition is ulcerative colitis.

8. The method of claim 7, wherein the inhibitor of IL-12 comprises ustekinumab, and the inhibitor of TNF comprises infliximab.

9. The method of claim 1, further comprising:
(a) determining that the subject has a high risk of having or developing a non-response to an inhibitor of Tumor Necrosis Factor (TNF), interleukin 12 (IL-12), or interleukin 23 (IL-23), when (i) the expression level of the biomarker in the biological sample is lower than the expression level of the biomarker in the control sample and (ii) the inflammatory, fibrostenotic, or fibrotic disease or condition is Crohn's disease; or (b) determining that the subject has a high risk of a non-response to an inhibitor of TNF, IL-12, or IL-23 when (i) the expression level of the biomarker in the biological sample is higher than the expression level of the biomarker in the control sample and (ii) the inflammatory, fibrostenotic, or fibrotic disease or condition is ulcerative colitis.

10. The method of claim 9, wherein the inhibitor of IL-12 comprises ustekinumab, and the inhibitor of TNF comprises infliximab.

11. The method of claim 1, wherein the biological sample is a tissue sample obtained from the small intestine or large intestine of the subject.

12. The method of claim 1, wherein the biological sample is a tissue sample obtained from the ileum of the subject.

13. The method of claim 1, wherein the biological sample is a tissue sample obtained from the colon.

14. The method of claim 1, wherein the expression level of the biomarker in the biological sample that is lower than the expression level of the biomarker in the control sample is indicative of a severe form of the inflammatory, fibrostenotic, or fibrotic disease or condition characterized by a high risk for (i) relapse of the inflammatory, fibrostenotic, or fibrotic disease or condition, (ii) or developing intestinal fibrosis.

15. The method of claim 1, wherein the expression level of the biomarker in the biological sample that is higher than the expression level of the biomarker in the control sample is indicative of a severe form of the inflammatory, fibrostenotic, or fibrotic disease or condition characterized by a high risk for (i) relapse of the inflammatory, fibrostenotic, or fibrotic disease or condition, or (ii) developing intestinal fibrosis.

16. The method of claim 1, wherein the expression of the biomarker is determined using quantitative polymerase chain reaction (qPCR), nucleic acid sequencing, gene array analysis, single molecule detection, immunohistochemistry (IHC), enzyme linked-immunosorbent assay (ELISA), or flow cytometry.

17. The method of claim 1, wherein the therapeutic agent is a modulator of Tumor Necrosis Factor (TNF), interleukin 12 (IL-12), interleukin 23 (IL-23), ACE2, angiotensin-converting enzyme (ACE), angiotensin-2 receptor (AGTR1), TMPRSS2, TMPRSS4, SLC6A19, or JAK1, or a combination thereof.

18. The method of claim 16, wherein the modulator of IL-12 comprises ustekinumab.

19. The method of claim 17, wherein the modulator of TNF comprises infliximab.

20. The method of claim 1, wherein the subject is a human subject.


SEQUENCE LISTING (49 sequences)

SEQ ID NO: 1 - DNA, 3339 bp, Homo sapiens
agtctagggaaagtcattcagtggatgtgatcttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactc

SEQ ID NO: 2 - DNA, 3141 bp, Homo sapiens
agtctagggaaagtcattcagtggatgtgatcttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagccaactccactcttgggaaaaagttggctgacagccatcttgaaagattgagggctgaaaatccaagaactgaggatcaagatctctcccctgtcataaaactacatatggatctgcccttcagtaggaaattcctaaaagtctcccatgagataaagaatcagtgctggaaaactcactccgataccaccaccaccaaatcatgatagaaacagctatgtgtgtctttttttaattagacctcatcttccttggaactaactctgaaagggccatgaatctcagcccccccaaaatccctccccaaaagcatgctgccaggtgatgcaggcccaagctaggtgacagatgtttaacttggaatgatgtttgcagtcatgtgataataacattggatggaacaattcagaggctgttcttatgattacaagtaatggggacatttttatcatttgagaatgactgcaaaactatggaatttggcaaagactttatttggaagcagggaagaaagcccactgaatagctttgaagggataatggagggaaagaattatgttgttttctgcttttgtcctatagagtttcatttcaacaccaggatacttccacaaagcagtcttggccatgttgatggtaaggaaagaatgacagctaataacagctgcctgttatgtgtgatgccatcttaaggacatctcccgcatgcacccattttttctttttttttttttggtgactatttatgggcttactggctaggaaaagacacaacaatgaaa

SEQ ID NO: 3 - DNA, 3006 bp, Homo sapiens
agtctagggaaagtcattcagtggatgtgatcttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactc

SEQ ID NO: 4 - DNA, 2360 bp, Homo sapiens
gtaattcccaggttgcaggcttgtgagagccttaggttggattccctagcttgaaaaggagatcgttttacaagtgcttcattgaggagagctctgaggcagaggggaatgagggaagcaggctgggacaaaggagggaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactc

SEQ ID NO: 5 - DNA, 3135 bp, Homo sapiens
ttagaactttttaaaagaggcaaaggcagaggagaacaaaggaaggaggaagtaacttgtggaatgttgagaaagcgcccaacccaagttcaaaggctgataagagagaaaatctcatgaggaggttttagtctagggaaagtcattcagtggatgtgatcttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactc

SEQ ID NO: 6 - DNA, 3596 bp, Homo sapiens
ggcactcatacatacactctggcaatgaggacactgagctcgcttctgaaatttgacaagataaccactaaaatctctttgaattctatgttgttgtgatcccatggctacagaggatcaggagttgacatagatactctttggatttcataccatgtggaggctttcttacttccacgtgaccttgactgagttttgaatagcgcccaacccaagttcaaaggctgataagagagaaaatctcatgaggaggttttagtctagggaaagtcattcagtggatgtgatcttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactc

SEQ ID NO: 7 - protein, 805 aa, Homo sapiens [sequence omitted]

SEQ ID NO: 8 - protein, 694 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLLEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSF

SEQ ID NO: 9 - protein, 459 aa, Homo sapiens
MREAGWDKGGRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSF

SEQ ID NO: 10 - protein, 694 aa, Homo sapiens; identical to SEQ ID NO: 8

SEQ ID NO: 11 - protein, 805 aa, Homo sapiens [sequence omitted]

SEQ ID NO: 12 - DNA, 3250 bp, Homo sapiens
accagggtcccggctcggggtccgggctggggaggggaacctgggcgcctgggacccgccgatgccccctgccccgcccggaggtgaaagcgggtgtgaggagcgcggcgcggcaggtcatattgaacattccagatacctatcattactcgatgctgttgataacagcaagatggctttgaactcagggtcaccaccagctattggaccttactatgaaaaccatggataccaaccggaaaacccctatcccgcacagcccactgtggtccccactgtctacgaggtgcatccggctcagtactacccgtcccccgtgccccagtacgccccgagggtcctgacgcaggcttccaaccccgtcgtctgcacgcagcccaaatccccatccgggacagtgtgcacctcaaagactaagaaagcactgtgcatcaccttgaccctggggaccttcctcgtgggagctgcgctggccgctggcctactctggaagttcatgggcagcaagtgctccaactctgggatagagtgcgactcctcaggtacctgcatcaacccctctaactggtgtgatggcgtgtcacactgccccggcggggaggacgagaatcggtgtgttcgcctctacggaccaaacttcatccttcaggtgtactcatctcagaggaagtcctggcaccctgtgtgccaagacgactggaacgagaactacgggcgggcggcctgcagggacatgggctataagaataatttttactctagccaaggaatagtggatgacagcggatccaccagctttatgaaactgaacacaagtgccggcaatgtcgatatctataaaaaactgtaccacagtgatgcctgttcttcaaaagcagtggtttctttacgctgtatagcctgcggggtcaacttgaactcaagccgccagagcaggattgtgggcggcgagagcgcgctcccgggggcctggccctggcaggtcagcctgcacgtccagaacgtccacgtgtgcggaggctccatcatcacccccgagtggatcgtgacagccgcccactgcgtggaaaaacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggcagacggctaatccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccattctgtgcaggctgcagtggctcccctgcccagcctgctctccctaaccccttgtccgcaaggggtgatggccggctggttgtgggcactggcggtcaagtgtggaggagaggggtggaggctgccccattgagatcttcctgctgagtcctttccaggggccaattttggatgagcatggagctgtcacctctcagctgctggatgacttgagatgaaaaaggagagacatggaaagggagacagccaggtggcacctgcagcggctgccctctggggccacttggtagtgtccccagcctacctctccacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgcacatgccccttggtcctggcagggcgccaagtctggcaccatgttggcctcttcaggcctgctagtcactggaaattgaggtccatgggggaaatcaaggatgctcagtttaaggtacactgtttccatgttatgtttctacacattgctacctcagtgctcctggaaacttagcttttgatgtctccaagtagtccaccttcatttaactctttgaaactgtatcatctttgccaagtaagagtggtggcctatttcagctgctttgacaaaatgactggctcctgacttaacgttctataaatgaatgtgctgaagcaaagtgcccatggtggcggcgaagaagagaaagatgtgttttgttttggactctctgtggtcccttccaatgctgtgggtttccaaccaggggaagggtcccttttgcattgccaagtgccataaccatgagcactactctaccatggttctgcctcctggccaagcaggctggtttgcaagaatgaaatgaatgattctacagctaggacttaaccttgaaatggaaagtcatgcaatcccatttgcaggatctgtctgtgcacatgcctctgtagagagcagcattcccagggaccttggaaacagttggcactgtaaggtgcttgctccccaagacacatcctaaaaggtgttgtaatggtgaaaacgtcttccttctttattgccccttcttatttatgtgaacaactgtttgtctttttttgtatcttttttaaactgtaaagttcaattgtgaaaatgaatatcatgcaaataaattatgcaatttttttttcaaagtaaaaaaaaaa

SEQ ID NO: 13 - DNA, 3200 bp, Homo sapiens
gagtaggcgcgagctaagcaggaggcggaggcggaggcggagggcgaggggcggggagcgccgcctggagcgcggcaggtcatattgaacattccagatacctatcattactcgatgctgttgataacagcaagatggctttgaactcagggtcaccaccagctattggaccttactatgaaaaccatggataccaaccggaaaacccctatcccgcacagcccactgtggtccccactgtctacgaggtgcatccggctcagtactacccgtcccccgtgccccagtacgccccgagggtcctgacgcaggcttccaaccccgtcgtctgcacgcagcccaaatccccatccgggacagtgtgcacctcaaagactaagaaagcactgtgcatcaccttgaccctggggaccttcctcgtgggagctgcgctggccgctggcctactctggaagttcatgggcagcaagtgctccaactctgggatagagtgcgactcctcaggtacctgcatcaacccctctaactggtgtgatggcgtgtcacactgccccggcggggaggacgagaatcggtgtgttcgcctctacggaccaaacttcatccttcaggtgtactcatctcagaggaagtcctggcaccctgtgtgccaagacgactggaacgagaactacgggcgggcggcctgcagggacatgggctataagaataatttttactctagccaaggaatagtggatgacagcggatccaccagctttatgaaactgaacacaagtgccggcaatgtcgatatctataaaaaactgtaccacagtgatgcctgttcttcaaaagcagtggtttctttacgctgtatagcctgcggggtcaacttgaactcaagccgccagagcaggattgtgggcggcgagagcgcgctcccgggggcctggccctggcaggtcagcctgcacgtccagaacgtccacgtgtgcggaggctccatcatcacccccgagtggatcgtgacagccgcccactgcgtggaaaaacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgaggacggctaatccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccattctgtgcaggctgcagtggctcccctgcccagcctgctctccctaaccccttgtccgcaaggggtgatggccggctggttgtgggcactggcggtcaagtgtggaggagaggggtggaggctgccccattgagatcttcctgctgagtcctttccaggggccaattttggatgagcatggagctgtcacctctcagctgctggatgacttgagatgaaaaaggagagacatggaaagggagacagccaggtggcacctgcagcggctgccctctggggccacttggtagtgtccccagcctacctctccacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgcacatgccccttggtcctggcagggcgccaagtctggcaccatgttggcctcttcaggcctgctagtcactggaaattgaggtccatgggggaaatcaaggatgctcagtttaaggtacactgtttccatgttatgtttctacacattgctacctcagtgctcctggaaacttagcttttgatgtctccaagtagtccaccttcatttaactctttgaaactgtatcatctttgccaagtaagagtggtggcctatttcagctgctttgacaaaatgactggctcctgacttaacgttctataaatgaatgtgctgaagcaaagtgcccatggtggcggcgaagaagagaaagatgtgttttgttttggactctctgtggtcccttccaatgctgtgggtttccaaccaggggaagggtcccttttgcattgccaagtgccataaccatgagcactactctaccatggttctgcctcctggccaagcaggctggtttgcaagaatgaaatgaatgattctacagctaggacttaaccttgaaatggaaagtcatgcaatcccatttgcaggatctgtctgtgcacatgcctctgtagagagcagcattcccagggaccttggaaacagttggcactgtaaggtgcttgctccccaagacacatcctaaaaggtgttgtaatggtgaaaacgtcttccttctttattgccccttcttatttatgtgaacaactgtttgtctttttttgtatcttttttaaactgtaaagttcaattgtgaaaatgaatatcatgcaaataaattatgcaatttttttttcaaagtaa

SEQ ID NO: 14 - DNA, 3450 bp, Homo sapiens
gagtaggcgcgagctaagcaggaggcggaggcggaggcggagggcgaggggcggggagcgccgcctggagcgcggcaggtcatattgaacattccagatacctatcattactcgatgctgttgataacagcaagatggctttgaactcagggtcaccaccagctattggaccttactatgaaaaccatggataccaaccggaaaacccctatcccgcacagcccactgtggtccccactgtctacgaggtgcatccggctcagtactacccgtcccccgtgccccagtacgccccgagggtcctgacgcaggcttccaaccccgtcgtctgcacgcagcccaaatccccatccgggacagtgtgcacctcaaagactaagaaagcactgtgcatcaccttgaccctggggaccttcctcgtgggagctgcgctggccgctggcctactctggaagttcatgggcagcaagtgctccaactctgggatagagtgcgactcctcaggtacctgcatcaacccctctaactggtgtgatggcgtgtcacactgccccggcggggaggacgagaatcggtgtgttcgcctctacggaccaaacttcatccttcaggtgtactcatctcagaggaagtcctggcaccctgtgtgccaagacgactggaacgagaactacgggcgggcggcctgcagggacatgggctataagaataatttttactctagccaaggaatagtggatgacagcggatccaccagctttatgaaactgaacacaagtgccggcaatgtcgatatctataaaaaactgtaccacagtgatgcctgttcttcaaaagcagtggtttctttacgctgtatagcctgcggggtcaacttgaactcaagccgccagagcaggattgtgggcggcgagagcgcgctcccgggggcctggccctggcaggtcagcctgcacgtccagaacgtccacgtgtgcggaggctccatcatcacccccgagtggatcgtgacagccgcccactgcgtggaaaaacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggcagacggctaatccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccattctgtgcaggctgcagtggctcccctgcccagcctgctctccctaaccccttgtccgcaaggggtgatggccggctggttgtgggcactggcggtcaagtgtggaggagaggggtggaggctgccccattgagatcttcctgctgagtcctttccaggggccaattttggatgagcatggagctgtcacctctcagctgctggatgacttgagatgaaaaaggagagacatggaaagggagacagccaggtggcacctgcagcggctgccctctggggccacttggtagtgtccccagcctacctctccacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgcacatgccccttggtcctggcagggcgccaagtctggcaccatgttggcctcttcaggcctgctagtcactggaaattgaggtccatgggggaaatcaaggatgctcagtttaaggtacactgtttccatgttatgtttctacacattgctacctcagtgctcctggaaacttagcttttgatgtctccaagtagtccaccttcatttaactctttgaaactgtatcatctttgccaagtaagagtggtggcctatttcagctgctttgacaaaatgactggctcctgacttaacgttctataaatgaatgtgctgaagcaaagtgcccatggtggcggcgaagaagagaaagatgtgttttgttttggactctctgtggtcccttccaatgctgtgggtttccaaccaggggaagggtcccttttgcattgccaagtgccataaccatgagcactactctaccatggttctgcctcctggccaagcaggctggtttgcaagaatgaaatgaatgattctacagctaggacttaaccttgaaatggaaagtcatgcaatcccatttgcaggatctgtctgtgcacatgcctctgtagagagcagcattcccagggaccttggaaacagttggcactgtaaggtgcttgctccccaagacacatcctaaaaggtgttgtaatggtgaaaacgtcttccttctttattgccccttcttatttatgtgaacaactgtttgtctttttttgtatcttttttaaactgtaaagttcaattgtgaaaatgaatatcatgcaaataaattatgcaatttttttttcaaagtaactactgcatctttgaagttctgcctggtgagtaggaccagcctccatttccttataagggggtgatgttgaggctgctggtcagaggaccaaaggtgaggcaaggccagacttggtgctcctgtggttggtgccctcagttcctgcagcctgtcctgttggagaggtccctcaaatgactccttcttattattctattagtctgtttccatgctcctaataaagacatacccaagactgcaattta

SEQ ID NO: 15 - protein, 529 aa, Homo sapiens
MPPAPPGGESGCEERGAAGHIEHSRYLSLLDAVDNSKMALNSGSPPAIGPYYENHGYQPENPYPAQPTVVPTVYEVHPAQYYPSPVPQYAPRVLTQASNPVVCTQPKSPSGTVCTSKTKKALCITLTLGTFLVGAALAAGLLWKFMGSKCSNSGIECDSSGTCINPSNWCDGVSHCPGGEDENRCVRLYGPNFILQVYSSQRKSWHPVCQDDWNENYGRAACRDMGYKNNFYSSQGIVDDSGSTSFMKLNTSAGNVDIYKKLYHSDACSSKAVVSLRCIACGVNLNSSRQSRIVGGESALPGAWPWQVSLHVQNVHVCGGSIITPEWIVTAAHCVEKPLNNPWHWTAFAGILRQSFMFYGAGYQVEKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRADG

SEQ ID NO: 16 - protein, 498 aa, Homo sapiens
MALNSGSPPAIGPYYENHGYQPENPYPAQPTVVPTVYEVHPAQYYPSPVPQYAPRVLTQASNPVVCTQPKSPSGTVCTSKTKKALCITLTLGTFLVGAALAAGLLWKFMGSKCSNSGIECDSSGTCINPSNWCDGVSHCPGGEDENRCVRLYGPNFILQVYSSQRKSWHPVCQDDWNENYGRAACRDMGYKNNFYSSQGIVDDSGSTSFMKLNTSAGNVDIYKKLYHSDACSSKAVVSLRCIACGVNLNSSRQSRIVGGESALPGAWPWQVSLHVQNVHVCGGSIITPEWIVTAAHCVEKPLNNPWHWTAFAGILRQSFMFYGAGYQVEKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRTANPHGLRP

SEQ ID NO: 17 - protein, 492 aa, Homo sapiens
MALNSGSPPAIGPYYENHGYQPENPYPAQPTVVPTVYEVHPAQYYPSPVPQYAPRVLTQASNPVVCTQPKSPSGTVCTSKTKKALCITLTLGTFLVGAALAAGLLWKFMGSKCSNSGIECDSSGTCINPSNWCDGVSHCPGGEDENRCVRLYGPNFILQVYSSQRKSWHPVCQDDWNENYGRAACRDMGYKNNFYSSQGIVDDSGSTSFMKLNTSAGNVDIYKKLYHSDACSSKAVVSLRCIACGVNLNSSRQSRIVGGESALPGAWPWQVSLHVQNVHVCGGSIITPEWIVTAAHCVEKPLNNPWHWTAFAGILRQSFMFYGAGYQVEKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRADG

SEQ ID NO: 18 - DNA, 5501 bp, Homo sapiens
actcctggaatacacagagagaggcagcagcttgctcagcggacaaggatgctgggcgtgagggaccaaggcctgccctgcactcgggcctcctccagccagtgctgaccagggacttctgacctgctggccagccaggacctgtgtggggaggccctcctgctgccttggggtgacaatctcagctccaggctacagggagaccgggaggatcacagagccagcatgttacaggatcctgacagtgatcaacctctgaacagcctcgatgtcaaacccctgcgcaaaccccgtatccccatggagaccttcagaaaggtggggatccccatcatcatagcactactgagcctggcgagtatcatcattgtggttgtcctcatcaaggtgattctggataaatactacttcctctgcgggcagcctctccacttcatcccgaggaagcagctgtgtgacggagagctggactgtcccttgggggaggacgaggagcactgtgtcaagagcttccccgaagggcctgcagtggcagtccgcctctccaaggaccgatccacactgcaggtgctggactcggccacagggaactggttctctgcctgtttcgacaacttcacagaagctctcgctgagacagcctgtaggcagatgggctacagcagagctgtggagattggcccagaccaggatctggatgttgttgaaatcacagaaaacagccaggagcttcgcatgcggaactcaagtgggccctgtctctcaggctccctggtctccctgcactgtcttgcctgtgggaagagcctgaagaccccccgtgtggtgggtgtggaggaggcctctgtggattcttggccttggcaggtcagcatccagtacgacaaacagcacgtctgtggagggagcatcctggacccccactgggtcctcacggcagcccactgcttcaggaaacataccgatgtgttcaactggaaggtgcgggcaggctcagacaaactgggcagcttcccatccctggctgtggccaagatcatcatcattgaattcaaccccatgtaccccaaagacaatgacatcgccctcatgaagctgcagttcccactcactttctcaggcacagtcaggcccatctgtctgcccttctttgatgaggagctcactccagccaccccactctggatcattggatggggctttacgaagcagaatggagggaagatgtctgacatactgctgcaggcgtcagtccaggtcattgacagcacacggtgcaatgcagacgatgcgtaccagggggaagtcaccgagaagatgatgtgtgcaggcatcccggaagggggtgtggacacctgccagggtgacagtggtgggcccctgatgtaccaatctgaccagtggcatgtggtgggcatcgttagttggggctatggctgcgggggcccgagcaccccaggagtatacaccaaggtctcagcctatctcaactggatctacaatgtctggaaggctgagctgtaatgctgctgcccctttgcagtgctgggagccgcttccttcctgccctgcccacctggggatcccccaaagtcagacacagagcaagagtccccttgggtacacccctctgcccacagcctcagcatttcttggagcagcaaagggcctcaattcctataagagaccctcgcagcccagaggcgcccagaggaagtcagcagccctagctcggccacacttggtgctcccagcatcccagggagagacacagcccactgaacaaggtctcaggggtattgctaagccaagaaggaactttcccacactactgaatggaagcaggctgtcttgtaaaagcccagatcactgtgggctggagaggagaaggaaagggtctgcgccagccctgtccgtcttcacccatccccaagcctactagagcaagaaaccagttgtaatataaaatgcactgccctactgttggtatgactaccgttacctactgttgtcattgttattacagctatggccactattattaaagagctgtgtaacatctctggcataggctagctggaatgcttgataagaactgagctgggatgattgaactttcattctttggcttggggagaaaagaagtcctggggaagcaattgagtctcaaagtagaggcaggggaaaaaagagttagggagaccagatctgctgagtggcagcaagagtgagctgcagattacagaaaccagggtgagcaagtttgagtcccacacagggccttctccctttgcctctttccctccctccctgcctgtgataatcagccaggagccagggataacctatgacttgggaaagagatgagttaggcagtcaagggtgacattcaatcagggatccacaagtggctggaaagaaatgctggtcctgtgtcctaactttttccgcctggagagccctcagtgtggcttcttacatttaaaaaacaaaaaggatcagctgccaggtgtgaggcagtccccaagctgagttgtgaggatgtaagcatgaataagtccctgcactcaaaatggtcaaagaattaaaccccatggacttttttggcatctgtatgaaagcttgggttttctgaggactgtcttgctatagttaagtcagatcctagatgaaatatacttgttcatactgtactaggttcttaggaaacaacagaattcctcaaatgccaaaaacaaagaaaatagaaacccagaaaacaaaacaaaataaaacaaaaccatcagaactgtgagtggaaactaaggtgatgatctgggagcaatacactaaaatcttgggtcgagacctatatgaaggctggcagtggagctaaacctggacacactgaagacaagggagctgaaccagggctcctacatgaagcagggataactgatggcagtaaatgtggtctcaaattgcagatggtctggaggaaaatttcccaaatttagagcctcaggattcccaaagatcctccaaatatgagctcacaatcaaagatcagagacgttgaaaaataaaaaacaccttaagtgggcagcataaaaaacagctaatttagaaccccaaaggcttcagatgtcagaatattagagacttatgataataagcaatatttgcagagtatttgtatgtgccagacactattgtaagtgcttcatcatgtactgattcatttaatactcacagaaatctgtgagatgggtattattcttatcctcactctatggattaaaaaaactaaggcacaaagtggttaagctccttgcctgagattatagactgtaagttgaacgtgagcacttggaatacagagttcatgctgtaaactaccacactatagggcctccaatatgataatttataaaatatttgaataaaaaatgaatactagttccacattttaaaatcatgtttaactgtggtcaaatgcacataacacaagttgccatcttcaccatttttaggtgtatagttcagtggtgttatgtacattcacactattgtgcagtcatcaccaccatccatctccagaacagaaactcagtacccatcaaacaactctccatttccccctcctcccaatctctggcaaccaccattgtgctttcagtctctgtgaactggattactctgggtacctcatttaagtgaagtcatgcagtattggtctttttgtacttgttttatttcacttcacattgtgtcttcaagtttcacccatgttgtagcatgtgtcagaatttcttccctttttagactaaataatattctattgtttatacgaacattcaggttacttctatcttttggctattgtgaattatgctgctgtgaacatgggtgtacaagtatctctttgaggccctgctttcaattctcttgggtatattcccagaagtggaattgctggatcatatggtaattctattttgaattttttgaggaactgatatattgctttccatagagactgcaccattttacattcccatcaacagtttgcaggagttactatttctccatatcccccctaacacttgctattttctgttaaaaatggatatcttaataatcaagcaaaaataacaggcagatttgaaaaagaactgaatacagcttttagaaataaaaactataattataaaaataaaaaactaagtggatggggtaaataacaattaaaacaccaattaagagagaacaaatgaactggaagataaattgaagaagtgactaggcttaacagcagagagagataaggagattaaaaatatgaaaacaaggccaggagcaatgaagcctagaatggtaaattctaacatatccagaatcccagaaagagagaatcaagacaatgagagagagacagtaccaaagagataagagctgagaatgttccagaattgataaaaggtgtgaatccacagaacatacaccaccatagtgtacacgcatacaaccaaggtggaaaaattagaataaatccacacctatgtacattataatgaaactgcagaacaccaaagacaaaaagaaactccttatagcagcagagagaaaacccagaccacccacagtaccacaaatctaccacaattagactgacaacaggctttcccacagcaataaaggagctagaagtcagtggaagtatatctccagcatgccaaaagataacaatcaatcagggattgtgaaccctacaaaactatctttcaagaataaaggcattttcaagaaaacaaaaacagactttaccatcaacaaaccttctctaaaagaatatataaagcatttactttaggaagaaggaaaatgatcctaaaaggaagaaccaagaagcaagtagcaatagtgaggcaattgtgaaaatgtaggtaagtctaaacacactctgtctacttcttcttcttcttcttcttcttcttcttcttattttgagactgagtcttgccctgtcacccagactggagtgcagtggcaggatcttggctcactgctatctccacctcccaggttcaagtgattcttctgcctcagcctcccgagtagctgggattacatgcacatgccaccatatccggctaatttttgaatttttagtagagatggggtttcactgtgttggccaggccggtctcaaactcccgacctcaagtgatccccccgcctcggcctcccaaagtgctgggattacaggcgtgtctacatattattaaaataacaataatatttattttgtgggttaattttttttgaaacagatattgaatttattggttggctatgagtagaaaaatacatcagtaaagaaaaaagaccctgtatataaatataatactagctagttaaaatttgaccaagaagtttccattgtgggttaatttttaaaggcctaactgaaatatggagtaaccacagcatgcagcatgtaaattaaaggggatagctgg

SEQ ID NO: 19 - DNA, 5510 bp, Homo sapiens
actcctggaatacacagagagaggcagcagcttgctcagcggacaaggatgctgggcgtgagggaccaaggcctgccctgcactcgggcctcctccagccagtgctgaccagggacttctgacctgctggccagccaggacctgtgtggggaggccctcctgctgccttggggtgacaatctcagctccaggctacagggagaccgggaggatcacagagccagcatggatcctgacagtgatcaacctctgaacagcctcgatgtcaaacccctgcgcaaaccccgtatccccatggagaccttcagaaaggtggggatccccatcatcatagcactactgagcctggcgagtatcatcattgtggttgtcctcatcaaggtgattctggataaatactacttcctctgcgggcagcctctccacttcatcccgaggaagcagctgtgtgacggagagctggactgtcccttgggggaggacgaggagcactgtgtcaagagcttccccgaagggcctgcagtggcagtccgcctctccaaggaccgatccacactgcaggtgctggactcggccacagggaactggttctctgcctgtttcgacaacttcacagaagctctcgctgagacagcctgtaggcagatgggctacagcagcaaacccactttcagagctgtggagattggcccagaccaggatctggatgttgttgaaatcacagaaaacagccaggagcttcgcatgcggaactcaagtgggccctgtctctcaggctccctggtctccctgcactgtcttgcctgtgggaagagcctgaagaccccccgtgtggtgggtgtggaggaggcctctgtggattcttggccttggcaggtcagcatccagtacgacaaacagcacgtctgtggagggagcatcctggacccccactgggtcctcacggcagcccactgcttcaggaaacataccgatgtgttcaactggaaggtgcgggcaggctcagacaaactgggcagcttcccatccctggctgtggccaagatcatcatcattgaattcaaccccatgtaccccaaagacaatgacatcgccctcatgaagctgcagttcccactcactttctcaggcacagtcaggcccatctgtctgcccttctttgatgaggagctcactccagccaccccactctggatcattggatggggctttacgaagcagaatggagggaagatgtctgacatactgctgcaggcgtcagtccaggtcattgacagcacacggtgcaatgcagacgatgcgtaccagggggaagtcaccgagaagatgatgtgtgcaggcatcccggaagggggtgtggacacctgccagggtgacagtggtgggcccctgatgtaccaatctgaccagtggcatgtggtgggcatcgttagttggggctatggctgcgggggcccgagcaccccaggagtatacaccaaggtctcagcctatctcaactggatctacaatgtctggaaggctgagctgtaatgctgctgcccctttgcagtgctgggagccgcttccttcctgccctgcccacctggggatcccccaaagtcagacacagagcaagagtccccttgggtacacccctctgcccacagcctcagcatttcttggagcagcaaagggcctcaattcctataagagaccctcgcagcccagaggcgcccagaggaagtcagcagccctagctcggccacacttggtgctcccagcatcccagggagagacacagcccactgaacaaggtctcaggggtattgctaagccaagaaggaactttcccacactactgaatggaagcaggctgtcttgtaaaagcccagatcactgtgggctggagaggagaaggaaagggtctgcgccagccctgtccgtcttcacccatccccaagcctactagagcaagaaaccagttgtaatataaaatgcactgccctactgttggtatgactaccgttacctactgttgtcattgttattacagctatggccactattattaaagagctgtgtaacatctctggcataggctagctggaatgcttgataagaactgagctgggatgattgaactttcattctttggcttggggagaaaagaagtcctggggaagcaattgagtctcaaagtagaggcaggggaaaaaagagttagggagaccagatctgctgagtggcagcaagagtgagctgcagattacagaaaccagggtgagcaagtttgagtcccacacagggccttctccctttgcctctttccctccctccctgcctgtgataatcagccaggagccagggataacctatgacttgggaaagagatgagttaggcagtcaagggtgacattcaatcagggatccacaagtggctggaaagaaatgctggtcctgtgtcctaactttttccgcctggagagccctcagtgtggcttcttacatttaaaaaacaaaaaggatcagctgccaggtgtgaggcagtccccaagctgagttgtgaggatgtaagcatgaataagtccctgcactcaaaatggtcaaagaattaaaccccatggacttttttggcatctgtatgaaagcttgggttttctgaggactgtcttgctatagttaagtcagatcctagatgaaatatacttgttcatactgtactaggttcttaggaaacaacagaattcctcaaatgccaaaaacaaagaaaatagaaacccagaaaacaaaacaaaataaaacaaaaccatcagaactgtgagtggaaactaaggtgatgatctgggagcaatacactaaaatcttgggtcgagacctatatgaaggctggcagtggagctaaacctggacacactgaagacaagggagctgaaccagggctcctacatgaagcagggataactgatggcagtaaatgtggtctcaaattgcagatggtctggaggaaaatttcccaaatttagagcctcaggattcccaaagatcctccaaatatgagctcacaatcaaagatcagagacgttgaaaaataaaaaacaccttaagtgggcagcataaaaaacagctaatttagaaccccaaaggcttcagatgtcagaatattagagacttatgataataagcaatatttgcagagtatttgtatgtgccagacactattgtaagtgcttcatcatgtactgattcatttaatactcacagaaatctgtgagatgggtattattcttatcctcactctatggattaaaaaaactaaggcacaaagtggttaagctccttgcctgagattatagactgtaagttgaacgtgagcacttggaatacagagttcatgctgtaaactaccacactatagggcctccaatatgataatttataaaatatttgaataaaaaatgaatactagttccacattttaaaatcatgtttaactgtggtcaaatgcacataacacaagttgccatcttcaccatttttaggtgtatagttcagtggtgttatgtacattcacactattgtgcagtcatcaccaccatccatctccagaacagaaactcagtacccatcaaacaactctccatttccccctcctcccaatctctggcaaccaccattgtgctttcagtctctgtgaactggattactctgggtacctcatttaagtgaagtcatgcagtattggtctttttgtacttgttttatttcacttcacattgtgtcttcaagtttcacccatgttgtagcatgtgtcagaatttcttccctttttagactaaataatattctattgtttatacgaacattcaggttacttctatcttttggctattgtgaattatgctgctgtgaacatgggtgtacaagtatctctttgaggccctgctttcaattctcttgggtatattcccagaagtggaattgctggatcatatggtaattctattttgaattttttgaggaactgatatattgctttccatagagactgcaccattttacattcccatcaacagtttgcaggagttactatttctccatatcccccctaacacttgctattttctgttaaaaatggatatcttaataatcaagcaaaaataacaggcagatttgaaaaagaactgaatacagcttttagaaataaaaactataattataaaaataaaaaactaagtggatggggtaaataacaattaaaacaccaattaagagagaacaaatgaactggaagataaattgaagaagtgactaggcttaacagcagagagagataaggagattaaaaatatgaaaacaaggccaggagcaatgaagcctagaatggtaaattctaacatatccagaatcccagaaagagagaatcaagacaatgagagagagacagtaccaaagagataagagctgagaatgttccagaattgataaaaggtgtgaatccacagaacatacaccaccatagtgtacacgcatacaaccaaggtggaaaaattagaataaatccacacctatgtacattataatgaaactgcagaacaccaaagacaaaaagaaactccttatagcagcagagagaaaacccagaccacccacagtaccacaaatctaccacaattagactgacaacaggctttcccacagcaataaaggagctagaagtcagtggaagtatatctccagcatgccaaaagataacaatcaatcagggattgtgaaccctacaaaactatctttcaagaataaaggcattttcaagaaaacaaaaacagactttaccatcaacaaaccttctctaaaagaatatataaagcatttactttaggaagaaggaaaatgatcctaaaaggaagaaccaagaagcaagtagcaatagtgaggcaattgtgaaaatgtaggtaagtctaaacacactctgtctacttcttcttcttcttcttcttcttcttcttcttattttgagactgagtcttgccctgtcacccagactggagtgcagtggcaggatcttggctcactgctatctccacctcccaggttcaagtgattcttctgcctcagcctcccgagtagctgggattacatgcacatgccaccatatccggctaatttttgaatttttagtagagatggggtttcactgtgttggccaggccggtctcaaactcccgacctcaagtgatccccccgcctcggcctcccaaagtgctgggattacaggcgtgtctacatattattaaaataacaataatatttattttgtgggttaattttttttgaaacagatattgaatttattggttggctatgagtagaaaaatacatcagtaaagaaaaaagaccctgtatataaatataatactagctagttaaaatttgaccaagaagtttccattgtgggttaatttttaaaggcctaactgaaatatggagtaaccacagcatgcagcatgtaaattaaaggggatagctgg

SEQ ID NO: 20 - DNA, 5396 bp, Homo sapiens
actcctggaatacacagagagaggcagcagcttgctcagcggacaaggatgctgggcgtgagggaccaaggcctgccctgcactcgggcctcctccagccagtgctgaccagggacttctgacctgctggccagccaggacctgtgtggggaggccctcctgctgccttggggtgacaatctcagctccaggctacagggagaccgggaggatcacagagccagcatggatcctgacagtgatcaacctctgaacagcctcgtcaaggtgattctggataaatactacttcctctgcgggcagcctctccacttcatcccgaggaagcagctgtgtgacggagagctggactgtcccttgggggaggacgaggagcactgtgtcaagagcttccccgaagggcctgcagtggcagtccgcctctccaaggaccgatccacactgcaggtgctggactcggccacagggaactggttctctgcctgtttcgacaacttcacagaagctctcgctgagacagcctgtaggcagatgggctacagcagcaaacccactttcagagctgtggagattggcccagaccaggatctggatgttgttgaaatcacagaaaacagccaggagcttcgcatgcggaactcaagtgggccctgtctctcaggctccctggtctccctgcactgtcttgcctgtgggaagagcctgaagaccccccgtgtggtgggtgtggaggaggcctctgtggattcttggccttggcaggtcagcatccagtacgacaaacagcacgtctgtggagggagcatcctggacccccactgggtcctcacggcagcccactgcttcaggaaacataccgatgtgttcaactggaaggtgcgggcaggctcagacaaactgggcagcttcccatccctggctgtggccaagatcatcatcattgaattcaaccccatgtaccccaaagacaatgacatcgccctcatgaagctgcagttcccactcactttctcaggcacagtcaggcccatctgtctgcccttctttgatgaggagctcactccagccaccccactctggatcattggatggggctttacgaagcagaatggagggaagatgtctgacatactgctgcaggcgtcagtccaggtcattgacagcacacggtgcaatgcagacgatgcgtaccagggggaagtcaccgagaagatgatgtgtgcaggcatcccggaagggggtgtggacacctgccagggtgacagtggtgggcccctgatgtaccaatctgaccagtggcatgtggtgggcatcgttagttggggctatggctgcgggggcccgagcaccccaggagtatacaccaaggtctcagcctatctcaactggatctacaatgtctggaaggctgagctgtaatgctgctgcccctttgcagtgctgggagccgcttccttcctgccctgcccacctggggatcccccaaagtcagacacagagcaagagtccccttgggtacacccctctgcccacagcctcagcatttcttggagcagcaaagggcctcaattcctataagagaccctcgcagcccagaggcgcccagaggaagtcagcagccctagctcggccacacttggtgctcccagcatcccagggagagacacagcccactgaacaaggtctcaggggtattgctaagccaagaaggaactttcccacactactgaatggaagcaggctgtcttgtaaaagcccagatcactgtgggctggagaggagaaggaaagggtctgcgccagccctgtccgtcttcacccatccccaagcctactagagcaagaaaccagttgtaatataaaatgcactgccctactgttggtatgactaccgttacctactgttgtcattgttattacagctatggccactattattaaagagctgtgtaacatctctggcataggctagctggaatgcttgataagaactgagctgggatgattgaactttcattctttggcttggggagaaaagaagtcctggggaagcaattgagtctcaaagtagaggcaggggaaaaaagagttagggagaccagatctgctgagtggcagcaagagtgagctgcagattacagaaaccagggtgagcaagtttgagtcccacacagggccttctccctttgcctctttccctccctccctgcctgtgataatcagccaggagccagggataacctatgacttgggaaagagatgagttaggcagtcaagggtgacattcaatcagggatccacaagtggctggaaagaaatgctggtcctgtgtcctaactttttccgcctggagagccctcagtgtggcttcttacatttaaaaaacaaaaaggatcagctgccaggtgtgaggcagtccccaagctgagttgtgaggatgtaagcatgaataagtccctgcactcaaaatggtcaaagaattaaaccccatggacttttttggcatctgtatgaaagcttgggttttctgaggactgtcttgctatagttaagtcagatcctagatgaaatatacttgttcatactgtactaggttcttaggaaacaacagaattcctcaaatgccaaaaacaaagaaaatagaaacccagaaaacaaaacaaaataaaacaaaaccatcagaactgtgagtggaaactaaggtgatgatctgggagcaatacactaaaatcttgggtcgagacctatatgaaggctggcagtggagctaaacctggacacactgaagacaagggagctgaaccagggctcctacatgaagcagggataactgatggcagtaaatgtggtctcaaattgcagatggtctggaggaaaatttcccaaatttagagcctcaggattcccaaagatcctccaaatatgagctcacaatcaaagatcagagacgttgaaaaataaaaaacaccttaagtgggcagcataaaaaacagctaatttagaaccccaaaggcttcagatgtcagaatattagagacttatgataataagcaatatttgcagagtatttgtatgtgccagacactattgtaagtgcttcatcatgtactgattcatttaatactcacagaaatctgtgagatgggtattattcttatcctcactctatggattaaaaaaactaaggcacaaagtggttaagctccttgcctgagattatagactgtaagttgaacgtgagcacttggaatacagagttcatgctgtaaactaccacactatagggcctccaatatgataatttataaaatatttgaataaaaaatgaatactagttccacattttaaaatcatgtttaactgtggtcaaatgcacataacacaagttgccatcttcaccatttttaggtgtatagttcagtggtgttatgtacattcacactattgtgcagtcatcaccaccatccatctccagaacagaaactcagtacccatcaaacaactctccatttccccctcctcccaatctctggcaaccaccattgtgctttcagtctctgtgaactggattactctgggtacctcatttaagtgaagtcatgcagtattggtctttttgtacttgttttatttcacttcacattgtgtcttcaagtttcacccatgttgtagcatgtgtcagaatttcttccctttttagactaaataatattctattgtttatacgaacattcaggttacttctatcttttggctattgtgaattatgctgctgtgaacatgggtgtacaagtatctctttgaggccctgctttcaattctcttgggtatattcccagaagtggaattgctggatcatatggtaattctattttgaattttttgaggaactgatatattgctttccatagagactgcaccattttacattcccatcaacagtttgcaggagttactatttctccatatcccccctaacacttgctattttctgttaaaaatggatatcttaataatcaagcaaaaataacaggcagatttgaaaaagaactgaatacagcttttagaaataaaaactataattataaaaataaaaaactaagtggatggggtaaataacaattaaaacaccaattaagagagaacaaatgaactggaagataaattgaagaagtgactaggcttaacagcagagagagataaggagattaaaaatatgaaaacaaggccaggagcaatgaagcctagaatggtaaattctaacatatccagaatcccagaaagagagaatcaagacaatgagagagagacagtaccaaagagataagagctgagaatgttccagaattgataaaaggtgtgaatccacagaacatacaccaccatagtgtacacgcatacaaccaaggtggaaaaattagaataaatccacacctatgtacattataatgaaactgcagaacaccaaagacaaaaagaaactccttatagcagcagagagaaaacccagaccacccacagtaccacaaatctaccacaattagactgacaacaggctttcccacagcaataaaggagctagaagtcagtggaagtatatctccagcatgccaaaagataacaatcaatcagggattgtgaaccctacaaaactatctttcaagaataaaggcattttcaagaaaacaaaaacagactttaccatcaacaaaccttctctaaaagaatatataaagcatttactttaggaagaaggaaaatgatcctaaaaggaagaaccaagaagcaagtagcaatagtgaggcaattgtgaaaatgtaggtaagtctaaacacactctgtctacttcttcttcttcttcttcttcttcttcttcttattttgagactgagtcttgccctgtcacccagactggagtgcagtggcaggatcttggctcactgctatctccacctcccaggttcaagtgattcttctgcctcagcctcccgagtagctgggattacatgcacatgccaccatatccggctaatttttgaatttttagtagagatggggtttcactgtgttggccaggccggtctcaaactcccgacctcaagtgatccccccgcctcggcctcccaaagtgctgggattacaggcgtgtctacatattattaaaataacaataatatttattttgtgggttaattttttttgaaacagatattgaatttattggttggctatgagtagaaaaatacatcagtaaagaaaaaagaccctgtatataaatataatactagctagttaaaatttgaccaagaagtttccattgtgggttaatttttaaaggcctaactgaaatatggagtaaccacagcatgcagcatgtaaattaaaggggatagctgg

SEQ ID NO: 21 - DNA, 5520 bp, Homo sapiens
actcctggaatacacagagagaggcagcagcttgctcagcggacaaggatgctgggcgtgagggaccaaggcctgccctgcactcgggcctcctccagccagtgctgaccagggacttctgacctgctggccagccaggacctgtgtggggaggccctcctgctgccttggggtgacaatctcagctccaggctacagggagaccgggaggatcacagagccagcatggatcctgacagtgatcaacctctgaacagcctcggtaagttcagatgtcaaacccctgcgcaaaccccgtatccccatggagaccttcagaaaggtggggatccccatcatcatagcactactgagcctggcgagtatcatcattgtggttgtcctcatcaaggtgattctggataaatactacttcctctgcgggcagcctctccacttcatcccgaggaagcagctgtgtgacggagagctggactgtcccttgggggaggacgaggagcactgtgtcaagagcttccccgaagggcctgcagtggcagtccgcctctccaaggaccgatccacactgcaggtgctggactcggccacagggaactggttctctgcctgtttcgacaacttcacagaagctctcgctgagacagcctgtaggcagatgggctacagcagcaaacccactttcagagctgtggagattggcccagaccaggatctggatgttgttgaaatcacagaaaacagccaggagcttcgcatgcggaactcaagtgggccctgtctctcaggctccctggtctccctgcactgtcttgcctgtgggaagagcctgaagaccccccgtgtggtgggtgtggaggaggcctctgtggattcttggccttggcaggtcagcatccagtacgacaaacagcacgtctgtggagggagcatcctggacccccactgggtcctcacggcagcccactgcttcaggaaacataccgatgtgttcaactggaaggtgcgggcaggctcagacaaactgggcagcttcccatccctggctgtggccaagatcatcatcattgaattcaaccccatgtaccccaaagacaatgacatcgccctcatgaagctgcagttcccactcactttctcaggcacagtcaggcccatctgtctgcccttctttgatgaggagctcactccagccaccccactctggatcattggatggggctttacgaagcagaatggagggaagatgtctgacatactgctgcaggcgtcagtccaggtcattgacagcacacggtgcaatgcagacgatgcgtaccagggggaagtcaccgagaagatgatgtgtgcaggcatcccggaagggggtgtggacacctgccagggtgacagtggtgggcccctgatgtaccaatctgaccagtggcatgtggtgggcatcgttagttggggctatggctgcgggggcccgagcaccccaggagtatacaccaaggtctcagcctatctcaactggatctacaatgtctggaaggctgagctgtaatgctgctgcccctttgcagtgctgggagccgcttccttcctgccctgcccacctggggatcccccaaagtcagacacagagcaagagtccccttgggtacacccctctgcccacagcctcagcatttcttggagcagcaaagggcctcaattcctataagagaccctcgcagcccagaggcgcccagaggaagtcagcagccctagctcggccacacttggtgctcccagcatcccagggagagacacagcccactgaacaaggtctcaggggtattgctaagccaagaaggaactttcccacactactgaatggaagcaggctgtcttgtaaaagcccagatcactgtgggctggagaggagaaggaaagggtctgcgccagccctgtccgtcttcacccatccccaagcctactagagcaagaaaccagttgtaatataaaatgcactgccctactgttggtatgactaccgttacctactgttgtcattgttattacagctatggccactattattaaagagctgtgtaacatctctggcataggctagctggaatgcttgataagaactgagctgggatgattgaactttcattctttggcttggggagaaaagaagtcctggggaagcaattgagtctcaaagtagaggcaggggaaaaaagagttagggagaccagatctgctgagtggcagcaagagtgagctgcagattacagaaaccagggtgagcaagtttgagtcccacacagggccttctccctttgcctctttccctccctccctgcctgtgataatcagccaggagccagggataacctatgacttgggaaagagatgagttaggcagtcaagggtgacattcaatcagggatccacaagtggctggaaagaaatgctggtcctgtgtcctaactttttccgcctggagagccctcagtgtggcttcttacatttaaaaaacaaaaaggatcagctgccaggtgtgaggcagtccccaagctgagttgtgaggatgtaagcatgaataagtccctgcactcaaaatggtcaaagaattaaaccccatggacttttttggcatctgtatgaaagcttgggttttctgaggactgtcttgctatagttaagtcagatcctagatgaaatatacttgttcatactgtactaggttcttaggaaacaacagaattcctcaaatgccaaaaacaaagaaaatagaaacccagaaaacaaaacaaaataaaacaaaaccatcagaactgtgagtggaaactaaggtgatgatctgggagcaatacactaaaatcttgggtcgagacctatatgaaggctggcagtggagctaaacctggacacactgaagacaagggagctgaaccagggctcctacatgaagcagggataactgatggcagtaaatgtggtctcaaattgcagatggtctggaggaaaatttcccaaatttagagcctcaggattcccaaagatcctccaaatatgagctcacaatcaaagatcagagacgttgaaaaataaaaaacaccttaagtgggcagcataaaaaacagctaatttagaaccccaaaggcttcagatgtcagaatattagagacttatgataataagcaatatttgcagagtatttgtatgtgccagacactattgtaagtgcttcatcatgtactgattcatttaatactcacagaaatctgtgagatgggtattattcttatcctcactctatggattaaaaaaactaaggcacaaagtggttaagctccttgcctgagattatagactgtaagttgaacgtgagcacttggaatacagagttcatgctgtaaactaccacactatagggcctccaatatgataatttataaaatatttgaataaaaaatgaatactagttccacattttaaaatcatgtttaactgtggtcaaatgcacataacacaagttgccatcttcaccatttttaggtgtatagttcagtggtgttatgtacattcacactattgtgcagtcatcaccaccatccatctccagaacagaaactcagtacccatcaaacaactctccatttccccctcctcccaatctctggcaaccaccattgtgctttcagtctctgtgaactggattactctgggtacctcatttaagtgaagtcatgcagtattggtctttttgtacttgttttatttcacttcacattgtgtcttcaagtttcacccatgttgtagcatgtgtcagaatttcttccctttttagactaaataatattctattgtttatacgaacattcaggttacttctatcttttggctattgtgaattatgctgctgtgaacatgggtgtacaagtatctctttgaggccctgctttcaattctcttgggtatattcccagaagtggaattgctggatcatatggtaattctattttgaattttttgaggaactgatatattgctttccatagagactgcaccattttacattcccatcaacagtttgcaggagttactatttctccatatcccccctaacacttgctattttctgttaaaaatggatatcttaataatcaagcaaaaataacaggcagatttgaaaaagaactgaatacagcttttagaaataaaaactataattataaaaataaaaaactaagtggatggggtaaataacaattaaaacaccaattaagagagaacaaatgaactggaagataaattgaagaagtgactaggcttaacagcagagagagataaggagattaaaaatatgaaaacaaggccaggagcaatgaagcctagaatggtaaattctaacatatccagaatcccagaaagagagaatcaagacaatgagagagagacagtaccaaagagataagagctgagaatgttccagaattgataaaaggtgtgaatccacagaacatacaccaccatagtgtacacgcatacaaccaaggtggaaaaattagaataaatccacacctatgtacattataatgaaactgcagaacaccaaagacaaaaagaaactccttatagcagcagagagaaaacccagaccacccacagtaccacaaatctaccacaattagactgacaacaggctttcccacagcaataaaggagctagaagtcagtggaagtatatctccagcatgccaaaagataacaatcaatcagggattgtgaaccctacaaaactatctttcaagaataaaggcattttcaagaaaacaaaaacagactttaccatcaacaaaccttctctaaaagaatatataaagcatttactttaggaagaaggaaaatgatcctaaaaggaagaaccaagaagcaagtagcaatagtgaggcaattgtgaaaatgtaggtaagtctaaacacactctgtctacttcttcttcttcttcttcttcttcttcttcttattttgagactgagtcttgccctgtcacccagactggagtgcagtggcaggatcttggctcactgctatctccacctcccaggttcaagtgattcttctgcctcagcctcccgagtagctgggattacatgcacatgccaccatatccggctaatttttgaatttttagtagagatggggtttcactgtgttggccaggccggtctcaaactcccgacctcaagtgatccccccgcctcggcctcccaaagtgctgggattacaggcgtgtctacatattattaaaataacaataatatttattttgtgggttaattttttttgaaacagatattgaatttattggttggctatgagtagaaaaatacatcagtaaagaaaaaagaccctgtatataaatataatactagctagttaaaatttgaccaagaagtttccattgtgggttaatttttaaaggcctaactgaaatatggagtaaccacagcatgcagcatgtaaattaaaggggatagctgg

SEQ ID NO: 22 - DNA, 5431 bp, Homo sapiens
actcctggaatacacagagagaggcagcagcttgctcagcggacaaggatgctgggcgtgagggaccaaggcctgccctgcactcgggcctcctccagccagtgctgaccagggacttctgacctgctggccagccaggacctgtgtggggaggccctcctgctgccttggggtgacaatctcagctccaggctacagggagaccgggaggatcacagagccagcatggatcctgacagtgatcaacctctgaacagcctcgatgtcaaacccctgcgcaaaccccgtatccccatggagaccttcagaaagtcaaggtgattctggataaatactacttcctctgcgggcagcctctccacttcatcccgaggaagcagctgtgtgacggagagctggactgtcccttgggggaggacgaggagcactgtgtcaagagcttccccgaagggcctgcagtggcagtccgcctctccaaggaccgatccacactgcaggtgctggactcggccacagggaactggttctctgcctgtttcgacaacttcacagaagctctcgctgagacagcctgtaggcagatgggctacagcagagctgtggagattggcccagaccaggatctggatgttgttgaaatcacagaaaacagccaggagcttcgcatgcggaactcaagtgggccctgtctctcaggctccctggtctccctgcactgtcttgcctgtgggaagagcctgaagaccccccgtgtggtgggtgtggaggaggcctctgtggattcttggccttggcaggtcagcatccagtacgacaaacagcacgtctgtggagggagcatcctggacccccactgggtcctcacggcagcccactgcttcaggaaacataccgatgtgttcaactggaaggtgcgggcaggctcagacaaactgggcagcttcccatccctggctgtggccaagatcatcatcattgaattcaaccccatgtaccccaaagacaatgacatcgccctcatgaagctgcagttcccactcactttctcaggcacagtcaggcccatctgtctgcccttctttgatgaggagctcactccagccaccccactctggatcattggatggggctttacgaagcagaatggagggaagatgtctgacatactgctgcaggcgtcagtccaggtcattgacagcacacggtgcaatgcagacgatgcgtaccagggggaagtcaccgagaagatgatgtgtgcaggcatcccggaagggggtgtggacacctgccagggtgacagtggtgggcccctgatgtaccaatctgaccagtggcatgtggtgggcatcgttagttggggctatggctgcgggggcccgagcaccccaggagtatacaccaaggtctcagcctatctcaactggatctacaatgtctggaaggctgagctgtaatgctgctgcccctttgcagtgctgggagccgcttccttcctgccctgcccacctggggatcccccaaagtcagacacagagcaagagtccccttgggtacacccctctgcccacagcctcagcatttcttggagcagcaaagggcctcaattcctataagagaccctcgcagcccagaggcgcccagaggaagtcagcagccctagctcggccacacttggtgctcccagcatcccagggagagacacagcccactgaacaaggtctcaggggtattgctaagccaagaaggaactttcccacactactgaatggaagcaggctgtcttgtaaaagcccagatcactgtgggctggagaggagaaggaaagggtctgcgccagccctgtccgtcttcacccatccccaagcctactagagcaagaaaccagttgtaatataaaatgcactgccctactgttggtatgactaccgttacctactgttgtcattgttattacagctatggccactattattaaagagctgtgtaacatctctggcataggctagctggaatgcttgataagaactgagctgggatgattgaactttcattctttggcttggggagaaaagaagtcctggggaagcaattgagtctcaaagtagaggcaggggaaaaaagagttagggagaccagatctgctgagtggcagcaagagtgagctgcagattacagaaaccagggtgagcaagtttgagtcccacacagggccttctccctttgcctctttccctccctccctgcctgtgataatcagccaggagccagggataacctatgacttgggaaagagatgagttaggcagtcaagggtgacattcaatcagggatccacaagtggctggaaagaaatgctggtcctgtgtcctaactttttccgcctggagagccctcagtgtggcttcttacatttaaaaaacaaaaaggatcagctgccaggtgtgaggcagtccccaagctgagttgtgaggatgtaagcatgaataagtccctgcactcaaaatggtcaaagaattaaaccccatggacttttttggcatctgtatgaaagcttgggttttctgaggactgtcttgctatagttaagtcagatcctagatgaaatatacttgttcatactgtactaggttcttaggaaacaacagaattcctcaaatgccaaaaacaaagaaaatagaaacccagaaaacaaaacaaaataaaacaaaaccatcagaactgtgagtggaaactaaggtgatgatctgggagcaatacactaaaatcttgggtcgagacctatatgaaggctggcagtggagctaaacctggacacactgaagacaagggagctgaaccagggctcctacatgaagcagggataactgatggcagtaaatgtggtctcaaattgcagatggtctggaggaaaatttcccaaatttagagcctcaggattcccaaagatcctccaaatatgagctcacaatcaaagatcagagacgttgaaaaataaaaaacaccttaagtgggcagcataaaaaacagctaatttagaaccccaaaggcttcagatgtcagaatattagagacttatgataataagcaatatttgcagagtatttgtatgtgccagacactattgtaagtgcttcatcatgtactgattcatttaatactcacagaaatctgtgagatgggtattattcttatcctcactctatggattaaaaaaactaaggcacaaagtggttaagctccttgcctgagattatagactgtaagttgaacgtgagcacttggaatacagagttcatgctgtaaactaccacactatagggcctccaatatgataatttataaaatatttgaataaaaaatgaatactagttccacattttaaaatcatgtttaactgtggtcaaatgcacataacacaagttgccatcttcaccatttttaggtgtatagttcagtggtgttatgtacattcacactattgtgcagtcatcaccaccatccatctccagaacagaaactcagtacccatcaaacaactctccatttccccctcctcccaatctctggcaaccaccattgtgctttcagtctctgtgaactggattactctgggtacctcatttaagtgaagtcatgcagtattggtctttttgtacttgttttatttcacttcacattgtgtcttcaagtttcacccatgttgtagcatgtgtcagaatttcttccctttttagactaaataatattctattgtttatacgaacattcaggttacttctatcttttggctattgtgaattatgctgctgtgaacatgggtgtacaagtatctctttgaggccctgctttcaattctcttgggtatattcccagaagtggaattgctggatcatatggtaattctattttgaattttttgaggaactgatatattgctttccatagagactgcaccattttacattcccatcaacagtttgcaggagttactatttctccatatcccccctaacacttgctattttctgttaaaaatggatatcttaataatcaagcaaaaataacaggcagatttgaaaaagaactgaatacagcttttagaaataaaaactataattataaaaataaaaaactaagtggatggggtaaataacaattaaaacaccaattaagagagaacaaatgaactggaagataaattgaagaagtgactaggcttaacagcagagagagataaggagattaaaaatatgaaaacaaggccaggagcaatgaagcctagaatggtaaattctaacatatccagaatcccagaaagagagaatcaagacaatgagagagagacagtaccaaagagataagagctgagaatgttccagaattgataaaaggtgtgaatccacagaacatacaccaccatagtgtacacgcatacaaccaaggtggaaaaattagaataaatccacacctatgtacattataatgaaactgcagaacaccaaagacaaaaagaaactccttatagcagcagagagaaaacccagaccacccacagtaccacaaatctaccacaattagactgacaacaggctttcccacagcaataaaggagctagaagtcagtggaagtatatctccagcatgccaaaagataacaatcaatcagggattgtgaaccctacaaaactatctttcaagaataaaggcattttcaagaaaacaaaaacagactttaccatcaacaaaccttctctaaaagaatatataaagcatttactttaggaagaaggaaaatgatcctaaaaggaagaaccaagaagcaagtagcaatagtgaggcaattgtgaaaatgtaggtaagtctaaacacactctgtctacttcttcttcttcttcttcttcttcttcttcttattttgagactgagtcttgccctgtcacccagactggagtgcagtggcaggatcttggctcactgctatctccacctcccaggttcaagtgattcttctgcctcagcctcccgagtagctgggattacatgcacatgccaccatatccggctaatttttgaatttttagtagagatggggtttcactgtgttggccaggccggtctcaaactcccgacctcaagtgatccccccgcctcggcctcccaaagtgctgggattacaggcgtgtctacatattattaaaataacaataatatttattttgtgggttaattttttttgaaacagatattgaatttattggttggctatgagtagaaaaatacatcagtaaagaaaaaagaccctgtatataaatataatactagctagttaaaatttgaccaagaagtttccattgtgggttaatttttaaaggcctaactgaaatatggagtaaccacagcatgcagcatgtaaattaaaggggatagctgg

SEQ ID NO: 23 - DNA, 5516 bp, Homo sapiens
actcctggaatacacagagagaggcagcagcttgctcagcggacaaggatgctgggcgtgagggaccaaggcctgccctgcactcgggcctcctccagccagtgctgaccagggacttctgacctgctggccagccaggacctgtgtggggaggccctcctgctgccttggggtgacaatctcagctccaggctacagggagaccgggaggatcacagagccagcatgttacaggatcctgacagtgatcaacctctgaacagcctcgatgtcaaacccctgcgcaaaccccgtatccccatggagaccttcagaaaggtggggatccccatcatcatagcactactgagcctggcgagtatcatcattgtggttgtcctcatcaaggtgattctggataaatactacttcctctgcgggcagcctctccacttcatcccgaggaagcagctgtgtgacggagagctggactgtcccttgggggaggacgaggagcactgtgtcaagagcttccccgaagggcctgcagtggcagtccgcctctccaaggaccgatccacactgcaggtgctggactcggccacagggaactggttctctgcctgtttcgacaacttcacagaagctctcgctgagacagcctgtaggcagatgggctacagcagcaaacccactttcagagctgtggagattggcccagaccaggatctggatgttgttgaaatcacagaaaacagccaggagcttcgcatgcggaactcaagtgggccctgtctctcaggctccctggtctccctgcactgtcttgcctgtgggaagagcctgaagaccccccgtgtggtgggtgtggaggaggcctctgtggattcttggccttggcaggtcagcatccagtacgacaaacagcacgtctgtggagggagcatcctggacccccactgggtcctcacggcagcccactgcttcaggaaacataccgatgtgttcaactggaaggtgcgggcaggctcagacaaactgggcagcttcccatccctggctgtggccaagatcatcatcattgaattcaaccccatgtaccccaaagacaatgacatcgccctcatgaagctgcagttcccactcactttctcaggcacagtcaggcccatctgtctgcccttctttgatgaggagctcactccagccaccccactctggatcattggatggggctttacgaagcagaatggagggaagatgtctgacatactgctgcaggcgtcagtccaggtcattgacagcacacggtgcaatgcagacgatgcgtaccagggggaagtcaccgagaagatgatgtgtgcaggcatcccggaagggggtgtggacacctgccagggtgacagtggtgggcccctgatgtaccaatctgaccagtggcatgtggtgggcatcgttagttggggctatggctgcgggggcccgagcaccccaggagtatacaccaaggtctcagcctatctcaactggatctacaatgtctggaaggctgagctgtaatgctgctgcccctttgcagtgctgggagccgcttccttcctgccctgcccacctggggatcccccaaagtcagacacagagcaagagtccccttgggtacacccctctgcccacagcctcagcatttcttggagcagcaaagggcctcaattcctataagagaccctcgcagcccagaggcgcccagaggaagtcagcagccctagctcggccacacttggtgctcccagcatcccagggagagacacagcccactgaacaaggtctcaggggtattgctaagccaagaaggaactttcccacactactgaatggaagcaggctgtcttgtaaaagcccagatcactgtgggctggagaggagaaggaaagggtctgcgccagccctgtccgtcttcacccatccccaagcctactagagcaagaaaccagttgtaatataaaatgcactgccctactgttggtatgactaccgttacctactgttgtcattgttattacagctatggccactattattaaagagctgtgtaacatctctggcataggctagctggaatgcttgataagaactgagctgggatgattgaactttcattctttggcttggggagaaaagaagtcctggggaagcaattgagtctcaaagtagaggcaggggaaaaaagagttagggagaccagatctgctgagtggcagcaagagtgagctgcagattacagaaaccagggtgagcaagtttgagtcccacacagggccttctccctttgcctctttccctccctccctgcctgtgataatcagccaggagccagggataacctatgacttgggaaagagatgagttaggcagtcaagggtgacattcaatcagggatccacaagtggctggaaagaaatgctggtcctgtgtcctaactttttccgcctggagagccctcagtgtggcttcttacatttaaaaaacaaaaaggatcagctgccaggtgtgaggcagtccccaagctgagttgtgaggatgtaagcatgaataagtccctgcactcaaaatggtcaaagaattaaaccccatggacttttttggcatctgtatgaaagcttgggttttctgaggactgtcttgctatagttaagtcagatcctagatgaaatatacttgttcatactgtactaggttcttaggaaacaacagaattcctcaaatgccaaaaacaaagaaaatagaaacccagaaaacaaaacaaaataaaacaaaaccatcagaactgtgagtggaaactaaggtgatgatctgggagcaatacactaaaatcttgggtcgagacctatatgaaggctggcagtggagctaaacctggacacactgaagacaagggagctgaaccagggctcctacatgaagcagggataactgatggcagtaaatgtggtctcaaattgcagatggtctggaggaaaatttcccaaatttagagcctcaggattcccaaagatcctccaaatatgagctcacaatcaaagatcagagacgttgaaaaataaaaaacaccttaagtgggcagcataaaaaacagctaatttagaaccccaaaggcttcagatgtcagaatattagagacttatgataataagcaatatttgcagagtatttgtatgtgccagacactattgtaagtgcttcatcatgtactgattcatttaatactcacagaaatctgtgagatgggtattattcttatcctcactctatggattaaaaaaactaaggcacaaagtggttaagctccttgcctgagattatagactgtaagttgaacgtgagcacttggaatacagagttcatgctgtaaactaccacactatagggcctccaatatgataatttataaaatatttgaataaaaaatgaatactagttccacattttaaaatcatgtttaactgtggtcaaatgcacataacacaagttgccatcttcaccatttttaggtgtatagttcagtggtgttatgtacattcacactattgtgcagtcatcaccaccatccatctccagaacagaaactcagtacccatcaaacaactctccatttccccctcctcccaatctctggcaaccaccattgtgctttcagtctctgtgaactggattactctgggtacctcatttaagtgaagtcatgcagtattggtctttttgtacttgttttatttcacttcacattgtgtcttcaagtttcacccatgttgtagcatgtgtcagaatttcttccctttttagactaaataatattctattgtttatacgaacattcaggttacttctatcttttggctattgtgaattatgctgctgtgaacatgggtgtacaagtatctctttgaggccctgctttcaattctcttgggtatattcccagaagtggaattgctggatcatatggtaattctattttgaattttttgaggaactgatatattgctttccatagagactgcaccattttacattcccatcaacagtttgcaggagttactatttctccatatcccccctaacacttgctattttctgttaaaaatggatatcttaataatcaagcaaaaataacaggcagatttgaaaaagaactgaatacagcttttagaaataaaaactataattataaaaataaaaaactaagtggatggggtaaataacaattaaaacaccaattaagagagaacaaatgaactggaagataaattgaagaagtgactaggcttaacagcagagagagataaggagattaaaaatatgaaaacaaggccaggagcaatgaagcctagaatggtaaattctaacatatccagaatcccagaaagagagaatcaagacaatgagagagagacagtaccaaagagataagagctgagaatgttccagaattgataaaaggtgtgaatccacagaacatacaccaccatagtgtacacgcatacaaccaaggtggaaaaattagaataaatccacacctatgtacattataatgaaactgcagaacaccaaagacaaaaagaaactccttatagcagcagagagaaaacccagaccacccacagtaccacaaatctaccacaattagactgacaacaggctttcccacagcaataaaggagctagaagtcagtggaagtatatctccagcatgccaaaagataacaatcaatcagggattgtgaaccctacaaaactatctttcaagaataaaggcattttcaagaaaacaaaaacagactttaccatcaacaaaccttctctaaaagaatatataaagcatttactttaggaagaaggaaaatgatcctaaaaggaagaaccaagaagcaagtagcaatagtgaggcaattgtgaaaatgtaggtaagtctaaacacactctgtctacttcttcttcttcttcttcttcttcttcttcttattttgagactgagtcttgccctgtcacccagactggagtgcagtggcaggatcttggctcactgctatctccacctcccaggttcaagtgattcttctgcctcagcctcccgagtagctgggattacatgcacatgccaccatatccggctaatttttgaatttttagtagagatggggtttcactgtgttggccaggccggtctcaaactcccgacctcaagtgatccccccgcctcggcctcccaaagtgctgggattacaggcgtgtctacatattattaaaataacaataatatttattttgtgggttaattttttttgaaacagatattgaatttattggttggctatgagtagaaaaatacatcagtaaagaaaaaagaccctgtatataaatataatactagctagttaaaatttgaccaagaagtttccattgtgggttaatttttaaaggcctaactgaaatatggagtaaccacagcatgcagcatgtaaattaaaggggatagctgg

SEQ ID NO: 24 - protein, 432 aa, Homo sapiens
MLQDPDSDQPLNSLDVKPLRKPRIPMETFRKVGIPIIIALLSLASIIIVVVLIKVILDKYYFLCGQPLHFIPRKQLCDGELDCPLGEDEEHCVKSFPEGPAVAVRLSKDRSTLQVLDSATGNWFSACFDNFTEALAETACRQMGYSRAVEIGPDQDLDVVEITENSQELRMRNSSGPCLSGSLVSLHCLACGKSLKTPRVVGVEEASVDSWPWQVSIQYDKQHVCGGSILDPHWVLTAAHCFRKHTDVFNWKVRAGSDKLGSFPSLAVAKIIIIEFNPMYPKDNDIALMKLQFPLTFSGTVRPICLPFFDEELTPATPLWIIGWGFTKQNGGKMSDILLQASVQVIDSTRCNADDAYQGEVTEKMMCAGIPEGGVDTCQGDSGGPLMYQSDQWHVVGIVSWGYGCGGPSTPGVYTKVSAYLNWIYNVWKAEL

SEQ ID NO: 25 - protein, 435 aa, Homo sapiens
MDPDSDQPLNSLDVKPLRKPRIPMETFRKVGIPIIIALLSLASIIIVVVLIKVILDKYYFLCGQPLHFIPRKQLCDGELDCPLGEDEEHCVKSFPEGPAVAVRLSKDRSTLQVLDSATGNWFSACFDNFTEALAETACRQMGYSSKPTFRAVEIGPDQDLDVVEITENSQELRMRNSSGPCLSGSLVSLHCLACGKSLKTPRVVGVEEASVDSWPWQVSIQYDKQHVCGGSILDPHWVLTAAHCFRKHTDVFNWKVRAGSDKLGSFPSLAVAKIIIIEFNPMYPKDNDIALMKLQFPLTFSGTVRPICLPFFDEELTPATPLWIIGWGFTKQNGGKMSDILLQASVQVIDSTRCNADDAYQGEVTEKMMCAGIPEGGVDTCQGDSGGPLMYQSDQWHVVGIVSWGYGCGGPSTPGVYTKVSAYLNWIYNVWKAEL

SEQ ID NO: 26 - protein, 397 aa, Homo sapiens
MDPDSDQPLNSLVKVILDKYYFLCGQPLHFIPRKQLCDGELDCPLGEDEEHCVKSFPEGPAVAVRLSKDRSTLQVLDSATGNWFSACFDNFTEALAETACRQMGYSSKPTFRAVEIGPDQDLDVVEITENSQELRMRNSSGPCLSGSLVSLHCLACGKSLKTPRVVGVEEASVDSWPWQVSIQYDKQHVCGGSILDPHWVLTAAHCFRKHTDVFNWKVRAGSDKLGSFPSLAVAKIIIIEFNPMYPKDNDIALMKLQFPLTFSGTVRPICLPFFDEELTPATPLWIIGWGFTKQNGGKMSDILLQASVQVIDSTRCNADDAYQGEVTEKMMCAGIPEGGVDTCQGDSGGPLMYQSDQWHVVGIVSWGYGCGGPSTPGVYTKVSAYLNWIYNVWKAEL

SEQ ID NO: 27 - protein, 412 aa, Homo sapiens
METFRKVGIPIIIALLSLASIIIVVVLIKVILDKYYFLCGQPLHFIPRKQLCDGELDCPLGEDEEHCVKSFPEGPAVAVRLSKDRSTLQVLDSATGNWFSACFDNFTEALAETACRQMGYSSKPTFRAVEIGPDQDLDVVEITENSQELRMRNSSGPCLSGSLVSLHCLACGKSLKTPRVVGVEEASVDSWPWQVSIQYDKQHVCGGSILDPHWVLTAAHCFRKHTDVFNWKVRAGSDKLGSFPSLAVAKIIIIEFNPMYPKDNDIALMKLQFPLTFSGTVRPICLPFFDEELTPATPLWIIGWGFTKQNGGKMSDILLQASVQVIDSTRCNADDAYQGEVTEKMMCAGIPEGGVDTCQGDSGGPLMYQSDQWHVVGIVSWGYGCGGPSTPGVYTKVSAYLNWIYNVWKAEL

SEQ ID NO: 28 - protein, 290 aa, Homo sapiens
MGYSRAVEIGPDQDLDVVEITENSQELRMRNSSGPCLSGSLVSLHCLACGKSLKTPRVVGVEEASVDSWPWQVSIQYDKQHVCGGSILDPHWVLTAAHCFRKHTDVFNWKVRAGSDKLGSFPSLAVAKIIIIEFNPMYPKDNDIALMKLQFPLTFSGTVRPICLPFFDEELTPATPLWIIGWGFTKQNGGKMSDILLQASVQVIDSTRCNADDAYQGEVTEKMMCAGIPEGGVDTCQGDSGGPLMYQSDQWHVVGIVSWGYGCGGPSTPGVYTKVSAYLNWIYNVWKAEL

SEQ ID NO: 29 - protein, 437 aa, Homo sapiens
MLQDPDSDQPLNSLDVKPLRKPRIPMETFRKVGIPIIIALLSLASIIIVVVLIKVILDKYYFLCGQPLHFIPRKQLCDGELDCPLGEDEEHCVKSFPEGPAVAVRLSKDRSTLQVLDSATGNWFSACFDNFTEALAETACRQMGYSSKPTFRAVEIGPDQDLDVVEITENSQELRMRNSSGPCLSGSLVSLHCLACGKSLKTPRVVGVEEASVDSWPWQVSIQYDKQHVCGGSILDPHWVLTAAHCFRKHTDVFNWKVRAGSDKLGSFPSLAVAKIIIIEFNPMYPKDNDIALMKLQFPLTFSGTVRPICLPFFDEELTPATPLWIIGWGFTKQNGGKMSDILLQASVQVIDSTRCNADDAYQGEVTEKMMCAGIPEGGVDTCQGDSGGPLMYQSDQWHVVGIVSWGYGCGGPSTPGVYTKVSAYLNWIYNVWKAEL

SEQ ID NO: 30 - DNA, 5168 bp, Homo sapiens
actcgccctccagcttctgccctgcctgctgtgtgcggagccgtccagcgaccaccatggtgaggctcgtgctgcccaaccccggcctagacgcccggatcccgtccctggctgagctggagaccatcgagcaggaggaggccagctcccggccgaagtgggacaacaaggcgcagtacatgctcacctgcctgggcttctgcgtgggcctcggcaacgtgtggcgcttcccctacctgtgtcagagccacggaggaggagccttcatgatcccgttcctcatcctgctggtcctggagggcatccccctgctgtacctggagttcgccatcgggcagcggctgcggcggggcagcctgggtgtgtggagctccatccacccggccctgaagggcctaggcctggcctccatgctcacgtccttcatggtgggactgtattacaacaccatcatctcctggatcatgtggtacttattcaactccttccaggagcctctgccctggagcgactgcccgctcaacgagaaccagacagggtatgtggacgagtgcgccaggagctcccctgtggactacttctggtaccgagagacgctcaacatctccacgtccatcagcgactcgggctccatccagtggtggatgctgctgtgcctggcctgcgcatggagcgtcctgtacatgtgcaccatccgcggcatcgagaccaccgggaaggccgtgtacatcacctccacgctgccctatgtcgtcctgaccatcttcctcatccgaggcctgacgctgaagggcgccaccaatggcatcgtcttcctcttcacgcccaacgtcacggagctggcccagccggacacctggctggacgcgggcgcacaggtcttcttctccttctccctggccttcgggggcctcatctccttctccagctacaactctgtgcacaacaactgcgagaaggactcggtgattgtgtccatcatcaacggcttcacatcggtgtatgtggccatcgtggtctactccgtcattgggttccgcgccacacagcgctacgacgactgcttcagcacgaacatcctgaccctcatcaacgggttcgacctgcctgaaggcaacgtgacccaggagaactttgtggacatgcagcagcggtgcaacgcctccgaccccgcggcctacgcgcagctggtgttccagacctgcgacatcaacgccttcctctcagaggccgtggagggcacaggcctggccttcatcgtcttcaccgaggccatcaccaagatgccgttgtccccactgtggtctgtgctcttcttcattatgctcttctgcctggggctgtcatctatgtttgggaacatggagggcgtcgttgtgcccctgcaggacctcagagtcatccccccgaagtggcccaaggaggtgctcacaggcctcatctgcctggggacattcctcattggcttcatcttcacgctgaactccggccagtactggctctccctgctggacagctatgccggctccattcccctgctcatcatcgccttctgcgagatgttctctgtggtctacgtgtacggtgtggacaggttcaataaggacatcgagttcatgatcggccacaagcccaacatcttctggcaagtcacgtggcgcgtggtcagccccctgctcatgctgatcatcttcctcttcttcttcgtggtagaggtcagtcaggagctgacctacagcatctgggaccctggctacgaggaatttcccaaatcccagaagatctcctacccgaactgggtgtatgtggtggtggtgattgtggctggagtgccctccctcaccatccctggctatgccatctacaagctcatcaggaaccactgccagaagccaggggaccatcaggggctggtgagcacactgtccacagcctccatgaacggggacctgaagtactgagaaggcccatcccacggcgtgccatacactggtgtcagggaaggaggaaccagcaagacctgtggggtgggggccgggctgcacctgcatgtgtgtaagcgtgagtgtatgctcgtgtgtgagtgtgtgtattgtacacgcatgtgccatgtgtgcagatatgtatcgtgtgtgcatgtacatgcatgggcactgtgtgagtgtgcacgtgtatgcacacatatacatgtgtgtgggtgtgtgtattgtatgtgcatgtgccatgtgtgcagatgtgtcatgttgtgtgtgtgcatgtacatgtatggacattgtgtgagtgtgcaagtgtgcatgcatatacatgtgtgcgatatttgctgcccgtgtgtgtgcatgtatatatagacatacatgcctatgttgtgtgtggtgtgcatatgtgtgaacacacacgtgtatacatgcatgcacatgtgctcgtacaatgggtgtccacatgcacgtgtatatgtatatctgtgagtgtatatacatgcatgcaattgtgtgtatgtgtgttctgtgtgtgcgtttgcaagtatatatgcacatgtgtatatgtacatgtatgcctgtgtgacgtgtgtatatgtgagcatgtgtacgtgtgtgtatacgtgtgttgtgtatatgtgtgtgtctgtacctgtttgtgtatatgtgtgtgatgtgtgctcgtgtgtgtgcatattcaggcaggtgtgcatttgtgcatgccagtgtgtatgtatgtgcgcatatggacacgcatggacacgcatatggacacatatggacacacatatggacacgtgtggatatgtgtgcgtacacgtcgctgggacacatgcctgccactcggggcccagctgccctctgtgtttgtccttgccacagtcacggggtgcatgtgcagaggggagcagaccactggggacgtgctgtgccctgcacgtgcccgggggaagcggaagctgcagctggggtgggggcagcacctctatgcttcatctctgtgggtggcaggagacaaaagcacagggtactatcttggctcctgggagcgactcttgctacccacccccacccatccccttccccttggtgttgacctttgacctgggggttcccagagccctgtagccctcgacccggagcagcctctcggaagccggagtgggcagttgctggcgattctgagaaaacttggccgcatccaccggggccctgcctccagtcggccgctgccgagtctctgcgttctggccgcttcccggcttaatgaatgccagccatttaatcattgctcctgccaccacaaatagatgagcagttaaataaaactcaacttggcataattcaaggcaaataccactctgtgcattttcttaagaggacatgagctgtgtgaatttttagccagcctttggaaaagatgggttacagggtaactcaaccctggctgccatccttgggcactgtgtgtgtccagggcaccttggaggaccgtgcagcccccagaagcttccagctcccgcaccactcagtgaagcccagcctggcgcctgccctgcccccgtcacgggatgggcccccattggggttcaacattccatcgcagccaaaggcagtcggcacttgggacatctgcttccacggacaggtcacctccgctttgcacggaagaatctggatgcttacattaaactggtgttctgagagttcctacggacaggtcacctccgctttgcatggaagaatctggatgcttacattaaactggtgttctgagagttcctacggacaggtcacctctgctttccatagaagaatctggacgcttacattaaactgatgttctgagaattcctacaggcaggactgaaagcctggtgtgtgccagtatgatgttccacccacagaaacctggtcacaatcgtcccttccagcaccccatccagcagtgactgcacacactgagtcccctaccagcccctttcaccctgctgactgtcactgggccctgggatgcgcaagactccacagcagcagaggtggggggacatatcacagcctctgcccccggctgtgatgccaccgaggggctcgcctgctgatggcttcaacagggtctcacctcatcttttcctgctctttggccctggatcgagaaaatttccatcagtgccccattaatatgctgccctgtggcatctgcccaggaggccctgccaggcgtgcacaggtgtgcattggtgtaccctggcatgcacaggtgtgcactgatgtgccctggcatccattggtgtaccctggtgtgcctgccataggaccctgggcgggagctcccatctcatctacatctcctgattcatgcgttgtttcataggtttcaatgtctctgtaaatgtggtagaaatgcaggctttatgggcataaagtgtacatttctaaataaatcccttctattgagtatgctcaccctagaagttactgttgtccagacgtagagggatgagtgagccagtgacctcagacgggatggtggggacggcaggtccagctcctgcctcctcctggggggtctggctttgggggcttgctccgaagaggccatggcccaggcctgtggcctcacaatggggaccaaccagctcttctcatcttcttccctcacacttcctctcactcaaataagaaccttccaaaaatgtgtccacctgggcccctgccctgggactcatggatttggagttgtggccacacggttgaggggtgcagtgtccagtggaatggggcaattgcgggcctgggggcccttggcctgtccgtggcgggagcatctgcaaggaggagccccagagtccagggagcactgtggggagctccttagagctgaactcacccggcgtcaactcatcaaccctccacccatggacaggggtgcccccagcacaggagaggactcagccctctgcccccacgcacggtgggtgcctgtcaccctgtcctgcccagcggcccgagggcagcagtgggtgtgagggcagcccccggcctcccaagagcagctgagaggatccctgcgggaatccgggcttcgggtgcatgcgatctgatctgagttgtttctgacagtgacagagtgacaatctataagtatctcaagatcaaatggttaaataaaacataagaaatttaaaacga

SEQ ID NO: 31 - protein, 634 aa, Homo sapiens
MVRLVLPNPGLDARIPSLAELETIEQEEASSRPKWDNKAQYMLTCLGFCVGLGNVWRFPYLCQSHGGGAFMIPFLILLVLEGIPLLYLEFAIGQRLRRGSLGVWSSIHPALKGLGLASMLTSFMVGLYYNTIISWIMWYLFNSFQEPLPWSDCPLNENQTGYVDECARSSPVDYFWYRETLNISTSISDSGSIQWWMLLCLACAWSVLYMCTIRGIETTGKAVYITSTLPYVVLTIFLIRGLTLKGATNGIVFLFTPNVTELAQPDTWLDAGAQVFFSFSLAFGGLISFSSYNSVHNNCEKDSVIVSIINGFTSVYVAIVVYSVIGFRATQRYDDCFSTNILTLINGFDLPEGNVTQENFVDMQQRCNASDPAAYAQLVFQTCDINAFLSEAVEGTGLAFIVFTEAITKMPLSPLWSVLFFIMLFCLGLSSMFGNMEGVVVPLQDLRVIPPKWPKEVLTGLICLGTFLIGFIFTLNSGQYWLSLLDSYAGSIPLLIIAFCEMFSVVYVYGVDRFNKDIEFMIGHKPNIFWQVTWRVVSPLLMLIIFLFFFVVEVSQELTYSIWDPGYEEFPKSQKISYPNWVYVVVVIVAGVPSLTIPGYAIYKLIRNHCQKPGDHQGLVSTLSTASMNGDLKY

SEQ ID NO: 32 - DNA, 5015 bp, Homo sapiens
agaagcggagcgtatacggaggaggcgggatgcatttctgcatcgagcgcacaaagttatctaaaacagttcatgctgctgaaaacctccttcctggcagatgtccctcaaccctactggtgcctggcttctgagacacacgcttctctgaagtagctttggaaagtagagaagaaaatccagtttgcttcttggagaacactggacagctgaataaatgcagtatctaaatataaaagaggactgcaatgccatggctttctgtgctaaaatgaggagctccaagaagactgaggtgaacctggaggcccctgagccaggggtggaagtgatcttctatctgtcggacagggagcccctccggctgggcagtggagagtacacagcagaggaactgtgcatcagggctgcacaggcatgccgtatctctcctctttgtcacaacctctttgccctgtatgacgagaacaccaagctctggtatgctccaaatcgcaccatcaccgttgatgacaagatgtccctccggctccactaccggatgaggttctatttcaccaattggcatggaaccaacgacaatgagcagtcagtgtggcgtcattctccaaagaagcagaaaaatggctacgagaaaaaaaagattccagatgcaacccctctccttgatgccagctcactggagtatctgtttgctcagggacagtatgatttggtgaaatgcctggctcctattcgagaccccaagaccgagcaggatggacatgatattgagaacgagtgtctagggatggctgtcctggccatctcacactatgccatgatgaagaagatgcagttgccagaactgcccaaggacatcagctacaagcgatatattccagaaacattgaataagtccatcagacagaggaaccttctcaccaggatgcggataaataatgttttcaaggatttcctaaaggaatttaacaacaagaccatttgtgacagcagcgtgtccacgcatgacctgaaggtgaaatacttggctaccttggaaactttgacaaaacattacggtgctgaaatatttgagacttccatgttactgatttcatcagaaaatgagatgaattggtttcattcgaatgacggtggaaacgttctctactacgaagtgatggtgactgggaatcttggaatccagtggaggcataaaccaaatgttgtttctgttgaaaaggaaaaaaataaactgaagcggaaaaaactggaaaataaacacaagaaggatgaggagaaaaacaagatccgggaagagtggaacaatttttcttacttccctgaaatcactcacattgtaataaaggagtctgtggtcagcattaacaagcaggacaacaagaaaatggaactgaagctctcttcccacgaggaggccttgtcctttgtgtccctggtagatggctacttccggctcacagcagatgcccatcattacctctgcaccgacgtggcccccccgttgatcgtccacaacatacagaatggctgtcatggtccaatctgtacagaatacgccatcaataaattgcggcaagaaggaagcgaggaggggatgtacgtgctgaggtggagctgcaccgactttgacaacatcctcatgaccgtcacctgctttgagaagtctgagcaggtgcagggtgcccagaagcagttcaagaactttcagatcgaggtgcagaagggccgctacagtctgcacggttcggaccgcagcttccccagcttgggagacctcatgagccacctcaagaagcagatcctgcgcacggataacatcagcttcatgctaaaacgctgctgccagcccaagccccgagaaatctccaacctgctggtggctactaagaaagcccaggagtggcagcccgtctaccccatgagccagctgagtttcgatcggatcctcaagaaggatctggtgcagggcgagcaccttgggagaggcacgagaacacacatctattctgggaccctgatggattacaaggatgacgaaggaacttctgaagagaagaagataaaagtgatcctcaaagtcttagaccccagccacagggatatttccctggccttcttcgaggcagccagcatgatgagacaggtctcccacaaacacatcgtgtacctctatggcgtctgtgtccgcgacgtggagaatatcatggtggaagagtttgtggaagggggtcctctggatctcttcatgcaccggaaaagcgatgtccttaccacaccatggaaattcaaagttgccaaacagctggccagtgccctgagctacttggaggataaagacctggtccatggaaatgtgtgtactaaaaacctcctcctggcccgtgagggcatcgacagtgagtgtggcccattcatcaagctcagtgaccccggcatccccattacggtgctgtctaggcaagaatgcattgaacgaatcccatggattgctcctgagtgtgttgaggactccaagaacctgagtgtggctgctgacaagtggagctttggaaccacgctctgggaaatctgctacaatggcgagatccccttgaaagacaagacgctgattgagaaagagagattctatgaaagccggtgcaggccagtgacaccatcatgtaaggagctggctgacctcatgacccgctgcatgaactatgaccccaatcagaggcctttcttccgagccatcatgagagacattaataagcttgaagagcagaatccagatattgtttcagaaaaaaaaccagcaactgaagtggaccccacacattttgaaaagcgcttcctaaagaggatccgtgacttgggagagggccactttgggaaggttgagctctgcaggtatgaccccgaaggggacaatacaggggagcaggtggctgttaaatctctgaagcctgagagtggaggtaaccacatagctgatctgaaaaaggaaatcgagatcttaaggaacctctatcatgagaacattgtgaagtacaaaggaatctgcacagaagacggaggaaatggtattaagctcatcatggaatttctgccttcgggaagccttaaggaatatcttccaaagaataagaacaaaataaacctcaaacagcagctaaaatatgccgttcagatttgtaaggggatggactatttgggttctcggcaatacgttcaccgggacttggcagcaagaaatgtccttgttgagagtgaacaccaagtgaaaattggagacttcggtttaaccaaagcaattgaaaccgataaggagtattacaccgtcaaggatgaccgggacagccctgtgttttggtatgctccagaatgtttaatgcaatctaaattttatattgcctctgacgtctggtcttttggagtcactctgcatgagctgctgacttactgtgattcagattctagtcccatggctttgttcctgaaaatgataggcccaacccatggccagatgacagtcacaagacttgtgaatacgttaaaagaaggaaaacgcctgccgtgcccacctaactgtccagatgaggtttatcaacttatgaggaaatgctgggaattccaaccatccaatcggacaagctttcagaaccttattgaaggatttgaagcacttttaaaataagaagcatgaataacatttaaattccacagattatcaagtccttctcctgcaacaaatgcccaagtcattttttaaaaatttctaatgaaagaagtttgtgttctgtccaaaaagtcactgaactcatacttcagtacatatacatgtataaggcacactgtagtgcttaatatgtgtaaggacttcctctttaaatttggtaccagtaacttagtgacacataatgacaaccaaaatatttgaaagcacttaagcactcctccttgtggaaagaatataccaccatttcatctggctagttcaccatcacaactgcattaccaaaaggggatttttgaaaacgaggagttgaccaaaataatatctgaagatgattgcttttccctgctgccagctgatctgaaatgttttgctggcacattaatcatagataaagaaagattgatggacttagccctcaaatttcagtatctatacagtactagaccatgcattcttaaaatattagataccaggtagtatatattgtttctgtacaaaaatgactgtattctctcaccagtaggacttaaactttgtttctccagtggcttagctcctgttcctttgggtgatcactagcacccatttttgagaaagctggttctacatggggggatagctgtggaatagataatttgctgcatgttaattctcaagaactaagcctgtgccagtgctttcctaagcagtatacctttaatcagaactcattcccagaacctggatgctattacacatgcttttaagaaacgtcaatgtatatccttttataactctaccactttggggcaagctattccagcactggttttgaatgctgtatgcaaccagtctgaataccacatacgctgcactgttcttagagggtttccatacttaccaccgatctacaagggttgatccctgtttttaccatcaatcatcaccctgtggtgcaacacttgaaagacccggctagaggcactatggacttcaggatccactagacagttttcagtttgcttggaggtagctgggtaatcaaaaatgtttagtcattgattcaatgtgaacgattacggtctttatgaccaagagtctgaaaatctttttgttatgctgtttagtattcgtttgatattgttacttttcacctgttgagcccaaattcaggattggttcagtggcagcaatgaagttgccatttaaatttgttcatagcctacatcaccaaggtctctgtgtcaaacctgtggccactctatatgcactttgtttactctttatacaaataaatatactaaagacttta

SEQ ID NO: 33 - DNA, 5018 bp, Homo sapiens
atctatcacatggcagagatagaataaaaacagaaaaatggcgacggtcacgttgtggcgagccttgctgcgtcattagataatcctcatgcaaatagcgggaagaacaaaggaaggggagcccgggacccccgggggcgcagcgcttctctgaagtagctttggaaagtagagaagaaaatccagtttgcttcttggagaacactggacagctgaataaatgcagtatctaaatataaaagaggactgcaatgccatggctttctgtgctaaaatgaggagctccaagaagactgaggtgaacctggaggcccctgagccaggggtggaagtgatcttctatctgtcggacagggagcccctccggctgggcagtggagagtacacagcagaggaactgtgcatcagggctgcacaggcatgccgtatctctcctctttgtcacaacctctttgccctgtatgacgagaacaccaagctctggtatgctccaaatcgcaccatcaccgttgatgacaagatgtccctccggctccactaccggatgaggttctatttcaccaattggcatggaaccaacgacaatgagcagtcagtgtggcgtcattctccaaagaagcagaaaaatggctacgagaaaaaaaagattccagatgcaacccctctccttgatgccagctcactggagtatctgtttgctcagggacagtatgatttggtgaaatgcctggctcctattcgagaccccaagaccgagcaggatggacatgatattgagaacgagtgtctagggatggctgtcctggccatctcacactatgccatgatgaagaagatgcagttgccagaactgcccaaggacatcagctacaagcgatatattccagaaacattgaataagtccatcagacagaggaaccttctcaccaggatgcggataaataatgttttcaaggatttcctaaaggaatttaacaacaagaccatttgtgacagcagcgtgtccacgcatgacctgaaggtgaaatacttggctaccttggaaactttgacaaaacattacggtgctgaaatatttgagacttccatgttactgatttcatcagaaaatgagatgaattggtttcattcgaatgacggtggaaacgttctctactacgaagtgatggtgactgggaatcttggaatccagtggaggcataaaccaaatgttgtttctgttgaaaaggaaaaaaataaactgaagcggaaaaaactggaaaataaacacaagaaggatgaggagaaaaacaagatccgggaagagtggaacaatttttcttacttccctgaaatcactcacattgtaataaaggagtctgtggtcagcattaacaagcaggacaacaagaaaatggaactgaagctctcttcccacgaggaggccttgtcctttgtgtccctggtagatggctacttccggctcacagcagatgcccatcattacctctgcaccgacgtggcccccccgttgatcgtccacaacatacagaatggctgtcatggtccaatctgtacagaatacgccatcaataaattgcggcaagaaggaagcgaggaggggatgtacgtgctgaggtggagctgcaccgactttgacaacatcctcatgaccgtcacctgctttgagaagtctgagcaggtgcagggtgcccagaagcagttcaagaactttcagatcgaggtgcagaagggccgctacagtctgcacggttcggaccgcagcttccccagcttgggagacctcatgagccacctcaagaagcagatcctgcgcacggataacatcagcttcatgctaaaacgctgctgccagcccaagccccgagaaatctccaacctgctggtggctactaagaaagcccaggagtggcagcccgtctaccccatgagccagctgagtttcgatcggatcctcaagaaggatctggtgcagggcgagcaccttgggagaggcacgagaacacacatctattctgggaccctgatggattacaaggatgacgaaggaacttctgaagagaagaagataaaagtgatcctcaaagtcttagaccccagccacagggatatttccctggccttcttcgaggcagccagcatgatgagacaggtctcccacaaacacatcgtgtacctctatggcgtctgtgtccgcgacgtggagaatatcatggtggaagagtttgtggaagggggtcctctggatctcttcatgcaccggaaaagcgatgtccttaccacaccatggaaattcaaagttgccaaacagctggccagtgccctgagctacttggaggataaagacctggtccatggaaatgtgtgtactaaaaacctcctcctggcccgtgagggcatcgacagtgagtgtggcccattcatcaagctcagtgaccccggcatccccattacggtgctgtctaggcaagaatgcattgaacgaatcccatggattgctcctgagtgtgttgaggactccaagaacctgagtgtggctgctgacaagtggagctttggaaccacgctctgggaaatctgctacaatggcgagatccccttgaaagacaagacgctgattgagaaagagagattctatgaaagccggtgcaggccagtgacaccatcatgtaaggagctggctgacctcatgacccgctgcatgaactatgaccccaatcagaggcctttcttccgagccatcatgagagacattaataagcttgaagagcagaatccagatattgtttcagaaaaaaaaccagcaactgaagtggaccccacacattttgaaaagcgcttcctaaagaggatccgtgacttgggagagggccactttgggaaggttgagctctgcaggtatgaccccgaaggggacaatacaggggagcaggtggctgttaaatctctgaagcctgagagtggaggtaaccacatagctgatctgaaaaaggaaatcgagatcttaaggaacctctatcatgagaacattgtgaagtacaaaggaatctgcacagaagacggaggaaatggtattaagctcatcatggaatttctgccttcgggaagccttaaggaatatcttccaaagaataagaacaaaataaacctcaaacagcagctaaaatatgccgttcagatttgtaaggggatggactatttgggttctcggcaatacgttcaccgggacttggcagcaagaaatgtccttgttgagagtgaacaccaagtgaaaattggagacttcggtttaaccaaagcaattgaaaccgataaggagtattacaccgtcaaggatgaccgggacagccctgtgttttggtatgctccagaatgtttaatgcaatctaaattttatattgcctctgacgtctggtcttttggagtcactctgcatgagctgctgacttactgtgattcagattctagtcccatggctttgttcctgaaaatgataggcccaacccatggccagatgacagtcacaagacttgtgaatacgttaaaagaaggaaaacgcctgccgtgcccacctaactgtccagatgaggtttatcaacttatgaggaaatgctgggaattccaaccatccaatcggacaagctttcagaaccttattgaaggatttgaagcacttttaaaataagaagcatgaataacatttaaattccacagattatcaagtccttctcctgcaacaaatgcccaagtcattttttaaaaatttctaatgaaagaagtttgtgttctgtccaaaaagtcactgaactcatacttcagtacatatacatgtataaggcacactgtagtgcttaatatgtgtaaggacttcctctttaaatttggtaccagtaacttagtgacacataatgacaaccaaaatatttgaaagcacttaagcactcctccttgtggaaagaatataccaccatttcatctggctagttcaccatcacaactgcattaccaaaaggggatttttgaaaacgaggagttgaccaaaataatatctgaagatgattgcttttccctgctgccagctgatctgaaatgttttgctggcacattaatcatagataaagaaagattgatggacttagccctcaaatttcagtatctatacagtactagaccatgcattcttaaaatattagataccaggtagtatatattgtttctgtacaaaaatgactgtattctctcaccagtaggacttaaactttgtttctccagtggcttagctcctgttcctttgggtgatcactagcacccatttttgagaaagctggttctacatggggggatagctgtggaatagataatttgctgcatgttaattctcaagaactaagcctgtgccagtgctttcctaagcagtatacctttaatcagaactcattcccagaacctggatgctattacacatgcttttaagaaacgtcaatgtatatccttttataactctaccactttggggcaagctattccagcactggttttgaatgctgtatgcaaccagtctgaataccacatacgctgcactgttcttagagggtttccatacttaccaccgatctacaagggttgatccctgtttttaccatcaatcatcaccctgtggtgcaacacttgaaagacccggctagaggcactatggacttcaggatccactagacagttttcagtttgcttggaggtagctgggtaatcaaaaatgtttagtcattgattcaatgtgaacgattacggtctttatgaccaagagtctgaaaatctttttgttatgctgtttagtattcgtttgatattgttacttttcacctgttgagcccaaattcaggattggttcagtggcagcaatgaagttgccatttaaatttgttcatagcctacatcaccaaggtctctgtgtcaaacctgtggccactctatatgcactttgtttactctttatacaaataaatatactaaagacttta

SEQ ID NO: 34 - DNA, 5277 bp, Homo sapiens
atctatcacatggcagagatagaataaaaacagaaaaatggcgacggtcacgttgtggcgagccttgctgcgtcattagataatcctcatgcaaatagcgggaagaacaaaggaaggggagcccgggacccccgggggcgcaggatccggcgggaggagtctaagaggaggaggcggcggtgccggaggaggaggaggagggagggagaagagaggaagaccggagtccccgcggcggcggcggtccggagagagggcgagccccgcgcggcgccggggaccgggcgctaccacgaggccgggacgctggagtctgggttatctaaaacagttcatgctgctgaaaacctccttcctggcagatgtccctcaaccctactggtgcctggcttctgagacacacgcttctctgaagtagctttggaaagtagagaagaaaatccagtttgcttcttggagaacactggacagctgaataaatgcagtatctaaatataaaagaggactgcaatgccatggctttctgtgctaaaatgaggagctccaagaagactgaggtgaacctggaggcccctgagccaggggtggaagtgatcttctatctgtcggacagggagcccctccggctgggcagtggagagtacacagcagaggaactgtgcatcagggctgcacaggcatgccgtatctctcctctttgtcacaacctctttgccctgtatgacgagaacaccaagctctggtatgctccaaatcgcaccatcaccgttgatgacaagatgtccctccggctccactaccggatgaggttctatttcaccaattggcatggaaccaacgacaatgagcagtcagtgtggcgtcattctccaaagaagcagaaaaatggctacgagaaaaaaaagattccagatgcaacccctctccttgatgccagctcactggagtatctgtttgctcagggacagtatgatttggtgaaatgcctggctcctattcgagaccccaagaccgagcaggatggacatgatattgagaacgagtgtctagggatggctgtcctggccatctcacactatgccatgatgaagaagatgcagttgccagaactgcccaaggacatcagctacaagcgatatattccagaaacattgaataagtccatcagacagaggaaccttctcaccaggatgcggataaataatgttttcaaggatttcctaaaggaatttaacaacaagaccatttgtgacagcagcgtgtccacgcatgacctgaaggtgaaatacttggctaccttggaaactttgacaaaacattacggtgctgaaatatttgagacttccatgttactgatttcatcagaaaatgagatgaattggtttcattcgaatgacggtggaaacgttctctactacgaagtgatggtgactgggaatcttggaatccagtggaggcataaaccaaatgttgtttctgttgaaaaggaaaaaaataaactgaagcggaaaaaactggaaaataaacacaagaaggatgaggagaaaaacaagatccgggaagagtggaacaatttttcttacttccctgaaatcactcacattgtaataaaggagtctgtggtcagcattaacaagcaggacaacaagaaaatggaactgaagctctcttcccacgaggaggccttgtcctttgtgtccctggtagatggctacttccggctcacagcagatgcccatcattacctctgcaccgacgtggcccccccgttgatcgtccacaacatacagaatggctgtcatggtccaatctgtacagaatacgccatcaataaattgcggcaagaaggaagcgaggaggggatgtacgtgctgaggtggagctgcaccgactttgacaacatcctcatgaccgtcacctgctttgagaagtctgagcaggtgcagggtgcccagaagcagttcaagaactttcagatcgaggtgcagaagggccgctacagtctgcacggttcggaccgcagcttccccagcttgggagacctcatgagccacctcaagaagcagatcctgcgcacggataacatcagcttcatgctaaaacgctgctgccagcccaagccccgagaaatctccaacctgctggtggctactaagaaagcccaggagtggcagcccgtctaccccatgagccagctgagtttcgatcggatcctcaagaaggatctggtgcagggcgagcaccttgggagaggcacgagaacacacatctattctgggaccctgatggattacaaggatgacgaaggaacttctgaagagaagaagataaaagtgatcctcaaagtcttagaccccagccacagggatatttccctggccttcttcgaggcagccagcatgatgagacaggtctcccacaaacacatcgtgtacctctatggcgtctgtgtccgcgacgtggagaatatcatggtggaagagtttgtggaagggggtcctctggatctcttcatgcaccggaaaagcgatgtccttaccacaccatggaaattcaaagttgccaaacagctggccagtgccctgagctacttggaggataaagacctggtccatggaaatgtgtgtactaaaaacctcctcctggcccgtgagggcatcgacagtgagtgtggcccattcatcaagctcagtgaccccggcatccccattacggtgctgtctaggcaagaatgcattgaacgaatcccatggattgctcctgagtgtgttgaggactccaagaacctgagtgtggctgctgacaagtggagctttggaaccacgctctgggaaatctgctacaatggcgagatccccttgaaagacaagacgctgattgagaaagagagattctatgaaagccggtgcaggccagtgacaccatcatgtaaggagctggctgacctcatgacccgctgcatgaactatgaccccaatcagaggcctttcttccgagccatcatgagagacattaataagcttgaagagcagaatccagatattgtttcagaaaaaaaaccagcaactgaagtggaccccacacattttgaaaagcgcttcctaaagaggatccgtgacttgggagagggccactttgggaaggttgagctctgcaggtatgaccccgaaggggacaatacaggggagcaggtggctgttaaatctctgaagcctgagagtggaggtaaccacatagctgatctgaaaaaggaaatcgagatcttaaggaacctctatcatgagaacattgtgaagtacaaaggaatctgcacagaagacggaggaaatggtattaagctcatcatggaatttctgccttcgggaagccttaaggaatatcttccaaagaataagaacaaaataaacctcaaacagcagctaaaatatgccgttcagatttgtaaggggatggactatttgggttctcggcaatacgttcaccgggacttggcagcaagaaatgtccttgttgagagtgaacaccaagtgaaaattggagacttcggtttaaccaaagcaattgaaaccgataaggagtattacaccgtcaaggatgaccgggacagccctgtgttttggtatgctccagaatgtttaatgcaatctaaattttatattgcctctgacgtctggtcttttggagtcactctgcatgagctgctgacttactgtgattcagattctagtcccatggctttgttcctgaaaatgataggcccaacccatggccagatgacagtcacaagacttgtgaatacgttaaaagaaggaaaacgcctgccgtgcccacctaactgtccagatgaggtttatcaacttatgaggaaatgctgggaattccaaccatccaatcggacaagctttcagaaccttattgaaggatttgaagcacttttaaaataagaagcatgaataacatttaaattccacagattatcaagtccttctcctgcaacaaatgcccaagtcattttttaaaaatttctaatgaaagaagtttgtgttctgtccaaaaagtcactgaactcatacttcagtacatatacatgtataaggcacactgtagtgcttaatatgtgtaaggacttcctctttaaatttggtaccagtaacttagtgacacataatgacaaccaaaatatttgaaagcacttaagcactcctccttgtggaaagaatataccaccatttcatctggctagttcaccatcacaactgcattaccaaaaggggatttttgaaaacgaggagttgaccaaaataatatctgaagatgattgcttttccctgctgccagctgatctgaaatgttttgctggcacattaatcatagataaagaaagattgatggacttagccctcaaatttcagtatctatacagtactagaccatgcattcttaaaatattagataccaggtagtatatattgtttctgtacaaaaatgactgtattctctcaccagtaggacttaaactttgtttctccagtggcttagctcctgttcctttgggtgatcactagcacccatttttgagaaagctggttctacatggggggatagctgtggaatagataatttgctgcatgttaattctcaagaactaagcctgtgccagtgctttcctaagcagtatacctttaatcagaactcattcccagaacctggatgctattacacatgcttttaagaaacgtcaatgtatatccttttataactctaccactttggggcaagctattccagcactggttttgaatgctgtatgcaaccagtctgaataccacatacgctgcactgttcttagagggtttccatacttaccaccgatctacaagggttgatccctgtttttaccatcaatcatcaccctgtggtgcaacacttgaaagacccggctagaggcactatggacttcaggatccactagacagttttcagtttgcttggaggtagctgggtaatcaaaaatgtttagtcattgattcaatgtgaacgattacggtctttatgaccaagagtctgaaaatctttttgttatgctgtttagtattcgtttgatattgttacttttcacctgttgagcccaaattcaggattggttcagtggcagcaatgaagttgccatttaaatttgttcatagcctacatcaccaaggtctctgtgtcaaacctgtggccactctatatgcactttgtttactctttatacaaataaatatactaaagacttta

SEQ ID NO: 35 - DNA, 5193 bp, Homo sapiens
atctatcacatggcagagatagaataaaaacagaaaaatggcgacggtcacgttgtggcgagccttgctgcgtcattagataatcctcatgcaaatagcgggaagaacaaaggaaggggagcccgggacccccgggggcgcaggatccggcgggaggagtctaagaggaggaggcggcggtgccggaggaggaggaggagggagggagaagagaggaagaccggagtccccgcggcggcggcggtccggagagagggcgagccccgcgcggcgccggggaccgggcgctaccacgaggccgggacgctggagtctgggcgcttctctgaagtagctttggaaagtagagaagaaaatccagtttgcttcttggagaacactggacagctgaataaatgcagtatctaaatataaaagaggactgcaatgccatggctttctgtgctaaaatgaggagctccaagaagactgaggtgaacctggaggcccctgagccaggggtggaagtgatcttctatctgtcggacagggagcccctccggctgggcagtggagagtacacagcagaggaactgtgcatcagggctgcacaggcatgccgtatctctcctctttgtcacaacctctttgccctgtatgacgagaacaccaagctctggtatgctccaaatcgcaccatcaccgttgatgacaagatgtccctccggctccactaccggatgaggttctatttcaccaattggcatggaaccaacgacaatgagcagtcagtgtggcgtcattctccaaagaagcagaaaaatggctacgagaaaaaaaagattccagatgcaacccctctccttgatgccagctcactggagtatctgtttgctcagggacagtatgatttggtgaaatgcctggctcctattcgagaccccaagaccgagcaggatggacatgatattgagaacgagtgtctagggatggctgtcctggccatctcacactatgccatgatgaagaagatgcagttgccagaactgcccaaggacatcagctacaagcgatatattccagaaacattgaataagtccatcagacagaggaaccttctcaccaggatgcggataaataatgttttcaaggatttcctaaaggaatttaacaacaagaccatttgtgacagcagcgtgtccacgcatgacctgaaggtgaaatacttggctaccttggaaactttgacaaaacattacggtgctgaaatatttgagacttccatgttactgatttcatcagaaaatgagatgaattggtttcattcgaatgacggtggaaacgttctctactacgaagtgatggtgactgggaatcttggaatccagtggaggcataaaccaaatgttgtttctgttgaaaaggaaaaaaataaactgaagcggaaaaaactggaaaataaacacaagaaggatgaggagaaaaacaagatccgggaagagtggaacaatttttcttacttccctgaaatcactcacattgtaataaaggagtctgtggtcagcattaacaagcaggacaacaagaaaatggaactgaagctctcttcccacgaggaggccttgtcctttgtgtccctggtagatggctacttccggctcacagcagatgcccatcattacctctgcaccgacgtggcccccccgttgatcgtccacaacatacagaatggctgtcatggtccaatctgtacagaatacgccatcaataaattgcggcaagaaggaagcgaggaggggatgtacgtgctgaggtggagctgcaccgactttgacaacatcctcatgaccgtcacctgctttgagaagtctgagcaggtgcagggtgcccagaagcagttcaagaactttcagatcgaggtgcagaagggccgctacagtctgcacggttcggaccgcagcttccccagcttgggagacctcatgagccacctcaagaagcagatcctgcgcacggataacatcagcttcatgctaaaacgctgctgccagcccaagccccgagaaatctccaacctgctggtggctactaagaaagcccaggagtggcagcccgtctaccccatgagccagctgagtttcgatcggatcctcaagaaggatctggtgcagggcgagcaccttgggagaggcacgagaacacacatctattctgggaccctgatggattacaaggatgacgaaggaacttctgaagagaagaagataaaagtgatcctcaaagtcttagaccccagccacagggatatttccctggccttcttcgaggcagccagcatgatgagacaggtctcccacaaacacatcgtgtacctctatggcgtctgtgtccgcgacgtggagaatatcatggtggaagagtttgtggaagggggtcctctggatctcttcatgcaccggaaaagcgatgtccttaccacaccatggaaattcaaagttgccaaacagctggccagtgccctgagctacttggaggataaagacctggtccatggaaatgtgtgtactaaaaacctcctcctggcccgtgagggcatcgacagtgagtgtggcccattcatcaagctcagtgaccccggcatccccattacggtgctgtctaggcaagaatgcattgaacgaatcccatggattgctcctgagtgtgttgaggactccaagaacctgagtgtggctgctgacaagtggagctttggaaccacgctctgggaaatctgctacaatggcgagatccccttgaaagacaagacgctgattgagaaagagagattctatgaaagccggtgcaggccagtgacaccatcatgtaaggagctggctgacctcatgacccgctgcatgaactatgaccccaatcagaggcctttcttccgagccatcatgagagacattaataagcttgaagagcagaatccagatattgtttcagaaaaaaaaccagcaactgaagtggaccccacacattttgaaaagcgcttcctaaagaggatccgtgacttgggagagggccactttgggaaggttgagctctgcaggtatgaccccgaaggggacaatacaggggagcaggtggctgttaaatctctgaagcctgagagtggaggtaaccacatagctgatctgaaaaaggaaatcgagatcttaaggaacctctatcatgagaacattgtgaagtacaaaggaatctgcacagaagacggaggaaatggtattaagctcatcatggaatttctgccttcgggaagccttaaggaatatcttccaaagaataagaacaaaataaacctcaaacagcagctaaaatatgccgttcagatttgtaaggggatggactatttgggttctcggcaatacgttcaccgggacttggcagcaagaaatgtccttgttgagagtgaacaccaagtgaaaattggagacttcggtttaaccaaagcaattgaaaccgataaggagtattacaccgtcaaggatgaccgggacagccctgtgttttggtatgctccagaatgtttaatgcaatctaaattttatattgcctctgacgtctggtcttttggagtcactctgcatgagctgctgacttactgtgattcagattctagtcccatggctttgttcctgaaaatgataggcccaacccatggccagatgacagtcacaagacttgtgaatacgttaaaagaaggaaaacgcctgccgtgcccacctaactgtccagatgaggtttatcaacttatgaggaaatgctgggaattccaaccatccaatcggacaagctttcagaaccttattgaaggatttgaagcacttttaaaataagaagcatgaataacatttaaattccacagattatcaagtccttctcctgcaacaaatgcccaagtcattttttaaaaatttctaatgaaagaagtttgtgttctgtccaaaaagtcactgaactcatacttcagtacatatacatgtataaggcacactgtagtgcttaatatgtgtaaggacttcctctttaaatttggtaccagtaacttagtgacacataatgacaaccaaaatatttgaaagcacttaagcactcctccttgtggaaagaatataccaccatttcatctggctagttcaccatcacaactgcattaccaaaaggggatttttgaaaacgaggagttgaccaaaataatatctgaagatgattgcttttccctgctgccagctgatctgaaatgttttgctggcacattaatcatagataaagaaagattgatggacttagccctcaaatttcagtatctatacagtactagaccatgcattcttaaaatattagataccaggtagtatatattgtttctgtacaaaaatgactgtattctctcaccagtaggacttaaactttgtttctccagtggcttagctcctgttcctttgggtgatcactagcacccatttttgagaaagctggttctacatggggggatagctgtggaatagataatttgctgcatgttaattctcaagaactaagcctgtgccagtgctttcctaagcagtatacctttaatcagaactcattcccagaacctggatgctattacacatgcttttaagaaacgtcaatgtatatccttttataactctaccactttggggcaagctattccagcactggttttgaatgctgtatgcaaccagtctgaataccacatacgctgcactgttcttagagggtttccatacttaccaccgatctacaagggttgatccctgtttttaccatcaatcatcaccctgtggtgcaacacttgaaagacccggctagaggcactatggacttcaggatccactagacagttttcagtttgcttggaggtagctgggtaatcaaaaatgtttagtcattgattcaatgtgaacgattacggtctttatgaccaagagtctgaaaatctttttgttatgctgtttagtattcgtttgatattgttacttttcacctgttgagcccaaattcaggattggttcagtggcagcaatgaagttgccatttaaatttgttcatagcctacatcaccaaggtctctgtgtcaaacctgtggccactctatatgcactttgtttactctttatacaaataaatatactaaagacttta

SEQ ID NO: 36 - DNA, 5176 bp, Homo sapiens
gcgtcgctgagcgcaggccgcggcggccgcggagtatcctggagctgcagacagtgcgggcctgcgcccagtcccggctgtcctcgccgcgacccctcctcagccctgggcgcgcgcacgctggggccccgcggggctggccgcctagcgagcctgccggtcgaccccagccagcgcagcgacggggcgctgcctggcccaggcgcacacggaagtgttatctaaaacagttcatgctgctgaaaacctccttcctggcagatgtccctcaaccctactggtgcctggcttctgagacacacgcttctctgaagtagctttggaaagtagagaagaaaatccagtttgcttcttggagaacactggacagctgaataaatgcagtatctaaatataaaagaggactgcaatgccatggctttctgtgctaaaatgaggagctccaagaagactgaggtgaacctggaggcccctgagccaggggtggaagtgatcttctatctgtcggacagggagcccctccggctgggcagtggagagtacacagcagaggaactgtgcatcagggctgcacaggcatgccgtatctctcctctttgtcacaacctctttgccctgtatgacgagaacaccaagctctggtatgctccaaatcgcaccatcaccgttgatgacaagatgtccctccggctccactaccggatgaggttctatttcaccaattggcatggaaccaacgacaatgagcagtcagtgtggcgtcattctccaaagaagcagaaaaatggctacgagaaaaaaaagattccagatgcaacccctctccttgatgccagctcactggagtatctgtttgctcagggacagtatgatttggtgaaatgcctggctcctattcgagaccccaagaccgagcaggatggacatgatattgagaacgagtgtctagggatggctgtcctggccatctcacactatgccatgatgaagaagatgcagttgccagaactgcccaaggacatcagctacaagcgatatattccagaaacattgaataagtccatcagacagaggaaccttctcaccaggatgcggataaataatgttttcaaggatttcctaaaggaatttaacaacaagaccatttgtgacagcagcgtgtccacgcatgacctgaaggtgaaatacttggctaccttggaaactttgacaaaacattacggtgctgaaatatttgagacttccatgttactgatttcatcagaaaatgagatgaattggtttcattcgaatgacggtggaaacgttctctactacgaagtgatggtgactgggaatcttggaatccagtggaggcataaaccaaatgttgtttctgttgaaaaggaaaaaaataaactgaagcggaaaaaactggaaaataaacacaagaaggatgaggagaaaaacaagatccgggaagagtggaacaatttttcttacttccctgaaatcactcacattgtaataaaggagtctgtggtcagcattaacaagcaggacaacaagaaaatggaactgaagctctcttcccacgaggaggccttgtcctttgtgtccctggtagatggctacttccggctcacagcagatgcccatcattacctctgcaccgacgtggcccccccgttgatcgtccacaacatacagaatggctgtcatggtccaatctgtacagaatacgccatcaataaattgcggcaagaaggaagcgaggaggggatgtacgtgctgaggtggagctgcaccgactttgacaacatcctcatgaccgtcacctgctttgagaagtctgagcaggtgcagggtgcccagaagcagttcaagaactttcagatcgaggtgcagaagggccgctacagtctgcacggttcggaccgcagcttccccagcttgggagacctcatgagccacctcaagaagcagatcctgcgcacggataacatcagcttcatgctaaaacgctgctgccagcccaagccccgagaaatctccaacctgctggtggctactaagaaagcccaggagtggcagcccgtctaccccatgagccagctgagtttcgatcggatcctcaagaaggatctggtgcagggcgagcaccttgggagaggcacgagaacacacatctattctgggaccctgatggattacaaggatgacgaaggaacttctgaagagaagaagataaaagtgatcctcaaagtcttagaccccagccacagggatatttccctggccttcttcgaggcagccagcatgatgagacaggtctcccacaaacacatcgtgtacctctatggcgtctgtgtccgcgacgtggagaatatcatggtggaagagtttgtggaagggggtcctctggatctcttcatgcaccggaaaagcgatgtccttaccacaccatggaaattcaaagttgccaaacagctggccagtgccctgagctacttggaggataaagacctggtccatggaaatgtgtgtactaaaaacctcctcctggcccgtgagggcatcgacagtgagtgtggcccattcatcaagctcagtgaccccggcatccccattacggtgctgtctaggcaagaatgcattgaacgaatcccatggattgctcctgagtgtgttgaggactccaagaacctgagtgtggctgctgacaagtggagctttggaaccacgctctgggaaatctgctacaatggcgagatccccttgaaagacaagacgctgattgagaaagagagattctatgaaagccggtgcaggccagtgacaccatcatgtaaggagctggctgacctcatgacccgctgcatgaactatgaccccaatcagaggcctttcttccgagccatcatgagagacattaataagcttgaagagcagaatccagatattgtttcagaaaaaaaaccagcaactgaagtggaccccacacattttgaaaagcgcttcctaaagaggatccgtgacttgggagagggccactttgggaaggttgagctctgcaggtatgaccccgaaggggacaatacaggggagcaggtggctgttaaatctctgaagcctgagagtggaggtaaccacatagctgatctgaaaaaggaaatcgagatcttaaggaacctctatcatgagaacattgtgaagtacaaaggaatctgcacagaagacggaggaaatggtattaagctcatcatggaatttctgccttcgggaagccttaaggaatatcttccaaagaataagaacaaaataaacctcaaacagcagctaaaatatgccgttcagatttgtaaggggatggactatttgggttctcggcaatacgttcaccgggacttggcagcaagaaatgtccttgttgagagtgaacaccaagtgaaaattggagacttcggtttaaccaaagcaattgaaaccgataaggagtattacaccgtcaaggatgaccgggacagccctgtgttttggtatgctccagaatgtttaatgcaatctaaattttatattgcctctgacgtctggtcttttggagtcactctgcatgagctgctgacttactgtgattcagattctagtcccatggctttgttcctgaaaatgataggcccaacccatggccagatgacagtcacaagacttgtgaatacgttaaaagaaggaaaacgcctgccgtgcccacctaactgtccagatgaggtttatcaacttatgaggaaatgctgggaattccaaccatccaatcggacaagctttcagaaccttattgaaggatttgaagcacttttaaaataagaagcatgaataacatttaaattccacagattatcaagtccttctcctgcaacaaatgcccaagtcattttttaaaaatttctaatgaaagaagtttgtgttctgtccaaaaagtcactgaactcatacttcagtacatatacatgtataaggcacactgtagtgcttaatatgtgtaaggacttcctctttaaatttggtaccagtaacttagtgacacataatgacaaccaaaatatttgaaagcacttaagcactcctccttgtggaaagaatataccaccatttcatctggctagttcaccatcacaactgcattaccaaaaggggatttttgaaaacgaggagttgaccaaaataatatctgaagatgattgcttttccctgctgccagctgatctgaaatgttttgctggcacattaatcatagataaagaaagattgatggacttagccctcaaatttcagtatctatacagtactagaccatgcattcttaaaatattagataccaggtagtatatattgtttctgtacaaaaatgactgtattctctcaccagtaggacttaaactttgtttctccagtggcttagctcctgttcctttgggtgatcactagcacccatttttgagaaagctggttctacatggggggatagctgtggaatagataatttgctgcatgttaattctcaagaactaagcctgtgccagtgctttcctaagcagtatacctttaatcagaactcattcccagaacctggatgctattacacatgcttttaagaaacgtcaatgtatatccttttataactctaccactttggggcaagctattccagcactggttttgaatgctgtatgcaaccagtctgaataccacatacgctgcactgttcttagagggtttccatacttaccaccgatctacaagggttgatccctgtttttaccatcaatcatcaccctgtggtgcaacacttgaaagacccggctagaggcactatggacttcaggatccactagacagttttcagtttgcttggaggtagctgggtaatcaaaaatgtttagtcattgattcaatgtgaacgattacggtctttatgaccaagagtctgaaaatctttttgttatgctgtttagtattcgtttgatattgttacttttcacctgttgagcccaaattcaggattggttcagtggcagcaatgaagttgccatttaaatttgttcatagcctacatcaccaaggtctctgtgtcaaacctgtggccactctatatgcactttgtttactctttatacaaataaatatactaaagacttta

SEQ ID NO: 37 - DNA, 4931 bp, Homo sapiens
agaagcggagcgtatacggaggaggcgggatgcatttctgcatcgagcgcacaaagcgcttctctgaagtagctttggaaagtagagaagaaaatccagtttgcttcttggagaacactggacagctgaataaatgcagtatctaaatataaaagaggactgcaatgccatggctttctgtgctaaaatgaggagctccaagaagactgaggtgaacctggaggcccctgagccaggggtggaagtgatcttctatctgtcggacagggagcccctccggctgggcagtggagagtacacagcagaggaactgtgcatcagggctgcacaggcatgccgtatctctcctctttgtcacaacctctttgccctgtatgacgagaacaccaagctctggtatgctccaaatcgcaccatcaccgttgatgacaagatgtccctccggctccactaccggatgaggttctatttcaccaattggcatggaaccaacgacaatgagcagtcagtgtggcgtcattctccaaagaagcagaaaaatggctacgagaaaaaaaagattccagatgcaacccctctccttgatgccagctcactggagtatctgtttgctcagggacagtatgatttggtgaaatgcctggctcctattcgagaccccaagaccgagcaggatggacatgatattgagaacgagtgtctagggatggctgtcctggccatctcacactatgccatgatgaagaagatgcagttgccagaactgcccaaggacatcagctacaagcgatatattccagaaacattgaataagtccatcagacagaggaaccttctcaccaggatgcggataaataatgttttcaaggatttcctaaaggaatttaacaacaagaccatttgtgacagcagcgtgtccacgcatgacctgaaggtgaaatacttggctaccttggaaactttgacaaaacattacggtgctgaaatatttgagacttccatgttactgatttcatcagaaaatgagatgaattggtttcattcgaatgacggtggaaacgttctctactacgaagtgatggtgactgggaatcttggaatccagtggaggcataaaccaaatgttgtttctgttgaaaaggaaaaaaataaactgaagcggaaaaaactggaaaataaacacaagaaggatgaggagaaaaacaagatccgggaagagtggaacaatttttcttacttccctgaaatcactcacattgtaataaaggagtctgtggtcagcattaacaagcaggacaacaagaaaatggaactgaagctctcttcccacgaggaggccttgtcctttgtgtccctggtagatggctacttccggctcacagcagatgcccatcattacctctgcaccgacgtggcccccccgttgatcgtccacaacatacagaatggctgtcatggtccaatctgtacagaatacgccatcaataaattgcggcaagaaggaagcgaggaggggatgtacgtgctgaggtggagctgcaccgactttgacaacatcctcatgaccgtcacctgctttgagaagtctgagcaggtgcagggtgcccagaagcagttcaagaactttcagatcgaggtgcagaagggccgctacagtctgcacggttcggaccgcagcttccccagcttgggagacctcatgagccacctcaagaagcagatcctgcgcacggataacatcagcttcatgctaaaacgctgctgccagcccaagccccgagaaatctccaacctgctggtggctactaagaaagcccaggagtggcagcccgtctaccccatgagccagctgagtttcgatcggatcctcaagaaggatctggtgcagggcgagcaccttgggagaggcacgagaacacacatctattctgggaccctgatggattacaaggatgacgaaggaacttctgaagagaagaagataaaagtgatcctcaaagtcttagaccccagccacagggatatttccctggccttcttcgaggcagccagcatgatgagacaggtctcccacaaacacatcgtgtacctctatggcgtctgtgtccgcgacgtggagaatatcatggtggaagagtttgtggaagggggtcctctggatctcttcatgcaccggaaaagcgatgtccttaccacaccatggaaattcaaagttgccaaacagctggccagtgccctgagctacttggaggataaagacctggtccatggaaatgtgtgtactaaaaacctcctcctggcccgtgagggcatcgacagtgagtgtggcccattcatcaagctcagtgaccccggcatccccattacggtgctgtctaggcaagaatgcattgaacgaatcccatggattgctcctgagtgtgttgaggactccaagaacctgagtgtggctgctgacaagtggagctttggaaccacgctctgggaaatctgctacaatggcgagatccccttgaaagacaagacgctgattgagaaagagagattctatgaaagccggtgcaggccagtgacaccatcatgtaaggagctggctgacctcatgacccgctgcatgaactatgaccccaatcagaggcctttcttccgagccatcatgagagacattaataagcttgaagagcagaatccagatattgtttcagaaaaaaaaccagcaactgaagtggaccccacacattttgaaaagcgcttcctaaagaggatccgtgacttgggagagggccactttgggaaggttgagctctgcaggtatgaccccgaaggggacaatacaggggagcaggtggctgttaaatctctgaagcctgagagtggaggtaaccacatagctgatctgaaaaaggaaatcgagatcttaaggaacctctatcatgagaacattgtgaagtacaaaggaatctgcacagaagacggaggaaatggtattaagctcatcatggaatttctgccttcgggaagccttaaggaatatcttccaaagaataagaacaaaataaacctcaaacagcagctaaaatatgccgttcagatttgtaaggggatggactatttgggttctcggcaatacgttcaccgggacttggcagcaagaaatgtccttgttgagagtgaacaccaagtgaaaattggagacttcggtttaaccaaagcaattgaaaccgataaggagtattacaccgtcaaggatgaccgggacagccctgtgttttggtatgctccagaatgtttaatgcaatctaaattttatattgcctctgacgtctggtcttttggagtcactctgcatgagctgctgacttactgtgattcagattctagtcccatggctttgttcctgaaaatgataggcccaacccatggccagatgacagtcacaagacttgtgaatacgttaaaagaaggaaaacgcctgccgtgcccacctaactgtccagatgaggtttatcaacttatgaggaaatgctgggaattccaaccatccaatcggacaagctttcagaaccttattgaaggatttgaagcacttttaaaataagaagcatgaataacatttaaattccacagattatcaagtccttctcctgcaacaaatgcccaagtcattttttaaaaatttctaatgaaagaagtttgtgttctgtccaaaaagtcactgaactcatacttcagtacatatacatgtataaggcacactgtagtgcttaatatgtgtaaggacttcctctttaaatttggtaccagtaacttagtgacacataatgacaaccaaaatatttgaaagcacttaagcactcctccttgtggaaagaatataccaccatttcatctggctagttcaccatcacaactgcattaccaaaaggggatttttgaaaacgaggagttgaccaaaataatatctgaagatgattgcttttccctgctgccagctgatctgaaatgttttgctggcacattaatcatagataaagaaagattgatggacttagccctcaaatttcagtatctatacagtactagaccatgcattcttaaaatattagataccaggtagtatatattgtttctgtacaaaaatgactgtattctctcaccagtaggacttaaactttgtttctccagtggcttagctcctgttcctttgggtgatcactagcacccatttttgagaaagctggttctacatggggggatagctgtggaatagataatttgctgcatgttaattctcaagaactaagcctgtgccagtgctttcctaagcagtatacctttaatcagaactcattcccagaacctggatgctattacacatgcttttaagaaacgtcaatgtatatccttttataactctaccactttggggcaagctattccagcactggttttgaatgctgtatgcaaccagtctgaataccacatacgctgcactgttcttagagggtttccatacttaccaccgatctacaagggttgatccctgtttttaccatcaatcatcaccctgtggtgcaacacttgaaagacccggctagaggcactatggacttcaggatccactagacagttttcagtttgcttggaggtagctgggtaatcaaaaatgtttagtcattgattcaatgtgaacgattacggtctttatgaccaagagtctgaaaatctttttgttatgctgtttagtattcgtttgatattgttacttttcacctgttgagcccaaattcaggattggttcagtggcagcaatgaagttgccatttaaatttgttcatagcctacatcaccaaggtctctgtgtcaaacctgtggccactctatatgcactttgtttactctttatacaaataaatatactaaagacttta

SEQ ID NO: 38 - DNA, 5089 bp, Homo sapiens
gcgtcgctgagcgcaggccgcggcggccgcggagtatcctggagctgcagacagtgcgggcctgcgcccagtcccggctgtcctcgccgcgacccctcctcagccctgggcgcgcgcacgctggggccccgcggggctggccgcctagcgagcctgccggtcgaccccagccagcgcagcgacggggcgctgcctggcccaggcgcacacggaagtgcgcttctctgaagtagctttggaaagtagagaagaaaatccagtttgcttcttggagaacactggacagctgaataaatgcagtatctaaatataaaagaggactgcaatgccatggctttctgtgctaaaatgaggagctccaagaagactgaggtgaacctggaggcccctgagccaggggtggaagtgatcttctatctgtcggacagggagcccctccggctgggcagtggagagtacacagcagaggaactgtgcatcagggctgcacaggcatgccgtatctctcctctttgtcacaacctctttgccctgtatgacgagaacaccaagctctggtatgctccaaatcgcaccatcaccgttgatgacaagatgtccctccggctccactaccggatgaggttctatttcaccaattggcatggaaccaacgacaatgagcagtcagtgtggcgtcattctccaaagaagcagaaaaatggctacgagaaaaaaaagattccagatgcaacccctctccttgatgccagctcactggagtatctgtttgctcagggacagtatgatttggtgaaatgcctggctcctattcgagaccccaagaccgagcaggatggacatgatattgagaacgagtgtctagggatggctgtcctggccatctcacactatgccatgatgaagaagatgcagttgccagaactgcccaaggacatcagctacaagcgatatattccagaaacattgaataagtccatcagacagaggaaccttctcaccaggatgcggataaataatgttttcaaggatttcctaaaggaatttaacaacaagaccatttgtgacagcagcgtgtccacgcatgacctgaaggtgaaatacttggctaccttggaaactttgacaaaacattacggtgctgaaatatttgagacttccatgttactgatttcatcagaaaatgagatgaattggtttcattcgaatgacggtggaaacgttctctactacgaagtgatggtgactgggaatcttggaatccagtggaggcataaaccaaatgttgtttctgttgaaaaggaaaaaaataaactgaagcggaaaaaactggaaaataaacacaagaaggatgaggagaaaaacaagatccgggaagagtggaacaatttttcttacttccctgaaatcactcacattgtaataaaggagtctgtggtcagcattaacaagcaggacaacaagaaaatggaactgaagctctcttcccacgaggaggccttgtcctttgtgtccctggtagatggctacttccggctcacagcagatgcccatcattacctctgcaccgacgtggcccccccgttgatcgtccacaacatacagaatggctgtcatggtccaatctgtacagaatacgccatcaataaattgcggcaagaaggaagcgaggaggggatgtacgtgctgaggtggagctgcaccgactttgacaacatcctcatgaccgtcacctgctttgagaagtctgaggtgcagggtgcccagaagcagttcaagaactttcagatcgaggtgcagaagggccgctacagtctgcacggttcggaccgcagcttccccagcttgggagacctcatgagccacctcaagaagcagatcctgcgcacggataacatcagcttcatgctaaaacgctgctgccagcccaagccccgagaaatctccaacctgctggtggctactaagaaagcccaggagtggcagcccgtctaccccatgagccagctgagtttcgatcggatcctcaagaaggatctggtgcagggcgagcaccttgggagaggcacgagaacacacatctattctgggaccctgatggattacaaggatgacgaaggaacttctgaagagaagaagataaaagtgatcctcaaagtcttagaccccagccacagggatatttccctggccttcttcgaggcagccagcatgatgagacaggtctcccacaaacacatcgtgtacctctatggcgtctgtgtccgcgacgtggagaatatcatggtggaagagtttgtggaagggggtcctctggatctcttcatgcaccggaaaagcgatgtccttaccacaccatggaaattcaaagttgccaaacagctggccagtgccctgagctacttggaggataaagacctggtccatggaaatgtgtgtactaaaaacctcctcctggcccgtgagggcatcgacagtgagtgtggcccattcatcaagctcagtgaccccggcatccccattacggtgctgtctaggcaagaatgcattgaacgaatcccatggattgctcctgagtgtgttgaggactccaagaacctgagtgtggctgctgacaagtggagctttggaaccacgctctgggaaatctgctacaatggcgagatccccttgaaagacaagacgctgattgagaaagagagattctatgaaagccggtgcaggccagtgacaccatcatgtaaggagctggctgacctcatgacccgctgcatgaactatgaccccaatcagaggcctttcttccgagccatcatgagagacattaataagcttgaagagcagaatccagatattgtttcagaaaaaaaaccagcaactgaagtggaccccacacattttgaaaagcgcttcctaaagaggatccgtgacttgggagagggccactttgggaaggttgagctctgcaggtatgaccccgaaggggacaatacaggggagcaggtggctgttaaatctctgaagcctgagagtggaggtaaccacatagctgatctgaaaaaggaaatcgagatcttaaggaacctctatcatgagaacattgtgaagtacaaaggaatctgcacagaagacggaggaaatggtattaagctcatcatggaatttctgccttcgggaagccttaaggaatatcttccaaagaataagaacaaaataaacctcaaacagcagctaaaatatgccgttcagatttgtaaggggatggactatttgggttctcggcaatacgttcaccgggacttggcagcaagaaatgtccttgttgagagtgaacaccaagtgaaaattggagacttcggtttaaccaaagcaattgaaaccgataaggagtattacaccgtcaaggatgaccgggacagccctgtgttttggtatgctccagaatgtttaatgcaatctaaattttatattgcctctgacgtctggtcttttggagtcactctgcatgagctgctgacttactgtgattcagattctagtcccatggctttgttcctgaaaatgataggcccaacccatggccagatgacagtcacaagacttgtgaatacgttaaaagaaggaaaacgcctgccgtgcccacctaactgtccagatgaggtttatcaacttatgaggaaatgctgggaattccaaccatccaatcggacaagctttcagaaccttattgaaggatttgaagcacttttaaaataagaagcatgaataacatttaaattccacagattatcaagtccttctcctgcaacaaatgcccaagtcattttttaaaaatttctaatgaaagaagtttgtgttctgtccaaaaagtcactgaactcatacttcagtacatatacatgtataaggcacactgtagtgcttaatatgtgtaaggacttcctctttaaatttggtaccagtaacttagtgacacataatgacaaccaaaatatttgaaagcacttaagcactcctccttgtggaaagaatataccaccatttcatctggctagttcaccatcacaactgcattaccaaaaggggatttttgaaaacgaggagttgaccaaaataatatctgaagatgattgcttttccctgctgccagctgatctgaaatgttttgctggcacattaatcatagataaagaaagattgatggacttagccctcaaatttcagtatctatacagtactagaccatgcattcttaaaatattagataccaggtagtatatattgtttctgtacaaaaatgactgtattctctcaccagtaggacttaaactttgtttctccagtggcttagctcctgttcctttgggtgatcactagcacccatttttgagaaagctggttctacatggggggatagctgtggaatagataatttgctgcatgttaattctcaagaactaagcctgtgccagtgctttcctaagcagtatacctttaatcagaactcattcccagaacctggatgctattacacatgcttttaagaaacgtcaatgtatatccttttataactctaccactttggggcaagctattccagcactggttttgaatgctgtatgcaaccagtctgaataccacatacgctgcactgttcttagagggtttccatacttaccaccgatctacaagggttgatccctgtttttaccatcaatcatcaccctgtggtgcaacacttgaaagacccggctagaggcactatggacttcaggatccactagacagttttcagtttgcttggaggtagctgggtaatcaaaaatgtttagtcattgattcaatgtgaacgattacggtctttatgaccaagagtctgaaaatctttttgttatgctgtttagtattcgtttgatattgttacttttcacctgttgagcccaaattcaggattggttcagtggcagcaatgaagttgccatttaaatttgttcatagcctacatcaccaaggtctctgtgtcaaacctgtggccactctatatgcactttgtttactctttatacaaataaatatactaaagacttta

SEQ ID NO: 39 - DNA, 5092 bp, Homo sapiens
gcgtcgctgagcgcaggccgcggcggccgcggagtatcctggagctgcagacagtgcgggcctgcgcccagtcccggctgtcctcgccgcgacccctcctcagccctgggcgcgcgcacgctggggccccgcggggctggccgcctagcgagcctgccggtcgaccccagccagcgcagcgacggggcgctgcctggcccaggcgcacacggaagtgcgcttctctgaagtagctttggaaagtagagaagaaaatccagtttgcttcttggagaacactggacagctgaataaatgcagtatctaaatataaaagaggactgcaatgccatggctttctgtgctaaaatgaggagctccaagaagactgaggtgaacctggaggcccctgagccaggggtggaagtgatcttctatctgtcggacagggagcccctccggctgggcagtggagagtacacagcagaggaactgtgcatcagggctgcacaggcatgccgtatctctcctctttgtcacaacctctttgccctgtatgacgagaacaccaagctctggtatgctccaaatcgcaccatcaccgttgatgacaagatgtccctccggctccactaccggatgaggttctatttcaccaattggcatggaaccaacgacaatgagcagtcagtgtggcgtcattctccaaagaagcagaaaaatggctacgagaaaaaaaagattccagatgcaacccctctccttgatgccagctcactggagtatctgtttgctcagggacagtatgatttggtgaaatgcctggctcctattcgagaccccaagaccgagcaggatggacatgatattgagaacgagtgtctagggatggctgtcctggccatctcacactatgccatgatgaagaagatgcagttgccagaactgcccaaggacatcagctacaagcgatatattccagaaacattgaataagtccatcagacagaggaaccttctcaccaggatgcggataaataatgttttcaaggatttcctaaaggaatttaacaacaagaccatttgtgacagcagcgtgtccacgcatgacctgaaggtgaaatacttggctaccttggaaactttgacaaaacattacggtgctgaaatatttgagacttccatgttactgatttcatcagaaaatgagatgaattggtttcattcgaatgacggtggaaacgttctctactacgaagtgatggtgactgggaatcttggaatccagtggaggcataaaccaaatgttgtttctgttgaaaaggaaaaaaataaactgaagcggaaaaaactggaaaataaacacaagaaggatgaggagaaaaacaagatccgggaagagtggaacaatttttcttacttccctgaaatcactcacattgtaataaaggagtctgtggtcagcattaacaagcaggacaacaagaaaatggaactgaagctctcttcccacgaggaggccttgtcctttgtgtccctggtagatggctacttccggctcacagcagatgcccatcattacctctgcaccgacgtggcccccccgttgatcgtccacaacatacagaatggctgtcatggtccaatctgtacagaatacgccatcaataaattgcggcaagaaggaagcgaggaggggatgtacgtgctgaggtggagctgcaccgactttgacaacatcctcatgaccgtcacctgctttgagaagtctgagcaggtgcagggtgcccagaagcagttcaagaactttcagatcgaggtgcagaagggccgctacagtctgcacggttcggaccgcagcttccccagcttgggagacctcatgagccacctcaagaagcagatcctgcgcacggataacatcagcttcatgctaaaacgctgctgccagcccaagccccgagaaatctccaacctgctggtggctactaagaaagcccaggagtggcagcccgtctaccccatgagccagctgagtttcgatcggatcctcaagaaggatctggtgcagggcgagcaccttgggagaggcacgagaacacacatctattctgggaccctgatggattacaaggatgacgaaggaacttctgaagagaagaagataaaagtgatcctcaaagtcttagaccccagccacagggatatttccctggccttcttcgaggcagccagcatgatgagacaggtctcccacaaacacatcgtgtacctctatggcgtctgtgtccgcgacgtggagaatatcatggtggaagagtttgtggaagggggtcctctggatctcttcatgcaccggaaaagcgatgtccttaccacaccatggaaattcaaagttgccaaacagctggccagtgccctgagctacttggaggataaagacctggtccatggaaatgtgtgtactaaaaacctcctcctggcccgtgagggcatcgacagtgagtgtggcccattcatcaagctcagtgaccccggcatccccattacggtgctgtctaggcaagaatgcattgaacgaatcccatggattgctcctgagtgtgttgaggactccaagaacctgagtgtggctgctgacaagtggagctttggaaccacgctctgggaaatctgctacaatggcgagatccccttgaaagacaagacgctgattgagaaagagagattctatgaaagccggtgcaggccagtgacaccatcatgtaaggagctggctgacctcatgacccgctgcatgaactatgaccccaatcagaggcctttcttccgagccatcatgagagacattaataagcttgaagagcagaatccagatattgtttcagaaaaaaaaccagcaactgaagtggaccccacacattttgaaaagcgcttcctaaagaggatccgtgacttgggagagggccactttgggaaggttgagctctgcaggtatgaccccgaaggggacaatacaggggagcaggtggctgttaaatctctgaagcctgagagtggaggtaaccacatagctgatctgaaaaaggaaatcgagatcttaaggaacctctatcatgagaacattgtgaagtacaaaggaatctgcacagaagacggaggaaatggtattaagctcatcatggaatttctgccttcgggaagccttaaggaatatcttccaaagaataagaacaaaataaacctcaaacagcagctaaaatatgccgttcagatttgtaaggggatggactatttgggttctcggcaatacgttcaccgggacttggcagcaagaaatgtccttgttgagagtgaacaccaagtgaaaattggagacttcggtttaaccaaagcaattgaaaccgataaggagtattacaccgtcaaggatgaccgggacagccctgtgttttggtatgctccagaatgtttaatgcaatctaaattttatattgcctctgacgtctggtcttttggagtcactctgcatgagctgctgacttactgtgattcagattctagtcccatggctttgttcctgaaaatgataggcccaacccatggccagatgacagtcacaagacttgtgaatacgttaaaagaaggaaaacgcctgccgtgcccacctaactgtccagatgaggtttatcaacttatgaggaaatgctgggaattccaaccatccaatcggacaagctttcagaaccttattgaaggatttgaagcacttttaaaataagaagcatgaataacatttaaattccacagattatcaagtccttctcctgcaacaaatgcccaagtcattttttaaaaatttctaatgaaagaagtttgtgttctgtccaaaaagtcactgaactcatacttcagtacatatacatgtataaggcacactgtagtgcttaatatgtgtaaggacttcctctttaaatttggtaccagtaacttagtgacacataatgacaaccaaaatatttgaaagcacttaagcactcctccttgtggaaagaatataccaccatttcatctggctagttcaccatcacaactgcattaccaaaaggggatttttgaaaacgaggagttgaccaaaataatatctgaagatgattgcttttccctgctgccagctgatctgaaatgttttgctggcacattaatcatagataaagaaagattgatggacttagccctcaaatttcagtatctatacagtactagaccatgcattcttaaaatattagataccaggtagtatatattgtttctgtacaaaaatgactgtattctctcaccagtaggacttaaactttgtttctccagtggcttagctcctgttcctttgggtgatcactagcacccatttttgagaaagctggttctacatggggggatagctgtggaatagataatttgctgcatgttaattctcaagaactaagcctgtgccagtgctttcctaagcagtatacctttaatcagaactcattcccagaacctggatgctattacacatgcttttaagaaacgtcaatgtatatccttttataactctaccactttggggcaagctattccagcactggttttgaatgctgtatgcaaccagtctgaataccacatacgctgcactgttcttagagggtttccatacttaccaccgatctacaagggttgatccctgtttttaccatcaatcatcaccctgtggtgcaacacttgaaagacccggctagaggcactatggacttcaggatccactagacagttttcagtttgcttggaggtagctgggtaatcaaaaatgtttagtcattgattcaatgtgaacgattacggtctttatgaccaagagtctgaaaatctttttgttatgctgtttagtattcgtttgatattgttacttttcacctgttgagcccaaattcaggattggttcagtggcagcaatgaagttgccatttaaatttgttcatagcctacatcaccaaggtctctgtgtcaaacctgtggccactctatatgcactttgtttactctttatacaaataaatatactaaagacttta

SEQ ID NO: 40 - protein, 1154 aa, Homo sapiens
MQYLNIKEDCNAMAFCAKMRSSKKTEVNLEAPEPGVEVIFYLSDREPLRLGSGEYTAEELCIRAAQACRISPLCHNLFALYDENTKLWYAPNRTITVDDKMSLRLHYRMRFYFTNWHGTNDNEQSVWRHSPKKQKNGYEKKKIPDATPLLDASSLEYLFAQGQYDLVKCLAPIRDPKTEQDGHDIENECLGMAVLAISHYAMMKKMQLPELPKDISYKRYIPETLNKSIRQRNLLTRMRINNVFKDFLKEFNNKTICDSSVSTHDLKVKYLATLETLTKHYGAEIFETSMLLISSENEMNWFHSNDGGNVLYYEVMVTGNLGIQWRHKPNVVSVEKEKNKLKRKKLENKHKKDEEKNKIREEWNNFSYFPEITHIVIKESVVSINKQDNKKMELKLSSHEEALSFVSLVDGYFRLTADAHHYLCTDVAPPLIVHNIQNGCHGPICTEYAINKLRQEGSEEGMYVLRWSCTDFDNILMTVTCFEKSEQVQGAQKQFKNFQIEVQKGRYSLHGSDRSFPSLGDLMSHLKKQILRTDNISFMLKRCCQPKPREISNLLVATKKAQEWQPVYPMSQLSFDRILKKDLVQGEHLGRGTRTHIYSGTLMDYKDDEGTSEEKKIKVILKVLDPSHRDISLAFFEAASMMRQVSHKHIVYLYGVCVRDVENIMVEEFVEGGPLDLFMHRKSDVLTTPWKFKVAKQLASALSYLEDKDLVHGNVCTKNLLLAREGIDSECGPFIKLSDPGIPITVLSRQECIERIPWIAPECVEDSKNLSVAADKWSFGTTLWEICYNGEIPLKDKTLIEKERFYESRCRPVTPSCKELADLMTRCMNYDPNQRPFFRAIMRDINKLEEQNPDIVSEKKPATEVDPTHFEKRFLKRIRDLGEGHFGKVELCRYDPEGDNTGEQVAVKSLKPESGGNHIADLKKEIEILRNLYHENIVKYKGICTEDGGNGIKLIMEFLPSGSLKEYLPKNKNKINLKQQLKYAVQICKGMDYLGSRQYVHRDLAARNVLVESEHQVKIGDFGLTKAIETDKEYYTVKDDRDSPVFWYAPECLMQSKFYIASDVWSFGVTLHELLTYCDSDSSPMALFLKMIGPTHGQMTVTRLVNTLKEGKRLPCPPNCPDEVYQLMRKCWEFQPSNRTSFQNLIEGFEALLK

SEQ ID NO: 41 - protein, 1154 aa, Homo sapiens; identical to SEQ ID NO: 40

SEQ ID NO: 42 - protein, 1154 aa, Homo sapiens; identical to SEQ ID NO: 40

SEQ ID NO: 43 - protein, 1154 aa, Homo sapiens; identical to SEQ ID NO: 40

SEQ ID NO: 44 - protein, 1154 aa, Homo sapiens; identical to SEQ ID NO: 40

SEQ ID NO: 45 - protein, 1153 aa, Homo sapiens
MQYLNIKEDCNAMAFCAKMRSSKKTEVNLEAPEPGVEVIFYLSDREPLRLGSGEYTAEELCIRAAQACRISPLCHNLFALYDENTKLWYAPNRTITVDDKMSLRLHYRMRFYFTNWHGTNDNEQSVWRHSPKKQKNGYEKKKIPDATPLLDASSLEYLFAQGQYDLVKCLAPIRDPKTEQDGHDIENECLGMAVLAISHYAMMKKMQLPELPKDISYKRYIPETLNKSIRQRNLLTRMRINNVFKDFLKEFNNKTICDSSVSTHDLKVKYLATLETLTKHYGAEIFETSMLLISSENEMNWFHSNDGGNVLYYEVMVTGNLGIQWRHKPNVVSVEKEKNKLKRKKLENKHKKDEEKNKIREEWNNFSYFPEITHIVIKESVVSINKQDNKKMELKLSSHEEALSFVSLVDGYFRLTADAHHYLCTDVAPPLIVHNIQNGCHGPICTEYAINKLRQEGSEEGMYVLRWSCTDFDNILMTVTCFEKSEVQGAQKQFKNFQIEVQKGRYSLHGSDRSFPSLGDLMSHLKKQILRTDNISFMLKRCCQPKPREISNLLVATKKAQEWQPVYPMSQLSFDRILKKDLVQGEHLGRGTRTHIYSGTLMDYKDDEGTSEEKKIKVILKVLDPSHRDISLAFFEAASMMRQVSHKHIVYLYGVCVRDVENIMVEEFVEGGPLDLFMHRKSDVLTTPWKFKVAKQLASALSYLEDKDLVHGNVCTKNLLLAREGIDSECGPFIKLSDPGIPITVLSRQECIERIPWIAPECVEDSKNLSVAADKWSFGTTLWEICYNGEIPLKDKTLIEKERFYESRCRPVTPSCKELADLMTRCMNYDPNQRPFFRAIMRDINKLEEQNPDIVSEKKPATEVDPTHFEKRFLKRIRDLGEGHFGKVELCRYDPEGDNTGEQVAVKSLKPESGGNHIADLKKEIEILRNLYHENIVKYKGICTEDGGNGIKLIMEFLPSGSLKEYLPKNKNKINLKQQLKYAVQICKGMDYLGSRQYVHRDLAARNVLVESEHQVKIGDFGLTKAIETDKEYYTVKDDRDSPVFWYAPECLMQSKFYIASDVWSFGVTLHELLTYCDSDSSPMALFLKMIGPTHGQMTVTRLVNTLKEGKRLPCPPNCPDEVYQLMRKCWEFQPSNRTSFQNLIEGFEALLK

SEQ ID NO: 46 - protein, 1154 aa, Homo sapiens; identical to SEQ ID NO: 40

SEQ ID NO: 47 - DNA, 1672 bp, Homo sapiens
gtgggcagccggcgggctccgaggccgtgagcgcaaagcctcaggccccggctccctcctgagctgcgccgtgccaggccgcccgccgggatgcagtgggccgtgggccggcggtgggcgtgggccgcgctgctcctggctgtcgcagcggtgctgacccaggtcgtctggctctggctgggtacgcagagcttcgtcttccagcgcgaagagatagcgcagttggcgcggcagtacgctgggctggaccacgagctggccttctctcgtctgatcgtggagctgcggcggctgcacccaggccacgtgctgcccgacgaggagctgcagtgggtgttcgtgaatgcgggtggctggatgggcgccatgtgccttctgcacgcctcgctgtccgagtatgtgctgctcttcggcaccgccttgggctcccgcggccactcggggcgctactgggctgagatctcggataccatcatctctggcaccttccaccagtggagagagggcaccaccaaaagtgaggtcttctacccaggggagacggtagtacacgggcctggtgaggcaacagctgtggagtgggggccaaacacatggatggtggagtacggccggggcgtcatcccatccaccctggccttcgcgctggccgacactgtcttcagcacccaggacttcctcaccctcttctatactcttcgctcctatgctcggggcctccggcttgagctcaccacctacctctttggccaggacccttgaccagccaggcctgaaggaagacctgcggatagacaggagcgggcaggcccgcacatatccacttgctggagcccatgtttacagacagggacatacaccatgcagatcctgagttcctgctgtatgagcagggatatccatgcttatgtatccaaacacagagacccatgggaacaaatgagacacatatagatactgagacctgtgtgtacagtaggaccatgcactcacacccatctggagagggagcccccggtataccaagggagccagttgtgttcagacacacacatcacagcttgactcactaactgaggcctttccatagctccacagcttcccacctcctccccaccaaaccggggttctagagttaaggatgggggagggtattatactgcctcagtctgactcctcaacccagcagcaatttgaggggatgagggggaagaggagctgccttttggaggcccccttcacctgcagctatgatgcccttccccttctcccctgtcctcaccatatgccttatccccattctactcccctgctatgcaagtgcccctgtggcttgtccccaaccccctcagcaacaaagctcagctggggaacgagagtaatttgaagaatgcttgaagtcagcgtcttccattccagaaagacccccattcttcctttgggggtatgatgtggaagctggtttcagcccaggacccaccactgaggagaggatctagacaggtgggcctaattccaaggggcccttcctggcctggagaaggccttttacacacacacaacacatacacacacacacacacacacacatatcacagttttcacacagcccctgctgcattctctgtccatctgtctgtttctattaataaagatttgttgatctgttcca

SEQ ID NO: 48 - protein, 223 aa, Homo sapiens
MQWAVGRRWAWAALLLAVAAVLTQVVWLWLGTQSFVFQREEIAQLARQYAGLDHELAFSRLIVELRRLHPGHVLPDEELQWVFVNAGGWMGAMCLLHASLSEYVLLFGTALGSRGHSGRYWAEISDTIISGTFHQWREGTTKSEVFYPGETVVHGPGEATAVEWGPNTWMVEYGRGVIPSTLAFALADTVFSTQDFLTLFYTLRSYARGLRLELTTYLFGQDP

SEQ ID NO: 49 - protein, 1154 aa, Homo sapiens; identical to SEQ ID NO: 40